(19) United States
(12) Patent Application Publication
(10) Pub. No.: US 2023/0144779 A1
(43) Pub. Date: May 11, 2023

(54) FORMULATIONS AND METHOD FOR TREATMENT OF INFLAMMATORY DISEASES

(71) Applicant: VYOME THERAPEUTICS INC., Princeton, NJ (US)

(72) Inventors: Ashwani SINGH RAWAT, Delhi (IN); Charles CHAVDARIAN, San Ramon, CA (US); Jitesh BEHERA, Berhampur (IN); Mau SINHA, Noida (IN); Mukesh KUMAR GARG, Gurgaon (IN); Shamik GHOSH, Princeton, NJ (US); Shiladitya SENGUPTA, Cambridge, MA (US); Sumana GHOSH, Delhi (IN)

(73) Assignee: VYOME THERAPEUTICS INC., Princeton, NJ (US)

(21) Appl. No.: 17/787,303

(22) PCT Filed: Dec. 20, 2020
(86) PCT No.: PCT/IB2020/062260

§ 371 Date: Jun. 18, 2022

(30) Foreign Application Priority Data
Dec. 20, 2019 (IN) 201941053263

(51) Int. Cl. A61K 31/5377; A61K 31/343; A61K 9/00; A61K 47/06; A61K 47/44; A61K 47/26; A61K 47/02; A61K 47/18; A61K 47/10; A61P 27/02

(52) U.S. Cl. CPC A61K 31/5377; A61K 31/343; A61K 9/0048; A61K 47/06; A61K 47/44; A61K 47/26; A61K 47/02; A61K 47/183; A61K 47/10; A61P 27/02

(57) ABSTRACT

The present invention relates to formulations and their use to treat ocular disorders such as uveitis. Particularly, the invention relates to a formulation comprising mycophenolic acid (MPA) or a pharmaceutically acceptable salt or derivative thereof; and at least one component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, suspending agent, and therapeutic agent. The disclosure further provides preparation method of said ophthalmic formulations, a pharmaceutical kit comprising said formulations, and method for treating ocular diseases by administering said formulations. The present formulations are advantageous and can be delivered to the eye with increased retention time in the eye and increased bioavailability of mycophenolic acid, or a pharmaceutically acceptable salt or derivative thereof in the eye.

### FIELD OF INVENTION

This invention relates to the field of inflammatory conditions. In particular, the invention relates to ophthalmic formulations, methods for preparing the same, and their uses for treating ocular diseases.

### BACKGROUND

Inflammation of the eye serves as an umbrella term for the various inflammatory diseases. Inflammatory diseases of the eye primarily occur de novo or as secondary complications to various systemic diseases such as autoimmune diseases or infections. General symptoms include redness, itching, burning, watery eyes, swelling etc. More specific symptoms include pain, clouding of vision, sensitivity to light, secretion of pus etc. In some instances, the ocular disorder is refractory to the effects of the topically applied steroid. Different types of eye inflammatory disorders include uveitis, keratitis, red eye, dry eye syndrome, conjunctivitis, Behcet's disease, granuloma, blepharitis etc.

Uveitis is the inflammation of the uvea, the pigmented layer that lies between the inner retina and the outer fibrous layer composed of the sclera and cornea. The uvea consists of the middle layer of pigmented vascular structures of the eye and includes the iris, ciliary body, and choroid. Uveitis is most often idiopathic but has been associated with traumatic, inflammatory, and infectious processes.

Uveitis can be further subdivided into anterior, intermediate, posterior, and panuveitis based on the primary anatomical location of the inflammation in the eye. It is categorized as anterior when it affects the iris and/or ciliary body, intermediate when inflammation is there in the anterior vitreous, portions of ciliary body and the peripheral retina, and posterior when specific regions of the posterior segment like pars plana (pars planitis, low grade/chronic), the retina and/or choroid (acute toxoplasma retinochoroiditis), the retinal or optic nerve vessels, the pigment epithelium of the retina etc. are affected. Posterior uveitis may be acute or chronic. Some other cases include global inflammation of the eye (panuveitis), severe forms of posterior uveitis presenting with bilateral exudative retinal detachments (Vogt-Koyanagi-Harada disease) or acute retinal necrosis. Anterior uveitis is the most common among all types. Posterior uveitis is the second most common form whereas, intermediate and panuveitis accounts for 1% to 10% of cases.

Various factors can contribute to onset of uveitis including immunologic factors, autoimmune or inflammatory disorder of the body, eye injury and/or surgery, exposure to toxic chemicals such as pesticides and/or acids and/or medication, an infection etc. There are some strong genetic factors that predispose disease onset. Very rarely, a cancer that affects the eye, such as lymphoma can also be a contributing factor.

A considerable number of uveitis patients get affected by severe visual impairment and blindness. Only about 1% to 4% of anterior uveitis cases leads to blindness, but this number goes up to 40% to 66% in other form of uveitis. Other complications include cataract, glaucoma, retinal detachment, optic neuropathy etc. One of the major causes of visual loss in uveitis patients is macular edema. Apart from macular edema, other causes of increase of macular thickness in uveitis patients include inflammatory choroidal neovascularization, inflammatory epiretinal membrane formation with associated vitreomacular traction, central serous chorioretinopathy exacerbated by steroid therapy and contiguity with papillary swelling.

The anatomy and physiology of the eye is one of the most complex and unique systems in the human body. Lachrymation, effective drainage by the nasolacrimal system, the inner and outer blood-retinal barrier, the impermeability of the cornea, and inability of other non-corneal structures to absorb compounds make the eye exceedingly impervious to foreign substances. Topical administration is employed most often in the form of eye drops, ointments, gels, or emulsions, to treat anterior segment diseases. For most of the topically applied drugs, the site of action is usually different layers of the cornea, conjunctiva, sclera, and other tissues of the anterior segment, such as the iris and the ciliary body (anterior uvea). Upon administration, precorneal factors and anatomical barriers negatively affect the bioavailability of topical formulations. Precorneal factors include solution drainage, blinking, tear film, tear turn over, and induced lacrimation. Considering all the precorneal factors, contact time with the absorptive membranes is low, which is the primary reason for less than 5% of the applied dose reaching the intraocular tissues. The cornea is a mechanical barrier that limits the entry of exogenous substances into the eye and protects the ocular tissues. Cornea is considered as a major barrier for ocular drug delivery. The cornea can be divided mainly into the epithelium, stroma, and endothelium. The highly hydrated structure of the stroma poses a significant barrier to permeation of lipophilic drugs.

There are two major pathways of drug absorption through the eye: corneal and non-corneal. In the corneal route, drug travels from the precorneal area through the cornea to the aqueous humor and then to the intraocular tissues. The corneal route is the primary route preferred by small and lipophilic drugs. Absorption in the corneal route is influenced by its aqueous solubility, molecular size, charge and degree of ionization. The noncorneal route also starts in the precorneal area, continues in the conjunctiva and the sclera, in the blood vessels and then also reaches the intraocular tissues (non-corneal absorption via the conjunctiva). The non-corneal conjunctival and scleral routes are preferred by large and more hydrophilic drugs. The cornea consists of three main layers, but the epithelium and stroma seem to be the most decisive barrier for ophthalmic drug delivery.

Standard ophthalmic treatments, such as the use of topically applied steroids, are directed to controlling the inflammatory symptoms in the eye. However, a complication with steroid treatments is that a significant percentage of treated subjects suffer from increased intraocular pressure, which can exacerbate eye disorders, such as glaucoma and cataracts. Currently available treatment for ocular diseases, particularly uveitis, include local corticosteroids which is most effective agent for anterior uveitis. But for intermediate uveitis, posterior uveitis or panuveitis, systemic dosing along with immunosuppressant is necessary. For certain conditions such as mild scleritis, other anti-inflammatory agents may be used instead of corticosteroids. The treatment of uveitis often requires the use of more than one immunosuppressive agent.

Systemic administration of drug is not preferred for the treatment of an ophthalmic condition, such as uveitis or keratoplasty, owing to high side effects. Rather, a localized treatment with increased retention time of the drug in the eye accompanied by increased bioavailability would be more effective and devoid of systemic side effects. Thus, there is a need in the art to develop topical treatment modalities to treat ocular disorders.

### SUMMARY

The present invention relates to a formulation comprising mycophenolic acid (MPA) or a pharmaceutically acceptable salt or derivative thereof; and at least one component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, potassium removing agent, suspending agent, and therapeutic agent. Generally, the formulations comprise mycophenolic acid, or a pharmaceutically acceptable salt or a derivative thereof as the active agent. In one aspect, the present invention provides said formulations for treating various disorders associated with inflammatory and autoimmune conditions.

In some embodiments, the formulation is for ophthalmic use, i.e., the formulation is an ophthalmic formulation. Formulations of the invention can be used for delivery to the eye with increased retention time in the eye and increased bioavailability of mycophenolic acid, or a pharmaceutically acceptable salt or derivative thereof in the eye.

In some embodiments, the ophthalmic formulation is an ointment formulation.

In some embodiments, the ophthalmic formulation is a suspension formulation.

In some embodiments, the ophthalmic formulation is an injectable formulation.

In some embodiments, the ophthalmic formulation is in a form of solution, suspension, ointment, emulsion, ocular injection, nanoparticulate system, nano suspension, eye or intraocular implant, ocular insert, pellet, gel, colloidal system, or hydrogel.

The present invention further provides a method for preparing the ophthalmic formulation as defined above, said method comprising combining: a) the mycophenolic acid (MPA) or a pharmaceutically acceptable salt or derivative thereof with b) the one or more component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, potassium removing agent, suspending agent, and therapeutic agent.

In one aspect, the present invention provides a pharmaceutical kit or package comprising the ophthalmic formulation as defined above and an instruction manual for application of the ophthalmic formulation thereof.

The ophthalmic formulations described herein can be used to treat various de novo inflammatory eye disorders, those associated with autoimmune diseases, or infections affecting the eye. Accordingly, in one aspect, the present invention provides a method for treating an ocular disease, the method comprising administering the ophthalmic formulation as described herein to a subject in need thereof. In some embodiments, the formulations of the invention can be used to treat uveitis. In some embodiments, the formulations of the invention can be used to treat anterior uveitis, intermediate uveitis, posterior uveitis or panuveitis. In other embodiments, the formulations of the invention can be used in keratoplasty, particularly high risk keratoplasty.

In yet another embodiment, the formulations of the invention can be used to treat Lichen sclerosus.

### BRIEF DESCRIPTION OF FIGURES

FIG. 1 illustrates ex-vivo study set up using whole goat eye.

FIG. 2 illustrates the in vitro study of solution formulation using goat cornea.

FIG. 3 provides representative photos of rabbit eye showing uveitis scoring and grading system.

FIG. 4 illustrates the efficacy of mycophenolate topical formulations (ointment and suspension) in BSA sensitized uveitis model in vivo.

FIG. 5 shows representative images of rabbit eye in a BSA induced uveitis model after treatment with mycophenolate formulations.

FIG. 6 illustrates total leucocytes (WBC) and differential count from aqueous humor of rabbit eye.

FIG. 7 illustrates histopathology leucocytes infiltration score from aqueous humor of rabbit eye in a rabbit uveitis model.

### DETAILED DESCRIPTION

The present invention relates to a formulation comprising mycophenolic acid (MPA) or a pharmaceutically acceptable salt or derivative thereof; and at least one component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, suspending agent, and therapeutic agent, wherein the formulation is an ophthalmic formulation.

In some embodiments of the ophthalmic formulation described herein, the pharmaceutically acceptable derivative is an ester or an analog of mycophenolic acid.

In some embodiments of the ophthalmic formulation described herein, the pharmaceutically acceptable derivative is mycophenolate mofetil (MMF) or an analog thereof.

In some embodiments of the ophthalmic formulation described herein, the pharmaceutically acceptable salt is selected from the group consisting of mycophenolate sodium (MPS), mycophenolate mofetil hydrochloride (MMF.HCl), mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, and combinations thereof.

In some embodiments of the ophthalmic formulation described herein, the pharmaceutically acceptable salt is mycophenolate sodium (MPS) or mycophenolate mofetil hydrochloride (MMF.HCl).

In some embodiments of the ophthalmic formulation described herein, the mycophenolic acid (MPA) or a pharmaceutically acceptable salt or derivative thereof has a particle size of about 10 nm to about 100 μm, including all values or ranges therefrom.

In some embodiments of the ophthalmic formulation described herein, the mycophenolic acid (MPA) or a pharmaceutically acceptable salt or derivative thereof has a particle size of about 10 nm to 50 μm, including all values or ranges therefrom.

In some embodiments of the ophthalmic formulation described herein, the mycophenolic acid (MPA) or a pharmaceutically acceptable salt or derivative thereof has a particle size of about 5 μm to 100 μm, including all values or ranges therefrom.

In some embodiments of the ophthalmic formulation described herein, the mycophenolic acid (MPA) or a pharmaceutically acceptable salt or derivative thereof has a particle size of about 5 μm to 50 μm, including all values or ranges therefrom.

In some embodiments of the ophthalmic formulation described herein, the mycophenolic acid (MPA) or a pharmaceutically acceptable salt or derivative thereof has a particle size of about 5 μm to 25 μm, including all values or ranges therefrom.

In embodiments of the ophthalmic formulation described herein, the mycophenolic acid (MPA) or a pharmaceutically acceptable salt or derivative thereof has a particle size of about 20 μm to 30 μm, including all values or ranges therefrom.

In embodiments of the ophthalmic formulation described herein, the mycophenolic acid (MPA) or a pharmaceutically acceptable salt or derivative thereof has a particle size of about 20 μm to 25 μm, including all values or ranges therefrom.

In some embodiments of the ophthalmic formulation described herein, the mycophenolic acid (MPA) or a pharmaceutically acceptable salt or derivative thereof in the formulation has a particle size of about 25 μm.

In some embodiments, the ophthalmic formulation is an ointment formulation.

In some embodiments, the ophthalmic formulation is an ointment formulation comprising mycophenolate mofetil hydrochloride (MMF.HCl).

In some embodiments, the ophthalmic formulation is a suspension formulation.

In some embodiments, the ophthalmic formulation is a suspension formulation having a pH of 4-7.

In some embodiments, the ophthalmic formulation is a suspension formulation having a pH of 5-6.

In some embodiments, the ophthalmic formulation is a suspension formulation comprising mycophenolate sodium (MPS).

In some embodiments, the ophthalmic formulation is an injectable formulation.

In some embodiments, the ophthalmic formulation is a topical formulation having a pH ranging between 4 to 8, including all values and ranges thereof; and said formulation is stable for more than 3 months. Mycophenolic acid or its salts may be unstable at normal pH, such as a pH of below 4. Accordingly, in some embodiments, the ophthalmic formulations of mycophenolic acid (MPA) or a pharmaceutically acceptable salt or derivative thereof as described herein have a pH ranging between 4 to 8 which provide greater stability.

In some embodiments, the ophthalmic formulation is a topical formulation having a pH of 4, 4.5, 5, 5.5, 6, 6.5, 7, 7.7 or 8, and said formulation is stable for more than 3 months.

In some embodiments, the ophthalmic formulations described herein is stable for 3 months to 6 months.

In some embodiments of the ophthalmic formulation described herein, the pharmaceutically acceptable derivative is selected from the group consisting of:
Compound 1 having the structure:

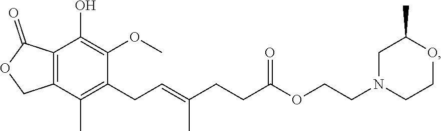 [SMILES: COc1c(O)c2c(c(C)c1C/C=C(\C)CCC(=O)OCCN1CCO[C@H](C)C1)COC2=O]

Compound 2 having the structure:

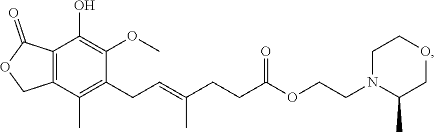 [SMILES: COc1c(O)c2c(c(C)c1C/C=C(\C)CCC(=O)OCCN1CCOC[C@H]1C)COC2=O]

Compound 3 having the structure:

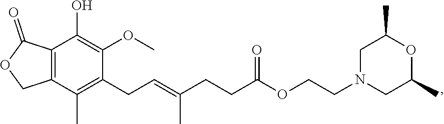 [SMILES: COc1c(O)c2c(c(C)c1C/C=C(\C)CCC(=O)OCCN1C[C@@H](C)O[C@@H](C)C1)COC2=O]

Compound 4 having the structure:

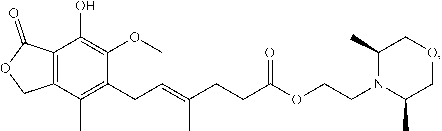 [SMILES: COc1c(O)c2c(c(C)c1C/C=C(\C)CCC(=O)OCCN1[C@H](C)COC[C@@H]1C)COC2=O]

Compound 5 having the structure:

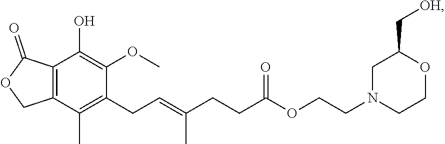 [SMILES: COc1c(O)c2c(c(C)c1C/C=C(\C)CCC(=O)OCCN1CCO[C@H](CO)C1)COC2=O]

Compound 6 having the structure:

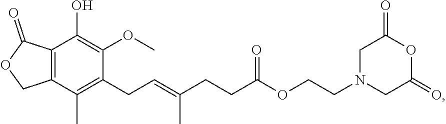 [SMILES: COc1c(O)c2c(c(C)c1C/C=C(\C)CCC(=O)OCCN1CC(=O)OC(=O)C1)COC2=O]

Compound 7 having the structure:

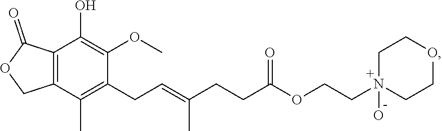

Compound 8 having the structure:

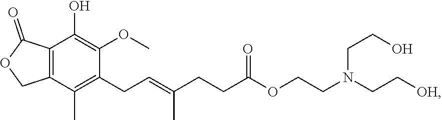 [SMILES: COc1c(O)c2c(c(C)c1C/C=C(\C)CCC(=O)OCCN(CCO)CCO)COC2=O]

and
Compound 9 having the structure:

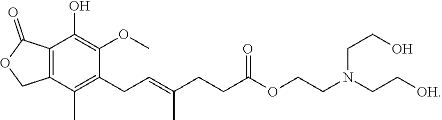 [SMILES: COc1c(O)c2c(c(C)c1C/C=C(\C)CCC(=O)OCCN(CCO)CCO)COC2=O]

In some embodiments of the ophthalmic formulation described herein, the preservative is selected from the group consisting of boric acid, benzalkonium chloride, benzethonium chloride, benzododecinium bromide, cetylpyridinium chloride, chlorobutanol, thimerosol, phenylmercuric nitrate, phenylmercuric acetate, methylparaben, propylparaben, butylparaben, phenylethyl alcohol, sodium benzoate, sodium propionate, sorbic acid, sodium sorbate, sodium borate, sodium perborate and combinations thereof.

In some embodiments, the ophthalmic formulations described herein is preservative free i.e. does not comprise any preservative. In some embodiments, formulations comprising mycophenolic acid (MPA) or a pharmaceutically acceptable salt or derivative thereof described herein are in preservative-free sterile dosage form for ophthalmic use.

In some embodiments of the ophthalmic formulation described herein, the chelating agent is selected from the group consisting of ethylenediamine tetracetic acid (EDTA), edetate disodium (disodium EDTA salt), edetate sodium (EDTA tetrasodium salt), sodium EDTA and combinations thereof.

In some embodiments of the ophthalmic formulation described herein, the buffering agent or the pH modifier is selected from the group consisting of acetic acid, boric acid, anhydrous citric acid, citric acid monohydrate, hydrochloric acid, phosphoric acid, potassium phosphate monobasic, sodium acetate, sodium acetate anhydrous, sodium carbonate, sodium carbonate monohydrate, sodium hydroxide, sodium phosphate, sodium phosphate (heptahydrate), sodium phosphate dibasic, sodium phosphate dibasic (anhydrous), sodium phosphate dibasic (dihydrate), sodium phosphate dibasic (dodecahydrate), sodium phosphate monobasic, sodium phosphate monobasic (anhydrous), sodium phosphate monobasic (dihydrate), sodium phosphate monobasic (monohydrate), sulfuric acid, trisodium citrate dihydrate, tromethamine and combinations thereof.

In some embodiments of the ophthalmic formulation described herein, the thickening agent, the viscosity modifier or the viscosity enhancer is selected from the group consisting of acrylic acid polymer (carbopol), dextran 40 (molecular weight of 40,000 Daltons), dextran 70 (molecular weight of 70,000 Daltons), gelatin, glycerin, carboxymethylcellulose (CMC), hydroxyethyl cellulose (HEC), hydroxypropyl methylcellulose (HPMC), methyl cellulose, ethyl cellulose, polyethylene glycol (PEG), poloxamer 407, polysorbate 80, propylene glycol, polyvinyl alcohol (PVA), polyvinylpyrrolidone (povidone), povidone K30, povidone K90, carbomer 940, carbomer copolymer Type A (allyl pentaerythritol crosslinked), carbomer copolymer Type B (allyl pentaerythritol crosslinked), carbomer homopolymer Type B (allyl pentaerythritol crosslinked), carbomer homopolymer Type B (allyl sucrose crosslinked), a crosslinked copolymer of acrylic acid and a hydrophobic C10-30 alkyl acrylate co-monomer, carboxymethyl cellulose sodium, crospovidone, dextran, guar gum, hydroxypropyl cellulose (HPC), hydroxymethyl cellulose (HMC), hydroxymethyl cellulose (2000 mPa·S at 1%), hydroxymethyl cellulose (4000 mPa·S at 1%), hypromellose, hypromellose 2906 (4000 mPa·S), hypromellose 2910 (15000 mPa·S), hypromellose 2910 (3 mPa·S), hypromellose 2910 (5 mPa·S), polycarbophil, xanthan gum, sodium hyaluronate, and combinations thereof.

In some embodiments of the ophthalmic formulation described herein, the antioxidant is selected from the group consisting of alpha-tocopherol, EDTA, sulfate, sodium bisulfite, sodium metabisulfite, sodium sulfate (anhydrous), sodium thiosulfate, sodium thiosulfite, thimerosal, thiourea, and combinations thereof.

In some embodiments of the ophthalmic formulation described herein, the tonicity modifier is selected from the group consisting of dextrose, glycerin, potassium chloride, propylene glycol, sodium chloride, calcium chloride, magnesium chloride, mannitol, and combinations thereof.

In some embodiments of the ophthalmic formulation described herein, the surfactant is selected from the group consisting of non-ionic surfactant, amphoteric surfactant, anionic surfactant, cationic surfactant and combinations thereof.

In some embodiments of the ophthalmic formulation described herein, the surfactant is selected from the group consisting of polyoxyethylene (POE)-polyoxypropylene (POP) block copolymer, poloxamer 407, poloxamer 235, poloxamer 188, ethylenediamine POE-POP block copolymer adduct, poloxamine, POE sorbitan fatty acid ester, polysorbate 20, polysorbate 60, polysorbate 65, polysorbate 80, POE hydrogenated castor oil, POE (5) hydrogenated castor oil, POE (10) hardened castor oil, POE (20) hardened castor oil, POE (40) hardened castor oil, POE (50) hardened castor oil, POE (60) hardened castor oil, POE (100) hardened castor oil, POE castor oil, POE (3) castor oil, POE (10) castor oil, POE (35) castor oil, POE (40) castor oil, polyoxyl 40 hydrogenated castor oil, POE alkyl ether, polyoxyethylene (9) lauryl ether, polyoxyethylene (20), polyoxypropylene (4), cetyl ether, POE alkylphenyl ether, POE (10) nonylphenyl ether, polyoxyl stearate, polyoxyl 40 stearate, polyoxydiethyl castor oil, polyoxyl-35 castor oil, cremophor, glycine type amphoteric surfactant, alkyldiaminoethylglycine, alkylpolyaminoethylglycine, betaine type amphoteric surfactant, lauryldimethylaminoacetic acid betaine, imidazolinium betaine, quaternary ammonium salt, alkyl tertiary ammonium salt, benzalkonium chloride, benzethonium chloride, polydronium chloride, biguanide compound, polyhexamethylene biguanide hydrochloride, sodium alkylbenzene sulfonate, tyloxapol, poloxomer 407, tween 20, and combinations thereof.

In some embodiments of the ophthalmic formulation described herein, the humectant is selected from the group consisting of glycerin, hyaluronic acid, sorbitol, urea, alpha hydroxy acid, sugar, lactic acid, polyethylene glycol (PEG), PEG-4, PEG-8, propylene glycol, glyceryl triacetate, lithium chloride, polyol, sorbitol, xylitol, maltitol, polydextrose, quillaia, hexadecyl, myristyl, isodecyl and isopropyl esters of adipic, lactic, oleic, stearic, isostearic, myristic and linoleic acids, sodium isostearoyl-2-lactylate, sodium capryl lactylate, hydrolyzed protein, collagen-derived protein, aloe vera gel, acetamide monoethanolamide (MEA), sodium pyrrolidone carboxylic acid, L-proline, guanidine, pyrrolidone, acetamido propyl trimmonium chloride, calcium stearoyl lactylate, chitosan pyrrolidone carboxylic acid (PCA), diglycerol lactate, ethyl ester of hydrolyzed silk, fatty quaternary amine chloride complex, glycereth-7, glycereth-12, glycereth-26, glycereth-4.5 lactate, diglycerin, polyglycerin, hydrolyzed fibronectin, lactamide MEA, lactamide N-(2-hydroxyetheryl), mannitol, methyl gluceth-10, methyl gluceth-20, panthenol, pyrrolidone carboxylic acid (PCA), methylsilanol PCA, polyamino sugar condensate, quaternium-22, sea salt, sodium caproyl lactylate, sodium hyaluronate, sodium isostearoyl lactylate, sodium lactate, sodiumlauroyl lactylate, sodium PCA, sodium polyglutamate, sodium stearoyl lactylate, soluble collagen, sorbitan laurate, sorbitan oleate, sorbitansesquiisostearate, sorbitan stearate, sphingolipids, TEA-PCA (5-oxo-DL-proline, compound with 2,2′,2″-nitrilotriethanol), ethylene glycol, diethylene glycol, triethylene glycol, dipropylene glycol, 1,3-butylene glycol, 1,4-butylene glycol, glycerol, diglycerol, polyglycerol, glycerol ethylene oxide (EO) and propylene oxide (PO) adduct, sugar alcohol EO and PO adduct, adduct of EO or PO and monosaccharide such as galactose and fructose, adduct of EO or PO and polysaccharide such as maltose and lactose, sodium pyrrolidone carboxylate, polyoxyethylene methyl glycoside, hexylene glycol, maltose, D-mannitol, gluten, glucose, fructose, lactose, sodium chondroitin sulfate, sodium adenosine phosphate, gallates, pyrrolidone carbonates, glucosamine, cyclodextrin, alpha hydroxy acids, 2-methyl-1,3-propane diol, and combinations thereof.

In some embodiments of the ophthalmic formulation described herein, the solvent or co-solvent is selected from the group consisting of water, alcohol, glycerin, propylene glycol, propylene glycol diacetate, polypropylene glycol, sorbitol and combinations thereof.

In some embodiments of the ophthalmic formulation described herein, the emulsifier or the co-emulsifier is selected from the group consisting of silicone-based emulsifier, a polyethylene glycol emulsifier, a polysiloxane emulsifier, a glycoside emulsifier, an acrylic-based emulsifier, and combinations thereof.

In some embodiments of the ophthalmic formulation described herein, the emulsifier or the co-emulsifier is selected from the group consisting of polysorbate, carbomer, castor oil, ethyl alcohol, isopropyl alcohol, ethyl carbonate, ethyl acetate, benzyl alcohol, benzyl benzoate, propylene glycol, polyethylene glycols, 1,3-butyleneglycol, dimethylformamide, sodium lauryl sulfate, sodium docusate, cholesterol, cholesterol esters, taurocholic acid, phosphatidylcholine, oils, cottonseed oil, groundnut oil, corn germ oil, olive oil, castor oil, sesame oil, glycerol, tetrahydrofurfuryl alcohol, fatty acid esters of sorbitan, cetyl alcohol, glyceryl monostearate, nonoxynol-9, octoxynol-40, poloxamer 188, poloxamer 407, polyethylene glycol 400, polyethylene glycol 8000, polyoxyl 35 castor oil, polyoxyl 40 hydrogenated castor oil, polyoxyl 15 hydroxystearate, polyoxyl 40 stearate, polysorbate 20, polysorbate 80, tyloxapol, l′EA/K stearate (triethanolamine/potassium stearate), sodium lauryl stearate, sodium cetearyl sulfate, beeswax/borax, glycerol di-stearate, PEG (polyethyleneglycol)-100 stearate, polysorbate 20, steareth 2, steareth 20, distearyldimethylammonium chloride, benzalkonium chloride, steapyrium chloride, acrylates/C 10-30 alkyl acrylate crosspolymer, polyacrylamide, polyquaternium-37, dicaprylate/dicaparate, PPG-1 Trideceth-6, alkyl modified dimethiconecopolyols, polyglyceryl esters, ethoxylated di-fatty esters, ionic polysorbate surfactant, nonylphenol polyethylene glycol ethers, alkylphenol-hydroxypolyoxyethylene, Poly(oxy-1,2-ethanediyl), alpha-(4-nonylphenol)-omega-hydroxy-, branched, nonylphenol polyethylene glycol ether mixtures, phenoxypolyethoxyethanols or polymers thereof, sodium dodecyl sulfate, and combinations thereof.

In some embodiments of the ophthalmic formulation described herein, the ointment base is selected from the group consisting of light liquid paraffin, white soft paraffin, petrolatum, lanolin, lanolin alcohol, chlorobutanol, methylparaben, propylparaben, and combinations thereof.

In some embodiments of the ophthalmic formulation described herein, the petrolatum has a concentration of 5% to 80% of microcrystalline wax based on the total composition of petrolatum.

In some embodiments of the ophthalmic formulation described herein, the petrolatum has a concentration of 20% to 60% of microcrystalline wax based on the total composition of petrolatum.

In some embodiments of the ophthalmic formulation described herein, the targeting agent is selected from the group consisting of didodecyldimethylammonium bromide, stearylamine, N-[1-(2,3-dioleoyloxy)propyl]-N,N,N-trimethylammonium chloride, and combinations thereof.

In some embodiments of the ophthalmic formulation described herein, the polymer is selected from the group consisting of poly (lactide), poly (lactideglycolide), poly (glycolide), poly (caprolactone), poly (amides), poly (anhydrides), poly (amino acids), poly (esters), poly (cyanoacrylates), poly (phosphazines), poly (phosphoesters), poly (esteramides), poly (dioxanones), poly (acetals), poly (cetals), poly (carbonates), poly (orthocarbonates), degradable poly (urethanes), chitins, chitosans, poly (hydroxybutyrates), poly (hydroxyvalerates), poly (maleic acid), poly (alkylene oxalates), poly (alkylene succinates), poly (hydroxybutyrates-co-hydroxyvalerates), copolymers, terpolymers or oxidised cellulose thereof, poly (e-caprolactone) (PCL), methacrylate acid copolymer, methacrylate esters, acrylic esters, poly (alkyl methacrylate), poly (methyl methacrylate), sodium alginate, chitosan and combinations thereof.

In some embodiments of the ophthalmic formulation described herein, the wetting agent is selected from the group consisting of hydrophilic polymers, polysorbate 20, polysorbate 80, poloxamer 282, tyloxapol, cellulose based polymers, HPMC, CMC, polyvinylpyrrolidone (PVP), polyvinyl alcohol (PVA), and combinations thereof.

In some embodiments of the ophthalmic formulation described herein, the lubricating agent is selected from the group consisting of non-phospholipid based agent, phospholipid based agent, petrolatum, mineral oil, propylene glycol, ethylene glycol, polyethylene glycol, hydroxypropylmethylcellulose (HPMC), carboxymethylcellulose (CMC), hydroxypropylcellulose, dextrans, dextran 70, water soluble proteins, gelatin, vinyl polymers, polyvinyl alcohol (PVA), polyvinylpyrrolidone (PVP), povidone, carbomers, carbomer 934P, carbomer 941, carbomer 940, carbomer 974P, vitamin E, and combinations thereof.

In some embodiments of the ophthalmic formulation described herein, the suspending agent is selected from the group consisting of pH independent polymers, pH dependent polymers and combinations thereof.

In some embodiments of the ophthalmic formulation described herein, the therapeutic agent is selected from the group consisting of an antibacterial agent, an anti-fungal agent, an anti-viral agent, anti-acanthamoebal agent, an anti-inflammatory agent, an immunosuppressive agent, an anti-glaucoma agent, an anti-VEGF agent, a growth factor, and combinations thereof.

In some embodiments of the ophthalmic formulation described herein, the antibacterial agent is selected from the group consisting of penicillins, cephalosporins, penems, carbapenems, monobactams, aminoglycosides, sulfonamides, macrolides, tetracyclines, lincosamides, quinolones, chloramphenicol, vancomycin, metronidazole, rifampin, isoniazid, spectinomycin, trimethoprim sulfamethoxazole, chitosan, ansamycins, daptomycin, nitrofurans, oxazolidinones, bacitracin, colistin, polymixin B, clindamycin, and combinations thereof; the anti-fungal agent is selected from the group consisting of amphotericin B, natamycin, candicin, filipin, hamycin, nystatin, rimocidin, voriconazole, imidazoles, triazoles, thiazoles, allylamines, echinocandins, benzoic acid, ciclopirox, flucytosine, griseofulvin, haloprogin, tolnaftate, undecylenic acid, povidone-iodine and combinations thereof; the anti-viral agent is selected from the group consisting of acyclovir, valacyclovir, famciclovir, penciclovir, trifluridine, vidarabine, and combinations thereof; the anti-acanthamoebal agent is selected from the group consisting of chlorohexidine, polyhexamethylen biguanide, propamidine, hexamidine, and combinations thereof; the anti-inflammatory agent is selected from the group consisting of corticosteroids, salicylates, propionic acid derivatives, acetic acid derivatives, enolic acid derivatives, anthranilic acid derivatives, selective cox-2 inhibitors, sulfonanilides, antibodies, tumor necrosis factor-alpha inhibitors, dominant negative ligands, interleukin-1 receptor antagonists, and combinations thereof; the immunosuppressive agent is selected from the group consisting of alkylating agents, antimetabolites, mycophenolate, cyclosporine, tacrolimus, rapamycin, and combinations thereof; the anti-glaucoma agent is selected from the group consisting of prostaglandin analogs, beta blockers, adrenergic agonists, carbonic anhydrase inhibitors, parasympathomimetic (miotic) agents, and combinations thereof; the anti-vascular endothelial growth factor (anti-VEGF) agent is selected from the group consisting of bevacizumab, ranibizumab, aflibercept, and combinations thereof; the growth factor is selected from the group consisting of epidermal growth factor (EGF), platelet-derived growth factor (PDGF), vitamin A, vitamin E, fibronectin, annexin a5, albumin, alpha-2 macroglobulin, fibroblast growth factor b, insulin-like growth factor-I, nerve growth factor, hepatocyte growth factor, and combinations thereof.

In some embodiments of the ophthalmic formulation described herein, the mycophenolic acid (MPA) or a pharmaceutically acceptable salt or derivative thereof is present in an amount up to 5% weight/volume (w/v) or weight/weight (w/w) of the formulation, including all values and ranges therefrom.

In some embodiments of the ophthalmic formulation described herein, the mycophenolic acid (MPA) or a pharmaceutically acceptable salt or derivative thereof is present in an amount of about 0.005% to 5% w/v or w/w of the formulation, including all values and ranges therefrom.

In some embodiments of the ophthalmic formulation described herein, the mycophenolic acid (MPA) or a pharmaceutically acceptable salt or derivative thereof is present in an amount of about 0.005% to 4% w/v or w/w of the formulation, including all values and ranges therefrom.

In some embodiments of the ophthalmic formulation described herein, the mycophenolic acid (MPA) or a pharmaceutically acceptable salt or derivative thereof is present in an amount of about 0.5% to 4% w/v or w/w of the formulation, including all values and ranges therefrom.

In some embodiments of the ophthalmic formulation described herein, each of the at least one component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, suspending agent and therapeutic agent is present in an amount up to 99.99% w/v or w/w of the formulation including all values and ranges therefrom.

In some embodiments of the ophthalmic formulation described herein, the preservative is present in an amount of about 0.005% to 10% w/v or w/w of the formulation, including all values and ranges therefrom.

In some embodiments of the ophthalmic formulation described herein, the chelating agent is present in an amount of about 0.005% to 5% w/v or w/w of the formulation, including all values and ranges therefrom.

In some embodiments of the ophthalmic formulation described herein, the buffering agent is present in an amount of about 0.005% to 5% w/v or w/w of the formulation, including all values and ranges therefrom.

In some embodiments of the ophthalmic formulation described herein, the pH modifier is present in an amount of about 0.005% to 5% w/v or w/w of the formulation, including all values and ranges therefrom.

In some embodiments of the ophthalmic formulation described herein, the thickening agent is present in an amount of about 0.005% to 5% w/v or w/w of the formulation, including all values and ranges therefrom.

In some embodiments of the ophthalmic formulation described herein, the viscosity enhancer or viscosity modifier is present in an amount of about 0.005% to 5% w/v or w/w of the formulation, including all values and ranges therefrom.

In some embodiments of the ophthalmic formulation described herein, the antioxidant is present in an amount of about 0.005% to 5% w/v or w/w of the formulation, including all values and ranges therefrom.

In some embodiments of the ophthalmic formulation described herein, the tonicity modifier is present in an amount of about 0.005% to 5% w/v or w/w of the formulation, including all values and ranges therefrom.

In some embodiments of the ophthalmic formulation described herein, the surfactant is present in an amount of about 0.005% to 5% w/v or w/w of the formulation, including all values and ranges therefrom.

In some embodiments of the ophthalmic formulation described herein, the humectant is present in an amount of about 0.005% to 5% w/v or w/w of the formulation, including all values and ranges therefrom.

In some embodiments of the ophthalmic formulation described herein, the solvent or co-solvent is present in an amount of about 0.005% to 20% w/v or w/w of the formulation, including all values and ranges therefrom.

In some embodiments of the ophthalmic formulation described herein, the emulsifier is present in an amount of about 0.005% to 10% w/v or w/w of the formulation, including all values and ranges therefrom.

In some embodiments of the ophthalmic formulation described herein, the co-emulsifier is present in an amount of about 0.005% to 10% w/v or w/w of the formulation, including all values and ranges therefrom.

In some embodiments of the ophthalmic formulation described herein, the ointment base is present in an amount of about 0.005% to 99.99% w/v or w/w of the formulation, including all values and ranges therefrom.

In some embodiments of the ophthalmic formulation described herein, the targeting agent is present in an amount of about 0.005% to 10% w/v or w/w of the formulation, including all values and ranges therefrom.

In some embodiments of the ophthalmic formulation described herein, the polymer is present in an amount of about 0.005% to 5% w/v or w/w of the formulation, including all values and ranges therefrom.

In some embodiments of the ophthalmic formulation described herein, the wetting agent is present in an amount of about 0.005% to 10% w/v or w/w of the formulation, including all values and ranges therefrom.

In some embodiments of the ophthalmic formulation described herein, the lubricating agent is present in an amount of about 0.005% to 30% w/v or w/w of the formulation, including all values and ranges therefrom.

In some embodiments of the ophthalmic formulation described herein, the suspending agent is present in an amount of about 0.005% to 5% w/v or w/w of the formulation, including all values and ranges therefrom.

In some embodiments of the ophthalmic formulation described herein, the therapeutic agent is present in an amount of about 0.005% to 5% w/v or w/w of the formulation, including all values and ranges therefrom.

In some embodiments of the ophthalmic formulation described herein, the preservative is present in an amount of about 0.005% to 10% w/v or w/w of the formulation, the chelating agent is present in an amount of about 0.005% to 5% w/v or w/w of the formulation, the buffering agent is present in an amount of about 0.005% to 5% w/v or w/w of the formulation, the pH modifier is present in an amount of about 0.005% to 5% w/v or w/w of the formulation, the thickening agent is present in an amount of about 0.005% to 5% w/v or w/w of the formulation, the viscosity enhancer or viscosity modifier is present in an amount of about 0.005% to 5% w/v or w/w of the formulation, the antioxidant is present in an amount of about 0.005% to 5% w/v or w/w of the formulation, the tonicity modifier is present in an amount of about 0.005% to 5% w/v or w/w of the formulation, the surfactant is present in an amount of about 0.005% to 5% w/v or w/w of the formulation, the humectant is present in an amount of about 0.005% to 5% w/v or w/w of the formulation, the solvent or co-solvent is present in an amount of about 0.005% to 20% w/v or w/w of the formulation, the emulsifier is present in an amount of about 0.005% to 10% w/v or w/w of the formulation, the co-emulsifier is present in an amount of about 0.005% to 10% w/v or w/w of the formulation, the ointment base is present in an amount of about 0.005% to 99.99% w/v or w/w of the formulation, the targeting agent is present in an amount of about 0.005% to 10% w/v or w/w of the formulation, the polymer is present in an amount of about 0.005% to 5% w/v or w/w of the formulation, the wetting agent is present in an amount of about 0.005% to 10% w/v or w/w of the formulation, the lubricating agent is present in an amount of about 0.005% to 30% w/v or w/w of the formulation, the suspending agent is present in an amount of about 0.005% to 5% w/v or w/w of the formulation, and the therapeutic agent is present in an amount of about 0.005% to 5% w/v or w/w of the formulation.

In some embodiments of the ophthalmic formulation described herein, the formulation is a non-aqueous formulation or an aqueous formulation.

In some embodiments, the ophthalmic formulation is a non-aqueous formulation.

In some embodiments, the ophthalmic formulation is an aqueous formulation.

In some embodiments of the ophthalmic formulation described herein, the non-aqueous formulation comprises water in an amount of less than 50% by weight of the formulation, including all values and ranges therefrom.

In some embodiments of the ophthalmic formulation described herein, the aqueous formulation comprises water in an amount of more than 50% by weight of the formulation, including all values and ranges therefrom.

In some embodiments of the ophthalmic formulation described herein, the formulation is in a form of liquid, fluid, emulsion, gel, semi-solid or solid.

In some embodiments of the ophthalmic formulation described herein, the formulation is in a form of solution, suspension, ointment, emulsion, ocular injection, nanoparticulate system, nano suspension, eye or intraocular implant, ocular insert, pellet, gel, colloidal system, or hydrogel.

In some embodiments of the ophthalmic formulation described herein, the formulation is in a form of self-emulsifying drug delivery system or an in situ gel-forming system.

In some embodiments of the ophthalmic formulation described herein, the formulation is an ointment having a viscosity of about 7000 to 20000 mPa and particle size ranging from about 1 μm to 10 μm.

In some embodiments of the ophthalmic formulation described herein, the formulation is a suspension having a viscosity less than 2000 mPa and particle size ranging less than 10 nm.

In some embodiments of the ophthalmic formulation described herein, the formulation is an ointment formulation selected from:
a) mycophenolate mofetil, light liquid paraffin, white soft paraffin, lanolin, and lanolin alcohol;
b) mycophenolate sodium, light liquid paraffin, white soft paraffin, lanolin, and lanolin alcohol; or
c) mycophenolate mofetil and white soft paraffin.

In some embodiments of the ophthalmic formulation described herein, the formulation is an emulsion formulation selected from:
a) mycophenolate mofetil, light liquid paraffin, polysorbate 80, polyoxyl 35 castor oil, and water;
b) mycophenolate mofetil, light liquid paraffin, polysorbate 80, polyoxyl 35 castor oil, hydroxyethyl cellulose, and water;
c) mycophenolate mofetil, polysorbate 80, lanolin alcohol, castor oil, crosslinked copolymer of acrylic acid and a hydrophobic C10-30 alkyl acrylate co-monomer, and water;
d) mycophenolate mofetil, polyoxyl 40 stearate, lanolin alcohol, castor oil, crosslinked copolymer of acrylic acid and a hydrophobic C10-30 alkyl acrylate co-monomer, and water;
e) mycophenolate mofetil, polysorbate 80, lanolin, lanolin alcohol, castor oil, crosslinked copolymer of acrylic acid and a hydrophobic C10-30 alkyl acrylate co-monomer, and water;
f) mycophenolate mofetil, polysorbate 80, lanolin alcohol, castor oil, light liquid paraffin, crosslinked copolymer of acrylic acid and a hydrophobic C10-30 alkyl acrylate co-monomer, and water;
g) mycophenolate mofetil, polyoxyl 40 stearate, lanolin alcohol, cetyl alcohol, glyceryl monostearate, castor oil, crosslinked copolymer of acrylic acid and a hydrophobic C10-30 alkyl acrylate co-monomer, and water; or
h) mycophenolate mofetil, polyoxyl 40 stearate, lanolin alcohol, cetyl alcohol, glyceryl monostearate, castor oil, ethanol, crosslinked copolymer of acrylic acid and a hydrophobic C10-30 alkyl acrylate co-monomer, and water.

In some embodiments of the ophthalmic formulation described herein, the formulation is a solution formulation selected from:
a) mycophenolate sodium, tween 80, boric acid, di-sodium EDTA, sodium chloride, hydroxypropyl methylcellulose, and water;
b) mycophenolate sodium, polysorbate 80, and water;
c) mycophenolate sodium, polyoxyl-35 castor oil, and water;
d) mycophenolate sodium, tween 20, and water;
e) mycophenolate sodium, polyoxyl 40 hydrogenated castor oil, and water;
f) mycophenolate sodium, poloxomer 407, and water;
g) mycophenolate sodium, polysorbate 80, polyvinyl alcohol, and water;
h) mycophenolate sodium, polysorbate 80, polyvinylpyrrolidone, and water;
i) mycophenolate sodium, polysorbate 80, acrylic acid polymer, and water;
j) mycophenolate sodium, polysorbate 80, hydroxyethyl cellulose, and water;
k) mycophenolate sodium, polysorbate 80, hydroxypropyl methylcellulose, and water;
l) mycophenolate sodium, polyoxyl-35 castor oil, and water;
m) mycophenolate sodium, polyoxyl-35 castor oil, acrylic acid polymer, and water;
n) mycophenolate sodium, polyoxyl-35 castor oil, hydroxyethyl cellulose, and water;
o) mycophenolate sodium, polyoxyl-35 castor oil, hydroxypropyl methylcellulose, and water;
p) mycophenolate sodium, polyoxyl-35 castor oil, polyvinyl alcohol, and water;
q) mycophenolate sodium, polyoxyl-35 castor oil, polyvinylpyrrolidone, and water;
r) mycophenolate sodium, polysorbate 80, polyoxyl-35 castor oil, and water;
s) mycophenolate sodium, polysorbate 80, polyoxyl-35 castor oil, polyvinyl alcohol, and water;
t) mycophenolate sodium, polysorbate 80, polyoxyl-35 castor oil, polyvinylpyrrolidone, and water;
u) mycophenolate sodium, polyoxyl 40 hydrogenated castor oil, polyoxyl-35 castor oil, polyvinyl alcohol, and water;
v) mycophenolate sodium, polyoxyl 40 hydrogenated castor oil, polyoxyl-35 castor oil, polyvinylpyrrolidone, and water;
w) mycophenolate sodium, polyoxyl 40 hydrogenated castor oil, polyoxyl-35 castor oil, hydroxypropyl methylcellulose, and water;
x) mycophenolate sodium, polysorbate 80, polyoxyl 40 hydrogenated castor oil, polyvinyl alcohol, and water;
y) mycophenolate sodium, polysorbate 80, polyoxyl 40 hydrogenated castor oil polyvinylpyrrolidone, and water;
z) mycophenolate sodium, polysorbate 80, polyoxyl 40 hydrogenated castor oil hydroxypropyl methylcellulose, and water;
aa) mycophenolate sodium, polysorbate 80, polyoxyl-35 castor oil, and water;
bb) mycophenolate sodium, polysorbate 80, polyoxyl-35 castor oil, acrylic acid polymer, and water;
cc) mycophenolate sodium, polysorbate 80, polyoxyl-35 castor oil, hydroxyethyl cellulose, and water;
dd) mycophenolate sodium, polysorbate 80, polyoxyl-35 castor oil, hydroxypropyl methylcellulose, and water;
ee) mycophenolate sodium, polysorbate 80, boric acid, hydroxyethyl cellulose, and water; or
ff) mycophenolate sodium, polysorbate 80, boric acid, hydroxypropyl methylcellulose, and water.

In some embodiments of the ophthalmic formulation described herein, the formulation is a suspension formulation selected from:
a) mycophenolic acid, polysorbate 80, acrylic acid polymer, glycerol, di-sodium EDTA, boric acid, and water;
b) mycophenolic acid, polysorbate 80, acrylic acid polymer, glycerol, boric acid, and water;
c) mycophenolic acid, polysorbate 80, acrylic acid polymer, glycerol, boric acid, citric acid, and water;
d) mycophenolic acid, polycarbophil, glycerol, boric acid, ortho phosphoric acid, and water; or
e) mycophenolic acid, polysorbate 80, di-sodium EDTA, polycarbophil, glycerol, boric acid, citric acid, and water.

In some embodiments of the ophthalmic formulation described herein, the formulation is a nanosuspension formulation comprising: mycophenolate sodium, glycerol, di-sodium EDTA, polysorbate 80, polycarbophil, boric acid, acrylate co-polymer, and water.

In some embodiments of the ophthalmic formulation described herein, the formulation is an ocular injection formulation selected from:
a) mycophenolate sodium, monobasic sodium phosphate monohydrate, dibasic sodium phosphate heptahydrate, sodium chloride, sucrose, polysorbate 20, sodium hydroxide, and water; or
b) mycophenolate mofetil hydrochloride, mannitol, polysorbate 20, dibasic sodium phosphate heptahydrate, sodium hydroxide, and water.

In some embodiments of the ophthalmic formulation described herein, the formulation is an eye implant or ocular insert formulation selected from:
a) mycophenolate mofetil or mycophenolate sodium or a combination thereof, sorbitan stearate, cholesterol, phosphate buffered saline, and vitamin E;
b) mycophenolate mofetil or mycophenolate sodium or a combination thereof, sodium alginate, glycerine, polyvinyl alcohol and chitosan;
c) mycophenolate mofetil or mycophenolate sodium or a combination thereof, sodium alginate, glycerine, and chitosan; or
d) mycophenolate mofetil or mycophenolate sodium or a combination thereof, sodium alginate, glycerine, and polyvinyl alcohol.

The present invention further provides a method for preparing the ophthalmic formulation defined in any of the preceding claims, said method comprising combining: a) the mycophenolic acid (MPA) or a pharmaceutically acceptable salt or derivative thereof with b) the one or more component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, suspending agent, and therapeutic agent.

The invention also relates to a pharmaceutical kit or package comprising the ophthalmic formulation defined herein and an instruction manual for application of the ophthalmic formulation thereof.

The present invention also relates to a method for treating an ocular disease, the method comprising administering the ophthalmic formulation defined herein to a subject in need thereof.

In some embodiments of the methods described herein, the ocular disease is a front-of-eye disease, back-of-eye disease, or a combination thereof.

In some embodiments of the methods described herein, the ocular disease is selected from the group consisting of uveitis, macular edema, angiographic cystoid macular edema, retinal ischemia, choroidal neovascularization, macular degeneration, retinal disease, diabetic retinopathy, diabetic retinal edema, retinal detachment, inflammatory disease, choroiditis, multifocal choroiditis, episcleritis, scleritis, birdshot retinochoroidopathy, vascular disease, retinal ischemia, retinal vasculitis, choroidal vascular insufficiency, choroidal thrombosis, neovascularization of the optic nerve, optic neuritis, blepharitis, keratitis, rubeosis iritis, Fuchs' heterochromic iridocyclitis, chronic uveitis, anterior uveitis, conjunctivitis, allergic conjunctivitis, keratoconjunctivitis sicca (dry eye syndrome), iridocyclitis, iritis, scleritis, episcleritis, corneal edema, scleral disease, ocular cicatricial pemphigoid, pars planitis, Posner Schlossman syndrome, Behcet's disease, Vogt-Koyanagi-Harada syndrome, hypersensitivity reactions, conjunctival edema, conjunctival venous congestion, periorbital cellulitis, acute dacryocystitis, non-specific vasculitis, sarcoidosis, and combinations thereof.

In some embodiments of the methods described herein, the subject is a mammal including a human.

In some embodiments of the methods described herein, the ocular disease is uveitis.

In some embodiments of the methods described herein, the uveitis is anterior, intermediate, posterior or panuveitis.

The present invention further provides ophthalmic formulation described herein, for use in treating ocular disease.

In some embodiments of the ophthalmic formulation or use described herein, the ocular disease is uveitis.

In some embodiments of the ophthalmic formulation or use described herein, uveitis is anterior, posterior, intermediate or panuveitis.

In some embodiments of the ophthalmic formulation described herein, said formulation is used as an ophthalmic insert.

In some embodiments of the ophthalmic formulation described herein, said formulation is used as an ophthalmic implant.

In some embodiments, the of the ophthalmic formulation is for use in keratoplasty or other ophthalmic surgery.

The present disclosure also provides use of mycophenolic acid, salts and derivatives thereof for the treatment of Lichen sclerosus. Lichen sclerosus is a rare skin disease where the immune cells attack the skin around the groin. In some embodiments, the mycophenolic acid, salts and derivatives thereof or their formulations as described in the present disclosure are used for the treatment of Lichen sclerosus.

The compounds mycophenolic acid (MPA) and derivatives such as its ester prodrug form, mycophenolate mofetil (MMF) have been used as immunosuppressive drugs to prevent rejection of allogenic organ transplants and for treatment of certain autoimmune diseases such as systemic lupus erythematosus and myasthenia gravis. The present invention is directed to formulations for ophthalmic use and particularly comprising mycophenolic acid (MPA) and/or MPA based compounds as the active pharmaceutical ingredient (API) along with at least one additional component/excipient as described above.

In some embodiments, the formulation comprises mycophenolate mofetil and at least one additional component/excipient described herein, wherein the mycophenolate mofetil (MMF) shows very low degradation and generates less than 3% mycophenolic acid (MPA). Thus, the MMF comprised in the present ophthalmic formulations show greater stability. Said property of the present ophthalmic formulations result in better permeation of the active (mycophenolic acid) into eye. Additionally, no complexation/complication is involved at the site of action and the active (mycophenolic acid) has a straight exposure to the eye.

MPA and MPA derived compounds which are useful in the present ophthalmic formulations are discussed above. In some embodiments, a summary of exemplary mycophenolate based APIs employed in the present ophthalmic formulations and their properties/advantages is provided in Table A.

TABLE A
Different mycophenolate based compounds

| | | |
|---|---|---|
| Mycophenolic acid (MPA) | 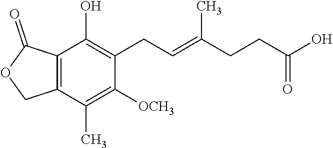 [SMILES: COc1c(C)c2c(c(O)c1C/C=C(\C)CCC(=O)O)C(=O)OC2] | Practical insoluble, 63 mcg/ml. MPA as such is not available in the market, it is available as salt form Hydrophilic nature as compared to its ester prodrug |
| Mycophenolate Mofetil (MMF) | 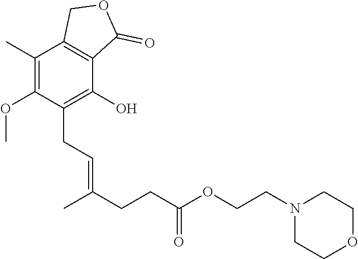 [SMILES: COc1c(C)c2c(c(O)c1C/C=C(\C)CCC(=O)OCCN1CCOCC1)C(=O)OC2] | Good affinity between MMF and corneal epithelium which is rich in esterase enzymes MMF is broken down by esterase then MPA is transported through stroma, endothelium and aqueous humor Prodrug are better suited for corneal delivery |
| Mycophenolate Sodium (MPS) | 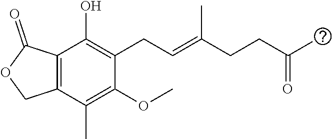 [SMILES: COc1c(C)c2c(c(O)c1C/C=C(\C)CCC(C)=O)C(=O)OC2] | Sodium salt is more soluble form of MPA |
| Mycophenolate Mofetil Hydrochloride | 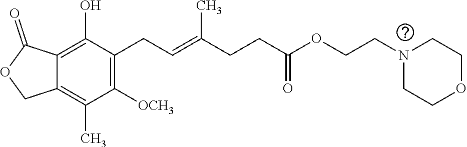 [SMILES: COc1c(C)c2c(c(O)c1C/C=C(\C)CCC(=O)OCCN1CCOCC1)C(=O)OC2] | MMF. HCl is used in injectable formulation It's a soluble fraction of MMF. Should be a preferred choice as both solubility and permeability is achieved using this salt. It avoids the complex formulation technologies. |

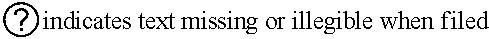

In some embodiments, the salt of the mycophenolic acid can be selected from the group consisting of sodium salt of mycophenolic acid, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, and hyaluronic acid salt of mycophenolate mofetil.

In some embodiments, the active agent can be mycophenolate mofetil or an analog or derivative of mycophenolic acid. In some embodiments, the analog or derivative of mycophenolic acid is one of the following:
(i) Compound 1 having the structure:

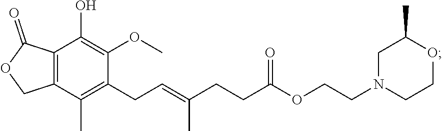 [SMILES: COc1c(O)c2c(c(C)c1C/C=C(\C)CCC(=O)OCCN1CCO[C@H](C)C1)COC2=O]

(ii) Compound 2 having the structure:

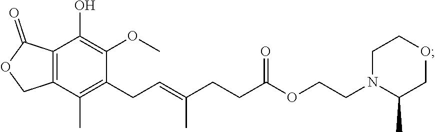 [SMILES: COc1c(O)c2c(c(C)c1C/C=C(\C)CCC(=O)OCCN1CCOC[C@H]1C)COC2=O]

(iii) Compound 3 having the structure:

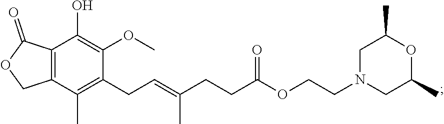 [SMILES: COc1c(O)c2c(c(C)c1C/C=C(\C)CCC(=O)OCCN1C[C@@H](C)O[C@@H](C)C1)COC2=O]

(iv) Compound 4 having the structure:

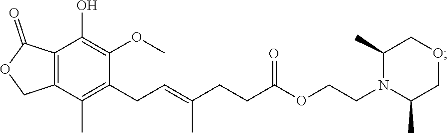 [SMILES: COc1c(O)c2c(c(C)c1C/C=C(\C)CCC(=O)OCCN1[C@H](C)COC[C@@H]1C)COC2=O]

(v) Compound 5 having the structure:

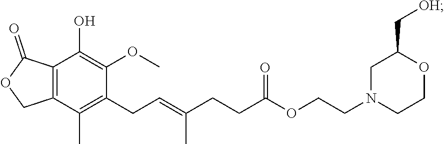 [SMILES: COc1c(O)c2c(c(C)c1C/C=C(\C)CCC(=O)OCCN1CCO[C@H](CO)C1)COC2=O]

(vi) Compound 6 having the structure:

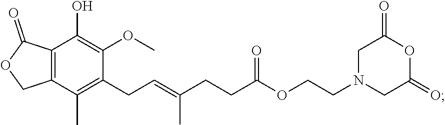 [SMILES: COc1c(O)c2c(c(C)c1C/C=C(\C)CCC(=O)OCCN1CC(=O)OC(=O)C1)COC2=O]

(vii) Compound 7 having the structure:

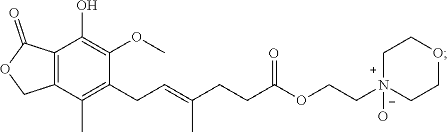 [SMILES: COc1c(O)c2c(c(C)c1C/C=C(\C)CCC(=O)OCC[N+]1([O-])CCOCC1)COC2=O]

(viii) Compound 8 having the structure:

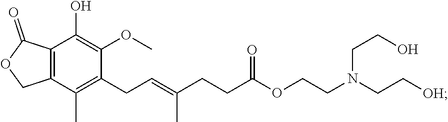 [SMILES: COc1c(O)c2c(c(C)c1C/C=C(\C)CCC(=O)OCCN(CCO)CCO)COC2=O]

or
(ix) Compound 9 having the structure:

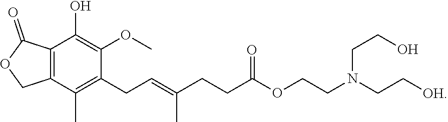 [SMILES: COc1c(O)c2c(c(C)c1C/C=C(\C)CCC(=O)OCCN(CCO)CCO)COC2=O]

In some embodiments, the active agent can be in the form of a prodrug. The term “prodrug” as used herein refers to compounds that can be converted via some chemical or physiological process (e.g., enzymatic processes and metabolic hydrolysis) to compounds described herein. Thus, the term “prodrug” also refers to a precursor of a biologically active compound that is pharmaceutically acceptable. A prodrug (eg., an ester) can be inactive when administered to a subject, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound, as described herein, can be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. For example, a compound comprising a hydroxy group can be administered as an ester that is converted by hydrolysis in vivo to the hydroxy compound. Suitable esters that can be converted in vivo into hydroxy compounds include acetates, citrates, lactates, tartrates, malonates, oxalates, salicylates, propionates, succinates, fumarates, formates, benzoates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the likes. Similarly, a compound comprising an amine group can be administered as an amide, e.g., acetamide, formamide and benzamide that is converted by hydrolysis in vivo to the amine compound. See Harper, “Drug Latentiation” in Jucker, ed. Progress in Drug Research 4:221-294 (1962); Morozowich et al., “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al., “Prodrug approaches to the improved delivery of peptide drug” in Curr. Pharm. Design. 5(4):265-287 (1999); Pauletti et al. (1997), Improvement in peptide bioavailability: Peptidomimetics and Prodrug Strategies, Adv. Drug. Delivery Rev. 27:235-256; Mizen et al. (1998), “The Use of Esters as Prodrugs for Oral Delivery of (3-Lactam antibiotics,” Pharm. Biotech. 11:345-365; Gaignault et al. (1996), “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv, Drug Delivery Rev. 8(1): 1-38 (1992); Fleisher et al., “Improved oral drug delivery: solubility limitations overcome by the use of prodrugs”, Arfv. Drug Delivery Rev. 19(2): 115-130 (1996); Fleisher et al., “Design of prodrugs for improved gastrointestinal absorption by intestinal enzyme targeting”, Methods Enzymol. 112 (Drug Enzyme Targeting, Pt. A): 360-81, (1985); Farquhar et al., “Biologically Reversible Phosphate-Protective Groups”, Pharm. Sci., 72(3): 324-325 (1983); Freeman et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alpha-acyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al., “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), contents of all of which are herein incorporated by reference in their entirety.

In some embodiments, an exemplary prodrug of mycophenolic acid is mycophenolate mofetil (MMF) and pharmaceutically acceptable salts thereof.

Transporter-targeted prodrugs offer several advantages including improving the stability of parent drug molecule, altering the physicochemical properties such as solubility and lipophilicity, improving the pharmacokinetics properties and reducing the systemic side effects, and improving the permeability of drugs as the prodrugs become substrates for the influx transporters and evades the efflux pumps. Accordingly, in some embodiments, the active agent, i.e., mycophenolate is in the form of prodrug with a transporter.

Exemplary transporter molecules include, but are not limited to, peptides, amino acids, nucleosides, glucose, vitamins (e.g., vitamin C), acid/base, and glutathione. Peptide transporters are classified into three types: PepT1, PepT2 (small peptides, β-lactam antibiotics and other peptidomimetic drugs) and peptide/histidine transporters (PHT1 and PHT2), with differences in their substrate specificity, transport capacity and affinity. P-gp appears to be the most sensitive, being affected by many, including Labrasol, Imwitor 742, Acconon E, Softigen767, Cremophor EL, Miglyol, Solutol HS 15, Sucrose monolaurate, Polysorbate 20, TPGS, and Polysorbate 80 in addition to Pluronic P85. Among these, cremophor and pluronic P85 have been used in ophthalmic formulations for topical administration. It is conceivable that if a P-gp substrate, for example, cyclosporine A, is formulated with an excipient that happens to modulate P-gp activity, its ocular bioavailability could be changed.

In some embodiments, the active agent is an ester. For example, the active agent is a mycophenolate amino acid ester.

In some embodiments, the active agent, i.e., mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9, is in the form of prodrug with hydroxy amino derivatives. For example, the prodrug is an amino acid derivative of mycophenolate. In some embodiments, the amino acid is an L amino acid. Amino acid derivatives of mycophenolate include, but are not limited to, L-alanine mycophenolate ester, L-serine mycophenolate ester, L-serinesuccinate mycophenolate ester, and L-cysteine mycophenolate ester. Some additional exemplary, amino acid esters are described in Iwaszkiewicz-Grzes, European Journal of Medicinal Chemistry (2013), vol. 69, pp. 863-871, content of which is incorporated herein by reference in its entirety.

In some embodiments, the active agent is a pharmaceutically acceptable salt. Since the active agent, i.e., mycophenolic acid, contains an acidic functional group, it is capable of forming pharmaceutically-acceptable salts with pharmaceutically-acceptable bases. The term “pharmaceutically-acceptable salts” refers to the relatively non-toxic, inorganic and organic base addition salts of mycophenolate. These salts can be prepared in situ in the administration vehicle, or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the sodium, lithium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al. (1977), “Pharmaceutical Salts”, J. Pharm. Sci. 66:1-19, content of which is incorporated herein by reference in its entirety.

A prodrug of the active agent may contain a basic functional group, such as amino or alkylamino, and is, thus, capable of forming pharmaceutically-acceptable salts with pharmaceutically-acceptable acids. The term “pharmaceutically-acceptable salts” in this respect, refers to the relatively non-toxic, inorganic and organic acid addition salts of the prodrug invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. See, for example, Berge et al. (1977), “Pharmaceutical Salts”, J. Pharm. Sci. 66:1-19. The pharmaceutically acceptable salts of the subject prodrugs include the conventional nontoxic salts or quaternary ammonium salts of the prodrugs, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In some embodiments, the active agent is Mycophenolic Acid. In other embodiments, the active agent is a salt or an ester of Mycophenolic Acid, including, but not limited to, Mycophenolate Mofetil, Mycophenolate Sodium, Mycophenolate Mofetil Hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, and hyaluronic acid salt of mycophenolate mofetil. In some embodiments, the active agent is Compound 1, Compound 2, Compound 3, Compound 4, Compound 5, Compound 6, Compound 7, Compound 8 or Compound 9 as described herein.

The amount of the active agent, e.g., mycophenolic acid or a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in the formulation can be any desired amount. The amounts selected can be based on the amounts required to achieve therapeutically beneficial levels in the eye. For example, the amount in the formulation can be up to 5% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.01% w/v or w/w to about 4.5% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.1% w/v or w/w to about 4.5% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.5% w/v or w/w to about 4.5% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.01% w/v or w/w to about 4.0% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.1% w/v or w/w to about 4.0% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.5% w/v or w/w to about 4.0% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.05% w/v or w/w to about 3.0% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.1% w/v or w/w to about 3.0% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.5% w/v or w/w to about 3.0% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.1% w/v or w/w to about 2.0% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 0.2% w/v or w/w to about 1.0% w/v or w/w. In some embodiments, the formulation can have mycophenolic acid, a salt or an ester thereof such as mycophenolic acid, mycophenolate mofetil, mycophenolate sodium (MPS), mycophenolate hydrochloride, mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, Compound 1, Compound 2, Compound 3, Compound 4, Compound 5, Compound 6, Compound 7, Compound 8 or Compound 9 in an amount from about 2% to about 4% w/v or w/w. In some embodiments, the formulation has levels of the drug mycophenolic acid or its salts or derivatives thereof selected from 0.05, 0.06, 0.08, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0% w/v or w/w. In some embodiments, the amount of the mycophenolic acid, a salt or an ester thereof levels are selected from 0.5, 1.0, 1.5, 2.0, 2.5, 3.0, 3.5, or 4.0% w/v or w/w.

The ophthalmic formulation of the invention comprises, in addition to the active agent, one or more of the following additional components: preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, potassium removing agent, suspending agent, and therapeutic agent.

In some embodiments, the formulation comprises a preservative, for example, to extend shelf life or limit bacterial growth in the formulations during storage as well as when administered therapeutically onto the eye. Preservatives that can be used, include, among others, boric acid, benzalkonium chloride, benzethonium chloride, benzododecinium bromide, cetylpyridinium chloride, chlorobutanol, chelating agents (e.g., ethylenediamine tetracetic acid (EDTA)), thimerosol, phenylmercuric nitrate, phenylmercuric acetate, methylparaben, propylparaben, butylparaben, phenylethyl alcohol, sodium benzoate, sodium propionate, sorbic acid, sodium sorbate, sodium borate, and sodium perborate. The amount of preservative in the formulation can be an amount that enhances the shelf life, limits bacterial growth, or otherwise preserves the formulation, with minimal toxicity to the tissues (see, e.g., The United States Pharmacopeia, 22nd rev., and The National Formulary, 17th ed. Rockville, Md.: The United States Pharmacopeia Convention; pages 1692-3 (1989)). Amount of preservative suitable for use in the formulations can be determined by the person skilled in the art. For example, the amount of the preservative in the formulation can be up to 5% w/v or w/w. In some embodiments, the formulation can comprise a preservative in an amount from about 0.01% w/v or w/w to about 4.5% w/v or w/w. In some embodiments, the formulation can comprise a preservative in an amount from about 0.1% w/v or w/w to about 4.5% w/v or w/w. In some embodiments, the formulation can comprise a preservative in an amount from about 0.5% w/v or w/w to about 4.5% w/v or w/w. In some embodiments, the formulation can comprise a preservative in an amount from about 0.01% w/v or w/w to about 4.0% w/v or w/w. In some embodiments, the formulation can comprise a preservative in an amount from about 0.1% w/v or w/w to about 4.0% w/v or w/w. In some embodiments, the formulation can comprise a preservative in an amount from about 0.5% w/v or w/w to about 4.0% w/v or w/w. In some embodiments, the formulation can comprise a preservative in an amount from about 0.05% w/v or w/w to about 3.0% w/v or w/w. In some embodiments, the formulation can comprise a preservative in an amount from about 0.1% w/v or w/w to about 3.0% w/v or w/w. In some embodiments, the formulation can comprise a preservative in an amount from about 0.5% w/v or w/w to about 3.0% w/v or w/w. In some embodiments, the formulation can comprise a preservative in an amount from about 0.1% w/v or w/w to about 2.0% w/v or w/w. In some embodiments, the formulation can comprise a preservative in an amount from about 0.2% w/v or w/w to about 1.0% w/v or w/w. In some embodiments, the formulation can comprise a preservative in an amount from about 2% to about 4% w/v or w/w. In some embodiments, the formulation can comprise a preservative in an amount of about 0.05, 0.06, 0.08, 0.1, 0.2, 0.5, 1.0, 1.5, 2.0, 2.5, 3.0, 3.5, 4.0, 4.5, or 5.0% w/v or w/w. In some embodiments, the amount of the preservative can be about 0.03, 0.1, 0.11, 0.3, 0.47, or 5% w/v or w/w. In some embodiments, the preservatives can be used at an amount of from about 0.001 to about 1.0% w/v or w/w. For example, the preservative can be present in an amount from about 0.005 to about 0.050% w/v or w/w, 0.005 to about 0.040% w/v or w/w, 0.010 to about 0.030% w/v or w/w, 0.010 to about 0.020% w/v or w/w, or from about 0.010 to about 0.015% w/v or w/w. In some embodiments, the amount of preservative in the formulation can be about 0.005, 0.01, 0.012, 0.014, 0.016, 0.018, 0.020, 0.030, 0.040, or 0.050% w/v or w/w. In some exemplary embodiments, the amount of the preservative in the formulation can be about 0.01, 0.01, 0.02, 0.03, 0.05, 0.05, 0.08, 0.1, 0.11, 0.2, 0.3, 0.47, 1.1, 1.7, 5 or 10% w/v or w/w.

In some embodiments, the formulation comprises a chelating agent. Exemplary chelating agents include, but are not limited to, edetate disodium (EDTA) and edetate sodium. Any desired amount of chelating agent can be included in the formulation. For example, the amount of the chelating agent in the formulation can be up to 5% w/v or w/w. In some embodiments, the formulation can comprise a chelating agent in an amount from about 0.01% w/v or w/w to about 4.5% w/v or w/w. In some embodiments, the formulation can comprise a chelating agent in an amount from about 0.1% w/v or w/w to about 4.5% w/v or w/w. In some embodiments, the formulation can comprise a chelating agent in an amount from about 0.5% w/v or w/w to about 4.5% w/v or w/w. In some embodiments, the formulation can comprise a chelating agent in an amount from about 0.01% w/v or w/w to about 4.0% w/v or w/w. In some embodiments, the formulation can comprise a chelating agent in an amount from about 0.1% w/v or w/w to about 4.0% w/v or w/w. In some embodiments, the formulation can comprise a chelating agent in an amount from about 0.5% w/v or w/w to about 4.0% w/v or w/w. In some embodiments, the formulation can comprise a chelating agent in an amount from about 0.05% w/v or w/w to about 3.0% w/v or w/w. In some embodiments, the formulation can comprise a chelating agent in an amount from about 0.1% w/v or w/w to about 3.0% w/v or w/w. In some embodiments, the formulation can comprise a chelating agent in an amount from about 0.5% w/v or w/w to about 3.0% w/v or w/w. In some embodiments, the formulation can comprise a chelating agent in an amount from about 0.1% w/v or w/w to about 2.0% w/v or w/w. In some embodiments, the formulation can comprise a chelating agent in an amount from about 0.2% w/v or w/w to about 1.0% w/v or w/w. In some embodiments, the formulation can comprise a chelating agent in an amount from about 2% to about 4% w/v or w/w. In some embodiments, the can comprise a chelating agent in an amount of about 0.05, 0.06, 0.08, 0.1, 0.2, 0.5, 1.0, 1.5, 2.0, 2.5, 3.0, 3.5, 4.0, 4.5, or 5.0% w/v or w/w.

In some embodiments, the chelating agent can be present in an amount from about 0.005 to about 0.050% w/v or w/w, 0.005 to about 0.040% w/v or w/w, 0.010 to about 0.030% w/v or w/w, 0.010 to about 0.020% w/or w/w, or from about 0.010 to about 0.015% w/v or w/w. In some embodiments, the chelating agent can be present at 0.005, 0.01, 0.012, 0.014, 0.016, 0.018, 0.020, 0.030, 0.040, or 0.050% w/v or w/w. In some embodiments, the chelating agent is present in an amount of about 0.02, 0.1, 0.13, 0.3, 0.55 w/v or w/w.

In some embodiments, the formulation can comprise one or more buffering agents or pH modifiers for adjusting and/or maintaining the pH of the formulation at a specified pH range. Buffering agents and pH modifiers are usually composed of a weak acid or base and its conjugate salt, where the “buffer capacity” β is defined as the ratio ΔB/ΔpH, where ΔB is the gram equivalent of strong acid/base to change pH of 1 liter of buffer solution, and ΔpH is the pH change caused by the addition of strong acid/base. The relationship between buffer capacity and buffer concentrations can be defined by the following formula:

$$β = 2.3 C \frac{Ka [ {H}_{3} {O}^{+} ]}{{( K a + [ {H}_{3} {O}^{+} ] )}^{2}}$$

where C is the total buffer concentration (i.e., the sum of the molar concentrations of acid and salt). Generally, buffering capacity should be large enough to maintain the product pH for a reasonably long shelf-life but also low enough to allow rapid readjustment of the product to physiologic pH upon administration. Generally, buffer capacities of from about 0.01 to 0.1 can be used for ophthalmic formulations particularly at concentrations that provide sufficient buffering capacity and minimizes adverse effects, e.g., irritation, to the eye. Exemplary buffering agents include, by way of example and not limitation, various salts (e.g., sodium, potassium, etc.), acids or bases, where appropriate, of the following: acetate, borate, phosphate, bicarbonate, carbonate, citrate, tetraborate, biphosphate, tromethamine, hydroxyethyl morpholine, and THAM (trishydroxymethylamino-methane). Some specific exemplary buffering agents or pH modifiers include, but are not limited to, acetic acid, anhydrous citric acid, citric acid monohydrate, hydrochloric acid, phosphoric acid, potassium phosphate monobasic, sodium acetate, sodium acetate anhydrous, sodium carbonate, sodium carbonate monohydrate, sodium hydroxide, sodium phosphate, sodium phosphate (heptahydrate), sodium phosphate dibasic, sodium phosphate dibasic (anhydrous), sodium phosphate dibasic (anhydrous), sodium phosphate dibasic (dihydrate), sodium phosphate dibasic (dodecahydrate), sodium phosphate monobasic, sodium phosphate monobasic (anhydrous), sodium phosphate monobasic (dihydrate), sodium phosphate monobasic (monohydrate), sulfuric acid, trisodium citrate dihydrate and tromethamine.

Amount of buffering agent or pH modifier suitable for use in the formulations can be determined by the person skilled in the art. For example, the amount of the buffering agent or pH modifier in the formulation can be up to 5% w/v or w/w. In some embodiments, the formulation can comprise a buffering agent or pH modifier in an amount from about 0.01% w/v or w/w to about 4.5% w/v or w/w. In some embodiments, the formulation can comprise a buffering agent or pH modifier in an amount from about 0.1% w/v or w/w to about 4.5% w/v or w/w. In some embodiments, the formulation can comprise a buffering agent or pH modifier in an amount from about 0.5% w/v or w/w to about 4.5% w/v or w/w. In some embodiments, the formulation can comprise a buffering agent or pH modifier in an amount from about 0.01% w/v or w/w to about 4.0% w/v or w/w. In some embodiments, the formulation can comprise a buffering agent or pH modifier in an amount from about 0.1% w/v or w/w to about 4.0% w/v or w/w. In some embodiments, the formulation can comprise a buffering agent or pH modifier in an amount from about 0.5% w/v or w/w to about 4.0% w/v or w/w. In some embodiments, the formulation can comprise a buffering agent or pH modifier in an amount from about 0.05% w/v or w/w to about 3.0% w/v or w/w. In some embodiments, the formulation can comprise a buffering agent or pH modifier in an amount from about 0.1% w/v or w/w to about 3.0% w/v or w/w. In some embodiments, the formulation can comprise a buffering agent or pH modifier in an amount from about 0.5% w/v or w/w to about 3.0% w/v or w/w. In some embodiments, the formulation can comprise a buffering agent or pH modifier in an amount from about 0.1% w/v or w/w to about 2.0% w/v or w/w. In some embodiments, the formulation can comprise a buffering agent or pH modifier in an amount from about 0.2% w/v or w/w to about 1.0% w/v or w/w. In some embodiments, the formulation can comprise a buffering agent or pH modifier in an amount from about 2% to about 4% w/v or w/w. In some embodiments, the formulation can comprise a buffering agent or pH modifier in an amount of about 0.05, 0.06, 0.08, 0.1, 0.2, 0.5, 1.0, 1.5, 2.0, 2.5, 3.0, 3.5, 4.0, 4.5, or 5.0% w/v or w/w. In some embodiments, the amount of the buffering agent or pH modifier can be about 0.03, 0.1, 0.11, 0.3, 0.47, or 5% w/v or w/w. In some embodiments, the buffering agent or pH modifiers can be used at an amount of from about 0.001 to about 1.0% w/v or w/w. For example, the buffering agent or pH modifier can be present in an amount from about 0.005 to about 0.050% w/v or w/w, 0.005 to about 0.040% w/v or w/w, 0.010 to about 0.030% w/v or w/w, 0.010 to about 0.020% w/v or w/w, or from about 0.010 to about 0.015% w/v or w/w. In some embodiments, the amount of buffering agent or pH modifier in the formulation can be about 0.01, 0.02, 0.03, 0.04, 0.05, 0.09, 0.2, 0.25, 0.3, 0.31, 0.39, 0.4, 0.43, 0.44, 0.45, 0.48, 0.51, 0.59, 0.59, 0.65, 0.78, 0.79, 0.81, 1.15, 1.18, 1.2, 1.24, 1.3, 1.4, 1.67 or 1.9% w/v or w/w.

The pH of formulation can be within 1.0 to 1.5 pH units from physiological pH, particularly the physiological pH in the external environment of the eye. The pH of human tears is approximately pH 7.4. Hence, the pH of the formulation can be about 1.0 to 1.5 pH units above or below pH 7.4. In some embodiments, the pH of the formulation is from about pH 6.0 to about pH 8.5. In some embodiments, the pH of the formulation is from about pH 5.0 to about pH 8.0. In some embodiments, the pH of the formulation is from about 6.5 to about 8.0. In some embodiments, the pH of the formulation is from about 7.0 to about 8.0. In some embodiments, the pH of the formulation is from about 7.0 to about 7.5. In some embodiments, the pH of the ophthalmic formulation is about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0. A person of skill in the art can select a pH that balances the stability and efficacy of the formulation at the indicated pH and the tolerability of the eye to differences in pH from the natural condition.

In some embodiments, the formulation can include one or more thickening or viscosity enhancing agents. The thickening or viscosity enhancing agent typically enhances the viscosity of the formulation to increase retention time of the solution on the eye, and in some instances, to provide a protective layer on the eye surface. Viscosity enhancing agents include, among others, carbopol gels, dextran 40 (molecular weight of 40,000 Daltons), dextran 70 (molecular weight of 70,000 Daltons), gelatin, glycerin, carboxymethycellulose (CMC), hydroxyethyl cellulose, hydroxypropyl methylcellulose, (HPMC) methylcellulose, ethylcellulose, polyethylene glycol, poloxamer 407, polysorbate 80, propylene glycol, polyvinyl alcohol, and polyvinylpyrrolodine (povidone), in various molecular weights and in various compatible combinations. Viscosity of a solution is given in poise units, with a viscosity between about 25 and 50 cps being suitable for ophthalmic formulations. The amount of agent for use in the formulations can be determined by one of skill in the art, and can provide residence times in the eye of 15 min or more, 30 min or more, 1 hour or more, 2 hours or more, 3 hours or more, 4 hours or more, 6 hours or more, 8 hours or more, 12 hours or more as would be suitable for the condition being treated and the desired retention time of the solution on the eye. Some specific exemplary thickening agents include, but are not limited to, carbomer 940, carbomer copolymer Type A (allyl pentaerythritol crosslinked), carbomer copolymer Type B (allyl pentaerythritol crosslinked), carbomer homopolymer Type B (allyl pentaerythritol crosslinked), carbomer homopolymer Type B (allyl sucrose crosslinked), carboxymethyl cellulose sodium, Crospovidone, dextran, guar gum, hydroxethyl cellulose, hydroxyethyl cellulose, hydroxymethyl cellulose, hydroxymethyl cellulose (2000 MPA·S at 1%), hydroxymethyl cellulose (4000 MPA·S at 1%), hypromellose 2906 (4000 MPA·S), hypromellose 2910 (15000 MPA·S), hypromellose 2910 (3 MPA·S), hypromellose 2910 (5 MPA·S), hypromellose, methyl cellulose, methylcellulose, polycarbophil, polyvinyl alcohol, povidone K30, povidone K90, povidones and xanthan gum.

In some embodiments, the viscosity modifier is a polymer. Exemplary polymeric viscosity modifiers include, but are not limited to, carbopol, hydroxypropyl cellulose, hydroxypropyl methyl cellulose, hydroxyethyl cellulose, and sodium hyaluronate. In some other embodiments, the viscosity modifier is a non-polymeric viscosity modifier or gelling agent.

Amount of thickening agent or viscosity enhancer suitable for use in the formulations can be determined by the person skilled in the art. For example, the amount of the thickening agent or viscosity enhancer in the formulation can be up to 5% w/v or w/w. In some embodiments, the formulation can comprise a thickening agent or viscosity enhancer in an amount from about 0.01% w/v or w/w to about 4.5% w/v or w/w. In some embodiments, the formulation can comprise a thickening agent or viscosity enhancer in an amount from about 0.1% w/v or w/w to about 4.5% w/v or w/w. In some embodiments, the formulation can comprise a thickening agent or viscosity enhancer in an amount from about 0.5% w/v or w/w to about 4.5% w/v or w/w. In some embodiments, the formulation can comprise a thickening agent or viscosity enhancer in an amount from about 0.01% w/v or w/w to about 4.0% w/v or w/w. In some embodiments, the formulation can comprise a thickening agent or viscosity enhancer in an amount from about 0.1% w/v or w/w to about 4.0% w/v or w/w. In some embodiments, the formulation can comprise a thickening agent or viscosity enhancer in an amount from about 0.5% w/v or w/w to about 4.0% w/v or w/w. In some embodiments, the formulation can comprise a thickening agent or viscosity enhancer in an amount from about 0.05% w/v or w/w to about 3.0% w/v or w/w. In some embodiments, the formulation can comprise a thickening agent or viscosity enhancer in an amount from about 0.1% w/v or w/w to about 3.0% w/v or w/w. In some embodiments, the formulation can comprise a thickening agent or viscosity enhancer in an amount from about 0.5% w/v or w/w to about 3.0% w/v or w/w. In some embodiments, the formulation can comprise a thickening agent or viscosity enhancer in an amount from about 0.1% w/v or w/w to about 2.0% w/v or w/w. In some embodiments, the formulation can comprise a thickening agent or viscosity enhancer in an amount from about 0.2% w/v or w/w to about 1.0% w/v or w/w. In some embodiments, the formulation can comprise a thickening agent or viscosity enhancer in an amount from about 2% to about 4% w/v or w/w. In some embodiments, the formulation can comprise a thickening agent or viscosity enhancer in an amount of about 0.05, 0.06, 0.08, 0.1, 0.2, 0.5, 1.0, 1.5, 2.0, 2.5, 3.0, 3.5, 4.0, 4.5, or 5.0% w/v or w/w. In some embodiments, the amount of the thickening agent or viscosity enhancer can be about 0.03, 0.1, 0.11, 0.3, 0.47, or 5% w/v or w/w. In some embodiments, the buffering agent or pH modifiers can be used at an amount of from about 0.001 to about 1.0% w/v or w/w. For example, the thickening agent or viscosity enhancer can be present in an amount from about 0.005 to about 0.050% w/v or w/w, 0.005 to about 0.040% w/v or w/w, 0.010 to about 0.030% w/v or w/w, 0.010 to about 0.020% w/v or w/w, or from about 0.010 to about 0.015% w/v or w/w. In some embodiments, the amount of thickening agent or viscosity enhancer in the formulation can be about 0.05, 0.05, 0.1, 0.16, 0.2, 0.25, 0.35, 0.45, 0.48, 0.48, 0.5, 0.5, 0.5, 0.6, 0.6, 0.86, 0.9, 1.2, 1.4, 1.4, 1.6, 1.8, 1.8, 2, 2.5, 3, 3.75 or 4% w/v or w/w.

In some embodiments, the formulation can include one or more antioxidants. Exemplary antioxidants include, but are not limited to, alpha-tocopherol, EDTA (e.g., disodium EDTA), sodium bisulfite, sodium bisulfite, sodium metabisulfite, sodium metabisulfite, sodium sulfate (anhydrous), sodium thiosulfate, sodium thiosulfate, sodium thiosulfite, sulfate, thimerosal and thiourea. Amount of antioxidant suitable for use in the formulations can be determined by the person skilled in the art. For example, the amount of the antioxidant in the formulation can be up to 5% w/v or w/w. In some embodiments, the formulation can comprise an antioxidant in an amount from about 0.01% w/v or w/w to about 4.5% w/v or w/w. In some embodiments, the formulation can comprise an antioxidant in an amount from about 0.1% w/v or w/w to about 4.5% w/v or w/w. In some embodiments, the formulation can comprise an antioxidant in an amount from about 0.5% w/v or w/w to about 4.5% w/v or w/w. In some embodiments, the formulation can comprise an antioxidant in an amount from about 0.01% w/v or w/w to about 4.0% w/v or w/w. In some embodiments, the formulation can comprise an antioxidant in an amount from about 0.1% w/v or w/w to about 4.0% w/v or w/w. In some embodiments, the formulation can comprise an antioxidant in an amount from about 0.5% w/v or w/w to about 4.0% w/v or w/w. In some embodiments, the formulation can comprise an antioxidant in an amount from about 0.05% w/v or w/w to about 3.0% w/v or w/w. In some embodiments, the formulation can comprise an antioxidant in an amount from about 0.1% w/v or w/w to about 3.0% w/v or w/w. In some embodiments, the formulation can comprise an antioxidant in an amount from about 0.5% w/v or w/w to about 3.0% w/v or w/w. In some embodiments, the formulation can comprise an antioxidant in an amount from about 0.1% w/v or w/w to about 2.0% w/v or w/w. In some embodiments, the formulation can comprise an antioxidant in an amount from about 0.2% w/v or w/w to about 1.0% w/v or w/w. In some embodiments, the formulation can comprise an antioxidant in an amount from about 2% to about 4% w/v or w/w. In some embodiments, the formulation can comprise an antioxidant in an amount of about 0.05, 0.06, 0.08, 0.1, 0.2, 0.5, 1.0, 1.5, 2.0, 2.5, 3.0, 3.5, 4.0, 4.5, or 5.0% w/v or w/w. In some embodiments, the amount of the antioxidant can be about 0.03, 0.1, 0.11, 0.3, 0.47, or 5% w/v or w/w. In some embodiments, the antioxidant can be used at an amount of from about 0.001 to about 1.0% w/v or w/w. For example, the antioxidant can be present in an amount from about 0.005 to about 0.050% w/v or w/w, 0.005 to about 0.040% w/v or w/w, 0.010 to about 0.030% w/v or w/w, 0.010 to about 0.020% w/v or w/w, or from about 0.010 to about 0.015% w/v or w/w. In some embodiments, the amount of antioxidant in the formulation can be about 0.01, 0.1, 0.17, 0.2, 0.32, 0.34, 0.5, 1.2 or 3.15% w/v or w/w.

In some embodiments, the formulation can include one or more tonicity modifiers, which can be used to adjust the tonicity of the composition, for example, to the tonicity of natural tears. Suitable tonicity modifiers include, but are not limited to, dextrans (e.g., dextran 40 or 70), dextrose, glycerin, potassium chloride, propylene glycol, and sodium chloride. Equivalent amounts of one or more salts made up of cations, for example, such as potassium, ammonium and anions such as chloride, citrate, ascorbate, borate, phosphate, bicarbonate, sulfate, thiosulfate, bisulfate; the salts sodium bisulfate and ammonium sulfate can also be used. Some specific exemplary tonicity modifiers include, but are not limited to, calcium chloride, magnesium chloride, mannitol, potassium chloride and sodium chloride.

The amount of tonicity modifier will vary, depending on the particular modifier to be added. In general, however, the formulations will have a tonicity modifier in an amount sufficient to cause the final composition to have an ophthalmically acceptable osmolarity, for example, about 250 to about 450 mOsM/L, or about 250 to about 350 mOsM/L. Amount of tonicity modifier suitable for use in the formulations can be determined by the person skilled in the art. For example, the amount of the tonicity modifier in the formulation can be up to 5% w/v or w/w. In some embodiments, the formulation can comprise a tonicity modifier in an amount from about 0.01% w/v or w/w to about 4.5% w/v or w/w. In some embodiments, the formulation can comprise a tonicity modifier in an amount from about 0.1% w/v or w/w to about 4.5% w/v or w/w. In some embodiments, the formulation can comprise a tonicity modifier in an amount from about 0.5% w/v or w/w to about 4.5% w/v or w/w. In some embodiments, the formulation can comprise a tonicity modifier in an amount from about 0.01% w/v or w/w to about 4.0% w/v or w/w. In some embodiments, the formulation can comprise a tonicity modifier in an amount from about 0.1% w/v or w/w to about 4.0% w/v or w/w. In some embodiments, the formulation can comprise a tonicity modifier in an amount from about 0.5% w/v or w/w to about 4.0% w/v or w/w. In some embodiments, the formulation can comprise a tonicity modifier in an amount from about 0.05% w/v or w/w to about 3.0% w/v or w/w. In some embodiments, the formulation can comprise a tonicity modifier in an amount from about 0.1% w/v or w/w to about 3.0% w/v or w/w. In some embodiments, the formulation can comprise a tonicity modifier in an amount from about 0.5% w/v or w/w to about 3.0% w/v or w/w. In some embodiments, the formulation can comprise a tonicity modifier in an amount from about 0.1% w/v or w/w to about 2.0% w/v or w/w. In some embodiments, the formulation can comprise a tonicity modifier in an amount from about 0.2% w/v or w/w to about 1.0% w/v or w/w. In some embodiments, the formulation can comprise a tonicity modifier in an amount from about 2% to about 4% w/v or w/w. In some embodiments, the formulation can comprise a tonicity modifier in an amount of about 0.05, 0.06, 0.08, 0.1, 0.2, 0.5, 1.0, 1.5, 2.0, 2.5, 3.0, 3.5, 4.0, 4.5, or 5.0% w/v or w/w. In some embodiments, the amount of the tonicity modifier can be about 0.03, 0.1, 0.11, 0.3, 0.47, or 5% w/v or w/w. In some embodiments, the buffering agent or pH modifiers can be used at an amount of from about 0.001 to about 1.0% w/v or w/w. For example, the tonicity modifier can be present in an amount from about 0.005 to about 0.050% w/v or w/w, 0.005 to about 0.040% w/v or w/w, 0.010 to about 0.030% w/v or w/w, 0.010 to about 0.020% w/v or w/w, or from about 0.010 to about 0.015% w/v or w/w. In some embodiments, the amount of tonicity modifier in the formulation can be about 0.01, 0.03, 0.05, 0.08, 0.22, 0.85, 0.9, 1.2, 3.3, 4.7, or 5% w/v or w/w.

In some embodiments, the formulation can include one or more humectants. Examples of suitable humectants include, but are not limited to, glycerin, hyaluronic acid, sorbitol, urea, alpha hydroxy acids, sugars, lactic acid, polyethylene glycol, propylene glycol, glyceryl triacetate, lithium chloride, polyols like sorbitol, xylitol and maltitol, polymeric polyols like polydextrose, natural extracts like quillaia, hexadecyl, myristyl, isodecyl and isopropyl esters of adipic, lactic, oleic, stearic, isostearic, myristic and linoleic acids, as well as many of their corresponding alcohol esters (e.g., sodium isostearoyl-2-lactylate, sodium capryl lactylate), hydrolyzed protein and other collagen-derived proteins, aloe vera gel, acetamide monoethanolamide (MEA), sodium pyrrolidone carboxylic acid, lactic acid, urea, L-proline, guanidine and pyrrolidone, acetamide MEA, acetamido propyl trimmonium chloride, calcium stearoyl lactylate, chitosan pyrrolidone carboxylic acid (PCA), diglycerol lactate, ethyl ester of hydrolyzed silk, fatty quaternary amine chloride complex, glycereth-7, glycereth-12, glycereth-26, glycereth-4.5 lactate, glycerin, diglycerin, polyglycerin, hydrolyzed fibronectin, lactamide MEA, lactamide N-(2-hydroxyetheryl), mannitol, methyl gluceth-10, methyl gluceth-20, methylsilanol PCA, panthenol, PCA, polyethylene glycol (PEG), PEG-4, PEG-8, polyamino sugar condensate, quaternium-22, sea salts, sodium caproyl lactylate, sodium hyaluronate, sodium isostearoyl lactylate, sodium lactate, sodiumlauroyl lactylate, sodium PCA, sodium polyglutamate, sodium stearoyl lactylate, soluble collagen, sorbitan laurate, sorbitan oleate, sorbitansesquiisostearate, sorbitan stearate, sorbitol, sphingolipids, TEA-PCA, ethylene glycol, diethylene glycol, triethylene glycol, and other polyethylene glycols, propylene glycol, dipropylene glycol and other propylene glycols, 1,3-butylene glycol, 1,4-butylene glycol and other butylene glycols, glycerol, diglycerol and other polyglycerols, mannitol, xylitol, maltitol and other sugar alcohols, glycerol ethylene oxide (EO) and propylene oxide (PO) adducts, sugar alcohol EO and PO adducts, adducts of EO or PO and monosaccharides such as galactose and fructose, adducts of EO or PO and polysaccharides such as maltose and lactose, sodium pyrrolidone carboxylate, polyoxyethylene methyl glycoside, polyethylene glycol, propylene glycol, dipropylene glycol, 1,3-butylene glycol, hexylene glycol, xylitol, maltitol, maltose, D-mannitol, gluten, glucose, fructose, lactose, sodium chondroitin sulfate, sodium hyaluronate, sodium adenosine phosphate, gallates, pyrrolidone carbonates, glucosamine, cyclodextrin, alpha hydroxy acids, and 2-methyl-1,3-propane diol. In some embodiments, humectant is glycerin or polyethylene glycol. The amount of humectant will vary, depending on the particular humectant to be added. In general, humectant be present in an amount from about 1 to about 15% w/v or w/w. In some embodiments, humectant is present in an amount of about 4.4 or 8.8% w/v or w/w.

In some embodiments, the formulation can include one or more surfactants. Any suitable surfactant or combination of surfactants can be used. The surfactant can be any one of a non-ionic surfactant, an amphoteric surfactant, an anionic surfactant, or a cationic surfactant. Exemplary surfactants include, but are not limited to, polyoxyethylene (hereinafter also referred to as “POE”)-polyoxypropylene (hereinafter also referred to as “POP”) block copolymer (poloxamer 407, poloxamer 235, poloxamer 188, etc.), ethylenediamine POE-POP Block copolymer adducts (such as poloxamine), POE sorbitan fatty acid esters (such as polysorbate 20, polysorbate 60, polysorbate 65, polysorbate 80 (TO-10, etc.)), POE hydrogenated castor oil (POE (5) hydrogenated castor oil, POE (10) Hardened castor oil, POE (20) hardened castor oil, POE (40) hardened castor oil, POE (50) hardened castor oil, POE (60) hardened castor oil (HCO-60, etc.), POE (100) hardened castor oil Etc.), POE castor oil (POE (3) castor) Oil, POE (10) castor oil, POE (35) castor oil, POE (40) castor oil, etc.), POE alkyl ether (polyoxyethylene (9) lauryl ether, polyoxyethylene (20) polyoxypropylene (4), nonionic surfactants such as cetyl ether, POE alkylphenyl ether (POE (10) nonylphenyl ether, etc.), polyoxyl stearate (polyoxyl 40 stearate), polyoxydiethyl castor oil (Cremophor); glycine type Amphoteric surfactants such as amphoteric surfactants (e.g., alkyldiaminoethylglycine, alkylpolyaminoethylglycine, or salts thereof, and betaine-type amphoteric surfactants (e.g., lauryldimethylaminoacetic acid betaine, imidazolinium betaine); cations such as quaternary ammonium salts or alkyl tertiary ammonium salts (for example, benzalkonium chloride, benzethonium chloride, polydronium chloride, biguanide compounds (specifically, polyhexamethylene biguanide hydrochloride or its hydrochloride)) Surfactant: Anionic surfactants such as sodium alkylbenzene sulfonate, etc. In the compounds listed above, each of the numbers in the parentheses shows the number of moles of the added compounds. In some embodiments, surfactant is poloxamer 407 or Tyloxapol (a nonionic liquid polymer of alkyl aryl polyether alcohol).

The amount of the surfactant will vary, depending on the particular surfactant or combination of surfactants to be added. For example, the total amount of the surfactant in the formulation can be up to 5% w/v or w/w. In some embodiments, the formulation can comprise total surfactant in an amount from about 0.01% w/v or w/w to about 4.5% w/v or w/w. In some embodiments, the formulation can comprise total surfactant in an amount from about 0.1% w/v or w/w to about 4.5% w/v or w/w. In some embodiments, the formulation can comprise total surfactant in an amount from about 0.5% w/v or w/w to about 4.5% w/v or w/w. In some embodiments, the formulation can comprise total surfactant in an amount from about 0.01% w/v or w/w to about 4.0% w/v or w/w. In some embodiments, the formulation can comprise total surfactant in an amount from about 0.1% w/v or w/w to about 4.0% w/v or w/w. In some embodiments, the formulation can comprise total surfactant in an amount from about 0.5% w/v or w/w to about 4.0% w/v or w/w. In some embodiments, the formulation can comprise total surfactant in an amount from about 0.05% w/v or w/w to about 3.0% w/v or w/w. In some embodiments, the formulation can comprise total surfactant in an amount from about 0.1% w/v or w/w to about 3.0% w/v or w/w. In some embodiments, the formulation can comprise total surfactant in an amount from about 0.5% w/v or w/w to about 3.0% w/v or w/w. In some embodiments, the formulation can comprise total surfactant in an amount from about 0.1% w/v or w/w to about 2.0% w/v or w/w. In some embodiments, the formulation can comprise total surfactant in an amount from about 0.2% w/v or w/w to about 1.0% w/v or w/w. In some embodiments, the formulation can comprise total surfactant in an amount from about 2% to about 4% w/v or w/w. In some embodiments, the formulation can comprise total surfactant in an amount of about 0.05, 0.06, 0.08, 0.1, 0.2, 0.5, 1.0, 1.5, 2.0, 2.5, 3.0, 3.5, 4.0, 4.5, or 5.0% w/v or w/w. In some embodiments, the surfactant present in the ophthalmic formulation is between about 0.01 to about 0.5% (w/v or w/w). In another embodiment, the surfactant is present from about 0.01 to about 0.1% (w/v or w/w). In another embodiment, the surfactant is present from about 0.01 to about 0.05% (w/v or w/w)). In some embodiments, the surfactant is present in an amount of about 0.05 or 2% w/v or w/w.

In some embodiments, the formulation is substantially free of benzalkonium chloride.

In some embodiments, the formulation can include one or more emulsifiers. The emulsifier can be selected from the group consisting of a silicone-based emulsifier, a polyethylene glycol emulsifier, a polysiloxane emulsifier, a glycoside emulsifier, an acrylic-based emulsifier and combinations thereof. The emulsifier can comprise polysorbate, carbomer and/or castor oil. Exemplary emulsifiers include, but are not limited to, ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium docusate, cholesterol, cholesterol esters, taurocholic acid, phosphatidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like. In some embodiments, emulsifier can be selected from the group consisting of castor oil, cetyl alcohol, glyceryl monostearate, nonoxynol-9, octoxynol-40, poloxamer 188, poloxamer 407, polyethylene glycol 400, polyethylene glycol 8000, polyoxyl 35 castor oil, polyoxyl 40 hydrogenated castor oil, polyoxyl 15 hydroxystearate, polyoxyl 40 stearate, polysorbate 20, polysorbate 80, and tyloxapol.

Additional suitable emulsifying agents which can be used include, but are not limited to anionic (TEA/K stearate (triethanolamine/potassium stearate), sodium lauryl stearate, sodium cetearyl sulfate, and beeswax/Borax), nonionic (glycerol di-stearate, PEG (polyethyleneglycol)-100 Stearate, Polysorbate 20, steareth 2 and steareth 20), and cationic (distearyldimethylammonium chloride, behenalkonium chloride and steapyrium chloride), polymeric (acrylates/C 10-30 alkyl acrylate crosspolymer, polyacrylamide, polyquaternium-37, propylene glycol, dicaprylate/dicaparate and PPG-1 Trideceth-6), and silicone based materials (alkyl modified dimethiconecopolyols), and polyglyceryl esters, and ethoxylated di-fatty esters. Additional suitable emulsifiers/surfactant can include one or more of ionic polysorbate surfactant, Tween® 20, Tween® 40, Tween® 60, Tween® 80, Nonylphenol Polyethylene Glycol Ethers, (alkylphenol-hydroxypolyoxyethylene), Poly(oxy-1,2-ethanediyl), alpha-(4-nonylphenol)-omega-hydroxy-, branched (i.e. Tergitol® NP-40 Surfactant), Nonylphenol Polyethylene Glycol Ether mixtures (i.e. Tergitol® NP-70 (70% AQ) Surfactant), phenoxypolyethoxyethanols and polymers thereof such as Triton®, Poloxamer®, Spans®, Tyloxapol®, different grades of Brij, sodium dodecyl sulfate and the like.

The amount of the emulsifier will vary, depending on the emulsifier to be added. For example, the total amount of the emulsifier in the formulation can be up to 10% w/v or w/w or up to 30% w/v or w/w. In some embodiments, the formulation can comprise an emulsifier in an amount from about 0.01% w/v or w/w to about 4.5% w/v or w/w. In some embodiments, the formulation can comprise an emulsifier in an amount from about 0.1% w/v or w/w to about 4.5% w/v or w/w. In some embodiments, the formulation can comprise an emulsifier in an amount from about 0.5% w/v or w/w to about 4.5% w/v or w/w. In some embodiments, the formulation can comprise an emulsifier in an amount from about 0.01% w/v or w/w to about 4.0% w/v or w/w. In some embodiments, the formulation can comprise an emulsifier in an amount from about 0.1% w/v or w/w to about 4.0% w/v or w/w. In some embodiments, the formulation can comprise an emulsifier in an amount from about 0.5% w/v or w/w to about 4.0% w/v or w/w. In some embodiments, the formulation can comprise an emulsifier in an amount from about 0.05% w/v or w/w to about 3.0% w/v or w/w. In some embodiments, the formulation can comprise an emulsifier in an amount from about 0.1% w/v or w/w to about 3.0% w/v or w/w. In some embodiments, the formulation can comprise an emulsifier in an amount from about 0.5% w/v or w/w to about 3.0% w/v or w/w. In some embodiments, the formulation can comprise an emulsifier in an amount from about 0.1% w/v or w/w to about 2.0% w/v or w/w. In some embodiments, the formulation can comprise an emulsifier in an amount from about 0.2% w/v or w/w to about 1.0% w/v or w/w. In some embodiments, the formulation can comprise an emulsifier in an amount from about 2% to about 4% w/v or w/w. In some embodiments, the formulation can comprise an emulsifier in an amount of about 0.05, 0.1, 0.13, 0.2, 0.25, 0.3, 0.5, 1, 2, 4, 5 or 7% w/v or w/w.

The formulation can also include solvents and co-solvents such as alcohols, glycerin, propylene glycol, propylene glycol diacetate, polypropylene glycol, and sorbitol. The amount of the solvent can vary. For example, the amount of the solvent in the formulation can be up to 20 w/v or w/w, either alone or in combination. In some embodiments, the formulation can comprise a solvent in an amount from about 0.01% w/v or w/w to about 4.5% w/v or w/w. In some embodiments, the formulation can comprise a solvent in an amount from about 0.1% w/v or w/w to about 4.5% w/v or w/w. In some embodiments, the formulation can comprise a solvent in an amount from about 0.5% w/v or w/w to about 4.5% w/v or w/w. In some embodiments, the formulation can comprise a solvent in an amount from about 0.01% w/v or w/w to about 4.0% w/v or w/w. In some embodiments, the formulation can comprise a solvent in an amount from about 0.1% w/v or w/w to about 4.0% w/v or w/w. In some embodiments, the formulation can comprise a solvent in an amount from about 0.5% w/v or w/w to about 4.0% w/v or w/w. In some embodiments, the formulation can comprise a solvent in an amount from about 0.05% w/v or w/w to about 3.0% w/v or w/w. In some embodiments, the formulation can comprise a solvent in an amount from about 0.1% w/v or w/w to about 3.0% w/v or w/w. In some embodiments, the formulation can comprise a solvent in an amount from about 0.5% w/v or w/w to about 3.0% w/v or w/w. In some embodiments, the formulation can comprise a solvent in an amount from about 0.1% w/v or w/w to about 2.0% w/v or w/w. In some embodiments, the formulation can comprise a solvent in an amount from about 0.2% w/v or w/w to about 1.0% w/v or w/w. In some embodiments, the formulation can comprise a solvent in an amount from about 2% to about 4% w/v or w/w. In some embodiments, the formulation can comprise a solvent in an amount of about 0.05, 0.1, 0.13, 0.2, 0.25, 0.3, 0.5, 1, 2, 4, 5 or 7% w/v or w/w.

Without wishing to be bound by a theory, lipophilic co-solvents have good corneal permeability. Thus, in some embodiments, the formulation comprises one or more lipophilic cosolvents.

In some embodiments, the formulation can include a targeting agent. Exemplary targeting agents include, but are not limited to, didodecyldimethylammonium bromide, stearylamine, and N-[1-(2,3-dioleoyloxy)propyl]-N,N,N-trimethylammonium chloride. The amount of the targeting agent can vary. For example, the amount of the targeting agent in the formulation can be up to 10% w/v or w/w. In some embodiments, the formulation can comprise a targeting agent in an amount from about 0.01% w/v or w/w to about 4.5% w/v or w/w. In some embodiments, the formulation can comprise a targeting agent in an amount from about 0.1% w/v or w/w to about 4.5% w/v or w/w. In some embodiments, the formulation can comprise a targeting agent in an amount from about 0.5% w/v or w/w to about 4.5% w/v or w/w. In some embodiments, the formulation can comprise a targeting agent in an amount from about 0.01% w/v or w/w to about 4.0% w/v or w/w. In some embodiments, the formulation can comprise a targeting agent in an amount from about 0.1% w/v or w/w to about 4.0% w/v or w/w. In some embodiments, the formulation can comprise a targeting agent in an amount from about 0.5% w/v or w/w to about 4.0% w/v or w/w. In some embodiments, the formulation can comprise a targeting agent in an amount from about 0.05% w/v or w/w to about 3.0% w/v or w/w. In some embodiments, the formulation can comprise a targeting agent in an amount from about 0.1% w/v or w/w to about 3.0% w/v or w/w. In some embodiments, the formulation can comprise a targeting agent in an amount from about 0.5% w/v or w/w to about 3.0% w/v or w/w. In some embodiments, the formulation can comprise a targeting agent in an amount from about 0.1% w/v or w/w to about 2.0% w/v or w/w. In some embodiments, the formulation can comprise a targeting agent in an amount from about 0.2% w/v or w/w to about 1.0% w/v or w/w. In some embodiments, the formulation can comprise a targeting agent in an amount from about 2% to about 4% w/v or w/w.

In some embodiments, the formulation can comprise a polymer. Exemplary polymers include, but are not limited, poly (lactide), poly (lactideglycolide), poly (glycolide), poly (caprolactone), poly (amides), poly (anhydrides), poly (amino acids), poly (esters), poly (cyanoacrylates), poly (phosphazines), poly (phosphoesters), poly (esteramides), poly (dioxanones), poly (acetals), poly (cetals), poly (carbonates), poly (orthocarbonates), degradable poly (urethanes), chitins, chitosans, poly (hydroxybutyrates), poly (hydroxyvalerates), poly (maleic acid), poly (alkylene oxalates), poly (alkylene succinates), poly (hydroxybutyrates-co-hydroxyvalerates), and copolymers, terpolymers, oxidised cellulose, or combinations or mixtures of these materials. Some polymers that prove to be especially interesting are poly (e-caprolactone) (PCL; for example, poly (E-caprolactone) 65 Kd\ddot{ }Sigma Aldrich); methacrylate acid copolymers and methacrylate or acrylic esters (e.g. EUDRAGITSID); poly (alkyl methacrylate); poly (methyl methacrylate) (e.g. PMM).

In some embodiments, the formulation is in form of an in-situ gel forming formulation. In situ gel-forming systems can be described as low viscosity solutions that undergo phase transition in the conjunctival cul-de-sac to form viscoelastic gels due to conformational changes of polymers in response to the physiological environment. Generally, such formulations comprise one of more of poloxamers, carbopols, pluronics, celluloses (e.g., methyl cellulose), Gelrite gellan gum, pluronic-g-poly(acrylic acid), cellulose acetate phthalate latex, cross-linked polyacrylic derivatives of carbomers, Gelrite sodium alginate and hydroxypropylmethyl cellulose.

In some embodiments, the formulation can comprise one or more wetting agents. Generally, wetting agents can hydrate and limit drying of the eye. Wetting agents generally are hydrophilic polymers, including, by way of example and not limitation, polysorbate 20 and 80, poloxamer 282, and tyloxapol. In some embodiments, wetting agents also include, among others, cellulose based polymers, such as HPMC and CMC; polyvinylpyrrolidone; and polyvinyl alcohol. The amount of the wetting agent can vary. For example, the amount of the wetting agent in the formulation can be up to 10% w/v or w/w. In some embodiments, the formulation can comprise a wetting agent in an amount from about 0.01% w/v or w/w to about 4.5% w/v or w/w. In some embodiments, the formulation can comprise a wetting agent in an amount from about 0.1% w/v or w/w to about 4.5% w/v or w/w. In some embodiments, the formulation can comprise a wetting agent in an amount from about 0.5% w/v or w/w to about 4.5% w/v or w/w. In some embodiments, the formulation can comprise a wetting agent in an amount from about 0.01% w/v or w/w to about 4.0% w/v or w/w. In some embodiments, the formulation can comprise a wetting agent in an amount from about 0.1% w/v or w/w to about 4.0% w/v or w/w. In some embodiments, the formulation can comprise a wetting agent in an amount from about 0.5% w/v or w/w to about 4.0% w/v or w/w. In some embodiments, the formulation can comprise a wetting agent in an amount from about 0.05% w/v or w/w to about 3.0% w/v or w/w. In some embodiments, the formulation can comprise a wetting agent in an amount from about 0.1% w/v or w/w to about 3.0% w/v or w/w. In some embodiments, the formulation can comprise a wetting agent in an amount from about 0.5% w/v or w/w to about 3.0% w/v or w/w. In some embodiments, the formulation can comprise a wetting agent in an amount from about 0.1% w/v or w/w to about 2.0% w/v or w/w. In some embodiments, the formulation can comprise a wetting agent in an amount from about 0.2% w/v or w/w to about 1.0% w/v or w/w. In some embodiments, the formulation can comprise a wetting agent in an amount from about 2% to about 4% w/v or w/w. In some embodiments, the formulation can comprise a wetting agent in an amount of about 0.05, 0.1, 0.13, 0.2, 0.25, 0.3, 0.5, 1, 2, 4, 5 or 7% w/v or w/w.

In some embodiments, the formulation can comprise one or more lubricating agents. Ocular lubricants can approximate the consistency of endogenous tears and aid in natural tear build-up. Lubricating agents can include non-phospholipid and phospholipid-based agents. Ocular lubricants that are non-phospholipid based include, but are not limited to, propylene glycol; ethylene glycol; polyethylene glycol; hydroxypropylmethylcellulose; carboxymethylcellulose; hydroxypropylcellulose; dextrans, such as, dextran 70; water soluble proteins, such as gelatin; vinyl polymers, such as polyvinyl alcohol, polyvinylpyrrolidone, povidone; petrolatum; mineral oil; and carbomers, such as, carbomer 934P, carbomer 941, carbomer 940, and carbomer 974P. Non-phospholipid lubricants can also include compatible mixtures of any of the foregoing agents.

The amount of the lubricating agent can vary. For example, the amount of the lubricating agent in the formulation can be up to 10% w/v or w/w or up to 30% w/v or w/w. In some embodiments, the formulation can comprise a lubricating agent in an amount from about 0.01% w/v or w/w to about 4.5% w/v or w/w. In some embodiments, the formulation can comprise a lubricating agent in an amount from about 0.1% w/v or w/w to about 4.5% w/v or w/w. In some embodiments, the formulation can comprise a lubricating agent in an amount from about 0.5% w/v or w/w to about 4.5% w/v or w/w. In some embodiments, the formulation can comprise a lubricating agent in an amount from about 0.01% w/v or w/w to about 4.0% w/v or w/w. In some embodiments, the formulation can comprise a lubricating agent in an amount from about 0.1% w/v or w/w to about 4.0% w/v or w/w. In some embodiments, the formulation can comprise a lubricating agent in an amount from about 0.5% w/v or w/w to about 4.0% w/v or w/w. In some embodiments, the formulation can comprise a lubricating agent in an amount from about 0.05% w/v or w/w to about 3.0% w/v or w/w. In some embodiments, the formulation can comprise a lubricating agent in an amount from about 0.1% w/v or w/w to about 3.0% w/v or w/w. In some embodiments, the formulation can comprise a lubricating agent in an amount from about 0.5% w/v or w/w to about 3.0% w/v or w/w. In some embodiments, the formulation can comprise a lubricating agent in an amount from about 0.1% w/v or w/w to about 2.0% w/v or w/w. In some embodiments, the formulation can comprise a lubricating agent in an amount from about 0.2% w/v or w/w to about 1.0% w/v or w/w. In some embodiments, the formulation can comprise a lubricating agent in an amount from about 2% to about 4% w/v or w/w.

In some embodiments, the ocular lubricating agent is a phospholipid-based lubricant. As used herein, “phospholipid lubricant” refers to aqueous compositions which comprise one or more phospholipids. Tear film has been shown to comprise a lipid layer, which is secreted by tear glands and is composed of various types of phospholipids (see, e.g., McCulley and Shine, 2003, The Ocular Surface 1:97-106). Examples of phospholipid lubricant formulations include those disclosed in U.S. Pat. Nos. 4,804,539; 4,883,658; 4,914,088; 5,075,104; 5,278,151; 5,294,607; 5,371,108; and 5,578,586; all of which are incorporated herein by reference. Lubricating compositions based on liposomes are described in U.S. Pat. Nos. 4,818,537 and 5,800,807, the disclosures of which are incorporated by reference herein.

Form of the formulations of the present invention is not particularly limited. For example, the formulation can be in form of liquid, fluid, emulsion, gel, semi-solid, or solid. In addition, liquids, fluids, emulsions, gels, semi-solids, solids, and the like that have been prepared at the time of use are also included. The semi-solid state refers to a property having plasticity that can be deformed by applying force, such as an ointment. Form for ophthalmic use is also not particularly limited. For example, eye drops (including eye drops when wearing contact lenses), eye wash, contact lens mounting liquid, contact lens liquid (cleaning liquid, preservative liquid, disinfecting liquid, multipurpose solution, package solution), transplant cornea, etc. Preservatives for isolated ocular tissues, perfusate during surgery, ointment (water-soluble ointment, oil-soluble ointment), intraocular injection (e.g., intravitreal injection), sustained-release intraocular implant, and Examples include sustained-release contact lens preparations. Of these, eye drops, intraocular injections, eye ointments, and eye washes are preferable, and eye drops are more preferable in terms of good transferability to the affected area. The sustained-release intraocular implant includes preparations such as solid, semi-solid, gel, fluid, and liquid, and the sustained-release contact lens preparation includes solid, semi-solid, gelled and other preparations are included.

The formulation can be an aqueous composition (mainly including an aqueous or hydrophilic base or carrier), or a non-aqueous/oily composition (an oil or hydrophobic base or carrier is mainly used). The content of water in the case of the aqueous composition is preferably 50% by weight or more, more preferably 75% by weight or more, and still more preferably 90% by weight or more based on the total amount of the preparation. In addition, according to some embodiments, the base or carrier can be composed only of water. The content of water in the case of an oily or non-aqueous composition is preferably 50% by weight or less, more preferably 30% by weight or less, and still more preferably 20% by weight or less based on the total amount of the formulation. Some exemplary non-aqueous bases or carriers include, but are not limited to, lanolin, lanolin alcohols, lanolin oil, mineral oil and petrolatum.

In some embodiments, the composition further comprises a therapeutic agent. Non-limiting examples of therapeutic agents include an antibacterial, an anti-fungal, an anti-viral, an anti-inflammatory, an immunosuppressive, an anti-glaucoma, an anti-VEGF, a growth factor, or any combination thereof. Non-limiting examples of antibacterial agents include: penicillins, cephalosporins, penems, carbapenems, monobactams, aminoglycosides, sulfonamides, macrolides, tetracyclines, lincosamides, quinolones, chloramphenicol, vancomycin, metronidazole, rifampin, isoniazid, spectinomycin, trimethoprim sulfamethoxazole, chitosan, ansamycins, daptomycin, nitrofurans, oxazolidinones, bacitracin, colistin, polymixin B, and clindamycin. Non-limiting examples of anti-fungal agents include: amphotericin B, natamycin, candicin, filipin, hamycin, nystatin, rimocidin, voriconazole, imidazoles, triazoles, thiazoles, allylamines, echinocandins, benzoic acid, ciclopirox, flucytosine, griseofulvin, haloprogin, tolnaftate, undecylenic acid, and povidone-iodine. Non-limiting examples of anti-viral agents include: acyclovir, valacyclovir, famciclovir, penciclovir, trifluridine, and vidarabine. Non-limiting examples of anti-acanthamoebal agents include: chlorohexidine, polyhexamethylen biguanide, propamidine, and hexamidine. Non-limiting examples of anti-inflammatory agents include: corticosteroids; non-steroidal anti-inflammatory drugs including salicylates, propionic acid derivatives, acetic acid derivatives, enolic acid derivatives, anthranilic acid derivatives, selective cox-2 inhibitors, and sulfonanilides; biologicals including antibodies (such as tumor necrosis factor-alpha inhibitors) and dominant negative ligands (such as interleukin-1 receptor antagonists). Non-limiting examples of immunosuppressive agents include: alkylating agents, antimetabolites, mycophenolate, cyclosporine, tacrolimus, and rapamycin. Non-limiting examples of anti-glaucoma agents include: prostaglandin analogs, beta blockers, adrenergic agonists, carbonic anhydrase inhibitors, parasympathomimetic (miotic) agents. Non-limiting examples of anti-vascular endothelial growth factor (anti-VEGF) agents include: bevacizumab, ranibizumab, and aflibercept. Non-limiting examples of growth factors include: epidermal growth factor, platelet-derived growth factor, vitamin A, fibronectin, annexin a5, albumin, alpha-2 macroglobulin, fibroblast growth factor b, insulin-like growth factor-I, nerve growth factor, and hepatocyte growth factor.

In some embodiments, the formulation is in the form of an emulsion. The emulsion can be water in oil (W/O) or oil in water (0/W). Generally, O/W emulsions are preferred for ocular delivery. Without wishing to be bound by a theory, charged droplets can enhance retention over negatively charged corneal cells.

In some embodiments, the formulation can be in the form of a hydrogel emulsion. For example, the active agent can be formulated into a hydrogel and then encapsulated into emulsion. Without wishing to be bound by a theory, loading into hydrogel can provide stability to formulation and can increase corneal retention.

Emulsion formulations can comprise, in addition to the active agent, one or more of the following components: co-solvents, emulsifiers, co-emulsifiers, thickeners, chelating agents, preservatives, buffering agents, pH modifiers, oils, and aqueous solvents.

Hydrocarbon ointments bases are the most common base for ophthalmic preparations. They are almost inert and have little tendency to rancidity. They also are suitable for sterile products because they are stable to dry heat sterilization. Thus, in some embodiments, the formulation is a non-aqueous ointment.

In the embodiments herein, a formulation for ophthalmic use can be formulated in accordance with methods known in the art. Guidance can be found in Duvall and Kershner, Ophthalmic Medications and Pharmacology 2^{nd} Ed, Slack Incorporated (2006); Ophthalmic Drug Facts® 18^{th} Ed, Wolters Kluwer (2007); Remington's Pharmaceutical Sciences, 19th ed. Gennaro A R, ed. Easton, Pa.: Mack Publishing, pages 1581-1959 (1990); and Reynolds L., 1991, “Guidelines for preparation of sterile ophthalmic products,” Am. J. Hosp. Pharm. 48:2438-9; the disclosures of which are incorporated by reference herein.

In some embodiments, the formulation is in the form of a nanoparticulate system. In some embodiments, the formulation is in the form of a nano suspension. In some embodiments, the formulation is in the form of a nano emulsion. In some embodiments, the formulation is in the form of a nano gel. In some embodiments, the formulation is in the form of a nano cream.

In some embodiments, the formulation in the form of a nanoparticulate system refers to the particle size or globule size of the formulation in nano-scale range. Such formulations may also be referred to as nano formulations according to the present invention.

In some embodiments, the particle size or globule size of the nano formulation is in the range of about 1 nm to about 10,000 nm. In some embodiments, the particle size or globule size range is about 10 nm to about 1000 nm.

Further, the active agent of the present formulations when present in the form of either nanoparticle or nano-globule form show higher interaction and higher drug penetration in comparison to their non-nano form. This fact overall enhances drug delivery/deposition in the desired portions of eye leading to improved therapeutic effects. The particle or globular size between 100-900 nm is considered to be optimum for very good retention in the eye or its parts.

Mycophenolic acid derivative such as mycophenolate mofetil blocks de novo biosynthesis of purine nucleotides by inhibition of the enzyme inosine monophosphate dehydrogenase. Mycophenolic acid prevents the proliferation of T-cells, lymphocytes and the formation of antibodies from B-cells. Further, mycophenolic acid inhibits recruitment of leukocytes to inflammatory sites. Mycophenolic acid also prevents the glycosylation of lymphocyte and monocyte glycoproteins that are involved in intercellular adhesion to endothelial cells and may inhibit recruitment of leukocytes to sites of inflammation. In autoimmune uveitis, it is observed that activated T cells penetrate through blood and tissue barriers and affect retina proteins. In experimental condition, when autoimmune uveitis developed in mice, increased level of activated T cells was observed in intestine over spleen. In the case of infectious uveitis, microbial antigens are responsible for activating T cells, affecting retina proteins.

Mycophenolic acid targets the de novo purine biosynthesis pathway, which is critical for lymphocytes proliferation. Cell death in most cases is induced by a novel necrotic pathway. Mycophenolic acid or its salts/derivatives have a high therapeutic window ranging from 0.05 μg/mL to 100 μg/mL, with some toxicity beginning to appear at doses above 100 μg/mL. Owing to its selective action, the drug shows fewer and milder side effects compared to other immunosuppressive agents.

Some exemplary formulations of the invention are shown in the following tables.

TABLE B
Ophthalmic gel

| | 1 | 2 |
|---|---|---|
| Active | Mycophenolate sodium | Mycophenolate sodium |
| Inactives | Boric acid | Hypromellose |
| | Sodium chloride | Sodium chloride |
| | Edetate disodium dihydrate | Purified Water |
| | Glycerin | Hydrochloric Acid and/or |
| | Hypromellose | Sodium Hydroxide. |
| | Poloxamer | (to adjust pH) |
| | Polycarbophil | |
| | Propylene glycol | |
| | Sodium chloride | |
| | Sodium hydroxide (to adjust pH) | |
| | Water for injection | |
| Preservative | Benzalkonium chloride | Benzalkonium chloride |

TABLE C
Ophthalmic Suspension (Dry Powder for reconstitution)

| | 1 |
|---|---|
| Active | Mycophenolate mofetil hydrochloride |
| Inactives | Mannitol (lyophilisation agent) |
| | Modified diluent of sodium acetate trihydrate, potassium |
| | chloride, magnesium chloride hexahydrate, calcium chloride |
| | dihydrate and sterile water for injection. |

Note:
the reconstituted liquid will be a sterile isotonic solution. Further, mannitol is used in the process of lyophilizing MMF, and is not considered an active ingredient.

TABLE D
Non-aqueous Formulation (ointment)

| | 1 | 2 | 3 |
|---|---|---|---|
| Active | Mycophenolate | Mycophenolate | Mycophenolate |
| | mofetil | mofetil | mofetil |
| Inactives | Mineral oil | White petrolatum | Mineral oil |
| | White petrolatum | Anhydrous liquid | White petrolatum |
| | | lanolin | lanolin alcohol |
| Preservative | Chlorobutanol | Methylparaben | Phenylmercuric |
| | 0.5% | 0.05%, | acetate (0.0008%). |
| | | Propylparaben | |
| | | 0.01% | |

TABLE E
Ophthalmic Solution

| | 1 | 2 | 3 | 4 | 5 |
|---|---|---|---|---|---|
| Active | Mycophenolate | Mycophenolate | Mycophenolate | Mycophenolate | Mycophenolate |
| | sodium | sodium | sodium | sodium | sodium |
| Inactives | Boric acid | Monobasic | Sodium | Hypromellose | Edetate |
| | | sodium | acetate | 2910 | disodium |
| | | phosphate, | | | |
| | Sodium | Edetate | Sodium | Boric acid | Sodium |
| | chloride | disodium | chloride | | chloride |
| | Tyloxapol | Sodium | Sodium | Sodium | Sodium |
| | | chloride | hydroxide | citrate | hydroxide |
| | | | and/or | | and/or |
| | | | hydrochloric | | hydrochloric |
| | | | acid (pH | | acid (to |
| | | | adjustment) | | adjust pH) |
| | Sodium | Sodium | Purified | Sodium | Purified |
| | hydroxide | hydroxide | water | chloride | water |
| | and/or sulfuric | and/or | | | |
| | acid (to adjust | hydrochloric | | | |
| | pH) | acid (to | | | |
| | | adjust pH) | | | |
| | Purified | Purified | | Hydrochloric | |
| | water | water | | acid and/or | |
| | | | | sodium | |
| | | | | hydroxide (to | |
| | | | | adjust pH) | |
| | | | | Purified | |
| | | | | water | |
| Preservative | Benzalkonium | Benzalkonium | Benzalkonium | Benzalkonium | Benzalkonium |
| | chloride | chloride | chloride | chloride | chloride |

TABLE F
Emulsion Formulation (O/W)

| | | | | 3 | |
|---|---|---|---|---|---|
| Active | Mycophenolate | Mycophenolate | | | |
| | mofetil | mofetil | | | |
| Oily Vehicle | Castor oil | Mineral Oil | | Oelic acid | |
| | | | | IPM | |
| | | | | Labrafac PG | |
| | | | | Labrafac 1349 | |
| Co-solvent | Glycerin | | | | |
| | Propylene Glycol | | | | |
| Emulsifier | Polysorbate 80 | Polyoxyl 40 Stearate | | Lauroglycol ™ FCC | Sefsol-218 |
| | Polyoxyl 15 | Polysorbate 20 | | Labrafil ® M 2125 | Lauroglycol ™ 90 |
| | Hydroxystearate | Tyloxapol | | Transcutol P | Laureth-4 |
| | | Glyceryl Monostearate | | Capryol ™ 90 | Span 80 |
| Co- | | Polyoxyl 40 Hydrogenated castor oil | | Capryol ™ PGMC | Span 20 |
| Emulsifier | | Polyoxyl 35 castor oil | | Labrafil ® M 1944 | LABRASOL |
| | | Poloxomer 407/188 | | CS | |
| Thickener | Carbomer | Carbomer 940 | Hypromellose 2910 | Chitosan | |
| | copolymer type A | Carbomer | Methylcellulose | | |
| | Carbomer | homopolymer | Polyvinyl | | |
| | copolymer type B | type B | Alcohol | | |
| | | Hypromellose 2910 | Povidone | | |
| | | Polycarbophil | Povidone K30 | | |
| | | Guar gum | Xanthan gum | | |
| | | Hydroxyethyl | Ethyl Cellulose | | |
| | | cellulose | Dextran | | |
| Chelating | Edetate Sodium | | | | |
| agent | Edetate Disodium | | | | |
| Preservative | Sorbic acid | Benzalkonium Chloride | | | |
| | Boric Acid | | | | |
| | Sodium borate | | | | |
| | Potassium | | | | |
| | sorbate | | | | |
| Buffering | Sodium acetate | Sodium phosphate | | | |
| agent | | Sodium phosphate monobasic | | | |
| | | Potassium phosphate | | | |
| pH Modifier | Sodium | | | | |
| | hydroxide | | | | |
| | Hydrochloric | | | | |
| | acid | | | | |
| Aqueous | Purified water | | | | |
| Phase | | | | | |

In some embodiments, the formulation can be in form of a self-emulsifying drug delivery system. Generally, such a formulation comprises one or more lipids, one or more co-solvents, one or more emulsifiers and co-emulsifiers, and an aqueous phase. In some embodiments, in a self-emulsifying drug delivery system the lipids, co-solvents, emulsifiers, and co-emulsifiers can be selected from those shown in the following table.

TABLE G
Self-emulsifying drug delivery system

| Active | Mycophenolate mofetil | |
|---|---|---|
| Lipids | Labrasol | Capmul GMS-50K |
| | Lauroglycol 90 | Labrafil M 1944 CS |
| | Labrafac CC | Brij |
| | Isopropyl myristate | Stepan GDL |
| | Capmul MCM | Caprol ET |
| | Maisine 35-1 | Labrafac 1349 |
| | Akoline MCM | Labrafac PG |
| | Capmul MCM C-8 | Sesame Oil |
| Co- | Ethanol | Benzyl alcohol |
| solvent | Pentanol | Capryol 90 |
| | Carbitol | Methoxy PEG |
| | Labrasol | PEG 400 |
| | Butanol | Transcutol P |
| | Glycerol | PEG ether (glycofurol) |
| | PG | Tetrahydrofurfuryl alcohol |
| | Ethylene glycol | Isopropanol |
| Emulsifier | Tween 85 | Labrasol |
| | Capryol 90 | Tween 80 |
| | Lauroglycol 9 | Pluronic F 127 |
| | Labrafil M 1944 C | Pluronic L 64 |
| | Tween 20 | Tagat TQ |
| | | Span 80 |
| Co- | Tween 85 | Acconon MC8 |
| Emulsifier | Akoline MCM | Cremophor RH40 |
| | Carbitol | Cremophor EL |
| | PG-dicaprylate/dicaprate | Ethanol |
| | PEG 600 | Octanol |
| | PEG 300 | Span 20 |
| | Capyrol PGMC | Hexanol |

| | |
|---|---|
| Aqueous | Sterile water for injection |
| Phase | |

TABLE H
Vesicular carrier for ocular drug delivery
Non-ionic surfactant vesicles (Simple and cost-effective techniques
with improved physical stability and enhance bioavailability)

| | |
|---|---|
| Active | Mycophenolate mofetil |
| Membrane composition | Non-ionic surfactants |
| | Brij 35, 78, 98, and 700 |
| | Polysorbate 80, polyoxyl 40 stearate, and |
| | polyoxyl 60 hydrogenated castor oil |
| | Pluronic F127 |
| | Spans 20, 40, 60, and 80 |
| | Cholesterol |
| Targeting agent | Didodecyldimethylammonium bromide |
| | Stearylamine |
| | N-[1-(2,3-dioleoyloxy)propyl]-N,N,N- |
| | trimethylammonium chloride |
| Aqueous Phase | Purified water |

TABLE I
Vesicular carrier for drug delivery
Spanlastics (Spanlastics are nano-size and elastic in
nature results in better corneal permeation)

| | |
|---|---|
| Active | Mycophenolate mofetil |
| Membrane composition | Span 60 and a edge activator (tween 80) |
| Targeting agent | Didodecyldimethylammonium bromide |
| | Stearylamine |
| | N-[1-(2,3-dioleoyloxy)propyl]-N,N,N- |
| | trimethylammonium chloride |
| Aqueous Phase | Purified water |

In situ gel-forming systems can be described as low viscosity solutions that undergo phase transition in the conjunctival cul-de-sac to form viscoelastic gels due to conformational changes of polymers in response to the physiological environment. Exemplary in situ gel forming formulation is shown in the following table.

TABLE J
In situ gel-forming systems for sustained ocular delivery
In situ gel-forming systems

| Active | | Mycophenolate mofetil |
|---|---|---|
| Polymers | Temperature induced | Poloxamer |
| | in situ gel systems | Carbopol (0.3%) and pluronic |
| | | (14%), 3% methyl cellulose and |
| | | a low concentration of Pluronic |
| | | Gelritegellan gum |
| | | pluronic-g-poly(acrylic acid) |
| | pH induced in situ | Cellulose acetate phthalate latex |
| | gel systems | cross-linked polyacrylic |
| | | derivatives of carbomers. |
| | Osmotically induced | Gelrite |
| | in situ gel systems | sodium alginate and |
| | | hydroxypropylmethyl cellulose |

| | |
|---|---|
| Aqueous Phase | Purified water |

The formulation can be in form of a polymeric nanoparticulate system. Generally, the polymeric nanoparticulate system comprises particles in the range of 1-1,000 nm in which the active agent, prodrug or salt thereof is adsorbed, entrapped, conjugated, or encapsulated. Aqueous or nonaqueous suspension of drug-loaded nanoparticles can be administered in the cul-de-sac to achieve sustained drug delivery, which can eliminate frequent drug administration.

TABLE K
Nano-particulate carrier for ocular drug delivery
In situ gel-forming systems

| | |
|---|---|
| Active | Mycophenolate mofetil |
| Temperature | Eudragit RS100 |
| induced | PLGA |
| in situ | polycaprolactone (PCL) |
| gel systems | Chitosan |
| | Cholesterol- conjugated hydrophobically modified chitosan |
| | physical mixture of poly lactic acid (PLA)/chitosan |
| | (HA)-coated Eudragit nanoparticles (RS 100 and RL 100) |
| | hyaluronic acid-chitosan oligomer based nanoparticles |
| Aqueous | Purified water |
| Phase | |

TABLE L
Nanomicelles carrier for ocular drug delivery
In situ gel-forming systems

| | |
|---|---|
| Active | Mycophenolate mofetil |
| Polymers | N-isopropylacrylamide, |
| | vinyl pyrrolidone, |
| | acrylic acid, |
| | polyhydroxyehtylaspartamide, |
| | poly(ethylene glycol)-hexylsubstituted |
| | poly(lactides) |
| | Pluronic F127 |
| | Vitamin E TPGS |
| | octoxynol-40 |
| | methoxy poly(ethylene glycol)-hexylsubstituted |
| | poly(lactide) |
| Aqueous | Purified water |
| Phase | |

Contact lenses for ophthalmic drug delivery have become very popular, due to their unique advantages like extended wear and more than 50% bioavailability. Accordingly, in some embodiments, the formulation can be comprised in a contact lens.

Methodology to design therapeutic contact lenses include the following:

Soaking method: Contact lenses have internal channels/cavity for accommodating the drug molecules. Their drug reservoir ability strongly depends on the water content, thickness of lenses, the molecular weight of the drug, soaking time period and concentration of drug in soaking solution. (Use of vitamin E).

Molecular imprinting: Molecular imprinting (MI) is one of the advanced methodologies explored by Alvarez-Lorenzo and co-workers using hydrogel contact lenses for high drug loading and controlled drug delivery.

Colloidal nanoparticles laden therapeutic contact lens: The technique is based on the ability of colloidal nanoparticles (polymeric nanoparticles, liposomes, niosomes, microemulsion, micelles, etc.) to entrap or encapsulate drug and control its release rate from contact lenses.

In some embodiments, the formulation can be included in a Soluble Ophthalmic Drug Inserts (SODI). A SODI is a soluble copolymer of acrylamide, N-vinyl pyrrolidone, and ethyl acrylate. It is in the form of sterile thin films or wafers of oval shape, weighing 15 to 16 mg. After introduction into the upper conjunctival sac, the SODI softens in 10 to 15 sec, conforming to the shape of the eyeball; in the next 10 to 15 min the film turns into a polymeric clot, which gradually dissolves within 1 hour, while releasing the drug.

In some embodiments, the formulation can be included in a Bioadhesive Ophthalmic Drug Inserts (BODI) or a collagen shield. Collagen shields are commercially available products on the market, e.g., MediLens (Chiron, Irvine, Calif.) and ProShield (Alcon, Fort Worth, Tex.) are prepared from bovine corium tissue and last between 24 and 48 hours on the cornea.

For ocular applications, the formulation can be in the form of solutions, suspensions, ointments, pellets, gels, colloidal systems, and hydrogels.

Suspensions: Included in this dosage form category are solid preparations that, when reconstituted according to the label instructions, result in a solution. The corneal contact time of topical ophthalmic solutions increases with the viscosity of the formulations up to 20 centipoise (cP). Further increases result in reflex tearing and blinking in order to regain the original viscosity of the lacrimal fluid (1.05-5.97 cP). The bioavailability increase associated with this longer precorneal permanence allows the frequency of drug application to be reduced. Synthetic polymers, such as polyvinylalcohol (PVA), polyvinylpyrrolidone (PVP), polyethylene glycol (PEG), polyacrylic acid (PAA), and many cellulose derivatives, are commonly employed as viscosity enhancers because of their physiologic compatibility and satisfactory physicochemical properties. A more sophisticated approach consists of using polymers that provide the liquid formulation with semisolid consistency only when it is placed in the conjunctival or corneal area. In this way, easy installation of the solution is followed by prolonged permanence as a result of the viscoelastic properties of the formed hydrogel. This in situ gelling phenomenon is caused by a change in the conformation of the polymer(s) that can be triggered by external stimuli such as temperature, pH, ionic content and lacrimal fluid upon delivery into the eye. Additionally, some polymers can interact, via noncovalent bonds, with conjunctival mucin and maintain the formulations in contact with corneal tissues until mucin turnover leads to their removal. Two of the major drawbacks of viscous and mucoadhesive formulations are blurring and an unpleasant sticky feeling in the eye.

Suspensions: Can be of those solid preparations that, when reconstituted according to the label instructions, result in a suspension or ready to use suspensions. An ophthalmic suspension may be required when the active ingredient is insoluble in the desired vehicle. Suspensions are required to be made with the insoluble drug in a micronized form to prevent irritation or scratching of the cornea. (Particle size <10 μm). Particle size in suspensions for ocular drug delivery is very important. An increase in drug particle size enhances the ocular bioavailability. Unfortunately, a particle size above 10 μm in diameter may result in a foreign body sensation in the eye following ocular application, causing reflex tearing. A reduction in particle size generally improves the patient comfort and acceptability of suspension formulations. Particle size determination on stability is a critical test which needs to be evaluated through stability testing. Surfactants help to disperse the drug effectively in the suspension formulation. (Nonionic surfactants are generally preferred as they are comparatively less toxic.) Suspension for ophthalmic use can be manufactured by terminal sterilization or lyophilized powder (more stable).

Ointments: Ointment bases utilized for ophthalmics have a melting or softening point close to body temperature. Ophthalmic ointments have a longer ocular contact time when compared to many ophthalmic solutions. Studies have shown that the ocular contact time is two to four times greater when ointments are used than when a saline solution is used. One disadvantage to ophthalmic ointments is the blurred vision after application. Generally, ocular ointment comprises of mixture of semisolid and a solid hydrocarbon (paraffin) that has a melting point at physiological ocular temperature (34° C.).

Gels: Normally mucoadhesive polymers are used as gelling agents in case of ophthalmic gel formulations. Such polymers have a property known as bioadhesion, which increase the contact time of the drug with the biological tissues and thereby improve ocular bioavailability. Few examples of bioadhesive polymers are; carboxymethylcellulose, carbopol, polycarbophil, and sodium alginate, etc.

Colloidal Systems include microemulsions, nanosuspensions, nanoparticles, liposomes, nanosomes and niosomes.

Nanocapsules or nanospheres: In nanocapsules, drug is enclosed inside the polymeric shell while in nanospheres; drug is uniformly distributed throughout polymeric matrix. Nanoparticles represents a promising candidate for ocular drug delivery because of small size leading to low irritation and sustained release property avoiding frequent administration. PLGA, Polyethylene glycol (PEG), chitosan and hyaluronic acid are commonly employed to improve precorneal residence time of nanoparticles.

Nanosuspensions are colloidal dispersion of submicron drug particles stabilized by polymer(s) or surfactant(s).

Liposomes are lipid vesicles with one or more phospholipid bilayers enclosing an aqueous core. The size of liposomes usually ranges from 0.08 to 10.00 μm and based on the size and phospholipid bilayers, liposomes can be classified as small unilamellar vesicles (10-100 nm), large unilamellar vesicles (100-300 nm) and multilamellar vesicles (contains more than one bilayer). For ophthalmic applications, liposomes represent ideal delivery systems due to excellent biocompatibility, cell membrane like structure and ability to encapsulate both hydrophilic and hydrophobic drugs.

Dendrimers: Dendrimers are characterized as nano-sized, highly branched, star shaped polymeric systems. These branched polymeric systems are available in different molecular weights with terminal end amine, hydroxyl or carboxyl functional group. The terminal functional group can be utilized to conjugate targeting moieties.

In situ gelling systems: In-situ hydrogels refer to the polymeric solutions which undergo sol-gel phase transition to form viscoelastic gel in response to environmental stimuli. Gelation can be elicited by changes in temperature, pH and ions or can also be induced by UV irradiation. Subcutaneous gel injection is an example of such an in situ gelling system.

Contact lens: Contact lenses are thin, and curved shape plastic disks which are designed to cover the cornea. After application, contact lens adheres to the film of tears over the cornea due to the surface tension. Drug loaded contact lens have been developed for ocular delivery of numerous drugs such as β-blockers, antihistamines and antimicrobials.

Implants: Intraocular implants are specifically designed to provide localized controlled drug release over an extended period. These devices help in circumventing multiple intraocular injections and associated complications. Usually for drug delivery to posterior ocular tissues, implants are placed intravitreally by making incision through minor surgery at pars plana which is located posterior to the lens and anterior to the retina. Vitrasert® (Bausch and Lomb Inc., Rochester, N.Y., United States) is a controlled-release intraocular implant of ganciclovir approved by Food and Drug Administration (FDA). Retisert® (Bausch and Lomb Inc., Rochester, N.Y., United States) is approved by FDA for the treatment of chronic uveitis which affects the posterior segment of the eye. Biodegradable implants for ocular delivery include Surodex™ and Ozurdex® which are designed for the sustained delivery of dexamethasone for the treatment of intraocular inflammation and macular edema (ME). The NOVADUR® system contains a PLGA polymer matrix which degrades slowly to lactic acid and glycolic acid allowing prolonged release of dexamethasone up to 6 months. Other routes of delivery include subconjunctival route as well as suprochoroidal injection.

Niosomes: Ocular delivery using niosomes aims to achieve localized drug action since their size and low penetrability through epithelium keeps the drug localized at the site of administration and increases precorneal residence time. Many studies reported the value of niosomes in topical ocular drug delivery. The main components of niosomes are nonionic surfactants and the additives (cholesterol and charged molecules). The nonionic surfactants form the vesicular layer and cholesterol improves the rigidity of the bilayer which affects bilayer fluidity and cell permeability.

Spanlastics is a novel, surfactants based elastic vesicular drug carrier system (spanlastics), for targeting topically applied drug(s) to the posterior segment of the eye. The system constituted span 60 and an edge activator (tween 80). Spanlastics are to niosomes what transfersomes are to liposomes.

Hydrogels are three-dimensional, hydrophilic, polymeric networks capable of taking in large amounts of water or biological fluids. Residence time can be significantly enhanced with a hydrogel formulation. The gelation can be obtained by changing temperature and pH. Poloxamers, the most widely used polymer, contains the hydrophobic part in the centre surrounded by a hydrophilic part. (weak mechanical strength, rapid erosion, and nonbiodegradability).

In some embodiments, the ophthalmic formulations of the disclosure is in the form of eye drops (including eye drops when wearing contact lenses), eye wash, contact lens mounting liquid, contact lens liquid (cleaning liquid, preservative liquid, disinfecting liquid, multipurpose solution, package solution), transplant cornea, or any combinations thereof.

The ophthalmic formulations described herein can be used to treat various ocular disorders amenable to treatment with the immunosuppressive and anti-inflammatory compound. The terms “ophthalmic disorder,” “ocular disorder,” “ocular disease”, “eye disease” and “eye disorder” are used interchangeably herein to include, among others, “back-of-eye” diseases involving the retina, macula, fovea, etc. in the posterior region of the eye; and “front-of-eye” diseases, such as those that involve tissues such as the cornea, iris, ciliary body, conjunctiva, lacrimal gland, etc. These conditions or diseases can manifest as pain, discomfort, tissue damage and compromised visual performance of the eyes in the afflicted subject.

Examples of “back-of-eye” disease include, among others, macular edema such as angiographic cystoid macular edema; retinal ischemia and choroidal neovascularization; macular degeneration; retinal diseases (e.g., diabetic retinopathy, diabetic retinal edema, retinal detachment); inflammatory diseases such as uveitis (including panuveitis) or choroiditis (including multifocal choroiditis) of unknown cause (idiopathic) or associated with a systemic (e.g., autoimmune) disease; episcleritis or scleritis; Birdshot retinochoroidopathy; vascular diseases (retinal ischemia, retinal vasculitis, choroidal vascular insufficiency, choroidal thrombosis); neovascularization of the optic nerve; and optic neuritis.

Examples of “front-of-eye” diseases include, among others, blepharitis; keratitis; rubeosis iritis; Fuchs' heterochromic iridocyclitis; chronic uveitis or anterior uveitis; conjunctivitis; allergic conjunctivitis (including seasonal or perennial, vernal, atopic, and giant papillary); keratoconjunctivitis sicca (dry eye syndrome); iridocyclitis; iritis; scleritis; episcleritis; corneal edema; scleral disease; ocular cicatricial pemphigoid; pars planitis; Posner Schlossman syndrome; Behcet's disease; Vogt-Koyanagi-Harada syndrome; hypersensitivity reactions; conjunctival edema; conjunctival venous congestion; periorbital cellulitis; acute dacryocystitis; non-specific vasculitis; and sarcoidosis.

In some embodiments, these eye conditions include “front of the eye” disorders such as blepharitis; keratitis; rubeosis iritis; Fuchs' heterochromic iridocyclitis; chronic uveitis or anterior uveitis; conjunctivitis; allergic conjunctivitis (including seasonal or perennial, vernal, atopic, and giant papillary); keratoconjunctivitis sicca (dry eye syndrome); iridocyclitis; iritis; scleritis; episcleritis; corneal edema; scleral disease; ocular cicatricial pemphigoid; pars planitis; Posner Schlossman syndrome; Behcet's disease; Vogt-Koyanagi-Harada syndrome; hypersensitivity reactions; conjunctival edema; conjunctival venous congestion; periorbital cellulitis; acute dacryocystitis; non-specific vasculitis; and sarcoidosis. In some embodiments, the eye conditions include “back of the eye” disorders such as macular edema; angiographic cystoid macular edema; retinal ischemia and choroidal neovascularization; macular degeneration; retinal diseases (e.g., diabetic retinopathy, diabetic retinal edema, retinal detachment); inflammatory diseases such as uveitis (including panuveitis) or choroiditis (including multifocal choroiditis) of unknown cause (idiopathic) or associated with a systemic (e.g., autoimmune) disease; episcleritis or scleritis; Birdshot retinochoroidopathy; vascular diseases (e.g., retinal ischemia, retinal vasculitis, choroidal vascular insufficiency, choroidal thrombosis); neovascularization of the optic nerve; and optic neuritis.

In some embodiments, the eye disorder is associated with an inflammatory condition of the eye. These conditions can include, but are not limited to, the various disorders described above for the back-of-eye and front-of-eye, such as, for example, inflammation associated with macular edema; retinal ischemia; choroidal neovascularization, macular degeneration; diabetic retinopathy; diabetic retinal edema; retinal detachment; inflammatory diseases such as uveitis (including panuveitis), recurrent uveitis, refractory uveitis, or choroiditis (including multifocal choroiditis) of unknown cause (idiopathic) or associated with a systemic (e.g., autoimmune) disease; episcleritis or scleritis; Birdshot retinochoroidopathy; vascular diseases (retinal ischemia, retinal vasculitis, choroidal vascular insufficiency, choroidal thrombosis); neovascularization of the optic nerve and optic neuritis; blepharitis; keratitis; rubeosis iritis; Fuchs' heterochromic iridocyclitis; chronic uveitis or anterior uveitis; conjunctivitis; allergic conjunctivitis (including seasonal or perennial, vernal, atopic, and giant papillary); keratoconjunctivitis sicca (dry eye syndrome); iridocyclitis; iritis; scleritis; episcleritis; corneal edema; scleral disease; ocular cicatricial pemphigoid; pars planitis; Posner Schlossman syndrome; Behcet's disease; Vogt-Koyanagi-Harada syndrome; hypersensitivity reactions; conjunctival edema; conjunctival venous congestion; periorbital cellulitis; acute dacryocystitis; non-specific vasculitis; and sarcoidosis.

In some embodiments, the eye disorder treatable with the formulations of the invention is keratoconjunctivitis sicca, a condition also known as dry-eye, keratitis sicca, sicca syndrome, xeropthalmia, and dry eye syndrome (DES), which can arise from decreased tear production and/or increased tear film evaporation due to abnormal tear composition. Although the disorder can be caused by environmental chemicals and infection, the disorder is also associated with the autoimmune diseases of rheumatoid arthritis, lupus erythematosus, diabetes mellitus, and Sjögren's syndrome.

In some embodiments, the formulations of the invention can be used in conjunction with keratoplasty, particularly high risk keratoplasty.

In some embodiments, the eye disorders that can be treated with the formulations of the invention are those associated with autoimmune disorders. These conditions can include, but are not limited to, the various disorders above described for the back-of-eye and front-of-eye, such as, for example, choroidal neovascularization; macular degeneration; diabetic retinopathy; diabetic retinal edema; uveitis (including panuveitis) or choroiditis (including multifocal choroiditis) of unknown cause (idiopathic) or associated with a systemic disorder (e.g., autoimmune disease); episcleritis or scleritis; Birdshot retinochoroidopathy; neovascularization of the optic nerve, and optic neuritis; blepharitis, keratitis, rubeosis iritis; Fuchs' heterochromic iridocyclitis; chronic uveitis or anterior uveitis; conjunctivitis; allergic conjunctivitis (including seasonal or perennial, vernal, atopic, and giant papillary); iridocyclitis; iritis; scleritis; episcleritis; corneal edema; scleral disease; ocular cicatricial pemphigoid; pars planitis; Posner Schlossman syndrome; Behcet's disease; Vogt-Koyanagi-Harada syndrome; hypersensitivity reactions; conjunctival edema; conjunctival venous congestion; periorbital cellulitis; acute dacryocystitis; non-specific vasculitis; and sarcoidosis.

In exemplary embodiments, the eye disorder to be treated by the presently described ophthalmic formulations is uveitis.

In some embodiments, the eye disorder associated with an autoimmune condition that can be treated with formulations described herein is uveitis, a general term used to describe inflammation of any component of the uveal tract. The uveal tract of the eye consists of the iris, ciliary body, and choroid. Inflammation of the underlying retina, called retinitis, or of the optic nerve, called optic neuritis, or overlying sclera called scleritis or episcleritis may occur with or without accompanying uveitis (including anterior uveitis or iritis, intermediate uveitis or cyclitis, posterior uveitis or retinitis or choroiditis, and diffuse uveitis or panuveitis). Uveitis can be classified based on the segment of the eye that is affected, such as anterior, intermediate, posterior, or diffuse, or on the specific anatomical part involved, such as iritis, iridocyclitis, or choroiditis. Posterior uveitis signifies any of a number of forms of retinitis, choroiditis, or optic neuritis, as further described below. Diffuse uveitis typically implicates inflammation involving all parts of the eye, including anterior, intermediate, and posterior structures. Uveitis is one of the most common ocular disorders associated with autoimmune diseases, including rheumatoid arthritis; juvenile idiopathic arthritis, systemic lupus erythematosus; syphilis, Sjogren's syndrome; diabetes mellitus; sarcoidosis; ankylosing spondylitis; Psoriasis; multiple sclerosis; Vogt-Koyanagi-Harada disease; Behcet's disease; polyarteritis nodosa; giant cell arteritis; and inflammatory bowel disease. Mechanistically, uveitis is driven by the underlying overactivity of immune response via T and B cells. This overactivity can be dampened with the use of mycophenolic acid or mycophenolate salt or ester.

In some embodiments, inflammatory eye conditions such as conjunctivitis, blepharitis; keratitis; vitritis; chorioretinitis; and uveitis is associated with systemic or local infections where an immunosuppressant drug such as mycophenolic acid or an ester or pharmaceutically acceptable salt thereof can be used topically to suppress the ocular inflammation. Infections may be due to bacterial (e.g., Borrelia species, Streptococcus pneumoniae, Staphylococcus aureus, Mycobacterium tuberculosis, Mycobacterium leprae, Neisseria gonorrheae, Chlamydia trachomatis, Pseudomonas aeruginosa, etc.), viral (e.g., Herpes simplex, Herpes zoster, cytomegalovirus, etc.), fungal (e.g., Aspergillus fumigatus, Candida albicans, Histoplasmosis capsulatum, Cryptococcus species, Pneumocystis carinii, etc.) or parasitic agents (e.g., Toxoplasmosis gondii, Trypanosome cruzi, Leishmania species, Acanthamoeba species, Giardia lamblia, Septata species, Dirofilariaimmitis, etc.).

Other than an ophthalmology application, formulations of the invention can also be used for the treatment of skin diseases and disorders, such as lichen sclerosus (rare skin disease—where the immune cells attack the skin around the groin).

In some embodiments, the formulations will generally be used in an amount effective to treat the particular ocular disorder or other disease in a subject in need thereof. The formulations of the invention can be administered therapeutically to achieve therapeutic benefit or prophylactically to achieve prophylactic benefit. In some embodiments, the therapeutic agents can be administered to a veterinary animal subject, such as, among others, mouse, rat, horse, cat, dog, cow, pig, monkey, chimpanzee, etc.

In the embodiments herein, a therapeutically effective amount is applied topically to the eye of a subject in need of treatment. A “therapeutically effective amount” refers to an amount of the therapeutic agent either as an individual compound or in combination with other compounds that is sufficient to induce a therapeutic effect or prophylactic benefit on the disease or condition being treated. This phrase should not be understood to mean that the dose must completely eradicate the ailment. A therapeutically effective amount will vary depending on, inter alia, the pharmacological properties of the compound used in the methods, the condition being treated, the frequency of administration, the mode of delivery, characteristics of the individual to be treated, the severity of the disease, and the response of the patient. A skilled artisan can take into account such factors when formulating compositions for the treatments described herein, a process which is well within the skill of those in the art.

To treat the ocular disease, the formulation can be applied topically to the affected eye(s). In some embodiments, the formulation can be applied in defined volumes, such about 10, 20, 50, 75, 100, 150, or 200 μL or more. The frequency of application will depend on, among others, the type of ocular disease being treated, the severity of the condition, age and sex of the patient, the amount of the active agent in the formulation, and the pharmacokinetic profile in the ocular tissue to be treated. In some embodiments, the formulation can be administered more than one times per day. When the compositions are administered more than once per day, the frequency of administration can be two, three, four, up to eight times per day. In some embodiments, the formulation can be administered one to four times daily. In some embodiments, the formulation can be applied once every two days. In some embodiments, the formulation can be applied once every four days. In some embodiments, the formulation can be administered once every week. In other embodiments, the formulation can be applied once a month or once every two to six months. Determining the frequency and amount to be administered for a particular ocular disorder is well within the skill and judgment of the attending practitioner.

While the precise regimen is left to the discretion of the clinician, it is recommended that the compositions of the present disclosure be topically applied by placing one to two drops, or more, in each eye 1 to 4 times daily. For example, the composition can be applied 1, 2, 3, 4, 5, 6, 7 or 8 times a day, or more. In some embodiments, the compositions are topically applied by placing one to two drops in each eye once or twice daily.

In some embodiments, the formulation can be provided in the form of a kit. As such, the kit can contain the formulation in a container, as single dose unit or as a single solution reservoir. The kit can also contain a dispenser for dispensing measured doses as well as instructions for dosing and use of the formulations.

The formulation can be packaged in a container (particularly an ophthalmic container). The kind of container is not specifically limited. For example, the formulation can be contained or filled in a plastic container, a metal container, a glass container, etc. Pharmaceutically acceptable packaging materials for the formulations include, but are not limited to, polypropylene, polystyrene, low density polyethylene (LDPE), high density polyethylene (HDPE), polycarbonate, polyvinylidine chloride, and other materials known to those skilled in the art.

The compositions can be packaged aseptically employing blow-fill-seal technology. Blow-fill-seal (BFS) describes an aseptic filling process in which hollow containers are blow molded, filled with sterile product, and sealed, all in one continuous machine cycle. The technology is an alternative to conventional aseptic filling and capping operations, often providing cost savings through high output and process efficiency. In some embodiments, the compositions of the present disclosure are filled to single-use bottles, packets, vials, ampoules, LDPE BFS containers, or HDPE BFS containers.

Benzalkonium chloride, added to ophthalmic products, can cause ocular irritant and allergic response in some patients. Hence, preservative-free formulations offer a significant medical advantage. Accordingly, in some embodiments of the present invention, the ophthalmic formulation is provided in unpreserved multi-dose systems to combine the advantages of both approaches.

The Ophthalmic Squeeze Dispenser (OSD) is an example of novel devices designed to eliminate the need for preservative in the formulation and can be used with existing filling technologies. A key advantage of OSD is the prevention of contamination entering through the tip of dispensing system. Thus, in some embodiments, the formulation is provided in an OSD.

In some embodiments, laminated tubes are preferred for packing of semisolid ophthalmic products over aluminum or plastic tubes.

In some embodiments, multiple doses can be supplied as a plurality of single-use packages. In another embodiment, the compositions are conveniently packaged in a bottle, container or device that allows for metered application, including containers equipped with a dropper for topical ophthalmic application.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Other than in the operating examples, or where otherwise indicated, all numbers expressing quantities of ingredients or reaction conditions used herein should be understood as modified in all instances by the term “about.” The term “about” when used to describe the present invention, in connection with percentages means±1%, ±1.5%, ±2%, ±2.5%, ±3%, ±3.5%, ±4%, ±4.5%, or ±5%.

The singular terms “a,” “an,” and “the” include plural referents unless context clearly indicates otherwise. Similarly, the word “or” is intended to include “and” unless the context clearly indicates otherwise.

As used herein the terms “comprising” or “comprises” means “including” or “includes” and are used in reference to compositions, methods, systems, and respective component(s) thereof, that are useful to the invention, yet open to the inclusion of unspecified elements, whether useful or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, systems, and respective components thereof as described herein, which are exclusive of any element not recited in that description of the embodiment.

The abbreviation, “e.g.” is derived from the Latin exempli gratia, and is used herein to indicate a non-limiting example. Thus, the abbreviation “e.g.” is synonymous with the term “for example.”

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, rabbits, deer, bison, buffalo, goats, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “individual,” “patient,” “subject,” and the like are used interchangeably herein. The terms do not denote a particular age, and thus encompass adults, children, and newborns. A subject can be a male or female.

Preferably, the subject is a mammal. The mammal can be a human, non-human primate, mouse, rat, dog, cat, horse, or cow, but is not limited to these examples. Mammals other than humans can be advantageously used as subjects in animal models of human treatment or disease. In addition, the methods and compositions described herein can be used for treatment of domesticated animals and/or pets. A human subject can be of any age, gender, race or ethnic group. In some embodiments, the subject can be a patient or other subject in a clinical setting. In some embodiments, the subject can already be undergoing treatment.

As used herein, the term “administer” refers to the placement of a composition into a subject by a method or route which results in at least partial localization of the composition at a desired site such that the desired effect is produced.

As used herein, the terms “treat,” “treatment,” “treating”, or “amelioration” are used herein to characterize a method or process that is aimed at (1) slowing down or stopping the progression, aggravation, or deterioration of the symptoms of the disease or condition; or (2) bringing about ameliorations of the symptoms of the disease or condition. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also slowing of progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased morbidity or mortality. The term “treatment” of a disease also includes providing relief from the symptoms or side-effects of the disease (including palliative treatment). A treatment can be administered prior to the onset of the disease, for a prophylactic or preventive action. Alternatively, or additionally, the treatment can be administered after initiation of the disease or condition, for a therapeutic action.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

As regards all the embodiments/examples characterized in this specification, in particular in the claims, it is intended that each embodiment mentioned in a dependent claim is combined with each embodiment of each claim (independent or dependent) said dependent claim depends from. As an example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations: A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; and C, F, I, unless specifically mentioned otherwise.

Similarly, and also in those cases where independent and/or dependent claims do not recite alternatives, it is understood that if dependent claims refer back to a plurality of preceding claims or plurality of embodiments, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to anyone of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all claims and embodiments of the present specification. To give a few examples, the combination of claims 3, 5 and 1 is clearly and unambiguously envisaged in view of the claim structure/claimed subject-matter. The same applies for the combinations of claims 12, 10, 3 and 5, and, to give a few further examples which are not limiting, the combination of claims 14, 13, 9 and 8 and the combination of claims 13, 9 and 8.

The disclosure is further illustrated by the following examples which should not be construed as limiting. The examples are illustrative only, and are not intended to limit, in any manner, any of the aspects described herein. The following examples do not in any way limit the invention.

### EXAMPLES

### Example 1

### Preparation of Mycophenolate Mofetil Suspended Ointment Formulations for Uveitis Treatment

Mycophenolate mofetil suspended ointment formulation was prepared by using a mixture of light liquid paraffin, Merkur 500 (white soft paraffin), lanolin and lanolin alcohol as ointment base (Table 1).

TABLE 1
Mycophenolate mofetil suspended ointment

| | | Composition in | |
|---|---|---|---|
| | | Percentage (% w/w) | |
| | | VLN-F-70/ | VLN-F-70/ |
| Ingredient | Function | ASR/063 | ASR/064 |
| Mycophenolate Mofetil | API | 1.0 | 2.0 |
| Light liquid paraffin | Ointment base | 27.3 | 26.3 |
| (Drakeol 19) | | | |
| White soft paraffin | Ointment base | 63.7 | 63.7 |
| (Merkur 500) | | | |
| Lanolin | Ointment base | 3.0 | 3.0 |
| Lanolin Alcohol | Ointment base | 5.0 | 5.0 |

Method of Preparation:
1. In a main mixing vessel, precisely weighed quantity of Merkur 500, lanolin and lanolin alcohol were taken in a SS beaker and maintained at a constant stirring using anchor stirrer over a water bath maintained at 60-65° C.
2. Weighed quantity of Mycophenolate Mofetil was added to a part of Drakeol 19 under high shear homogenization (10,000 rpm) and was allowed to form slurry for 20 min.
3. Slurry prepared in step 2 was added to molten phase of step 1, under stirring at 150 rpm using the anchor stirrer.
4. Remaining part of Drakeol 19 was used to rinse the high shear homogenizer and added to step 3 phase.
5. Formulation obtained in step 4 was allowed to congeal at room temperature with stirring.

TABLE 2
Stability Data

| | | 1% w/w Ointment | 2% w/w Ointment |
|---|---|---|---|
| Batch Number | | VLN-F-70/ASR/063 | VLN-F-70/ASR/064 |
| Initial | Assay | 100.39 | 99.88 |
| 1 Month | 25° C./60% RH | 96.41 | 96.21 |
| | 40° C./75% RH | 111.68 | 101.85 |
| 2 Month | 25° C./60% RH | 98.88 | 99.87 |
| | 40° C./75% RH | 108.70 | 100.22 |
| 3 Month | 25° C./60% RH | 99.01 | 98.91 |
| | 40° C./75% RH | 116.84 | 112.54 |
| 6 Month | 25° C./60% RH | 103.08 | 99.91 |
| | 40° C./75% RH | 102.35 | 109.12 |

TABLE 3
Degradation Product data

| | | VLN-F-70/ASR/063 | | | | VLN-F-70/ASR/064 | | | |
|---|---|---|---|---|---|---|---|---|---|
| | | (1% w/w ointment) | | | | (2% w/w ointment) | | | |
| | | 25° C./60% RH | | 40° C./75% RH | | 25° C./60% RH | | 40° C./75% RH | |
| Imp Name | Initial | 3 M | 6 M | 3 M | 6 M | 3 M | 6 M | 3 M | 6 M |
| Mycophenolic | ND | 0.141 | 0.113 | 0.465 | 1.047 | 0.082 | 0.080 | 0.177 | 0.255 |
| acid | | | | | | | | | |
| Total Impurity | ND | 0.401 | 0.344 | 0.835 | 1.332 | 0.333 | 0.275 | 0.478 | 0.523 |

Observation: The products was found to be stable in terms of degradation product profile. There was a variation in the assay of Mycophenolate Mofetil at accelerated stability condition, which could be due to melting of the ointment base at higher storage temperatures.

### Example 2

Different composition trials for ointment vehicle to enhance the melting point of formulation.

During stability studies there was bleeding and loss in viscosity (due to melting) in the samples kept at 40° C./75% RH stability samples. This led to the variation in assay. To resolve this problem further trials of vehicles were executed (Table 2).

TABLE 4
Composition trials for ointment vehicle to enhance the melting point of formulation

| | | Composition in Percentage (% w/w) | | | |
|---|---|---|---|---|---|
| | | VLN-F-70/ | VLN-F-70/ | VLN-F-70/ | VLN-F-70/ |
| Ingredient | Function | ASR/103A | ASR/103B | ASR/103C | ASR/103D |
| Drakeol 19 (Light | Ointment base | 27.3 | 22.3 | 15.3 | 5.3 |
| liquid paraffin) | | | | | |
| Merkur 500 (White | Ointment base | 63.631 | 63.631 | 70.631 | 80.631 |
| soft paraffin) | | | | | |
| Lanolin | Ointment base | 3.0 | 3.0 | 3.0 | 3.0 |
| Lanolin Alcohol | Ointment base | 5.0 | 10.0 | 10.0 | 10.0 |

Method of Preparation:
1. In a main mixing vessel, precisely weighed quantity of Merkur 500, lanolin and lanolin alcohol were taken in a SS beaker and maintained at a constant stirring using anchor stirrer over a water bath maintained at 60-65° C.
2. Weighed quantity of Drakeol 19 was collected in a separate beaker and maintained at 60-65° C. using a water bath.
3. Material prepared in step 2 was added to molten phase of step 1, under stirring at 150 rpm using the anchor stirrer.
4. Formulation obtained in step 3 was allowed to congeal at room temperature with stirring.

TABLE 5
Physical Observation

| | VLN-F-70/ | VLN-F-70/ | VLN-F-70/ | VLN-F-70/ |
|---|---|---|---|---|
| Ingredient | ASR/103A | ASR/103B | ASR/103C | ASR/103D |
| Viscosity | 6169.5 | 6360.7 | 7256.8 | 11603 |
| (mPas) | | | | |
| Behavior at | Flow | Flow | Consistency | Consistency |
| 40° C. | with | with | maintained | maintained |
| | tapping | tapping | | |
| Behavior at | Liquid | Liquid | Liquid | Liquid |
| 50° C. | | | | |

Observation: The formulations made with increased levels of White Petrolatum showed better consistency.

### Example 3

As per the results obtained during stability studies the was some increase in the mycophenolic acid content. Hence, trials were conducted with low temperature processing conditions in order to minimize the chance of degradation of mycophenolate mofetil to mycophenolic acid during processing. Trials were planned with reducing the processing temperature of Mycophenolate mofetil ointment.

TABLE 6
Mycophenolate mofetil suspended ointment
formulations at reduced temperature

| | | Composition in Percentage (% w/w) | |
|---|---|---|---|
| | | VLN-F-70/ | VLN-F-70/ |
| Ingredient | Function | ASR/118A | ASR/118B |
| Mycophenolate | API | 1.0 | 2.0 |
| Mofetil | | | |
| Drakeol 19 (Light | Ointment | 27.3 | 26.3 |
| liquid paraffin) | base | | |
| Merkur 500 (White | Ointment | 63.7 | 63.7 |
| soft paraffin) | base | | |
| Lanolin | Ointment | 3.0 | 3.0 |
| | base | | |
| Lanolin Alcohol | Ointment | 5.0 | 5.0 |
| | base | | |

Method of Preparation:
1. In a main mixing vessel, precisely weighed quantity of Merkur 500, lanolin and lanolin alcohol were taken in a SS beaker and maintained at a constant stirring using anchor stirrer over a water bath maintained at 60-65° C.
2. Weighed quantity of Mycophenolate mofetil was added to a part of Drakeol 19 under high shear homogenization (10,000 rpm) and was allowed to form slurry for 30 min.
3. Material of step 1 was allowed to cool down with stirring up to 50° C.
4. Slurry prepared in step 2 was added to molten phase of step 4, under stirring at 150 rpm using the anchor stirrer.
5. Remaining part of Drakeol 19 was used to rinse the high shear homogenizer and added to step 4 phase.
6. Formulation obtained in step 4 was allowed to congeal at room temperature with stirring.

TABLE 7
Stability Data

| | |
|---|---|
| Initial | Assay (%), 3 M |

| | Assay | 25° C./ | 40° C./ |
|---|---|---|---|
| Batch detail | (%) | 60% RH | 75% RH |
| 1% w/w Mycophenolate | 100.29 | 98.78 | 112.49 |
| Mofetil ointment | | | |
| (VLN-F70/ASR/118A) | | | |
| 2% w/w Mycophenolate | 98.06 | 97.13 | 118.35 |
| Mofetil ointment | | | |
| (VLN-F70/ASR/118B) | | | |

TABLE 8
Degradation product data

| | | | VLN-F-70/ | VLN-F-70/A |
|---|---|---|---|---|
| | | | ASR/118A | SR/118B |
| | Impurity Name | RRT | 1% w/w ointment | 2% w/w ointment |
| Initial | Mycophenolic acid | 0.27 | 0.062 | 0.057 |

| | | |
|---|---|---|
| Total Impurities | 0.156 | 0.160 |

| | | | VLN-F-70/ASR/118A | | VLN-F-70/ASR/118B | |
|---|---|---|---|---|---|---|
| | | | 1% w/w Ointment | | 2% w/w Ointment | |
| | Imp Name | RRT | 25° C./60% RH | 40° C./75% RH | 25° C./60% RH | 40° C./75% RH |
| 3 Month | Mycophenolic acid | 0.548 | 0.109 | 0.470 | 0.088 | 0.343 |
| | Total Impurity | | 0.283 | 0.742 | 0.255 | 0.588 |

### Example 4

An additional approach in ointment formulation is to use Mycophenolate sodium instead of Mycophenolate Mofetil and utilizing the understanding developed during the previous development trials. An ointment formulation was prepared by using a mixture of light liquid paraffin, Merkur 500 (white soft paraffin), lanolin and lanolin alcohol as ointment base (Table 1).

Preparation of mycophenolate sodium suspended ointment formulations at reduced temperature.

TABLE 9
Mycophenolate sodium suspended ointment

| | | Composition in |
|---|---|---|
| | | Percentage (% w/w) |
| Ingredient | Function | VLN-F-70/ASR/133 |
| Mycophenolate sodium | API | 2.0 |
| Drakeol 19 (Light | Ointment base | 26.3 |
| liquid paraffin) | | |
| Merkur 500 (White | Ointment base | 63.7 |
| soft paraffin) | | |
| Lanolin | Ointment base | 3.0 |
| Lanolin Alcohol | Ointment base | 5.0 |

Method of Preparation:
1. In a main mixing vessel, precisely weighed quantity of Merkur 500, lanolin and lanolin alcohol were taken in a SS beaker and maintained at a constant stirring using anchor stirrer over a water bath maintained at 60-65° C.
2. Weighed quantity of Mycophenolate sodium was added to a part of Drakeol 19 under high shear homogenization (10,000 rpm) and was allowed to form slurry for 30 min.
3. Material of step 1 was allowed to cool down with stirring up to 50° C.
4. Slurry prepared in step 2 was added to molten phase of step 4, under stirring at 150 rpm using the anchor stirrer.
5. Remaining part of Drakeol 19 was used to rinse the high shear homogenizer and added to step 4 phase.
6. Formulation obtained in step 4 was allowed to congeal at room temperature with stirring.
7. The formulation is placed on stability at real time and accelerated conditions.

### Example 5

To further improvise the formulation and to develop a formulation that does not loose consistency behavior at accelerated temperature some trials with improved composition and at low temperature processing condition were executed. To study the effect of packaging material compatibility with the ointment formulation, the final product was packed in two different types of tubes; Aluminum and Laminated tubes and placed on stability.

Preparation of mycophenolate mofetil suspended ointment formulations with improved composition and at low temperature processing condition

TABLE 10
Mycophenolate mofetil suspended ointment formulations with improved
composition and at low temperature processing condition

| | | Composition in | |
|---|---|---|---|
| | | Percentage (% w/w) | |
| | | VLN-F-70/ | VLN-F-70/ |
| Ingredient | Function | ASR/149 | ASR/155 |
| Mycophenolate Mofetil | API | 1.0 | 2.0 |
| Drakeol 19 (Light | Ointment base | 15.3 | 15.3 |
| liquid paraffin) | | | |
| Crolatum V (White | Ointment base | 70.7 | 82.7 |
| soft paraffin) | | | |
| Lanolin | Ointment base | 3.0 | — |
| Lanolin Alcohol | Ointment base | 10.0 | — |

Method of Preparation:
1. In a main mixing vessel, precisely weighed quantity of ointment bases were taken in a SS beaker and maintained at a constant stirring using anchor stirrer over a water bath maintained at 60-65° C.
2. Weighed quantity of Mycophenolate mofetil was added to a part of Drakeol 19 under high shear homogenization (10,000 rpm) and was allowed to form slurry for 30 min.
3. Material of step 1 was allowed to cool down with stirring up to 50° C.
4. Slurry prepared in step 2 was added to molten phase of step 4, under stirring at 150 rpm using the anchor stirrer.
5. Remaining part of Drakeol 19 was used to rinse the high shear homogenizer and added to step 4 phase.
6. Formulation obtained in step 4 was allowed to congeal at room temperature with stirring.

TABLE 11
Stability Data

| | | Assay of Mycophenolate | | |
|---|---|---|---|---|
| | | Mofetil (% w/w) | | |
| | | | 3 M | |
| | | | 25° C./ | 40° C./ |
| Sample B. No. | Pack type | Initial | 60% RH | 75% RH |
| VLN-F-70/ASR/ | Aluminium | 95.87 | 96.28 | 106.39 |
| 149 AL 1% Ointment | tube | | | |
| VLN-F-70/ASR/ | Laminated | | 98.29 | 113.43 |
| 149LA 1% Ointment | tube | | | |
| VLN-F-70/ASR/ | | 95.49 | 35.29 | 78.74 |
| 155 2% Ointment | | | | |

Observation: There was a uniformity issue observation at accelerated storage conditions.

### Example 6

With the use of white petrolatum, Van der Waal forces keep crystal structures in place due to the number of attraction points available. However, under relatively small force application, these crystals will shear and fracture. To resolve this problem, mycophenolate mofetil suspended ointment formulations with improved composition and controlled processing (low temperature and low stirring) condition were prepared. Mycophenolate mofetil suspended ointment formulations were accordingly prepared.

TABLE 12
Mycophenolate mofetil suspended ointment formulations
with improved composition and controlled processing
(low temperature and low stirring) condition

| | | Composition in |
|---|---|---|
| | | Percentage (% w/w) |
| Ingredient | Function | VLN-F-70/ASR/185 |
| Mycophenolate Mofetil | API | 2.0 |
| Vaportum (White | Ointment base | 98.0 |
| soft paraffin) | | |

Method of Preparation:
1. In a main mixing vessel, a part (approx. 80%) of Vaportum was precisely weighed and was taken in a SS beaker and maintained at a constant stirring of 30 rpm using anchor stirrer over a water bath maintained at 60-65° C.
2. Another part (approx. 10%) of Vaportum was precisely weighed melted in a separate container.
3. Mycophenolate mofetil was weighed and mixed with the molten phase of step 2 under high shear homogenization (10,000 rpm).
4. Slurry prepared in step 3 was added to molten phase of step 1, under slow stirring of 30 rpm using the anchor stirrer.
5. Remaining part of Vaportum (approx. 8%) was used to rinse the high shear homogenizer and added to step 4 phase.
6. Now temperature was allowed to come down till congealing starts with slow mixing at 30 rpm.
7. Congealing was observed at 45° C.
8. Stirring was stopped to prevent crystal breakage and system was allowed to cool down at room temperature.
9. The formulation was placed on stability for further study.

Observation: The physical observation of samples kept at accelerated condition has no change in consistency or melting of the formulation suggest a better formulation compared to all other earlier formulations.

In-Vitro Release Study Using Goat Cornea
Membrane used: Goat eye cornea was sandwiched between donor and receptor compartment.
Media: Phosphate buffer saline pH 7.4
Replication: Each formulation was applied on Six cells
Dose: Equivalent to 500 μg of active for 1% formulation and 1000 μg of active for 2% formulation.
Study Duration—2 hr

TABLE 13
Results

| | Cornea Top | | Cornea Extracted | | Receptor Media | | Flux | Recovery |
|---|---|---|---|---|---|---|---|---|
| | (μg) | | (μg) | | (μg) | | (μg/cm2) # | (%) |
| 1% w/w | MMF | 423.58 ± 171.03 | MMF | 6.42 ± 6.52 | MMF | 0.26 ± 0.13 | 11.01 | 74.80 |
| Mycophenolate | MPA | 16.72 ± 1.21 | MPA | 29.21 ± 4.62 | MPA | 19.97 ± 10.56 | 26.54 | 76.55 |
| Mofetil ointment | Total | 440.3 | Total | 35.63 | Total | 20.23 | | |
| (VLN-F70/ASR/063) | | | | | | | | |
| 2% w/w | MMF | 764.12 ± 215.28 | MMF | 10.65 ± 5.32 | MMF | 0.59 ± 0.48 | | |
| Mycophenolate | MPA | 17.89 ± 1.47 | MPA | 37.06 ± 3.49 | MPA | 48.17± 23.14 | | |
| Mofetil ointment | Total | 782.01 | Total | 47.71 | Total | 48.76 | | |
| (VLN-F70/ASR/064) | | | | | | | | |

#Considering 1.837 cm2 is the active permeation area
* MMF—Mycophenolate mofetil
MPA—Mycophenolic Acid

Observation: Flux increases with increase in strength of the formulation i.e. 1% to 2% w/w.

Ex-Vivo Studies Using Whole Goat Eye
Freshly excised complete goat eyeball was procured and adhering tissue was removed.
Franz Cells were filled with PBS 7.4 and connected to water circulator maintained at 37° C.
Complete eyeball wrapped up in cotton was placed over the Franz cells in a fashion that it will remain in contact with the PBS 7.4 media.
PBS 7.4 solution was infused at 10 μL/min to the anterior portion of eye using Micro infusion pump.
A needle was attached to the diagonally opposite end for sample collection.
A circular ring of 8.5 mm was placed over the cornea along with the cotton outside the ring and formulation was applied inside the ring.
After 7 min, formulation was wicked using cotton and washing was done using 1 ml PBS 7.4. Solution obtained during washing was also collected (from the cotton placed around the ring).
Media: Phosphate buffer saline pH 7.4
Replication: Each formulation was applied on Six cells. Dose: Equivalent to 500 μg of active for 1% formulation and 1000 μg of active for 2% formulation.

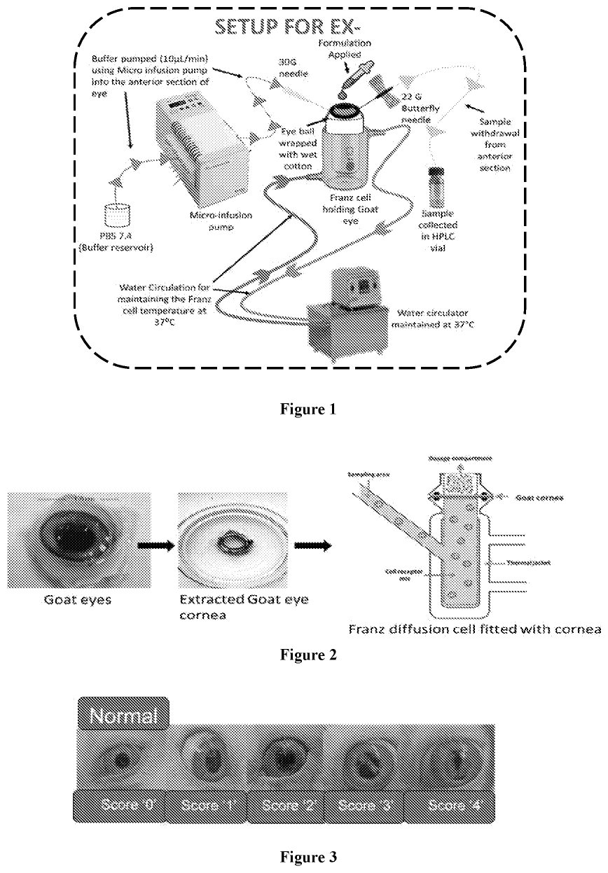

The above described ex-vivo study set up is illustrated in FIG. 1.

TABLE 14
Results

| | | | | | | | | Total | | |
|---|---|---|---|---|---|---|---|---|---|---|
| | | | | | Cornea | | | amount | Cumulative | |
| | No. of | | | | extraction | Lens | Iris | of drug | amt of drug | |
| Formulations | Replicates | Time | Cornea top | | (mcg) | (mcg) | (mcg) | retained | in receptor | |
| studied | (n) | points | (mcg) | | a | b | c | a + b + c | (mcg) | Recovery |
| 1.0% w/w | 6 | 1, 2, 3, | MMF | 422.3 ± 38.5 | 12.9 ± 4.1 | 0.4 ± 0.4 | 0.2 ± 0.2 | 13.5 | 0.06 ± 0.04 | 79.9% |
| Mycophenolate | | 4 & 5 hr | MPA | 14.6 ± 0.4 | 2.0 ± 0.3 | 0.3 ± 0.1 | 0.5 ± 0.3 | 2.9 | 0.78 ± 0.36 | |
| Mofetil ointment | | | Total | 436.9 | 14.9 | 0.7 | 0.7 | 16.4 | 0.84 | |
| (VLN-F70/ASR/063) | | | | | | | | | | |
| 2.0% w/w | 5 | 1, 2, 3, | MMF | 981.1 ± 218.1 | 14.2 ± 13.3 | 1.3 ± 1.5 | 0.6 ± 0.6 | 16.2 | 0.03 ± 0.01 | 85.9% |
| Mycophenolate | | 4 & 5 hr | MPA | Not | 3.3 ± 2.6 | 0.2 ± 0.3 | 0.3 ± 0.5 | 3.6 | 1.51 ± 0.79 | |
| Mofetil ointment | | | | detected | | | | | | |
| (VLN-F70/ASR/064) | | | Total | 981.1 | 17.5 | 1.5 | 0.9 | 19.9 | 1.54 | |

Results are shown as Mean ± SD
* MMF—Mycophenolate mofetil; MPA—Mycophenolic Acid

Observation: Cumulative amount of drug permeated increases with increase in strength of the ointment formulation.

### Example 7

An ophthalmic ointment formulation comprising Mycophenolate Mofetil as active, Petrolatum, lanolin alcohol, lanolin and light liquid paraffin as ointment base.

TABLE 15

| | Ingredient | Function | Composition (% w/w) |
|---|---|---|---|
| Phase A | Mycophenolate | Active | 1.0 |
| | Mofetil | | |
| | Lanolin | Solubilizer | 3.0 |
| | Lanolin Alcohol | Solubilizer | 10.0 |
| | Chlorobutanol | Preservative | 0.5 |
| Phase B | Petrolatum | Base | 57.0 |
| Phase C | Light Liquid | Base | 28.5 |
| | paraffin | | |

Method of Preparation:
1. Lanolin and Lanolin Alcohol are heated in a beaker between 70±2° C.
2. Mycophenolate Mofetil is added to this mixture and dissolved at the same temperature with constant stirring.
3. Chlorobutanol is added to the API phase and maintain temperature of the phase between 70±2° C.
4. Petrolatum is melted in a beaker using water bath maintained between 70±2° C.
5. Light Liquid paraffin is added to molten Petrolatum phase and maintain the temperature of the phase between 70±2° C.
6. API phase of step 3 is added to the petrolatum phase of step 5 with constant stirring. Mixing is continued for 30±5 minutes.
7. Heating is removed and the bulk is allowed to cool slowly.
8. Viscosity of the final ointment formulation is maintained at 4120 mPas.

### Example 8

An ophthalmic ointment formulation comprising Mycophenolate Mofetil as active. Petrolatum, lanolin oil and light liquid paraffin as ointment base.

TABLE 16

| | Ingredient | Function | Composition (% w/w) |
|---|---|---|---|
| Phase A | Mycophenolate | Active | 1.0 |
| | Mofetil | | |
| | Lanolin alcohol | Solubilizer | 3.0 |
| | Chlorobutanol | Preservative | 0.5 |
| Phase B | Petrolatum | Base | 23.5 |
| | Light Liquid | Base | 72.0 |
| | paraffin | | |

Method of Preparation:
1. Lanolin alcohol is taken in a beaker using water bath between 70±2° C.
2. Mycophenolate Mofetil is added to the heated Lanolin alcohol phase of step 1 and continue mixing to dissolve Mycophenolate Mofetil.
3. Chlorobutanol is added to the API phase and maintain temperature of the phase between 70±2° C.
4. Petrolatum is melted in a beaker using water bath maintained at 70±2° C.
5. Light Liquid paraffin is added to the molten Petrolatum phase and maintain the temperature of the phase between 70±2° C.
6. API phase of step 3 is added to the petrolatum phase of step 5 with constant stirring. Mixing continued for 30±5 minutes.
7. Heating is removed and the bulk is allowed to cool slowly.

### Example 9

An ophthalmic ointment formulation comprising Mycophenolate Mofetil as active, Glyceryl monostearate as solubilizer, Petrolatum and light liquid paraffin as ointment base.

TABLE 17

| | Ingredient | Function | Composition (% w/w) |
|---|---|---|---|
| Phase A | Mycophenolate | Active | 1.0 |
| | mofetil | | |
| | Light liquid | Vehicle | 4.0 |
| | paraffin | | |
| | Glyceryl | Solubilizer | 5.0 |
| | monostearate | | |
| Phase B | Petrolatum | Base | 90.0 |

Method of Preparation:
1. Glyceryl monostearate is heated in a beaker using water bath between 70±2° C.
2. Light liquid paraffin is added to step 1 followed by Mycophenolate Mofetil under continuous mixing to dissolve Mycophenolate Mofetil.
3. Petrolatum is melted in a beaker using water bath maintained at 70±2° C.
4. API phase of step 2 is added to the petrolatum phase of step 3 under constant stirring. Mixing is continued for 30±5 minutes.
5. Heating is removed and the bulk is allowed to cool slowly.

### Example 10

An ophthalmic ointment formulation comprising Mycophenolate Mofetil as active, Lanolin Alcohol and Petrolatum as base, Glycerin as humectant and chlorobutanol as preservative.

TABLE 18

| | Ingredient | Function | Composition (% w/w) |
|---|---|---|---|
| Phase A | Mycophenolate | Active | 1.0 |
| | mofetil | | |
| | Lanolin Alcohol | Vehicle | 10.0 |
| | Chlorobutanol | Preservative | 1.0 |
| | Glycerin | Humectant | 5.0 |
| Phase B | Petrolatum | Base | 83 |

Method of Preparation:
1. Lanolin alcohol is heated in a beaker using water bath between 70±2° C.
2. Chlorobutanol and glycerin are added to step 1 followed by Mycophenolate Mofetil under continuous mixing to dissolve Mycophenolate Mofetil.
3. Petrolatum is melted in a beaker using water bath maintained at 70±2° C.
4. API phase of step 2 is added to the petrolatum phase of step 3 under constant stirring. Mixing continued for 30±5 minutes.
5. Heating is removed and the bulk is allowed to cool slowly.

### Example 11

An ophthalmic ointment formulation comprising Mycophenolate Mofetil as active, Glyceryl monostearate as solubilizer, Petrolatum, castor oil and light liquid paraffin as ointment base.

TABLE 19

| | Ingredient | Function | Composition (% w/w) |
|---|---|---|---|
| Phase A | Mycophenolate | Active | 1.0 |
| | mofetil | | |
| | Castor oil | slippery action | 5.0 |
| | Glyceryl | Solubilizer | 5.0 |
| | monostearate | | |
| Phase B | Petrolatum | Ointment Base | 53 |
| | Light liquid | Ointment Base | 36 |
| | paraffin | | |

Method of Preparation:
1. Castor oil is heated in a beaker using water bath between 70±2° C.
2. Glyceryl monostearate is added to step 1 followed by Mycophenolate Mofetil under continuous mixing to dissolve Mycophenolate Mofetil.
3. In a separate beaker melt Petrolatum in a using water bath maintained at 70±2° C.
4. Light liquid paraffin is added to step 3 under constant stirring. Mixing continued for 30±5 minutes.
5. API phase of step 2 is added to the petrolatum phase of step 4 under constant stirring. Mixing continued for 30±5 minutes.
6. Heating is removed and the bulk is allowed to cool slowly.

### Example 12

An ophthalmic ointment formulation comprising Mycophenolate Mofetil as active, Petrolatum, and light liquid paraffin as ointment base, and lanolin as humectant and beeswax as vehicle.

TABLE 20

| | Ingredient | Function | Composition (% w/w) |
|---|---|---|---|
| Phase A | Mycophenolate | Active | 1.0 |
| | mofetil | | |
| | Beeswax | Vehicle | 5.0 |
| | Lanolin | Humectant | 10.0 |
| | Petrolatum | Ointment Base | 54 |
| | Light liquid | Ointment Base | 30 |
| | paraffin | | |

Method of Preparation:
1. Castor oil is heated in a beaker using water bath between 70±2° C.
2. Glyceryl monostearate is added to step 1 followed by Mycophenolate Mofetil under continuous mixing to dissolve Mycophenolate Mofetil.
3. In a separate beaker Petrolatum is melted using a water bath maintained at 70±2° C.
4. Light liquid paraffin is added to step 3 under constant stirring.
5. API phase of step 2 is added to the petrolatum phase of step 4 under constant stirring. Mixing continued for 30±5 minutes.
6. Heating is removed and the bulk is allowed to cool slowly.

### Example 13

An ophthalmic ointment formulation comprising Mycophenolate Mofetil as active, Ceresin wax and Petrolatum as ointment base, Dexpanthenol and glycerin as humectant.

TABLE 21

| | Ingredient | Function | Composition (% w/w) |
|---|---|---|---|
| Phase A | Mycophenolate | Active | 1.0 |
| | mofetil | | |
| | Ceresin Wax | Ointment Base | 10.0 |
| | Dexpanthenol | Humectant | 2 |
| | Glycerin | Humectant | 10 |
| Phase B | Petrolatum | Ointment Base | 77 |

Method of Preparation:
1. Ceresin wax is heated in a beaker using water bath between 70±2° C.
2. Dexpanthenol and Glycerin are added to step 1 followed by Mycophenolate Mofetil under continuous mixing to uniformly suspend Mycophenolate Mofetil.
3. In a separate beaker Petrolatum is melted using a water bath maintained at 70±2° C.
4. API phase of step 2 is added to the petrolatum phase of step 3 under constant stirring. Mixing continued for 30±5 minutes.
5. Heating is removed and the bulk is allowed to cool slowly.

### Example 14

An ophthalmic ointment formulation comprising Mycophenolate Mofetil as active, Petrolatum, Lanolin alcohol, cetyl alcohol, stearic acid, light liquid paraffin and Petrolatum as ointment base, steareth-2 as solubilizer, Glycerin and Squalene as humectant and D-Alpha-tocopherol acetate as antioxidant.

TABLE 22

| | Ingredient | Function | Composition (% w/w) |
|---|---|---|---|
| Phase A | Mycophenolate | Active | 1.0 |
| | mofetil | | |
| | Lanolin Alcohol | emollient | 10.0 |
| | Cetyl alcohol | emollient | 5 |
| | Glycerin | Humectant | 10 |
| | Steareth-2 | Solubilizer | 5 |
| | Squalene | Humectant | 5 |
| | Stearic acid | Base | 5 |
| | D-Alpha- | Antioxidant | 1 |
| | tocopherol acetate | | |
| Phase B | Light liquid | ointment base | 18 |
| | paraffin | | |
| | Petrolatum | ointment base | 40 |

Method of Preparation:
1. Lanolin alcohol is taken in a beaker and heated between 70±2° C. using a water bath.
2. Cetyl alcohol, steareth-2 and stearic acid are added to step 1 followed by Mycophenolate Mofetil and mixing is continued to get a clear solution.
3. Chlorobutanol is added to the API phase and temperature of the phase is maintained between 70±2° C.
4. Petrolatum is taken in a beaker and melted using water bath maintained at 70±2° C.
5. Light Liquid paraffin is added to molten Petrolatum phase and temperature of the phase is maintained between 70±2° C.
6. API phase of step 2 is added to the petrolatum phase of step 5 under constant stirring. Mixing continued for 30±5 minutes.
7. Heating is removed and the bulk is allowed to cool slowly.

### Example 15

An ophthalmic ointment formulation comprising Mycophenolate Mofetil as active, Petrolatum as ointment base, propylene glycol as humectant, glyceryl monostearate as solubilizer.

TABLE 23

| Ingredient | Function | Composition (% w/w) |
|---|---|---|
| Mycophenolate mofetil | Active | 1.0 |
| Petrolatum | Ointment Base | 84.0 |
| Propylene glycol | Humectant | 5 |
| Glyceryl monostearate | solubilizer | 10 |

Method of Preparation:
1. Glyceryl monostearate and Propylene glycol are taken in a beaker and heated between 70±2° C. using a water bath.
2. Mycophenolate Mofetil is to step 1 under continue mixing to get a clear solution.
3. Petrolatum is added to step 2 under mixing and maintaining the temperature between 70±2° C. Mixing continued for 30±5 minutes.
4. Heating is removed and the bulk is allowed to cool slowly.

### Example 16

An ophthalmic ointment formulation comprising Mycophenolate sodium as active and Polyethylene glycol 400 and Polyethylene glycol 1450 as ointment base and glycerin as co-solvent.

TABLE 24

| | Ingredient | Function | Composition (% w/w) |
|---|---|---|---|
| Phase A | Mycophenolate sodium | Active | 1.0 |
| | equivalent to | | |
| | Mycophenolic acid | | |
| | Polyethylene glycol | Ointment | 64 |
| | 400 | Base | |
| | Polyethylene glycol | Ointment | 30 |
| | 1450 | Base | |
| | Glycerin | Co-solvent | 5 |

Method of Preparation:
1. Polyethylene glycol 400 and Polyethylene glycol 1450 are taken in a beaker and heated between 70±2° C. using a water bath.
2. Mycophenolate sodium is added to step 1 under continue mixing to get a clear solution.
3. Glycerin is added to step 2 under mixing and maintaining the temperature between 70±2° C. Mixing is continued for 30±5 minutes.
4. Heating is removed and the bulk is allowed to cool slowly.

### Example 17

An ophthalmic ointment formulation comprising Mycophenolate sodium as active and Polyethylene glycol 400 and Polyethylene glycol 1450 as ointment base, glycerin and propylene glycol as co-solvent.

TABLE 25

| | Ingredient | Function | Composition (% w/w) |
|---|---|---|---|
| Phase A | Mycophenolate sodium | Active | 1.0 |
| | equivalent to | | |
| | Mycophenolic acid | | |
| | Polyethylene glycol | Ointment | 50 |
| | 400 | Base | |
| | Polyethylene glycol | Ointment | 29 |
| | 1450 | Base | |
| | Glycerin | Co-solvent | 10 |
| | Propylene glycol | Co-solvent | 10 |

Method of Preparation:
1. Polyethylene glycol 400 and Polyethylene glycol 1450 are taken in a beaker and heated between 70±2° C. using a water bath.
2. Mycophenolate sodium is added to step 1 under continue mixing to get a clear solution.
3. Propylene glycol and glycerin are added to step 2 under mixing and maintaining the temperature between 70±2° C. Mixing continued for 30±5 minutes.
4. Heating is removed and the bulk is allowed to cool slowly.

### Example 18

An ophthalmic ointment formulation comprising Mycophenolate sodium as active and Polyethylene glycol 200, Polyethylene glycol 300 and Polyethylene glycol 3350 as ointment base.

TABLE 26

| | Ingredient | Function | Composition (% w/w) |
|---|---|---|---|
| Phase A | Mycophenolate sodium | Active | 1.0 |
| | equivalent to | | |
| | Mycophenolic acid | | |
| | Polyethylene glycol | Ointment | 40 |
| | 300 | Base | |
| | Polyethylene glycol | Ointment | 19 |
| | 200 | Base | |
| | Polyethylene glycol | Solubilizer | 40 |
| | 3350 | | |

Method of Preparation:
1. Polyethylene glycol 300, Polyethylene glycol 200 and Polyethylene glycol 3350 are taken in a beaker and heated between 70±2° C. using a water bath.
2. Mycophenolate sodium is added to step 1 under continue mixing to get a clear solution.
3. Heating is removed and the bulk is allowed to cool slowly.

### Example 19

An ophthalmic ointment formulation comprising Mycophenolate sodium as active and Polyethylene glycol 200, Polyethylene glycol 300, Polyethylene glycol 3350 and lanolin alcohol as ointment base.

TABLE 27

| | Ingredient | Function | Composition (% w/w) |
|---|---|---|---|
| Phase A | Mycophenolate sodium | Active | 1.0 |
| | equivalent to | | |
| | Mycophenolic acid | | |
| | Lanolin alcohol | lubricant | |
| | Polyethylene glycol | Ointment | 40 |
| | 300 | Base | |
| | Polyethylene glycol | Ointment | 19 |
| | 200 | Base | |
| | Polyethylene glycol | Solubilizer | 40 |
| | 3350 | | |

Method of Preparation:
1. Polyethylene glycol 300, Polyethylene glycol 200 and Polyethylene glycol 3350 are taken in a beaker and heated between 70±2° C. using a water bath.
2. Lanolin alcohol is added to step 1 under continuous stirring to get a uniform phase.
3. Mycophenolate sodium is added to step 1 under continue mixing to get a clear solution.
4. Heating is removed and the bulk is allowed to cool slowly.

### Example 20

An ophthalmic emulsion formulation comprising Mycophenolate Mofetil as active, Light liquid paraffin as the oily vehicle, Polyoxyl 35 castor oil and Polysorbate 80 as emulsifier, Glycerin as co solvent, Hydroxyethyl cellulose as thickener and Boric acid as preservative.

TABLE 28

| | Ingredient | Function | Composition (% w/v) |
|---|---|---|---|
| Phase A | Mycophenolate | Active | 1.0 |
| | Mofetil | | |
| | Light liquid | Oil component | 10 |
| | paraffin | | |
| | Polysorbate 80 | Emulsifier | 4.0 |
| | Polyoxyl 35 | Emulsifier | 5.0 |
| | castor oil | | |
| Phase B | Boric Acid | Preservative | 0.1 |
| | Glycerin | Co solvent | 2.2 |
| | Hydroxyethyl | Thickener | 0.05 |
| | cellulose | | |
| | Sodium hydroxide | pH Modifier | QS |
| | Purified water | Aqueous Phase | Q.s 100% |

Method of Preparation:
1. Polyoxyl 35 castor oil, Light liquid paraffin and Polysorbate 80 are taken in a beaker and heated to a temperature between 70±2° C.
2. Mycophenolate Mofetil is added to step 1 under stirring to get a clear phase maintaining the temperature between 70±2° C.
3. Aqueous phase is prepared by dispersing Hydroxyethyl cellulose in purified water under continuous stirring.
4. Glycerin and Boric Acid are added to step 3 under continuous stirring and temperature of the phase is maintained between 70±2° C.
5. Emulsification is done by slowly adding the oil phase of step 2 to the aqueous phase of step 4 using a high shear emulsifier and maintaining the temperature between 70±2° C.
6. The emulsification process is continued for 30±5 minutes
7. The emulsion is slowly cooled and the pH adjusted using 0.1 M NaOH solution.
8. The final pH of the formulation is maintained at 6.8 and viscosity is maintained at 17 mPas.

### Example 21

An ophthalmic emulsion formulation comprising Castor Oil as the oily vehicle, Mycophenolate Mofetil as active, Polyoxyl 15 Hydroxystearate as emulsifier, Glycerin as co-solvent, Carbomer copolymer type A as thickener and Boric acid as preservative. Preferred emulsions comprise by weight based on the total weight of the composition:

TABLE 29

| | Ingredient | Function | Composition (% w/v) |
|---|---|---|---|
| Phase A | Mycophenolate | Active | 1.0 |
| | Mofetil | | |
| | Light liquid | Oil Component | 10 |
| | paraffin | | |
| | Polysorbate 80 | Emulsifier | 4.0 |
| | Polyoxyl 35 | Emulsifier | 5.0 |
| | castor oil | | |
| | Castor oil | Oily Vehicle | 0.15 |
| Phase B | Boric Acid | Preservative | 0.1 |
| | Glycerin | Co solvent | 2.2 |
| | Carbomer copolymer | Thickener | 0.05 |
| | type A | | |
| | Sodium hydroxide | pH Modifier | QS |
| | Purified water | Aqueous Phase | Q.s 100% |

Method of Preparation:
1. Polyoxyl 35 castor oil, Light liquid paraffin, Castor oil and Polysorbate 80 are taken in a beaker and heated to a temperature between 70±2° C.
2. Mycophenolate Mofetil is added to step 1 under stirring to get a clear phase maintaining the temperature between 70±2° C.
3. Aqueous phase is prepared by dispersing Carbomer copolymer type A in purified water under continuous stirring.
4. Glycerin and Boric Acid are added to step 3 under continuous stirring and temperature of the phase is maintained between 70±2° C.
5. Emulsification is done by slowly adding the oil phase of step 2 to the aqueous phase of step 4 using a high shear emulsifier and maintaining the temperature between 70±2° C.
6. The emulsification process is continued for 30±5 minutes
7. The emulsion is slowly cooled and the pH adjusted using 0.1 M NaOH solution.
8. The final pH of the formulation is maintained at 6.5 and viscosity is maintained at 62 mPas.

### Example 22

An ophthalmic emulsion formulation comprising Castor Oil as the oily vehicle, Mycophenolate Mofetil as active, Polyoxyl 15 Hydroxystearate as emulsifier, Propylene glycol as co solvent, Carbomer copolymer type A as thickener and Boric acid as preservative. Preferred emulsions comprise by weight based on the total weight of the composition:

TABLE 30

| | Ingredient | Function | Composition (% w/v) |
|---|---|---|---|
| Phase A | Mycophenolate | Active | 1.0 |
| | Mofetil | | |
| | Castor Oil | Oil component | 0.15 |
| | Polyoxyl 15 | Emulsifier | 0.25 |
| | Hydroxystearate | | |
| Phase B | Sodium hydroxide | pH Modifier | QS |
| | Boric Acid | Preservative | 0.1 |
| | Propylene glycol | Co solvent | 1.5 |
| | Carbomer copolymer | Thickener | 0.05 |
| | type A | | |
| | Purified water | Aqueous Phase | Q.s 100% |

Method of Preparation:
1. Castor oil and Polyoxyl 15 Hydroxystearate are taken in a beaker and heated to a temperature between 70±2° C.
2. Mycophenolate Mofetil is added to step 1 under stirring to get a clear phase maintaining the temperature between 70±2° C.
3. Aqueous phase is prepared by dispersing Carbomer homopolymer type A in purified water under continuous stirring.
4. Propylene glycol and Boric Acid are added to step 3 under continuous stirring and temperature of the phase is maintained between 70±2° C.
5. Emulsification is done by slowly adding the oil phase of step 2 to the aqueous phase of step 4 using a high shear emulsifier and maintaining the temperature between 70±2° C.
6. The emulsification process is continued for 30±5 minutes
7. The emulsion is slowly cooled and the pH is adjusted using 0.1 M NaOH solution.

### Example 23

An ophthalmic emulsion formulation comprising Light liquid paraffin as the oily vehicle, Mycophenolate Mofetil as active, Polyoxyl 35 castor oil as emulsifier, Glycerin as co solvent, Carbomer copolymer type A as thickener and Boric acid as preservative. Preferred emulsions comprise by weight based on the total weight of the composition:

TABLE 31

| | Ingredient | Function | Composition (% w/v) |
|---|---|---|---|
| Phase A | Mycophenolate | Active | 1.0 |
| | Mofetil | | |
| | Light liquid | Oil component | 5.0 |
| | paraffin | | |
| | Polyoxyl 35 | Co-Emulsifier | 5.0 |
| | castor oil | | |
| Phase B | Polysorbate 80 | Emulsifier | 4.0 |
| | Glycerin | Co-solvent | 2.0 |
| | Carbomer copolymer | Thickener | 0.05 |
| | type A | | |
| | Sodium hydroxide | pH Modifier | QS |
| | Boric Acid | Preservative | 0.1 |
| | Purified water | Aqueous Phase | q.s to 100% |

Method of Preparation:
1. Light liquid paraffin and Polyoxyl 35 castor oil are taken in a beaker and heated to a temperature between 70±2° C.
2. Mycophenolate Mofetil is added to step 1 under stirring to get a clear phase maintaining the temperature between 70±2° C.
3. Aqueous phase is prepared by dispersing Carbomer homopolymer type A in purified water under continuous stirring.
4. Glycerin and Boric Acid are added to step 3 under continuous stirring and temperature of the phase is maintained between 70±2° C.
5. Emulsification is done by slowly adding the oil phase of step 2 to the aqueous phase of step 4 using a high shear emulsifier and maintaining the temperature between 70±2° C.
6. The emulsification process is continued for 30±5 minutes
7. The emulsion is slowly cooled and the pH is adjusted using 0.1 M NaOH solution.

### Example 24

An ophthalmic emulsion formulation comprising Castor Oil as the oily vehicle, Mycophenolate Mofetil as active, Polyoxyl 35 castor oil as emulsifier, Glycerin as co solvent, Carbomer 940 as thickener, sodium chloride as tonicity adjusting agent and Boric acid as preservative.

A preferred emulgel formula comprises the following by weight based on the total weight of the composition:

TABLE 32

| | Ingredient | Function | Composition (% w/v) |
|---|---|---|---|
| Phase A | Mycophenolate | Active | 1.0 |
| | Mofetil | | |
| | Light liquid | Oil component | 10.0 |
| | paraffin | | |
| | Polyoxyl 35 | Co-Emulsifier | 5.0 |
| | castor oil | | |
| | Polysorbate 80 | Emulsifier | 4.0 |
| Phase B | Sodium hydroxide | pH Modifier | QS |
| | Boric Acid | Preservative | 0.1 |
| | Sodium Chloride | Tonicity modifier | 0.9 |
| | Glycerin | Co-solvent | 2.5 |
| | Purified water | Aqueous Phase | q.s to 100% |

Method of Preparation:
1. Castor oil, Polysorbate 80 and Polyoxyl 35 castor oil are taken in a beaker and heated to a temperature between 70±2° C.
2. Mycophenolate Mofetil is added to step 1 under stirring to get a clear phase.
3. Aqueous phase is prepared by adding Propylene glycol and Boric Acid in water to get a clear solution.
4. Temperature of the aqueous phase of step 2 is maintained between 70±2° C.
5. Emulsification is done by slowly adding the oil phase of step 2 to the aqueous phase of step 4 using a high shear emulsifier and maintaining the temperature between 70±2° C.
6. The emulsification process is continued for 30±5 minutes.
7. The emulsion is slowly cooled and the pH is adjusted using 0.1 M NaOH solution.
8. Tonicity is adjusted using Sodium chloride solution.

### Example 25

An ophthalmic emulsion formulation comprising Mycophenolate Mofetil as active, Oleic acid as oily vehicle, Polysorbate 80 and Polyoxyl 35 castor oil as emulsifier, glycerin as co-solvent, Boric acid as preservative and sodium hydroxide as pH adjusting agent.

TABLE 33

| | Ingredient | Function | Composition (% w/v) |
|---|---|---|---|
| Phase A | Mycophenolate | Active | 1.0 |
| | Mofetil | | |
| | Oleic acid | Oil component | 5.0 |
| Phase B | Polysorbate 80 | Emulsifier | 4.0 |
| | Polyoxyl 35 | Co-Emulsifier | 5.0 |
| | castor oil | | |
| | Glycerin | Co-solvent | 2.5 |
| | Sodium hydroxide | pH Modifier | QS |
| | Boric Acid | Preservative | 0.1 |
| | Purified water | Aqueous Phase | q.s to 100% |

Method of Preparation:
1. The active and Oleic acid are taken in a beaker and mixed together with heating until a homogeneous phase is obtained.
2. Aqueous phase components, such as Polysorbate 80, Polyoxyl 35 castor oil, Glycerin and Boric Acid, are added to purified water and then stirred with heating until a homogenous phase is obtained.
3. Phase of step 1 is added slowly to the phase of step 2 under homogenization to get an emulsion. Emulsification process continued for 30±5 minutes.
4. The emulsion is then slowly cooled to room temperature.
5. Final emulsion is obtained by passing the emulsion for several cycles for reducing the globule size through a high-pressure homogenizer.
6. The pH is adjusted using 0.1 M NaOH solution.

### Example 26

An ophthalmic emulsion formulation comprising Mycophenolate Mofetil as active, Labrafac PG as oily vehicle, Polyoxyl 35 castor oil and Polysorbate 80 as co emulsifier, Glycerin as co-solvent, sodium borate as preservative and sodium hydroxide as pH adjusting agent.

TABLE 34

| | Ingredient | Function | Composition (% w/v) |
|---|---|---|---|
| Phase A | Mycophenolate | Active | 1.0 |
| | Mofetil | | |
| | Labrafac PG | Solublizer/ | 5.0 |
| | | oil component | |
| Phase B | Polysorbate 80 | Emulsifier | 4.0 |
| | Polyoxyl 35 | Co-Emulsifier | 5.0 |
| | castor oil | | |
| | Glycerin | Co-solvent | 2.5 |
| | Sodium hydroxide | pH Modifier | QS |
| | Sodium borate | Preservative | 0.11 |
| | Purified water | Aqueous Phase | q.s to 100% |

Method of Preparation:
1. The active and Labrafac PG are taken in a beaker and mixed together with heating until a homogeneous phase is obtained.
2. Aqueous phase components, such as Polysorbate 80, Polyoxyl 35 castor oil, Glycerin and Sodium borate, are added to purified water and then stirred with heating until a homogenous phase is obtained.
3. Both phases are mixed together using a high shear emulsifier to get a coarse emulsion.
4. The emulsion is then slowly cooled to room temperature.
5. Final emulsion is obtained by passing the emulsion for several cycles for reducing the globule size through a high-pressure homogenizer.
6. The pH is adjusted using 0.1 M NaOH solution.

### Example 27

An ophthalmic emulsion formulation comprising Mycophenolate Mofetil as active, Labrafac1349 as oily vehicle, Polyoxyl 35 castor oil and Polysorbate 80 as co emulsifier, Glycerin as co-solvent, potassium borate as preservative and sodium hydroxide as pH adjusting agent.

TABLE 35

| | Ingredient | Function | Composition (% w/v) |
|---|---|---|---|
| Phase A | Mycophenolate | Active | 1.0 |
| | Mofetil | | |
| | Labrafac 1349 | oil component | 5.0 |
| Phase B | Polysorbate 80 | Emulsifier | 4.0 |
| | Polyoxyl 35 | Co-Emulsifier | 5.0 |
| | castor oil | | |
| | Glycerin | Co-solvent | 2.5 |
| | Sodium hydroxide | pH Modifier | QS |
| | Potassium sorbate | Preservative | 0.47 |
| | Purified water | Aqueous Phase | q.s to 100% |

Method of Preparation:
1. The active and Labrafac1349 are taken in a beaker and mixed together with heating until a homogeneous phase is obtained.
2. Aqueous phase components, such as Polysorbate 80, Polyoxyl 35 castor oil, Glycerin and potassium borate, are added to purified water and then stirred with heating until a homogenous phase is obtained.
3. Both phases are mixed together using a high shear emulsifier to get a coarse emulsion.
4. The emulsion is then slowly cooled to room temperature.
5. Final emulsion is obtained by passing the emulsion for several cycles for reducing the globule size through a high-pressure homogenizer.
6. The pH is adjusted using 0.1 M NaOH solution.

### Example 28

An ophthalmic emulsion formulation comprising Mycophenolate Mofetil as active, Labrafac1349 as oily vehicle, Polyoxyl 35 castor oil and Polysorbate 80 as co emulsifier, Glycerin as co-solvent, potassium borate as preservative and sodium hydroxide as pH adjusting agent.

TABLE 36

| | Ingredient | Function | Composition (% w/v) |
|---|---|---|---|
| Phase A | Mycophenolate | Active | 1.0 |
| | Mofetil | | |
| | Labrafac 1349 | Oil component | 5.0 |
| Phase B | Polysorbate 80 | Emulsifier | 4.0 |
| | Labrasol | Co-Emulsifier | 5.0 |
| | Glycerin | Co-solvent | 2.5 |
| | Sodium hydroxide | pH Modifier | QS |
| | Sorbic Acid | Preservative | 0.1 |
| | Purified water | Aqueous Phase | q.s to 100% |

Method of Preparation:
1. The active and Labrafac1349 are taken in a beaker and mixed together with heating until a homogeneous phase is obtained.
2. Aqueous phase components, such as Labrasol, Polysorbate 80, Glycerin and sorbic acid, are added to purified water and then stirred with heating until a homogenous phase is obtained.
3. Both phases are mixed together using a high shear emulsifier to get a coarse emulsion.
4. The emulsion is then slowly cooled to room temperature.
5. Final emulsion is obtained by passing the emulsion for several cycles for reducing the globule size through a high-pressure homogenizer.
6. The pH is adjusted using 0.1 M NaOH solution.

### Example 29

An ophthalmic emulsion formulation comprising Mycophenolate Mofetil as active, Labrafac1349 as oily vehicle, Polysorbate 80 and Labrasol as emulsifier, Transcutol P as co-solvent, Benzalkonium chloride as preservative and sodium hydroxide as pH adjusting agent.

TABLE 37

| | Ingredient | Function | Composition (% w/v) |
|---|---|---|---|
| Phase A | Mycophenolate | Active | 1.0 |
| | Mofetil | | |
| | Labrafac 1349 | oil component | 5.0 |
| Phase B | Polysorbate 80 | Emulsifier | 4.0 |
| | Labrasol | Co-Emulsifier | 5.0 |
| | Transcutol P | Co-solvent | 2.5 |
| | Sodium hydroxide | pH Modifier | QS |
| | Benzalkonium chloride | Preservative | 0.03 |
| | Purified water | Aqueous Phase | q.s to 100% |

Method of Preparation:
1. The active and Labrafac1349 are taken in a beaker and mixed together with heating until a homogeneous phase is obtained.
2. Aqueous phase components, such as Transcutol P, Labrasol, Polysorbate 80 and Benzalkonium chloride are added to purified water and then stirred with heating until a homogenous phase is obtained.
3. Both phases are mixed together using a high shear emulsifier to get a coarse emulsion.
4. The emulsion is then slowly cooled to room temperature.
5. Final emulsion is obtained by passing the emulsion for several cycles for reducing the globule size through a high-pressure homogenizer.
6. The pH is adjusted using 0.1 M NaOH solution.

### Example 30

An ophthalmic emulsion formulation comprising Mycophenolate Mofetil as active, Labrafac1349 as oily vehicle, Capryol 90 and Labrasol as emulsifier, Transcutol P as co-solvent, Propyl paraben as preservative and sodium hydroxide as pH adjusting agent.

TABLE 38

| | Ingredient | Function | Composition (% w/v) |
|---|---|---|---|
| Phase A | Mycophenolate | Active | 1.0 |
| | Mofetil | | |
| | Labrafac 1349 | Oil component | 5.0 |
| Phase B | Capryol 90 | Emulsifier | 4.0 |
| | Labrasol | Co-Emulsifier | 5.0 |
| | Transcutol P | Co-solvent | 2.5 |
| | Sodium hydroxide | pH Modifier | QS |
| | Propyl paraben | Preservative | 0.01 |
| | Purified water | Aqueous Phase | q.s to 100% |

Method of Preparation:
1. The active and Labrafac1349 are taken in a beaker and mixed together with heating until a homogeneous phase is obtained.
2. Aqueous phase components, such as Transcutol P, Labrasol, Capryol 90 and Propyl paraben are added to purified water and then stirred with heating until a homogenous phase is obtained.
3. Both phases are mixed together using a high shear emulsifier to get a coarse emulsion.
4. The emulsion is then slowly cooled to room temperature.
5. Final emulsion is obtained by passing the emulsion for several cycles for reducing the globule size through a high-pressure homogenizer.
6. The pH is adjusted using 0.1 M NaOH solution.

### Example 31

An ophthalmic emulsion formulation comprising Mycophenolate Mofetil as active, Labrafac1349 as oily vehicle, Capryol 90 and Capryol PGMC as emulsifier, Transcutol P as co-solvent, methyl paraben as preservative and sodium hydroxide as pH adjusting agent.

TABLE 39

| | Ingredient | Function | Composition (% w/v) |
|---|---|---|---|
| Phase A | Mycophenolate | Active | 1.0 |
| | Mofetil | | |
| | Labrafac 1349 | Oil component | 5.0 |
| Phase B | Capryol 90 | Emulsifier | 4.0 |
| | Capryol PGMC | Co-Emulsifier | 4.0 |
| | Transcutol P | Co-solvent | 2.5 |
| | Sodium hydroxide | pH Modifier | QS |
| | Methyl paraben | Preservative | 0.05 |
| | Purified water | Aqueous Phase | q.s to 100% |

Method of Preparation:
1. The active and Labrafac1349 are taken in a beaker and mixed together with heating until a homogeneous phase is obtained.
2. Aqueous phase components, such as Capryol PGMC, Capryol 90, Transcutol P and methyl paraben are added to purified water and then stirred with heating until a homogenous phase is obtained.
3. Both phases are mixed together using a high shear emulsifier to get a coarse emulsion.
4. The emulsion is then slowly cooled to room temperature.
5. Final emulsion is obtained by passing the emulsion for several cycles for reducing the globule size through a high-pressure homogenizer.
6. The pH is adjusted using 0.1 M NaOH solution.

### Example 32

An ophthalmic emulsion formulation comprising Mycophenolate Mofetil as active, Labrafac1349 as oily vehicle, Capryol 90 and Polyoxyl 35 castor oil as emulsifier, Transcutol P as co-solvent, Chlorobutanol as preservative and sodium hydroxide as pH adjusting agent.

TABLE 40

| | Ingredient | Function | Composition (% w/v) |
|---|---|---|---|
| Phase A | Mycophenolate | Active | 1.0 |
| | Mofetil | | |
| | Labrafac 1349 | Oil component | 5.0 |
| Phase B | Capryol 90 | Emulsifier | 4.0 |
| | Polyoxyl 35 | Emulsifier | 4.0 |
| | castor oil | | |
| | Transcutol P | Co-solvent | 2.5 |
| | Sodium hydroxide | pH Modifier | QS |
| | Chlorobutanol | Preservative | 0.55 |
| | Purified water | Aqueous Phase | q.s to 100% |

Method of Preparation:
1. The active and Labrafac1349 are taken in a beaker and mixed together with heating until a homogeneous phase is obtained.
2. Aqueous phase components, such as Polyoxyl 35 castor oil, Capryol 90, Transcutol P and Chlorobutanol are added to purified water and then stirred with heating until a homogenous phase is obtained.
3. Both phases are mixed together using a high shear emulsifier to get a coarse emulsion.
4. The emulsion is then slowly cooled to room temperature.
5. Final emulsion is obtained by passing the emulsion for several cycles for reducing the globule size through a high-pressure homogenizer.
6. The pH is adjusted using 0.1 M NaOH solution.

### Example 33

An ophthalmic emulsion formulation comprising Mycophenolate Mofetil as active, Oleic acid as oily vehicle, Lauroglycol 90 and Poloxamer 407 as emulsifier, Transcutol P as co-solvent, Benzalkonium chloride as preservative, Carbomer copolymer type B as thickener and sodium hydroxide as pH adjusting agent.

TABLE 41

| | Ingredient | Function | Composition (% w/v) |
|---|---|---|---|
| Phase A | Mycophenolate | Active | 1.0 |
| | Mofetil | | |
| | Oleic acid | Oil component | 5.0 |
| Phase B | Lauroglycol 90 | Emulsifier | 4.0 |
| | Poloxamer 407 | Co-Emulsifier | 4.0 |
| | Transcutol P | Co-solvent | 2.5 |
| | Sodium hydroxide | pH Modifier | QS |
| | Carbomer copolymer | Thickener | 0.05 |
| | type B | | |
| | Benzalkonium | Preservative | 10.0 |
| | chloride | | |
| | Purified water | Aqueous Phase | q.s to 100% |

Method of Preparation:
1. The active and Oleic acid are taken in a beaker and mixed together with heating until a homogeneous phase is obtained.
2. Aqueous phase components, such as Lauroglycol 90, Poloxamer 407, Transcutol P and Benzalkonium chloride are added to purified water and then stirred with heating until a homogenous phase is obtained.
3. Carbomer copolymer type B is dispersed in the phase step 2 under continuous stirring.
4. Both phases are mixed together using a high shear emulsifier to get a coarse emulsion.
5. The emulsion is then slowly cooled to room temperature.
6. Final emulsion is obtained by passing the emulsion for several cycles for reducing the globule size through a high-pressure homogenizer.
7. The pH is adjusted using 0.1 M NaOH solution.

### Example 34

An ophthalmic emulsion formulation comprising Mycophenolate Mofetil as active, Oleic acid as oily vehicle, Polysorbate 80 and Labrasol as emulsifier, Glycerin as co-solvent, Benzalkonium chloride as preservative, Carbomer copolymer type B as thickener and sodium hydroxide as pH adjusting agent.

TABLE 42

| | Ingredient | Function | Composition (% w/v) |
|---|---|---|---|
| Phase A | Mycophenolate | Active | 1.0 |
| | Mofetil | | |
| | Oleic acid | Oil component | 5.0 |
| Phase B | Polysorbate 80 | Emulsifier | 4.0 |
| | Labrasol | Co-Emulsifier | 4.0 |
| | Glycerin | Co-solvent | 2.5 |
| | Sodium hydroxide | pH Modifier | QS |
| | Carbomer copolymer | Thickener | 0.05 |
| | type B | | |
| | Benzalkonium | Preservative | 10.0 |
| | chloride | | |
| | Purified water | Aqueous Phase | q.s to 100% |

Method of Preparation:
1. The active and Oleic acid are taken in a beaker and mixed together with heating until a homogeneous phase is obtained.
2. Aqueous phase components, such as Polysorbate 80, Labrasol, Glycerin and Benzalkonium chloride are added to purified water and then stirred with heating until a homogenous phase is obtained.
3. Carbomer copolymer type B is dispersed in the phase step 2 under continuous stirring.
4. Both phases are mixed together using a high shear emulsifier to get a coarse emulsion.
5. The emulsion is then slowly cooled to room temperature.
6. Final emulsion is obtained by passing the emulsion for several cycles for reducing the globule size through a high-pressure homogenizer.
7. The pH is adjusted using 0.1 M NaOH solution.

### Example 35

An ophthalmic emulsion formulation comprising Mycophenolate Mofetil as active, Oleic acid as oily vehicle, Lauroglycol 90 and Poloxamer 407 as emulsifier, Transcutol P as co-solvent, Benzalkonium chloride as preservative, Carbomer 940 as thickener and sodium hydroxide as pH adjusting agent.

TABLE 43

| | Ingredient | Function | Composition (% w/v) |
|---|---|---|---|
| Phase A | Mycophenolate | Active | 1.0 |
| | Mofetil | | |
| | Oleic acid | Oil component | 5.0 |
| Phase B | Lauroglycol 90 | Emulsifier | 4.0 |
| | Poloxamer 407 | Co-Emulsifier | 4.0 |
| | Transcutol P | Co-solvent | 2.5 |
| | Sodium hydroxide | pH Modifier | QS |
| | Carbomer 940 | Thickener | 0.15 |
| | Benzalkonium | Preservative | 10.0 |
| | chloride | | |
| | Purified water | Aqueous Phase | q.s to 100% |

Method of Preparation:
1. The active and Oleic acid are taken in a beaker and mixed together with heating until a homogeneous phase is obtained.
2. Aqueous phase components, such as Lauroglycol 90, Poloxamer 407, Transcutol P and Benzalkonium chloride are added to purified water and then stirred with heating until a homogenous phase is obtained.
3. Carbomer 940 is dispersed in the phase step 2 under continuous stirring.
4. Both phases are mixed together using a high shear emulsifier to get a coarse emulsion.
5. The emulsion is then slowly cooled to room temperature.
6. Final emulsion is obtained by passing the emulsion for several cycles for reducing the globule size through a high-pressure homogenizer.
7. The pH is adjusted using 0.1 M NaOH solution.

### Example 36

An ophthalmic emulsion formulation comprising Mycophenolate Mofetil as active, Oleic acid as oily vehicle, Lauroglycol 90 and Poloxamer 407 as emulsifier, Transcutol P as co-solvent, Benzalkonium chloride as preservative, Polycarbophil as thickener and sodium hydroxide as pH adjusting agent.

TABLE 44

| | Ingredient | Function | Composition (% w/v) |
|---|---|---|---|
| Phase A | Mycophenolate | Active | 1.0 |
| | Mofetil | | |
| | Oleic acid | Oil component | 5.0 |
| Phase B | Lauroglycol 90 | Emulsifier | 4.0 |
| | Poloxamer 407 | Co-Emulsifier | 4.0 |
| | Transcutol P | Co-solvent | 2.5 |
| | Sodium hydroxide | pH Modifier | QS |
| | Polycarbophil | Thickener | 0.25 |
| | Benzalkonium | Preservative | 10.0 |
| | chloride | | |
| | Purified water | Aqueous Phase | q.s to 100% |

Method of Preparation:
1. The active and Oleic acid are taken in a beaker and mixed together with heating until a homogeneous phase is obtained.
2. Aqueous phase components, such as Lauroglycol 90, Poloxamer 407, Transcutol P and Benzalkonium chloride are added to purified water and then stirred with heating until a homogenous phase is obtained.
3. Polycarbophil is dispersed in the phase step 2 under continuous stirring.
4. Both phases are mixed together using a high shear emulsifier to get a coarse emulsion.
5. The emulsion is then slowly cooled to room temperature.
6. Final emulsion is obtained by passing the emulsion for several cycles for reducing the globule size through a high-pressure homogenizer.
7. The pH is adjusted using 0.1 M NaOH solution.

### Example 37

An ophthalmic emulsion formulation comprising Mycophenolate Mofetil as active, Labrafac 1349 as oily vehicle, Caproyl 90 and Polyoxyl 35 castor oil as emulsifier, Transcutol P as co-solvent, Benzalkonium chloride as preservative, Polycarbophil as thickener and sodium hydroxide as pH adjusting agent.

TABLE 45

| | Ingredient | Function | Composition (% w/v) |
|---|---|---|---|
| Phase A | Mycophenolate | Active | 1.0 |
| | Mofetil | | |
| | Labrafac 1349 | Oil component | 5.0 |
| Phase B | Capryol 90 | Emulsifier | 4.0 |
| | Polyoxyl 35 | Emulsifier | 4.0 |
| | castor oil | | |
| | Transcutol P | Co-solvent | 2.5 |
| | Sodium hydroxide | pH Modifier | QS |
| | Polycarbophil | Thickener | 0.25 |
| | Benzalkonium | Preservative | 10.0 |
| | chloride | | |
| | Purified water | Aqueous Phase | q.s to 100% |

Method of Preparation:
1. The active and Labrafac 1349 are taken in a beaker and mixed together with heating until a homogeneous phase is obtained.
2. Aqueous phase components, such as Capryol 90, Polyoxyl 35 castor oil, Transcutol P and Benzalkonium chloride are added to purified water and then stirred with heating until a homogenous phase is obtained.
3. Polycarbophil is dispersed in the phase step 2 under continuous stirring.
4. Both phases are mixed together using a high shear emulsifier to get a coarse emulsion.
5. The emulsion is then slowly cooled to room temperature.
6. Final emulsion is obtained by passing the emulsion for several cycles for reducing the globule size through a high-pressure homogenizer.
7. The pH is adjusted using 0.1 M NaOH solution.

### Example 38

An ophthalmic emulsion formulation comprising Mycophenolate Mofetil as active, Light liquid paraffin as oily vehicle, Polysorbate 80 and Polyoxyl 35 castor oil as emulsifier and sodium hydroxide as pH adjusting agent, as shown in Table 46 was prepared.

TABLE 46

| | Ingredient | Function | Composition (% w/v) |
|---|---|---|---|
| Phase A | Mycophenolate | Active | 1.0 |
| | Mofetil | | |
| | Light liquid | Oil component | 10.0 |
| | paraffin | | |
| Phase B | Polysorbate 80 | Emulsifier | 4.0 |
| | Polyoxyl 35 | Emulsifier | 5.0 |
| | castor oil | | |
| | Purified water | Aqueous Phase | q.s to 100% |

Method of Preparation:
1. The active and Light liquid paraffin were taken in a beaker and mixed together until a homogeneous phase is obtained.
2. Aqueous phase components, such as Polysorbate 80 and Polyoxyl 35 castor oil were added to purified water and then stirred until a homogenous phase is obtained.
3. Both phases of step 1 and 2 were mixed together using a high shear emulsifier to get a coarse emulsion.
4. Final emulsion was obtained by passing the emulsion for several cycles for reducing the globule size through a high-pressure homogenizer.
5. The pH was adjusted using 0.1 M NaOH solution.

### Example 39

An ophthalmic emulsion formulation comprising Mycophenolate Mofetil as active, Light liquid paraffin as oily vehicle, Polysorbate 80 and Polyoxyl 35 castor oil as emulsifier, Hydroxyethyl cellulose as thickener and sodium hydroxide as pH adjusting agent, as shown in Table 47 was prepared.

TABLE 47

| | Ingredient | Function | Composition (% w/v) |
|---|---|---|---|
| Phase A | Mycophenolate | Active | 1.0 |
| | Mofetil | | |
| | Light liquid | Oil component | 10.0 |
| | paraffin | | |
| Phase B | Polysorbate 80 | Emulsifier | 4.0 |
| | Polyoxyl 35 | Emulsifier | 5.0 |
| | castor oil | | |
| | Hydroxyethyl | Thickener | 0.5 |
| | cellulose | | |
| | Purified water | Aqueous Phase | q.s to 100% |

Method of Preparation:
1. The active and Light liquid paraffin were taken in a beaker and mixed together until a homogeneous phase is obtained.
2. Aqueous phase components, such as Polysorbate 80 and Polyoxyl 35 castor oil were added to purified water followed by addition of Hydroxyethyl cellulose along with stirring until a homogenous phase is obtained.
3. Both phases of step 1 and 2 were mixed together using a high shear emulsifier to get a coarse emulsion.
4. Final emulsion was obtained by passing the emulsion for several cycles for reducing the globule size through a high-pressure homogenizer.
5. The pH was adjusted using 0.1 M NaOH solution.

### Example 40

An ophthalmic solution formulation comprising Mycophenolate Sodium as active, Tyloxapol as solubilizer, Propylene glycol as co solvent, Hydroxyethyl cellulose as thickener, sodium chloride as tonicity adjusting agent and Benzalkonium chloride as preservative.

TABLE 48

| Ingredient | Function | Composition (% w/v) |
|---|---|---|
| Mycophenolate sodium | Active | 1.0 |
| equivalent to Mycophenolic | | |
| acid | | |
| Sodium Chloride | Tonicity modifier | 0.9 |
| Hydroxyethyl cellulose | Thickening agent | 0.5 |
| Tyloxapol | Solubilizer | 0.1 |
| Sodium hydroxide | pH Modifier | QS |
| Benzalkonium chloride | Preservative | 1.0 |
| Purified water | Aqueous Phase | q.s to 100% |

Method of Preparation:
1. Tyloxapol and Mycophenolate sodium are added to a part of purified water and stirred until a homogeneous phase is obtained.
2. Hydroxyethyl cellulose is added to a part of purified water under stirring to form a uniform solution.
3. Sodium Chloride, Boric Acid and Benzalkonium chloride are added to step 2 under stirring.
4. Both the phases of step 1 and step 3 are under stirring to get a clear phase.
5. pH is measured and then adjusted using 0.1 M NaOH solution.
6. Sterilization is done by filtration through a 0.22 um filter.

### Example 41

An ophthalmic solution formulation comprising Mycophenolate Sodium as active, Tyloxapol as solubilizer, Propylene glycol as co solvent, Hydroxyethyl cellulose as thickener, sodium chloride as tonicity adjusting agent and Benzalkonium chloride as preservative.

TABLE 49

| Ingredient | Function | Composition (% w/v) |
|---|---|---|
| Mycophenolate sodium | Active | 1.0 |
| equivalent to Mycophenolic | | |
| acid | | |
| Sodium Chloride | Tonicity modifier | 0.9 |
| Hydroxyethyl cellulose | Thickening agent | 0.5 |
| Tyloxapol | Solubilizer | 0.1 |
| Sodium hydroxide | pH Modifier | QS |
| Benzalkonium chloride | Preservative | 1.0 |
| Purified water | Aqueous Phase | q.s to 100% |

Method of Preparation:
1. Tyloxapol and Mycophenolate sodium are added to a part of purified water and stirred until a homogeneous phase is obtained.
2. Hydroxyethyl cellulose is added to a part of purified water under stirring to form a uniform solution.
3. Sodium Chloride, Boric Acid and Benzalkonium chloride are added to step 2 under stirring.
4. Both the phases of step 1 and step 3 are under stirring to get a clear phase.
5. pH is measured and then adjusted using 0.1 M NaOH solution.
6. Sterilization is done by filtration through a 0.22 um filter.

### Example 42

An ophthalmic solution formulation comprising Mycophenolate Sodium as active, Polyoxyl 40 stearate as solubilizer, Propylene glycol as co solvent, Sodium carboxymethyl cellulose as thickener, Sodium chloride as tonicity adjusting agent and Benzalkonium chloride as preservative.

TABLE 50

| Ingredient | Function | Composition (% w/v) |
|---|---|---|
| Mycophenolate sodium | Active | 1.0 |
| equivalent to Mycophenolic | | |
| acid | | |
| Sodium chloride | Tonicity modifier | 0.22 |
| Sodium Carboxymethyl | Thickening agent | 0.5 |
| cellulose | | |
| Boric Acid | Preservative | 1.0 |
| Polyoxyl 40 stearate | Solubilizer | 7.0 |
| Phosphoric acid | pH Modifier | q.s to pH |
| | | 5.5 to 6.5 |
| Purified water | Aqueous Phase | q.s to 100% |

Method of Preparation:
1. Polyoxyl 40 stearate and Mycophenolate sodium are added to a part of purified water and stirred until a homogeneous phase is obtained.
2. Sodium Carboxymethyl cellulose is added to a part of purified water under stirring to form a uniform solution.
3. Sodium Chloride and Boric Acid are added to the solution of step 1 to get a clear solution.
4. Both the phases of step 1 and step 3 are under stirring to get a clear phase.
5. pH is measured and then adjusted using 0.1 M Phosphoric acid solution.
6. Sterilization is done by filtration through a 0.22 um filter.

### Example 43

An ophthalmic solution formulation comprising Mycophenolate Sodium as active, Tyloxapol as solubilizer, Propylene glycol as co solvent, Hydroxyethyl cellulose as thickener, sodium chloride as tonicity adjusting agent and Benzalkonium chloride as preservative. A preferred solution comprises by weight based on the total weight of the composition:

TABLE 51

| Ingredient | Function | Composition (% w/v) |
|---|---|---|
| Mycophenolate sodium | Active | 1.0 |
| equivalent to Mycophenolic | | |
| acid | | |
| Sodium Chloride | Tonicity modifier | 0.9 |
| Hydroxyethyl cellulose | Thickening agent | 0.5 |
| Tyloxapol | Solubilizer | 0.1 |
| Sodium hydroxide | pH Modifier | QS |
| Benzalkonium chloride | Preservative | 1.0 |
| Purified water | Aqueous Phase | q.s to 100% |

Method of Preparation:
1. Tyloxapol and Mycophenolate sodium are added to a part of purified water and stirred until a homogeneous phase is obtained.
2. Hydroxyethyl cellulose is added to a part of purified water under stirring to form a uniform solution.
3. Sodium Chloride, Boric Acid and Benzalkonium chloride are added to step 2 under stirring.
4. Both the phases of step 1 and step 3 are under stirring to get a clear phase.
5. pH is measured and then adjusted using 0.1 M NaOH solution.
6. Sterilization is done by filtration through a 0.22 um filter.

### Example 44

An ophthalmic solution formulation comprising Mycophenolate Sodium as active, Tyloxapol as solubilizer, Propylene glycol as co solvent, Hydroxyethyl cellulose as thickener, sodium chloride as tonicity adjusting agent and Benzalkonium chloride as preservative.

A preferred solution comprises by weight based on the total weight of the composition:

TABLE 52

| Ingredient | Function | Composition (% w/v) |
|---|---|---|
| Mycophenolate sodium | Active | 1.0 |
| equivalent to Mycophenolic | | |
| acid | | |
| Sodium Chloride | Tonicity modifier | 0.9 |
| Boric Acid | Preservative | 1.7 |
| Tyloxapol | Solubilizer | 0.1 |
| Sodium hydroxide | pH Modifier | QS |
| Purified water | Aqueous Phase | q.s to 100% |

Method of Preparation:
1. Tyloxapol and Mycophenolate sodium are added to a part of purified water and stirred until a homogeneous phase is obtained.
2. Sodium Chloride, Boric Acid and Benzalkonium chloride are added to the solution of step 1, followed by volume make up using purified water.
3. pH is measured and then adjusted using 0.1 M NaOH solution.
4. Sterilization is done by filtration through a 0.22 um filter.

### Example 45

An ophthalmic solution formulation comprising Mycophenolate Sodium as active, Polyoxyl 40 stearate as solubilizer, Potassium chloride as tonicity adjusting agent and Benzalkonium chloride as preservative and Sulfuric acid as pH modifier.

TABLE 53

| Ingredient | Function | Composition (% w/v) |
|---|---|---|
| Mycophenolate sodium | Active | 1.0 |
| equivalent to Mycophenolic | | |
| acid | | |
| Potassium chloride | Tonicity modifier | 0.9 |
| Polyoxyl 40 stearate | Solubilizer | 7.0 |
| Sulfuric acid | pH Modifier | q.s |
| Benzalkonium chloride | Preservative | 10.0 |
| Purified water | Aqueous Phase | q.s to 100% |

Method of Preparation:
1. Polyoxyl 40 stearate and Mycophenolate sodium are added to a part of purified water and stirred until a homogeneous phase is obtained.
2. Sodium Chloride, Boric Acid and Benzalkonium chloride are added to the solution of step 1, followed by volume make up using purified water.
3. pH is measured and then adjusted using 0.1 M NaOH solution.
4. Sterilization is done by filtration through a 0.22 um filter.

### Example 46

An ophthalmic solution formulation comprising Mycophenolate Sodium as active, Polyoxyl 40 stearate as solubilizer, Potassium chloride as tonicity adjusting agent and Boric Acid as preservative and Sulfuric acid as pH modifier.

TABLE 54

| Ingredient | Function | Composition (% w/v) |
|---|---|---|
| Mycophenolate sodium | Active | 1.0 |
| equivalent to Mycophenolic | | |
| acid | | |
| Potassium chloride | Tonicity modifier | 0.9 |
| Boric Acid | Preservative | 0.1 |
| Polyoxyl 40 stearate | Solubilizer | 7.0 |
| Sulfuric acid | pH Modifier | q.s |
| Purified water | Aqueous Phase | q.s to 100% |

Method of Preparation:
1. Polyoxyl 40 stearate and Mycophenolate sodium are added to a part of purified water and stirred until a homogeneous phase is obtained.
2. Potassium Chloride, Boric Acid and Benzalkonium chloride are added to the solution of step 1, followed by volume make up using purified water.
3. pH is measured and then adjusted using Sulfuric acid solution.
4. Sterilization is done by filtration through a 0.22 um filter.

### Example 47

An ophthalmic solution formulation comprising Mycophenolate Sodium as active, Poloxomer 188 as solubilizer, Mannitol as tonicity adjusting agent and Potassium sorbate as preservative and Sodium acetate as pH modifier.

TABLE 55

| Ingredient | Function | Composition (% w/v) |
|---|---|---|
| Mycophenolate sodium | Active | 1.0 |
| equivalent to Mycophenolic | | |
| acid | | |
| Mannitol | Tonicity modifier | 4.7 |
| Poloxomer 188 | Solubilizer | 0.1 |
| Sodium acetate | pH Modifier | 1.24 |
| Potassium sorbate | Preservative | 0.47 |
| Purified water | Aqueous Phase | q.s to 100% |

Method of Preparation:
1. Poloxomer 188 and Mycophenolate sodium are added to a part of purified water and stirred until a homogeneous phase is obtained.
2. Mannitol and Potassium sorbate are added to the solution of step 1, followed by volume make up using purified water.
3. pH is measured and then adjusted using Sodium acetate.
4. Sterilization is done by filtration through a 0.22 um filter.

### Example 48

An ophthalmic solution formulation comprising Mycophenolate Sodium as active, Polyoxyl 35 castor oil and Polyethylene glycol 8000 as solubilizer, Calcium chloride as tonicity adjusting agent and Benzalkonium chloride as preservative and Sodium phosphate dibasic dodecahydrate as pH modifier.

TABLE 56

| Ingredient | Function | Composition (% w/v) |
|---|---|---|
| Mycophenolate sodium | Active | 1.0 |
| equivalent to Mycophenolic | | |
| acid | | |
| Calcium chloride | Tonicity modifier | 0.01 |
| Polyoxyl 35 castor oil | Solubilizer | 5.0 |
| Polyethylene glycol 8000 | Solubilizer | 2.0 |
| Sodium phosphate dibasic | pH Modifier | 1.67 |
| dodecahydrate | | |
| Benzalkonium chloride | Preservative | 10.0 |
| Purified water | Aqueous Phase | q.s to 100% |

Method of Preparation:
1. Polyoxyl 35 castor oil, Polyethylene glycol 8000 and Mycophenolate sodium are added to a part of purified water and stirred until a homogeneous phase is obtained.
2. Calcium Chloride and Benzalkonium chloride are added to the solution of step 1, followed by volume make up using purified water.
3. pH is measured and then adjusted using Sodium phosphate dibasic dodecahydrate.
4. Sterilization is done by filtration through a 0.22 um filter.

### Example 49

An ophthalmic solution formulation comprising Mycophenolate Sodium as active, Nonoxynol-9 and Polyethylene glycol 8000 as solubilizer, Calcium chloride as tonicity adjusting agent and Chlorobutanol as preservative and Monohydrate sodium nitrate as pH modifier.

TABLE 57

| Ingredient | Function | Composition (% w/v) |
|---|---|---|
| Mycophenolate sodium | Active | 1.0 |
| equivalent to Mycophenolic | | |
| acid | | |
| Calcium chloride | Tonicity modifier | 0.01 |
| Nonoxynol-9 | Solubilizer | 0.13 |
| Polyethylene glycol 8000 | Solubilizer | 2.0 |
| Monohydrate sodium nitrate | pH Modifier | 1.18 |
| Chlorobutanol | Preservative | 1.1 |
| Purified water | Aqueous Phase | q.s to 100% |

Method of Preparation:
1. Nonoxynol-9, Polyethylene glycol 8000 and Mycophenolate sodium are added to a part of purified water and stirred until a homogeneous phase is obtained.
2. Calcium Chloride and Chlorobutanol are added to the solution of step 1, followed by volume make up using purified water.
3. pH is measured and then adjusted using Monohydrate sodium nitrate.
4. Sterilization is done by filtration through a 0.22 um filter.

### Example 50

An ophthalmic solution formulation comprising Mycophenolate Sodium as active, Glyceryl Monostearate and Polyethylene glycol 8000 as solubilizer, Dextran as thickening agent, Mannitol as tonicity adjusting agent and Sodium borate as preservative, Sodium thiosulfate as antioxidant and Monohydrate sodium nitrate as pH modifier.

TABLE 58

| Ingredient | Function | Composition (% w/v) |
|---|---|---|
| Mycophenolate sodium | Active | 1.0 |
| equivalent to Mycophenolic | | |
| acid | | |
| Mannitol | Tonicity modifier | 5.0 |
| Dextran | Thickening agent | 20.00 |
| Glyceryl Monostearate | Solubilizer | 0.5 |
| Polyethylene glycol 8000 | Solubilizer | 2.0 |
| Monohydrate sodium nitrate | pH Modifier | 1.18 |
| Sodium thiosulfate | Antioxidant | 0.5 |
| Sodium borate | Preservative | 1.1 |
| Purified water | Aqueous Phase | q.s to 100% |

Method of Preparation:
1. Glyceryl Monostearate, Polyethylene glycol 8000 and Mycophenolate sodium are added to a part of purified water and stirred until a homogeneous phase is obtained.
2. Mannitol, Dextran, Sodium thiosulfate and Sodium borate are added to the solution of step 1, followed by volume make up using purified water.
3. pH is measured and then adjusted using Monohydrate sodium nitrate.
4. Sterilization is done by filtration through a 0.22 um filter.

### Example 51

An ophthalmic solution formulation comprising Mycophenolate Sodium as active, Glyceryl Monostearate and Polyethylene glycol 8000 as solubilizer, Polyvinyl Alcohol as thickening agent, Mannitol as tonicity adjusting agent and Sodium borate as preservative, Sodium thiosulfate as antioxidant and Monohydrate sodium nitrate as pH modifier.

TABLE 59

| Ingredient | Function | Composition (% w/v) |
|---|---|---|
| Mycophenolate sodium | Active | 1.0 |
| equivalent to Mycophenolic | | |
| acid | | |
| Mannitol | Tonicity modifier | 5.0 |
| Polyvinyl Alcohol | Thickening agent | 1.4 |
| Glyceryl Monostearate | Solubilizer | 0.5 |
| Polyethylene glycol 8000 | Solubilizer | 2.0 |
| Monohydrate sodium nitrate | pH Modifier | 1.18 |
| Sodium thiosulfate | Antioxidant | 0.5 |
| Sodium borate | Preservative | 1.1 |
| Purified water | Aqueous Phase | q.s to 100% |

Method of Preparation:
1. Glyceryl Monostearate, Polyethylene glycol 8000 and Mycophenolate sodium are added to a part of purified water and stirred until a homogeneous phase is obtained.
2. Mannitol, Polyvinyl Alcohol, Sodium thiosulfate and Sodium borate are added to the solution of step 1, followed by volume make up using purified water.
3. pH is measured and then adjusted using Monohydrate sodium nitrate.
4. Sterilization is done by filtration through a 0.22 um filter.

### Example 52

An ophthalmic solution formulation comprising Mycophenolate Sodium as active, Glyceryl Monostearate and Polypropylene glycol as solubilizer, Methyl cellulose as thickening agent, Sodium chloride as tonicity adjusting agent and Sodium borate as preservative, Sodium thiosulfate as antioxidant and Monohydrate sodium nitrate as pH modifier.

TABLE 60

| Ingredient | Function | Composition (% w/v) |
|---|---|---|
| Mycophenolate sodium | Active | 1.0 |
| equivalent to Mycophenolic | | |
| acid | | |
| Sodium chloride | Tonicity modifier | 0.9 |
| Methyl cellulose | Thickening agent | 0.5 |
| Glyceryl Monostearate | Solubilizer | 0.5 |
| Polypropylene glycol | Solubilizer | 15.0 |
| Monohydrate sodium nitrate | pH Modifier | 1.18 |
| Sodium thiosulfate | Antioxidant | 0.5 |
| Sodium borate | Preservative | 1.1 |
| Purified water | Aqueous Phase | q.s to 100% |

Method of Preparation:
1. Glyceryl Monostearate, Polypropylene glycol and Mycophenolate sodium are added to a part of purified water and stirred until a homogeneous phase is obtained.
2. Mannitol, Methyl cellulose, Sodium thiosulfate and Sodium borate are added to the solution of step 1, followed by volume make up using purified water.
3. pH is measured and then adjusted using Monohydrate sodium nitrate.
4. Sterilization is done by filtration through a 0.22 um filter.

### Example 53

An ophthalmic solution formulation comprising Mycophenolate Sodium as active, Glyceryl Monostearate and Polyoxyl 40 Hydrogenated castor oil as solubilizer, Povidone K30 as thickening agent, Mannitol as tonicity adjusting agent and Sodium borate as preservative, Sodium thiosulfate as antioxidant and Monohydrate sodium nitrate as pH modifier.

TABLE 61

| Ingredient | Function | Composition (% w/v) |
|---|---|---|
| Mycophenolate sodium | Active | 1.0 |
| equivalent to Mycophenolic | | |
| acid | | |
| Mannitol | Tonicity modifier | 5.0 |
| Povidone K30 | Thickening agent | 2 |
| Glyceryl Monostearate | Solubilizer | 0.5 |
| Polyoxyl 40 Hydrogenated | Solubilizer | 2.0 |
| castor oil | | |
| Monohydrate sodium nitrate | pH Modifier | 1.18 |
| Sodium thiosulfate | Antioxidant | 0.5 |
| Sodium borate | Preservative | 1.1 |
| Purified water | Aqueous Phase | q.s to 100% |

Method of Preparation:
1. Glyceryl Monostearate, Polyoxyl 40 Hydrogenated castor oil and Mycophenolate sodium are added to a part of purified water and stirred until a homogeneous phase is obtained.
2. Mannitol, Povidone K30, Sodium thiosulfate and Sodium borate are added to the solution of step 1, followed by volume make up using purified water.
3. pH is measured and then adjusted using Monohydrate sodium nitrate.
4. Sterilization is done by filtration through a 0.22 um filter.

### Example 54

An ophthalmic solution formulation comprising Mycophenolate Sodium as active, Sorbitol and Polyethylene glycol 8000 as solubilizer, Crospovidone as thickening agent, Mannitol as tonicity adjusting agent and Sodium borate as preservative, Sodium thiosulfate as antioxidant and Monohydrate sodium nitrate as pH modifier.

TABLE 62

| Ingredient | Function | Composition (% w/v) |
|---|---|---|
| Mycophenolate sodium | Active | 1.0 |
| equivalent to Mycophenolic | | |
| acid | | |
| Mannitol | Tonicity modifier | 5.0 |
| Crospovidone | Thickening agent | 3 |
| Sorbitol | Solubilizer | 6.7 |
| Polyethylene glycol 8000 | Solubilizer | 2.0 |
| Monohydrate sodium nitrate | pH Modifier | 1.18 |
| Sodium thiosulfate | Antioxidant | 0.5 |
| Sodium borate | Preservative | 1.1 |
| Purified water | Aqueous Phase | q.s to 100% |

Method of Preparation:
1. Sorbitol, Polyethylene glycol 8000 and Mycophenolate sodium are added to a part of purified water and stirred until a homogeneous phase is obtained.
2. Mannitol, Crospovidone, Sodium thiosulfate and Sodium borate are added to the solution of step 1, followed by volume make up using purified water.
3. pH is measured and then adjusted using Monohydrate sodium nitrate.
4. Sterilization is done by filtration through a 0.22 um filter.

### Example 55

An ophthalmic solution formulation comprising Mycophenolate Sodium as active, Tween 80 as solubilizer, HPMC K4M as thickening agent, boric acid as preservative, di-sodium EDTA as chelating agent and sodium chloride as tonicity modifier was prepared as per Table 63 below.

TABLE 63
Ophthalmic solution formulation

| | | Composition (% w/v) |
|---|---|---|
| | | VLN-F-70/ASR/065 |
| Ingredient | Function | 1% w/v Solution |
| Mycophenolate sodium | Active | 1.0 |
| equivalent to Mycophenolic | | |
| acid | | |
| Tween 80 | Surfactant | 4.0 |
| Boric acid | Preservative | 1.0 |
| Di-sodium EDTA | Chelating agent | 0.1 |
| Sodium chloride | Tonicity modifier | 0.23 |
| HPMC K4M | Thickening agent | 0.5 |
| Purified water | | Q.S. up to 100 ml |

Mycophenolate Sodium Solution Formulation Evaluation—

TABLE 64
Initial Evaluation Data:

| Sample | | | Viscosity | Assay | Osmolarity |
|---|---|---|---|---|---|
| B. No. | Description | pH | (mPas) | (% w/v) | (mOsm/kg) |
| VLN-F-70/ | Slight yellow | 7.25 | 17.11 | 97.84 | 268 |
| ASR/065 | aqueous | | | | |
| (1% w/v | solution | | | | |
| Solution) | | | | | |

In-Vitro Release Study of Mycophenolate Sodium Solution Formulation Using Goat Cornea:

Freshly excised goat cornea was used in order to evaluate the permeation capability of mycophenolate sodium from the solution formulation.

Details of the in-vitro study (FIG. 2) was as follows:
Membrane used: Goat eye cornea
Media: Phosphate buffer saline pH 7.4
Replication: Each formulation was applied on Six cells
Dose: Equivalent to 500 μg of Mycophenolic acid
Study Duration—2 hr
Setup was same as performed for in-vitro study for ointment formulations.

TABLE 65
In-vitro release from Mycophenolate sodium solution

| | Cornea | Cornea | Receptor | Amount | |
|---|---|---|---|---|---|
| | Top | Extracted | Media | permeated/area | Recovery |
| | (μg) | (μg) | (μg) | (μg/cm²)^{#} | (%) |
| 1% w/v Mycophenolate | 194.46 ± 38.14 | 123.93 ± 39.43 | 117.32 ± 50.66 | 63.86 | 87.14 |
| sodium solution | | | | | |
| (VLN-F70/ASR/065) | | | | | |

^{#}Considering 1.837 cm2 is the active permeation area

Ex-Vivo Study of Mycophenolate Sodium Solution Formulation Using Whole Goat Eye

With in-vitro studies, it was observed that mycophenolate sodium molecule has the capability to permeate through the goat eye. However, in order to further understand the permeability and retention of the mycophenolate molecule after it permeates through the cornea, ex-vivo study using the goat eye was planned. Ex-vivo study helps in understanding the targeting/retention of the mycophenolate molecule in the different parts of eye.
Whole Goat eye was used and setup was same as it was for ex-vivo study for Ointment formulations.
Media: Phosphate buffer saline pH 7.4
Replication: Each formulation was applied on Six cells
Dose: Equivalent to 500 μg of active for 1% formulation.

TABLE 66
Ex-vivo release from Mycophenolate sodium solution

| | | | | | | | Total | | |
|---|---|---|---|---|---|---|---|---|---|
| | | | | Cornea | | | amount | Cumulative | |
| | No. of | | Cornea | extraction | Lens | Iris | of drug | amount of drug | |
| Formulations | Replicates | Time | top | (mcg) | (mcg) | (mcg) | retained | in receptor | |
| studied | (n) | points | (mcg) | (a) | (b) | (c) | a + b + c | (mcg) | Recovery |
| 1.0% w/v | 4 | 1, 2, | 451.433 ± 112.8 0 | 2.89 ± 2.43 | Not | Not | 2.89 | 1.24 ± 0.64 | 91.11% |
| Mycophenolate | | 3 & 4 hr | | | detected | detected | | | |
| sodium solution | | | | | | | | | |

| | Initial | | | | | | | | | | |
|---|---|---|---|---|---|---|---|---|---|---|---|
| | % | | | | | | | | | | |
| Sample B. | Soluble | | 25° C./60% RH | | | | 40° C./75% RH | | | | 50° C. |
| No. | fraction | Assay | 1 M | 2 M | 3 M | 6 M | 1 M | 2 M | 3 M | 6 M | 1 M |
| VLN-F- | NA | 97.51 | 96.90 | 96.15 | 91.62 | 89.27 | 93.08 | 90.58 | 87.06 | 82.37 | 90.13 |
| 70/ASR/065 | | | | | | | | | | | |
| 1% w/v | | | | | | | | | | | |
| Solution | | | | | | | | | | | |

| VLN-F-70/ASR/065 (1% w/v Solution) | |
|---|---|
| 25° C./60% RH | 40° C./75% RH |

| Impurity | | 3 M | 6 M | 3 M | 6 M |
|---|---|---|---|---|---|
| Name | RRT | % w/v | % w/v | % w/v | % w/v |
| Unk-3 | 0.32 | 0.25 | 0.15 | 0.27 | 0.19 |
| Unk-5 | 0.35 | 0.18 | 0.21 | 0.22 | 0.27 |
| Unk-9 | 0.44 | 0.37 | 0.41 | 0.74 | 1.16 |
| Unk-13 | 0.50 | 0.14 | 0.15 | 0.20 | 0.28 |
| Unk-14 | 0.53 | 0.15 | 0.16 | 0.18 | 0.23 |
| Unk-16 | 0.58 | 1.17 | 1.30 | 1.15 | 0.89 |
| Unk-17 | 0.61 | 0.16 | 0.18 | 0.22 | 0.35 |
| Unk-18 | 0.62 | 0.02 | 0.07 | 0.24 | 0.53 |
| Unk-29 | 0.96 | 0.12 | 0.18 | 0.36 | 0.70 |
| Unk-31 | 1.20 | 0.01 | 0.02 | 0.05 | 0.11 |
| Unk-33 | 1.41 | 0.22 | 0.26 | 0.81 | 1.51 |
| Total Impurity | | 3.185 | 3.482 | 5.024 | 7.072 |

Conclusion:

There is a higher level of decrease in the assay of mycophenolate sodium observed on both, real time and accelerated condition. The decrease was higher in accelerated condition compared to real time stability condition. Degradation levels were also high owing to the 100% soluble form of the drug.

### Example 56

Suspension Formulation: Drug loaded suspension for Uveitis Treatment using acrylic acid polymer was prepared by using Carbopol 974P as a thickening agent. The final formulation was white to off white suspension having pH 6-7. Formulation prepared by using 0.3% w/v Carbopol 974P was evaluated at accelerated stability condition. The amount of drug present in the solubilized form was also estimated.

TABLE 67
Mycophenolate Suspension Formulation with Carbopol 974P.

| | | Composition | Composition |
|---|---|---|---|
| | | (1.0% w/v) | (2.0% w/v) |
| Ingredient | Function | VLN-F-70/ASR/060 | VLN-F-70/ASR/061 |
| Mycophenolic | API | 1.00 | 2.00 |
| acid | | | |
| Polysorbate 80 | Surfactant | 0.25 | 0.25 |
| Carbopol 974P | Thickening | 0.30 | 0.30 |
| | agent | | |
| Glycerol | Tonicity | 0.586 | 0.40 |
| | modifier | | |
| Di-sodium | Chelating | 0.10 | 0.10 |
| EDTA | agent | | |
| Boric acid | Preservative | 1.00 | 1.00 |

| | |
|---|---|
| Purified water | q.s. upto 100 ml |

| | | |
|---|---|---|
| Description | White aq. | White aq. |
| | suspension | suspension |
| Final pH Observed | 6.62 | 7.24 |
| Soluble fraction | 6.57 | 28.49 |
| of API (% w/v) | | |

Method of Preparation:
1. In a main mixing vessel, glycerol, disodium edetate, polysorbate 80 and boric acid were dissolved in purified water followed by addition of Carbopol 974P.
2. Mix the phase obtained in step 1 for 45 mins and pH was observed.
3. Mycophenolate sodium was dispersed in the phase obtained in step 2 with mixing using high shear homogeniser at 10,000 rpm.
4. Final weight makeup was done by purified water and pH of formulation was observed.

TABLE 68
Stability data

| | | Mycophenolate sodium suspension | |
|---|---|---|---|
| Assay of | | 1% w/v | 2% w/v |
| Mycophenolate Sodium | | VLN-F-70/ASR/060 | VLN-F-70/ASR/061 |
| Initial | | 97.84 | 100.85 |
| 1 Month | 25° C./60% RH | 97.04 | 103.02 |
| | 40° C./75% RH | 96.16 | 102.14 |
| 2 Month | 25° C./60% RH | 97.12 | 100.98 |
| | 40° C./75% RH | 94.40 | 99.55 |
| 3 Month | 25° C./60% RH | 95.12 | 97.57 |
| | 40° C./75% RH | 93.15 | 97.16 |
| 6 Month | 25° C./60% RH | 93.59 | 94.40 |
| | 40° C./75% RH | 91.44 | 95.51 |

Conclusion:

There is a decrease in assay of mycophenolate sodium with increase in stability time. Decrease is higher at accelerated condition compared to the real time condition. Also, the decrease in assay is higher in 1% w/v strength compared to the 2% w/v strength.

TABLE 69
Degradation product data

| | | Mycophenolate sodium suspension | | | | | | | |
|---|---|---|---|---|---|---|---|---|---|
| | | VLN-F-70/ASR/060, 1% w/v | | | | VLN-F-70/ASR/061, 2% w/v | | | |
| | | 25° C./60% RH | | 40° C./75% RH | | 25° C./60% RH | | 40° C./75% RH | |
| Impurity | | 3 Month | 6 Month | 3 Month | 6 Month | 3 Month | 6 Month | 3 Month | 6 Month |
| Name | RRT | % w/v | % w/v | % w/v | % w/v | % w/v | % w/v | % w/v | % w/v |

| | | | | | | | | |
|---|---|---|---|---|---|---|---|---|
| Total Impurity | 0.782 | 0.940 | 1.796 | 2.836 | 1.457 | 1.645 | 2.542 | 3.926 |

Conclusion:

Degradation product profile shows increase in degradation product level at 6 months accelerated conditions compared to 6-month real time stability conditions. Degradation product level increased with increase in the strength of mycophenolate sodium from 1% w/v to 2% w/v as the soluble fraction increased with increase in pH.

### Example 57

Preparation of drug loaded suspension formulation with increased thickening agent concentration for 2% w/v suspension, whereas for 1% w/v suspension the thickening agent concentration was kept constant.

As per the observation on stability, soluble fraction was directly proportional to the amount of degradation. Further trials were executed in order to reduce the soluble fraction of API. In present trial, the amount of Carbopol 794P was further increased.

TABLE 70
Mycophenolate Suspension Formulation with increased
polymer percentage for 2% w/v suspension.

| | | Mycophenolate Sodium Suspension | |
|---|---|---|---|
| | | 2.0% w/v | 1.0% w/v |
| Ingredient | Function | VLN-F70/ASR/120 | VLN-F70/ASR/121 |
| Mycophenolic | API | 2.000 | 1.000 |
| acid | | | |
| Polysorbate 80 | Surfactant | 0.250 | 0.250 |
| Boric acid | Preservative | 1.000 | 1.000 |
| Disodium | Chelating | 0.100 | 0.100 |
| EDTA | agent | | |
| Glycerol | Tonicity | 0.400 | 0.586 |
| | modifier | | |
| Carbopol 974P | Thickening | 0.450 | 0.300 |
| | agent | | |

| | |
|---|---|
| Purified water | q.s. upto 100 ml |

| | | |
|---|---|---|
| Description | White aq. | White to off |
| | suspension | white aq. |
| | | suspension |
| Final pH Observed | 6.87 | 6.51 |
| Soluble fraction | 15.55 | 12.21 |
| of API (% w/v) | | |

Manufacturing Procedure:
1. In a main mixing vessel, glycerol, disodium edetate, polysorbate 80 and boric acid were dissolved in purified water followed by addition of Carbopol 974P.
2. Mixing of the phase obtained in step 1 was performed for 45 mins and pH was observed.
3. Mycophenolate sodium was dispersed in the phase obtained in step 2 with mixing using high shear homogeniser at 10,000 rpm.
4. Final weight was made up with purified water and pH of formulation was observed.

TABLE 71
Stability data

| | | Mycophenolate sodium suspension | |
|---|---|---|---|
| Assay of | | 2% w/v | 1% w/v |
| Mycophenolate Sodium | | VLN-F70/ASR/120 | VLN-F70/ASR/121 |
| Initial | | 102.27 | 98.85 |
| 3 Month | 25° C./60% RH | 99.55 | 93.33 |
| | 40° C./75% RH | 97.48 | 92.30 |

TABLE 72
Degradation profile

1% w/v Mycophenolate sodium suspension
VLN-F-70/ASR/121

| | |
|---|---|
| 25° C./60% RH | 40° C./75% RH |

| Impurity | | | 3 | 6 | 3 | 6 |
|---|---|---|---|---|---|---|
| Name | RRT | Initial | Months | Months | Months | Months |
| Total | | 0.059 | 0.390 | 0.482 | 1.234 | 1.794 |
| Impurity | | | | | | |

| | | 2% w/v Mycophenolate sodium suspension | | | | |
|---|---|---|---|---|---|---|
| | | VLN-F-70/ASR/120 | | | | |
| Impurity | | | 25° C./60% RH | 25° C./60% RH | 40° C./75% RH | 40° C./75% RH |
| Name | RRT | Initial | 3 Months | 6 Months | 3 Months | 6 Months |
| Total | | 0.074 | 0.549 | 0.782 | 1.377 | 2.214 |
| Impurity | | | | | | |

It was understood from the stability study that the soluble fraction is directly proportional to the degradation product. Taking this learning ahead, further trials were executed in order to reduce the soluble fraction of API. With the decrease in pH of the formulation, there was a decrease in the soluble fraction of the API which resulted in decreased degradation product levels. Trials were planned to further reduce the soluble fraction of the drug to have a more stable product.

### Example 58

Preparation of drug loaded suspension with reduced level of soluble fraction by using different levels of pH modifier:

To further understand the effect of pH and surfactant concentration on the soluble fraction of API, further trials were planned using citric acid as pH modifier.

TABLE 73
Mycophenolate Suspension Formulation with different percentage
of citric acid as pH modifier and reduced surfactant percentage

| | | Mycophenolate Sodium | | |
|---|---|---|---|---|
| | | Suspension 2% w/w | | |
| | | VLN-F- | VLN-F- | VLN-F- |
| | | 70/ASR/ | 70/ASR/ | 70/ASR/ |
| Ingredient | Function | 142A | 142B | 142C |
| Mycophenolic | API | 2.000 | 2.000 | 2.000 |
| acid | | | | |
| Polysorbate 80 | Surfactant | 0.100 | 0.100 | 0.100 |
| Boric acid | Preservative | 1.000 | 1.000 | 1.000 |
| Citric acid | pH modifier | — | 0.100 | 0.200 |
| Glycerol | Tonicity | 0.400 | 0.400 | 0.400 |
| | modifier | | | |
| Carbopol 974P | Thickening | 0.450 | 0.450 | 0.450 |
| | agent | | | |
| Purified water | | q.s. upto 100 ml | | |
| Description | | White to off white aq. suspension | | |
| Final pH | | 6.91 | 6.53 | 5.94 |
| Observed | | | | |
| Soluble fraction | | 22.53 | 6.71 | 1.62 |
| of API (% w/v) | | | | |

Manufacturing Procedure:
1. In a main mixing vessel, glycerol, polysorbate 80 and boric acid were dissolved in purified water followed by addition of Carbopol 974P.
2. Mixing the phase obtained in step 1 was done for 45 mins and pH was observed.
3. Mycophenolate sodium was dispersed in the phase obtained in step 2 with mixing using high shear homogeniser at 10,000 rpm.
4. Citric acid was dissolved in purified water and was used to bring the suspension formulation to the desired pH levels.
5. Final weight was made up by purified water and pH of formulation was observed.

TABLE 74
Degradation product profile

| | | 3 Month | | | |
|---|---|---|---|---|---|
| | | 25° C./ | 40° C./ | 60° C. | |
| Batch Number | Initial | 60% RH | 75% RH | 1 week | 3 weeks |
| VLN-F-70/ASR/142C | 0.08 | 0.15 | 0.55 | 1.61 | 1.57 |
| VLN-F-70/ASR/142B | 0.132 | 0.373 | 1.325 | 1.648 | 2.952 |
| VLN-F-70/ASR/142A | 0.360 | 0.847 | 2.601 | 5.2 | 6.100 |

Conclusion:

Degradation product profile data shows that with the decrease in pH of the formulation there is a decrease in soluble fraction and ultimately decrease in the total degradation product level.

### Example 59

Preparation of drug loaded suspension with different percentage of orthophosphoric acid (pH modifier) and thickening agent:
Further trials were planned with using orthophosphoric acid and Polycarbophil. Also, surfactant was removed in this trial.

TABLE 75
Mycophenolate Suspension Formulation with different percentage
of orthophosphoric acid (pH modifier) and thickening agent

| | | Composition in Percentage (% w/v) | | | |
|---|---|---|---|---|---|
| | | (VLN-F-70/ | (VLN-F-70/ | (VLN-F-70/ | (VLN-F-70/ |
| Ingredient | Function | ASR/145A) | ASR/145B) | ASR/145C) | ASR/145D) |
| Mycophenolic acid | API | 2.000 | 2.000 | 2.000 | 2.000 |
| Boric acid | Preservative | 1.000 | 1.000 | 1.000 | 1.000 |
| Glycerol | Tonicity modifier | 0.400 | 0.400 | 0.400 | 0.400 |
| Polycarbophil | Thickening agent | 0.400 | 0.400 | 0.600 | 0.600 |
| Ortho Phosphoric acid | pH modifier | q.s | q.s | q.s | q.s |
| Purified water | | Q.S. upto | Q.S. upto | Q.S. upto | Q.S. upto |
| | | 100 ml | 100 ml | 100 ml | 100 ml |

| | | | | |
|---|---|---|---|---|
| Final pH observed | 7.01 | 6.55 | 6.04 | 5.48 |
| Soluble fraction | 11.36 | 2.73 | 0.78 | 0.23 |

Manufacturing Procedure:
1. In a main mixing vessel, glycerol and boric acid were dissolved in purified water followed by addition of polycarbophil.
2. Phase obtained in step 1 was mixed for 45 mins and pH was observed.
3. Mycophenolate sodium was dispersed in the phase obtained in step 2 with mixing using high shear homogeniser at 10,000 rpm.
4. Orthophosphoric acid was dilute with purified water up to desired strength and was used to bring the pH of suspension formulation to the desired level.
5. Final weight was made up with purified water and pH of formulation was observed.

TABLE 76
Stability Data

| | | | | VLN-F- | VLN-F- | VLN-F- | VLN-F- |
|---|---|---|---|---|---|---|---|
| | | | | 70/ASR/145A | 70/ASR/145B | 70/ASR/145C | 70/ASR/145D |
| Assay | Initial | | | 94.35 | 104.05 | 83.83 | 95.86 |
| | 60° | C. | 1 week | 90.13 | 102.05 | 81.17 | 91.73 |
| | | | 3 weeks | 85.91 | 83.32 | 71.52 | 84.01 |
| | 3 | Month | 25° C./60% RH | 93.12 | 102.72 | 82.58 | 89.28 |
| | | | 40° C./75% RH | 87.10 | 99.52 | 78.83 | 91.23 |

TABLE 77
Degradation product profile

| | | | 3 Month | | 60° C. | |
|---|---|---|---|---|---|---|
| Batch Number | pH | Initial | 25° C./60% RH | 40° C./75% RH | 1 week | 3 weeks |
| VLN-F-70/ASR/145A | 7.01 | 0.444 | 1.01 | 3.22 | 4.22 | 5.58 |
| VLN-F-70/ASR/145B | 6.55 | 0.175 | 0.41 | 1.52 | 3.17 | 4.05 |
| VLN-F-70/ASR/145C | 6.04 | 0.240 | 0.23 | 1.34 | 2.94 | 3.82 |
| VLN-F-70/ASR/145D | 5.48 | 0.109 | 0.25 | 1.14 | 2.06 | 2.57 |

For a suspended formulation, uniformity of dose is the critical quality attribute which can be achieved by preventing the sedimentation of the suspended particles in the formulation.

### Example 60

Preparation of drug loaded suspension with citric acid as pH modifier and different percentage of Thickening agent:

During stability studies at 25° C./60% RH and 40° C./75% RH, sedimentation of the suspended API was observed. This resulted in variation of the assay. Hence, further trials were planned with increased polycarbophil percentage along with citric acid as pH modifier (Table 78). Also, surfactant was reduced to 0.100% w/w in this trial.

TABLE 78
Mycophenolate Suspension Formulation with citric acid as
pH modifier and different percentages of thickening agent

| | | Composition in Percentage (% w/w) | | |
|---|---|---|---|---|
| | | VLN-F-70/ | VLN-F-70/ | VLN-F-70/ |
| Ingredient | Function | ASR/170A | ASR/170B | ASR/180 |
| Mycophenolic | API | 2.000 | 2.000 | 2.000 |
| acid | | | | |
| Polysorbate 80 | Surfactant | 0.100 | 0.100 | 0.100 |
| Boric acid | Preservative | 1.000 | 1.000 | 1.000 |
| Glycerol | Tonicity | 0.400 | 0.400 | 0.400 |
| | modifier | | | |
| Di-sodium | Chelating | 0.100 | 0.100 | 0.100 |
| EDTA | agent | | | |
| Polycarbophil | Thickening | 0.700 | 0.800 | 0.860 |
| | agent | | | |
| Citric acid | pH Modifier | 0.300 | 0.150 | 0.150 |
| Purified water | | q.s. upto 100 gm | | |
| Final pH | | 5.59 | 5.52 | 5.51 |
| observed | | | | |

Manufacturing Procedure:
1. In a main mixing vessel, glycerol, disodium edetate, polysorbate 80 and boric acid were dissolved in purified water followed by addition of Polycarbophil.
2. The phase obtained in step 1 was mixed for 45 mins and pH was observed.
3. Mycophenolate sodium was dispersed in the phase obtained in step 2 with mixing using high shear homogeniser at 10,000 rpm.
6. Citric acid was dissolved in purified water and was used to bring the suspension formulation to the desired pH levels.
4. Final weight was made up by purified water and pH of formulation was observed.

To understand the uniformity drug in the formulation, estimation of drug content was performed in every drop and the results are tabulated below.

TABLE 79
Drop assay

| | Drop | % Assay of |
|---|---|---|
| | weight | Mycophenolic |
| Sample B. No. | (mg) | Acid |
| VLN-F-70/ASR/180-DROP-1 | 48.24 | 101.24 |
| VLN-F-70/ASR/180-DROP-2 | 42.77 | 100.84 |
| VLN-F-70/ASR/180-DROP-3 | 43.25 | 101.19 |
| VLN-F-70/ASR/180-DROP-4 | 43.41 | 102.32 |
| VLN-F-70/ASR/180-DROP-5 | 44.79 | 102.12 |
| VLN-F-70/ASR/180-DROP-6 | 43.04 | 101.99 |

| | |
|---|---|
| Mean | 101.62 |
| SD | 0.602 |
| % RSD | 0.59 |

TABLE 80
Degradation product profile

| | | |
|---|---|---|
| | | VLN-F-70/ASR/180 |
| Impurity Name | RRT | Initial |

| | |
|---|---|
| Total Impurity | 0.082 |

In Vitro Release Study Using Goat Cornea
Membrane used: Goat eye cornea
Media: Phosphate buffer saline pH 7.4
Replication: Each formulation was applied on Six cells
Dose: Equivalent to 500 μg of active for 1% formulation and 1000 μg of active for 2% formulation.
Study Duration—2 hr

TABLE 81
Results

| | | | | Amount | |
|---|---|---|---|---|---|
| | Cornea Top | Cornea | Receptor | permeated/area | Recovery |
| | (μg) | Extracted (μg) | Media (μg) | (μg/cm2)# | (%) |
| 1% w/v Mycophenolate | 190.20 ± 38.04 | 158.39 ± 31.68 | 150.30 ± 30.06 | 81.81 | 99.78 |
| sodium suspension | | | | | |
| (VLN-F70/ASR/060) | | | | | |
| 2% w/v Mycophenolate | 511.67 ± 55.92 | 304.87 ± 44.80 | 257.96 ± 28.26 | 140.42 | 107.45 |
| sodium suspension | | | | | |
| (VLN-F70/ASR/061) | | | | | |

#Considering 1.837 cm² is the active permeation area

Observation:
Mycophenolate showed a good corneal permeation capability as significant amount of drug was found in the Receptor media.
Also, substantial amount of drug was retained in the corneal layers.

Ex Vivo Study Using Whole Goat Eye:
Freshly excised complete goat eyeball was procured and adhering tissue was removed.
Franz Cells were filled with PBS 7.4 and connected to water circulator maintained at 37° C.
Complete eyeball wrapped up in cotton was placed over the Franz cells in a fashion that it will remain in contact with the PBS 7.4 media.
PBS 7.4 solution was infused at 10 μL/min to the anterior portion of eye using Micro infusion pump.
A needle was attached to the diagonally opposite end for sample collection.
A circular ring of 8.5 mm was placed over the cornea along with the cotton outside the ring and formulation was applied inside the ring.
After 7 min, formulation was wicked using cotton and washing was done using 1 ml PBS 7.4. Solution obtained during washing was also collected (from the cotton placed around the ring).

The ex vivo set-up is further shown in FIG. 1.
Media: Biological salt solution
Replication: Each formulation was applied on Six cells
Dose: Equivalent to 500 μg of active for 1% formulation and 1000 μg of active for 2% formulation.

TABLE 82
Results

| | | | | | | | Total | | |
|---|---|---|---|---|---|---|---|---|---|
| | | | | Cornea | | | amount | Cumulative | |
| | No. of | | Cornea | extraction | Lens | Iris | of drug | amt of drug | |
| Formulations | Replicates | Time | top | (mcg) | (mcg) | (mcg) | retained | in receptor | |
| studied | (n) | points | (mcg) | (a) | (b) | (c) | a + b + c | (mcg) | Recovery |
| 1.0% w/v | 6 | 1, 2, | 435.81 ± 46.69 | 4.83 ± 2.60 | 0.61 ± 0.77 | 1.24 ± 1.43 | 6.68 | 4.40 ± 2.03 | 89.38% |
| Mycophenolate | | 3, 4 & | | | | | | | |
| sodium suspension | | 5 hr | | | | | | | |
| (VLN-F70/ASR/060) | | | | | | | | | |
| 2.0% w/v | | | 1179.64 ± 37.73 | 5.07 ± 1.91 | 3.39 ± 3.67 | 1.43 ± 0.51 | 9.89 | 4.66 ± 1.12 | 119.42% |
| Mycophenolate | | | | | | | | | |
| sodium suspension | | | | | | | | | |
| (VLN-F70/ASR/061) | | | | | | | | | |

Results are shown as Mean ± SD

Observation:
Both 1% and 2% w/v suspension formulations showed almost similar amount of drug permeated.
2% w/v formulation had higher amount of drug retained in the iris which is the site of infection in-case of Uveitis.

### Example 61

An ophthalmic suspension formulation comprising Mycophenolate Mofetilas active, Polyoxyl 40 stearate as suspending agent, Mannitol as tonicity adjusting agent and Boric acid as preservative, Sodium phosphate monobasic anhydrous as buffering agent, Sodium sulfate as antioxidant and Sodium hydroxide as pH modifier.

TABLE 83

| | | Composition |
|---|---|---|
| Ingredient | Function | (% w/v) |
| Mycophenolate Mofetil | Active | 1.0 |
| Mannitol | Tonicity modifier | 3.0 |
| Sodium sulfate | Antioxidant | 1.2 |
| Polyoxyl 40 stearate | Suspending agent | 0.5 |
| Sodium phosphate | Buffering agent | 2.0 |
| monobasic anhydrous | | |
| Sodium hydroxide | pH Modifier | QS |
| Boric acid | Preservative | 0.3 |
| Purified water | Aqueous Phase | q.s to 100% |

Method of Preparation:
1. Polyoxyl 40 stearate is dissolved in about 80 ml of purified water by effecting dispersion with warming, followed by cooling to room temperature.
2. Mannitol, Sodium sulfate, Sodium phosphate monobasic and Boric acid are added for dissolution.
3. The pH is adjusted by adding 0.1 M sodium hydroxide.
4. Mycophenolate Mofetil is added and uniform suspension is prepared using a homogenizer.
5. Purified water is added to make the whole volume.

### Example 62

An ophthalmic suspension formulation comprising Mycophenolate Mofetil as active, Poloxomer 407 as suspending agent, Mannitol as tonicity adjusting agent and Boric acid as preservative, Sodium phosphate monobasic anhydrous as buffering agent, Sodium sulfate as antioxidant and Sodium hydroxide as pH modifier.

TABLE 84

| | | Composition |
|---|---|---|
| Ingredient | Function | (% w/v) |
| Mycophenolate Mofetil | Active | 1.0 |
| Mannitol | Tonicity modifier | 3.0 |
| Sodium sulfate | Anti-oxidant | 1.2 |
| Poloxomer 407 | Suspending agent | 0.1 |
| Sodium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Poloxomer 407 is dissolved in about 80 ml of purified water by effecting dispersion with warming, followed by cooling to room temperature.
2. Mannitol, Sodium sulfate, Sodium phosphate monobasic and Boric acid are added for dissolution.
3. The pH is adjusted by adding 0.1 M sodium hydroxide.
4. Mycophenolate Mofetil is added and uniform suspension is prepared using a homogenizer.
5. Purified water is added to make the whole volume.

### Example 63

An ophthalmic suspension formulation comprising Mycophenolate Mofetil as active, Cetyl Alcohol as suspending agent, Mannitol as tonicity adjusting agent and Boric acid as preservative, Sodium phosphate monobasic anhydrous as buffering agent, Sodium sulfate as antioxidant and Sodium hydroxide as pH modifier.

TABLE 85

| | | Composition |
|---|---|---|
| Ingredient | Function | (% w/v) |
| Mycophenolate Mofetil | Active | 1.0 |
| Mannitol | Tonicity modifier | 3.0 |
| Sodium sulfate | Anti-oxidant | 1.2 |
| Cetyl Alcohol | Suspending agent | 0.5 |
| Sodium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Cetyl Alcohol is dissolved in about 80 ml of purified water by effecting dispersion with warming, followed by cooling to room temperature.
2. Mannitol, Sodium sulfate, Sodium phosphate monobasic and Boric acid are added for dissolution.
3. The pH is adjusted by adding 0.1 M sodium hydroxide.
4. Mycophenolate Mofetil is added and uniform suspension is prepared using a homogenizer.
5. Purified water is added to make the whole volume.

### Example 64

An ophthalmic suspension formulation comprising Mycophenolate Mofetil as active, Glyceryl Monostearate as suspending agent, Mannitol as tonicity adjusting agent and Boric acid as preservative, Sodium phosphate monobasic anhydrous as buffering agent, Sodium sulfate as antioxidant and Sodium hydroxide as pH modifier.

TABLE 86

| | | Composition |
|---|---|---|
| Ingredient | Function | (% w/v) |
| Mycophenolate Mofetil | Active | 1.0 |
| Mannitol | Tonicity modifier | 3.0 |
| Sodium sulfate | Anti-oxidant | 1.2 |
| Glyceryl Monostearate | Suspending agent | 0.5 |
| Sodium phosphate monobasic, | Buffering agent | 2.0 |
| anhydrous | | |
| Sodium hydroxide | pH Modifier | QS |
| Boric acid | Preservative | 0.3 |
| Purified water | Aqueous Phase | q.s to 100% |

Method of Preparation:
1. Glyceryl Monostearate is dissolved in about 80 ml of purified water by effecting dispersion with warming, followed by cooling to room temperature.
2. Mannitol, Sodium sulfate, Sodium phosphate monobasic and Boric acid are added for dissolution.
3. The pH is adjusted by adding 0.1 M sodium hydroxide.
4. Mycophenolate Mofetil is added and uniform suspension is prepared using a homogenizer.
5. Purified water is added to make the whole volume.

### Example 65

An ophthalmic suspension formulation comprising Mycophenolate Mofetilas active, Glyceryl Monostearate as suspending agent, Mannitol as tonicity adjusting agent and Boric acid as preservative, Sodium phosphate monobasic anhydrous as buffering agent, Sodium sulfate as antioxidant and Sodium hydroxide as pH modifier.

TABLE 87

| | | Composition |
|---|---|---|
| Ingredient | Function | (% w/v) |
| Mycophenolate Mofetil | Active | 1.0 |
| Mannitol | Tonicity modifier | 3.0 |
| Sodium sulfate | Anti-oxidant | 1.2 |
| Glyceryl Monostearate | Suspending agent | 0.5 |
| Sodium phosphate monobasic, | Buffering agent | 2.0 |
| anhydrous | | |
| Sodium hydroxide | pH Modifier | QS |
| Boric acid | Preservative | 0.3 |
| Purified water | Aqueous Phase | q.s to 100% |

Method of Preparation:
1. Glyceryl Monostearate is dissolved in about 80 ml of purified water by effecting dispersion with warming, followed by cooling to room temperature.
2. Mannitol, Sodium sulfate, Sodium phosphate monobasic and Boric acid are added for dissolution.
3. The pH is adjusted by adding 0.1 M sodium hydroxide.
4. Mycophenolate Mofetil is added and uniform suspension is prepared using a homogenizer.
5. Purified water is added to make the whole volume.

### Example 66

An ophthalmic suspension formulation comprising Mycophenolate Mofetilas active, Glyceryl Monostearate as suspending agent, Sodium chloride as tonicity adjusting agent and Boric acid as preservative, Potassium phosphate monobasic as buffering agent, Sodium sulfate as antioxidant, Carbopol 974P as thickening agent and Tromethamine as pH modifier.

TABLE 88

| | | Composition |
|---|---|---|
| Ingredient | Function | (% w/v) |
| Mycophenolate Mofetil | Active | 1.0 |
| Sodium chloride | Tonicity modifier | 0.9 |
| Sodium sulfate | Anti-oxidant | 1.2 |
| Carbopol 974P | Thickening agent | 0.45 |
| Potassium phosphate | Buffering agent | 0.44 |
| monobasic | | |
| Tromethamine | pH Modifier | QS |
| Boric acid | Preservative | 0.3 |
| Purified water | Aqueous Phase | q.s to 100% |

Method of Preparation:
1. Glyceryl Monostearate is dissolved in about 80 ml of purified water by effecting dispersion with warming, followed by cooling to room temperature.
2. Sodium chloride, Sodium sulfate, Potassium phosphate monobasic and Boric acid are added for dissolution.
3. Carbopol 974P is added to the solution.
4. The pH is adjusted by adding Tromethamine.
5. Mycophenolate Mofetil is added and uniform suspension is prepared using a homogenizer.
6. Purified water is added to make the whole volume.

### Example 67

An ophthalmic suspension formulation comprising Mycophenolate Mofetil as active, Glyceryl Monostearate as suspending agent, Sodium chloride as tonicity adjusting agent and Boric acid as preservative, Potassium phosphate monobasic as buffering agent, Sodium thiosulfate as antioxidant, Povidone as thickening agent and Tromethamine as pH modifier.

TABLE 89

| | | Composition |
|---|---|---|
| Ingredient | Function | (% w/v) |
| Mycophenolate Mofetil | Active | 1.0 |
| Sodium chloride | Tonicity modifier | 0.9 |
| Sodium thiosulfate | Anti-oxidant | 1.2 |
| Povidone | Thickening agent | 1.8 |
| Potassiumphosphate | Buffering agent | 0.44 |
| monobasic | | |
| Tromethamine | pH Modifier | QS |
| Boric acid | Preservative | 0.3 |
| Purified water | Aqueous Phase | q.s to 100% |

Method of Preparation:
1. Povidone is dissolved in about 80 ml of purified water by effecting dispersion with warming, followed by cooling to room temperature.
2. Mannitol, Sodium thiosulfate, Sodium phosphate monobasic and Boric acid are added for dissolution.
3. The pH is adjusted by adding Tromethamine.
4. Mycophenolate Mofetil is added and uniform suspension is prepared using a homogenizer.
5. Purified water is added to make the whole volume.

### Example 68

An ophthalmic suspension formulation comprising Mycophenolate Mofetilas active, Polysorbate 80 as suspending agent, Sodium chloride as tonicity adjusting agent and Boric acid as preservative, Potassium phosphate monobasic as buffering agent, Sodium thiosulfate as antioxidant, Polycarbophil as thickening agent and Tromethamine as pH modifier.

TABLE 90

| | | Composition |
|---|---|---|
| Ingredient | Function | (% w/v) |
| Mycophenolate Mofetil | Active | 1.0 |
| Sodium chloride | Tonicity modifier | 0.9 |
| Sodium thiosulfate | Anti-oxidant | 3.15 |
| Polysorbate 80 | Suspending agent | 0.1 |
| Polycarbophil | Thickening agent | 0.86 |
| Potassium phosphate | Buffering agent | 0.44 |
| monobasic | | |
| Sodium hydroxide | pH Modifier | QS |
| Boric acid | Preservative | 0.3 |
| Purified water | Aqueous Phase | q.s to 100% |

Method of Preparation:
1. Polycarbophil and Polysorbate 80 are dissolved in about 80 ml of purified water by effecting dispersion with warming, followed by cooling to room temperature.
2. Sodium chloride, Sodium thiosulfate, Potassium phosphate monobasic and Boric acid are added for dissolution.
3. The pH is adjusted by adding 0.1 M Sodium hydroxide.
4. Mycophenolate Mofetil is added and uniform suspension is prepared using a homogenizer.
5. Purified water is added to make the whole volume.

### Example 69

An ophthalmic suspension formulation comprising Mycophenolate Mofetil as active, Polysorbate 20 as suspending agent, Sodium chloride as tonicity adjusting agent and Benzalkonium chloride as preservative, Potassium phosphate monobasic as buffering agent, Sodium thiosulfate as antioxidant, Guar gum as thickening agent and Tromethamine as pH modifier.

TABLE 91

| | | Composition |
|---|---|---|
| Ingredient | Function | (% w/v) |
| Mycophenolate Mofetil | Active | 1.0 |
| Sodium chloride | Tonicity modifier | 0.9 |
| Sodium thiosulfate | Anti-oxidant | 3.15 |
| Polysorbate 20 | Suspending agent | 0.1 |
| Guar gum | Thickening agent | 0.2 |
| Potassium phosphate | Buffering agent | 0.44 |
| monobasic | | |
| Sodium hydroxide | pH Modifier | QS |
| Benzalkonium chloride | Preservative | 2.0 |
| Purified water | Aqueous Phase | q.s to 100% |

Method of Preparation:
1. Guar gum and Polysorbate 20 are dissolved in about 80 ml of purified water by effecting dispersion with warming, followed by cooling to room temperature.
2. Sodium chloride, Sodium thiosulfate, Potassium phosphate monobasic and Benzalkonium chloride are added for dissolution.
3. The pH is adjusted by adding 0.1M Sodium hydroxide.
4. Mycophenolate Mofetil is added and uniform suspension is prepared using a homogenizer.
5. Purified water is added to make the whole volume.

### Example 70

An ophthalmic nano-suspension formulation comprising Mycophenolate Sodium as active, Polycarbophil and Acrylate co-polymer as thickening agent, Glycerol as tonicity adjusting agent and boric acid as preservative, di-sodium EDTA as chelating agent and polysorbate 80 as a wetting agent.

TABLE 92

Composition (% w/v)

| Ingredient | Function | A | B | C |
|---|---|---|---|---|
| Mycophenolate | Active | 1.0 | 1.0 | 1.0 |
| Sodium | | | | |
| Glycerol | Tonicity modifier | 0.2 | 0.2 | 0.2 |
| Di-sodium | Chelating agent | 0.1 | 0.1 | 0.1 |
| EDTA | | | | |
| Polysorbate 80 | Wetting agent | 0.10 | 0.15 | 0.25 |
| Polycarbophil | Thickening agent | 0.86 | 0.25 | 0.50 |
| Boric acid | Preservative | 1.0 | 1.0 | 1.0 |
| Acrylate | Thickening agent | 0.2 | 0.5 | 0.8 |
| co-polymer | | | | |
| Purified water | Aqueous Phase | q.s to | q.s to | q.s to |
| | | 100% | 100% | 100% |

Method of Preparation:
1. Glycerol, Di-sodium EDTA, Polysorbate 80 and Boric acid are added to about 80 ml of purified water and dissolved completely.
2. Polycarbophil along with Acrylate co-polymer is hydrated in step 1 solution.
3. Mycophenolate sodium is added along with homogenization using high shear homogenizer to prepare a coarse suspension.
4. The coarse suspension was then to be passed through a high-pressure homogenizer/a ball mill for several cycles to obtain a Nano suspension.
5. Purified water is added to make the whole volume.

### Example 71

An ophthalmic suspension formulation comprising Mycophenolate Mofetilas active, Polysorbate 20 as suspending agent, Sodium chloride as tonicity adjusting agent and Benzalkonium chloride as preservative, Potassium phosphate monobasic as buffering agent, Sodium thiosulfate as antioxidant, Carbomer 934 as thickening agent and Tromethamine as pH modifier. The particle size of the suspension is less than 10 micron.

TABLE 93

| | | Composition |
|---|---|---|
| Ingredient | Function | (% w/v) |
| Mycophenolate Mofetil | Active | 1.0 |
| Sodium chloride | Tonicity modifier | 0.9 |
| Sodium thiosulfate | Anti-oxidant | 3.15 |
| Polysorbate 20 | Suspending agent | 0.1 |
| Carbomer 934 | Thickening agent | 0.45 |
| Guar gum | Thickening agent | 0.2 |
| Potassium phosphate | Buffering agent | 0.44 |
| monobasic | | |
| Sodium hydroxide | pH Modifier | QS |
| Benzalkonium chloride | Preservative | 2.0 |
| Purified water | Aqueous Phase | q.s to 100% |

Method of Preparation:
1. Carbomer 934 and Guar gum are dissolved in about 80 ml of purified water by effecting dispersion with warming, followed by cooling to room temperature.
2. Mannitol, Sodium thiosulfate, Sodium phosphate monobasic and Boric acid are added for dissolution.
3. The pH is adjusted by adding 0.1 M Sodium hydroxide.
4. Mycophenolate Mofetil is added and uniform suspension is prepared using a homogenizer.
5. Purified water is added to make the whole volume.

### Example 72

An ophthalmic Niosomal formulation comprising Mycophenolate Mofetil as active, Lanolin alcohol and Span 60 as membrane forming agents, Boric acid as preservative, Sodium sulfate as antioxidant, Sodium phosphate monobasic anhydrous as buffering agent and Sodium hydroxide as pH modifier.

TABLE 94

| | | Composition |
|---|---|---|
| Ingredient | Function | (% w/v) |
| Mycophenolate Mofetil | Active | 1.0 |
| Lanolin alcohol | Membrane forming agent | 0.76 |
| Span 60 | Membrane forming agent | 2.62 |
| Sodium sulfate | Anti-oxidant | 1.2 |
| Sodium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Lanolin alcohol, span 60 and Mycophenolate mofetil are dissolved in 20 ml of ethanol.
3. Solution is kept for evaporation in a rotary evaporator for 1 h, which resulted in the formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic and boric acid.
6. Sonication is done for 15 mins at 60° C.
7. pH of the resulting solution is adjusted using 0.1 M sodium hydroxide.
8. Volume makeup is done using purified water.

### Example 73

An ophthalmic niosomal formulation comprising Mycophenolate Mofetil as active, Lanolin alcohol and Span 40 as membrane forming agents, Boric acid as preservative, Sodium sulfate as antioxidant, Sodium phosphate monobasic anhydrous as buffering agent and Sodium hydroxide as pH modifier.

TABLE 95

| | | Composition |
|---|---|---|
| Ingredient | Function | (% w/v) |
| Mycophenolate Mofetil | Active | 1.0 |
| Lanolin alcohol | Membrane forming agent | 1.13 |
| Span 40 | Membrane forming agent | 3.93 |
| Sodium sulfate | Anti-oxidant | 1.2 |
| Sodium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Lanolin alcohol, span 40 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic and boric acid.
6. Sonication is done for 15 mins at 60° C.
7. pH of the resulting solution is adjusted using 0.1 M sodium hydroxide.
8. Volume makeup is done using purified water.

### Example 74

An ophthalmic niosomal formulation comprising Mycophenolate Mofetilas active, Cetyl alcohol and Tween 80 as membrane forming agents, Boric acid as preservative, Sodium sulfate as antioxidant, Sodium phosphate monobasic anhydrous as buffering agent and Sodium hydroxide as pH modifier.

TABLE 96

| | | Composition |
|---|---|---|
| Ingredient | Function | (% w/v) |
| Mycophenolate Mofetil | Active | 1.0 |
| Cetyl alcohol | Membrane forming agent | 2.00 |
| Tween 80 | Membrane forming agent | 4.00 |
| Sodium sulfate | Anti-oxidant | 1.2 |
| Sodium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Cetyl alcohol, tween 80 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic and boric acid.
6. Sonication is done for 15 mins at 60° C.
7. pH of the resulting solution is adjusted using 0.1 M sodium hydroxide.
8. Volume makeup is done using purified water.

### Example 75

An ophthalmic niosomal formulation comprising Mycophenolate Mofetilas active, Cetostearyl alcohol and Span 60 as membrane forming agents, Boric acid as preservative, Sodium sulfate as antioxidant, Sodium phosphate monobasic anhydrous as buffering agent, Polyoxyl 40 stearate as suspending agent and Sodium hydroxide as pH modifier.

TABLE 97

| | | Composition |
|---|---|---|
| Ingredient | Function | (% w/v) |
| Mycophenolate Mofetil | Active | 1.0 |
| Cetostearyl alcohol | Membrane forming agent | 2.00 |
| Span 60 | Membrane forming agent | 4.00 |
| Sodium sulfate | Anti-oxidant | 1.2 |
| Sodium phosphate | Buffering agent | 2.0 |
| monobasic, anhydrous | | |
| Polyoxyl 40 stearate | Suspending agent | 0.5 |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Cetostearyl alcohol, span 60 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic, polyoxyl 40 stearate and boric acid.
6. Sonication is done for 15 mins at 60° C.
7. pH of the resulting solution is adjusted using 0.1 M sodium hydroxide.
8. Volume makeup is done using purified water.

### Example 76

An ophthalmic niosomal formulation comprising Mycophenolate Mofetil as active, Cetostearyl alcohol and Nonoxynol-9 as membrane forming agents, Boric acid as preservative, Sodium sulfate as antioxidant, Sodium phosphate monobasic anhydrous as buffering agent, Polyoxyl 35 castor oil as suspending agent and Sodium hydroxide as pH modifier.

TABLE 98

| | | Composition |
|---|---|---|
| Ingredient | Function | (% w/v) |
| Mycophenolate Mofetil | Active | 1.0 |
| Cetostearyl alcohol | Membrane forming agent | 6.00 |
| Nonoxynol-9 | Membrane forming agent | 10.00 |
| Sodium sulfate | Anti-oxidant | 1.2 |
| Sodium phosphate | Buffering agent | 2.0 |
| monobasic, anhydrous | | |
| Polyoxyl 35 castor oil | Suspending agent | 1.0 |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Cetostearyl alcohol, nonoxynol-9 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic, polyoxyl 35 castor oil and boric acid.
6. Sonication is done for 15 mins at 60° C.
7. pH of the resulting solution is adjusted using 0.1 M sodium hydroxide.
8. Volume makeup is done using purified water.

### Example 77

An ophthalmic niosomal formulation comprising Mycophenolate Mofetil as active, Cetostearyl alcohol and Nonoxynol-9 as membrane forming agents, Boric acid as preservative, Sodium sulfate as antioxidant, Sodium phosphate monobasic anhydrous as buffering agent, Poloxomer 407 as suspending agent and Sodium hydroxide as pH modifier.

TABLE 99

| | | Composition |
|---|---|---|
| Ingredient | Function | (% w/v) |
| Mycophenolate Mofetil | Active | 1.0 |
| Cetostearyl alcohol | Membrane forming agent | 6.00 |
| Nonoxynol-9 | Membrane forming agent | 10.00 |
| Sodium sulfate | Anti-oxidant | 1.2 |
| Sodium phosphate | Buffering agent | 2.0 |
| monobasic, anhydrous | | |
| Poloxomer 407 | Suspending agent | 0.5 |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Cetostearyl alcohol, nonoxynol-9 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic, Poloxomer 407 and boric acid.
6. Sonication is done for 15 mins at 60° C.
7. pH of the resulting solution is adjusted using 0.1 M sodium hydroxide.
8. Volume makeup is done using purified water.

### Example 78

An ophthalmic niosomal formulation comprising Mycophenolate Mofetil as active, Cholesterol and span 60 as membrane forming agents, Boric acid as preservative, Sodium sulfate as antioxidant, Sodium phosphate monobasic anhydrous as buffering agent, Poloxomer 407 as suspending agent and Sodium hydroxide as pH modifier.

TABLE 100

| | | Composition |
|---|---|---|
| Ingredient | Function | (% w/v) |
| Mycophenolate Mofetil | Active | 1.0 |
| Cholesterol | Membrane forming agent | 1.00 |
| Span 60 | Membrane forming agent | 4.00 |
| Sodium sulfate | Anti-oxidant | 1.2 |
| Sodium phosphate | Buffering agent | 2.0 |
| monobasic, anhydrous | | |
| Poloxomer 407 | Suspending agent | 0.5 |
| Sodium hydroxide | pH Modifier | QS |
| Boric acid | Preservative | 0.3 |
| Purified water | Aqueous Phase | q. s to 100% |

Method of Preparation:
1. Drug loaded niosomes are synthesized using thin layer evaporation method with slight modifications.
2. Cholesterol, span 60 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic, Poloxomer 407 and boric acid.
6. Sonication is done for 15 mins at 60° C.
   pH of the resulting solution is adjusted using 0.1 M sodium hydroxide.
   Volume makeup is done using purified water.

### Example 79

An ophthalmic niosomal formulation comprising Mycophenolate Mofetil as active, Cholesterol and span 40 as membrane forming agents, Boric acid as preservative, Sodium sulfate as antioxidant, Potassium phosphate monobasic anhydrous as buffering agent, Tween 20 as suspending agent and Sodium hydroxide as pH modifier.

TABLE 101

| | | Composition |
|---|---|---|
| Ingredient | Function | (% w/v) |
| Mycophenolate Mofetil | Active | 1.0 |
| Cholesterol | Membrane forming agent | 1.00 |
| Span 40 | Membrane forming agent | 4.00 |
| Sodium sulfate | Anti-oxidant | 1.2 |
| Potassium phosphate | Buffering agent | 2.0 |
| monobasic, anhydrous | | |
| Tween 20 | Suspending agent | 0.5 |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Cholesterol, span 40 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, Potassium phosphate monobasic, Tween 20 and boric acid.
6. Sonication is done for 15 mins at 60° C.
7. pH of the resulting solution is adjusted using 0.1 M sodium hydroxide.
8. Volume makeup is done using purified water.

### Example 80

An ophthalmic niosomal formulation comprising Mycophenolate Mofetil as active, Stearic acid, Span 65 and Brij 20 as membrane forming agents, Boric acid as preservative, Sodium sulfate as antioxidant, Potassium phosphate monobasic as buffering agent, Povidone as suspending agent and Sodium hydroxide as pH modifier.

TABLE 102

| | | Composition |
|---|---|---|
| Ingredient | Function | (% w/v) |
| Mycophenolate Mofetil | Active | 1.0 |
| Stearic acid | Membrane forming agent | 3.00 |
| Span 65 | Membrane forming agent | 4.00 |
| Brij 20 | Membrane forming agent | 2.00 |
| Sodium sulfate | Anti-oxidant | 1.2 |
| Potassium phosphate | Buffering agent | 2.0 |
| monobasic | | |
| Povidone | Suspending agent | 0.5 |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Stearic acid, Span 65, Brij 20 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, Potassium phosphate monobasic, povidone and boric acid.
6. Sonication is done for 15 mins at 60° C.
7. pH of the resulting solution is adjusted using 0.1 M sodium hydroxide.
8. Volume makeup is done using purified water.

### Example 81

An ophthalmic niosomal formulation comprising Mycophenolate Mofetil as active, Cholesterol, Span 60 and Nonoxynol-9 as membrane forming agents, Boric acid as preservative, Sodium sulfate as antioxidant, Potassium phosphate monobasic as buffering agent, Pemulen TR 2 as suspending agent, Sodium chloride as tonicity modifier and Sodium hydroxide as pH modifier.

TABLE 103

| | | Composition |
|---|---|---|
| Ingredient | Function | (% w/v) |
| Mycophenolate Mofetil | Active | 1.0 |
| Cholesterol | Membrane forming agent | 3.00 |
| Span 60 | Membrane forming agent | 4.00 |
| Nonoxynol-9 | Membrane forming agent | 2.00 |
| Sodium sulfate | Anti-oxidant | 1.2 |
| Potassium phosphate | Buffering agent | 2.0 |
| monobasic | | |
| Pemulen TR 2 | Suspending agent | 0.5 |
| Sodium chloride | Tonicity modifier | 0.9 |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Cholesterol, span 60, Nonoxynol-9 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, Potassium phosphate monobasic, Pemulen TR 2 and boric acid.
6. Sonication is done for 15 mins at 60° C.
7. pH of the resulting solution is adjusted using 0.1 M sodium hydroxide.
8. Volume makeup is done using purified water.

### Example 82

An ophthalmic niosomal formulation comprising Mycophenolate Mofetil as active, Cholesterol, Span 60 and Nonoxynol-9 as membrane forming agents, Boric acid as preservative, Sodium sulfate as antioxidant, Potassium phosphate monobasic as buffering agent, Pemulen TR 2 as suspending agent, Sodium chloride as tonicity modifier and Sodium hydroxide as pH modifier.

TABLE 104

| | | Composition |
|---|---|---|
| Ingredient | Function | (% w/v) |
| Mycophenolate Mofetil | Active | 0 |
| Cholesterol | Membrane forming agent | 00 |
| Span 60 | Membrane forming agent | 00 |
| Nonoxynol-9 | Membrane forming agent | 00 |
| Sodium sulfate | Anti-oxidant | 2 |
| Potassium phosphate | Buffering agent | 0 |
| monobasic | | |
| Pemulen TR 2 | Suspending agent | 5 |
| Sodium chloride | Tonicity modifier | 9 |
| Sodium hydroxide | pH Modifier | S |
| Boric acid | Preservative | 3 |
| Purified water | Aqueous Phase | s to 100% |

Method of Preparation:
1. Drug loaded niosomes are synthesized using thin layer evaporation method with slight modifications.
2. Cholesterol, span 60, Nonoxynol-9 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, Potassium phosphate monobasic, Pemulen TR 2 and boric acid.
6. Sonication is done for 15 mins at 60° C.
7. pH of the resulting solution is adjusted using 0.1 M sodium hydroxide.
8. Volume makeup is done using purified water.

### Example 83

An ophthalmic niosomal formulation comprising Mycophenolate Mofetil as active, Cholesterol, Tween 80 and Nonoxynol-9 as membrane forming agents, Boric acid as preservative, Sodium sulfate as antioxidant, Potassium phosphate monobasic as buffering agent, Pemulen TR 1 as suspending agent, Sodium chloride as tonicity modifier and Sodium hydroxide as pH modifier.

TABLE 105

| | | Composition |
|---|---|---|
| Ingredient | Function | (% w/v) |
| Mycophenolate Mofetil | Active | 1.0 |
| Cholesterol | Membrane forming agent | 3.00 |
| Tween 80 | Membrane forming agent | 4.00 |
| Nonoxynol-9 | Membrane forming agent | 2.00 |
| Sodium sulfate | Anti-oxidant | 1.2 |
| Potassium phosphate | Buffering agent | 2.0 |
| monobasic | | |
| Pemulen TR 1 | Suspending agent | 0.5 |
| Sodium chloride | Tonicity modifier | 0.9 |
| Sodium hydroxide | pH Modifier | QS |
| Boric acid | Preservative | 0.3 |
| Purified water | Aqueous Phase | q.s to 100% |

Method of Preparation:
1. Drug loaded niosomes are synthesized using thin layer evaporation method with slight modifications.
2. Cholesterol, Tween 80, Nonoxynol-9 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, Potassium phosphate monobasic, Pemulen TR 1 and boric acid.
6. Sonication is done for 15 mins at 60° C.
7. pH of the resulting solution is adjusted using 0.1 M sodium hydroxide.
8. Volume makeup is done using purified water.

### Example 84

An ophthalmic vesicular formulation comprising Mycophenolate Mofetil as active, Lanolin alcohol as membrane forming agents, Tween 80 and Kolliphor EL as surfactants, Boric acid as preservative, Sodium sulfate as antioxidant, Potassium phosphate monobasic as buffering agent, Pemulen TR 1 as suspending agent, Sodium chloride as tonicity modifier and Sodium hydroxide as pH modifier.

TABLE 106

| | | Composition |
|---|---|---|
| Ingredient | Function | (% w/v) |
| Mycophenolate Mofetil | Active | 1.0 |
| Lanolin alcohol | Membrane forming agent | 10.00 |
| Tween 80 | Non-ionic Surfactant | 4.00 |
| Kolliphor EL | Non-ionic Surfactant | 5.00 |
| Sodium sulfate | Anti-oxidant | 1.2 |
| Potassium phosphate | Buffering agent | 2.0 |
| monobasic | | |
| Pemulen TR 1 | Suspending agent | 0.5 |
| Sodium chloride | Tonicity modifier | 0.9 |
| Sodium hydroxide | pH Modifier | QS |
| Boric acid | Preservative | 0.3 |
| Purified water | Aqueous Phase | q.sto 100% |

Method of Preparation:
1. Drug loaded vesicular system is synthesized using thin layer evaporation method with slight modifications.
2. Lanolin alcohol, Tween 80, Kolliphor EL and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, Potassium phosphate monobasic, Pemulen TR 1 and boric acid.
6. Sonication is done for 15 mins at 60° C.
7. pH of the resulting solution is adjusted using 0.1 M sodium hydroxide.
8. Volume makeup is done using purified water.

### Example 85

An ophthalmic vesicular formulation comprising Mycophenolate Mofetil as active, Span 60 and Tween 20 as membrane forming agents, Boric acid as preservative, Sodium sulfate as antioxidant, Potassium phosphate monobasic anhydrous as buffering agent and Sodium hydroxide as pH modifier.

TABLE 107

| | | Composition |
|---|---|---|
| Ingredient | Function | (% w/v) |
| Mycophenolate Mofetil | Active | 1.0 |
| Span 60 | membrane forming agents | 2.0 |
| Tween 20 | membrane forming agents | 3.0 |
| Potassium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Drug loaded vesicular system is synthesized using thin layer evaporation method with slight modifications.
2. Tween 20, span 60 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic and boric acid.
6. Sonication is done for 15 mins at 60° C.
7. pH of the resulting solution is adjusted using 0.1 M sodium hydroxide.
8. Volume makeup is done using purified water.

### Example 86

An ophthalmic vesicular formulation comprising Mycophenolate Mofetil as active, Lecithin and Tween 80 as membrane forming agent and edge activator, Boric acid as preservative, Sodium phosphate monobasic anhydrous as buffering agent and Sodium hydroxide as pH modifier.

TABLE 108

| | | Composition |
|---|---|---|
| Ingredient | Function | (% w/v) |
| Mycophenolate Mofetil | Active | 1.0 |
| Lecithin | Lipid | 8.5 |
| Tween 80 | Edge activator& | 1.5 |
| | membrane forming agent | |
| Sodium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Drug loaded vesicular system is synthesized using thin layer evaporation method with slight modifications.
2. Lecithin, Tween 80 and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic and boric acid.
6. Sonication is done for 15 mins at 60° C.
7. pH of the resulting solution is adjusted using 0.1 M sodium hydroxide.
8. Volume makeup is done using purified water.

### Example 87

An ophthalmic vesicular formulation comprising Mycophenolate Mofetil as active, Lecithin and Sodium deoxycholate as edge activator and membrane forming agents, Boric acid as preservative, Sodium phosphate monobasic anhydrous as buffering agent and Sodium hydroxide as pH modifier.

TABLE 109

| | | Composition |
|---|---|---|
| Ingredient | Function | (% w/v) |
| Mycophenolate Mofetil | Active | 1.0 |
| Lecithin | Lipid | 8.5 |
| Sodium deoxycholate | Edge activator & | 1.5 |
| | membrane forming agent | |
| Sodium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Drug loaded vesicular system is synthesized using thin layer evaporation method with slight modifications.
2. Briefly, Lecithin, Sodium deoxycholate and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic and boric acid.
6. Sonication is done for 15 mins at 60° C.
7. pH of the resulting solution is adjusted using 0.1 M sodium hydroxide.
8. Volume makeup is done using purified water.

### Example 88

An ophthalmic liposomal formulation comprising Mycophenolate Mofetil as active, Lecithin, and cholesterol as membrane forming agents, Tween 80 as edge activator, Boric acid as preservative, Sodium phosphate monobasic anhydrous as buffering agent and Sodium hydroxide as pH modifier.

TABLE 110

| | | Composition |
|---|---|---|
| Ingredient | Function | (% w/v) |
| Mycophenolate Mofetil | Active | 1.0 |
| Lecithin | membrane forming agent | 8.5 |
| Tween 80 | Edge activator | 1.5 |
| Cholesterol | membrane forming agents | 1.0 |
| Sodium phosphate | Buffering agent | 2.0 |
| monobasic, anhydrous | | |
| Sodium hydroxide | pH Modifier | QS |
| Boric acid | Preservative | 0.3 |
| Purified water | Aqueous Phase | q.s to 100% |

Method of Preparation:
1. Drug loaded vesicular system is synthesized using thin layer evaporation method with slight modifications.
2. Lecithin, Tween 80, cholesterol and Mycophenolate mofetil are dissolved in 20 ml of ethanol.
3. Solution is kept for evaporation in a rotary evaporator for 1 h, which results in formation of a thin film.
4. In order to get a completely dried film the flask is kept in a vacuum desiccator for 1 h.
5. The obtained thin film is then hydrated using 20 ml of distilled water containing sodium sulfate, sodium phosphate monobasic and boric acid.
6. Sonication is done for 15 mins at 60° C.
7. pH of the resulting solution is adjusted using 0.1 M sodium hydroxide.
8. Volume makeup is done using purified water.

### Example 89

An ophthalmic gel formulation comprising Mycophenolate Sodium as active, Poloxamer as solubilizer, Propylene glycol as humectant, Hypromellose as thickener, Mannitol as tonicity adjusting agent, Disodium edetate as chelating agent and Benzalkonium chloride as preservative. A preferred aqueous gel comprises by weight based on the total weight of the composition:

TABLE 111

| | | Composition |
|---|---|---|
| Ingredient | Function | (% w/v) |
| Mycophenolate sodium | Active | 1.0 |
| equivalent to Mycophenolic acid | | |
| Sodium chloride | Tonicity modifier | 5.0 |
| Hypromellose | Thickening agent | 2.5 |
| Benzalkonium chloride | Preservative | 0.02 |
| Edetate sodium | Chelating agent | 0.1 |
| Propylene glycol | Humectant | 4.4 |
| Poloxamer | Surfactant | 2.0 |
| Sodium Hydroxide | pH modifier | Q.s |
| Purified water | Aqueous Phase | Q.s to 100% |

Method of Preparation:
1. A hydrated polymeric gel phase is prepared by dissolving Poloxamer in deionized water followed by slowly dispersing Hypromellose into it under continuous stirring for one hour.
2. Propylene glycol, Mannitol, edetate disodium and Benzalkonium chloride are added to the gel followed by stirring for 15 minutes.
3. The pH is adjusted to between 6.5 to 7.0 using 0.1 M sodium hydroxide.

### Example 90

An ophthalmic gel formulation comprising Mycophenolate mofetil hydrochloride as active, Poloxamer as solubilizer, Glycerin as humectant, Carbomer 940 as thickener, Mannitol as tonicity adjusting agent, Disodium edetate as chelating agent and Benzalkonium chloride as preservative. A preferred aqueous gel comprises by weight based on the total weight of the composition:

TABLE 112

| Ingredient | Function | Composition (% w/v) |
|---|---|---|
| Mycophenolate mofetil | Active | 1.0 |
| hydrochloride | | |
| Mannitol | Tonicity modifier | 5.0 |
| Carbomer 940 | Thickening agent | 4.0 |
| Benzalkonium chloride | Preservative | 0.02 |
| Edetate disodium | Chelating agent | 0.13 |
| Glycerin | Humectant | 8.8 |
| Poloxamer | Surfactant | 2.0 |
| Sodium hydroxide | pH modifier | Q.s |
| Purified water | Aqueous Phase | q. s to 100% |

Method of Preparation:
1. A hydrated polymeric gel is prepared by dissolving Poloxamer in deionized water followed by slowly dispersing Carbomer 940 into a beaker fitted with an overhead stirred and stirring for one hour.
2. Glycerin, Mannitol, edetate disodium and Benzalkonium chloride are added to the gel followed by stirring for 15 minutes.
3. The pH is adjusted to between 6.5 to 7.0 using 0.1 M sodium hydroxide.

### Example 91

An ophthalmic lyophilized powder for reconstitution formulation comprising Mycophenolate mofetil hydrochloride as active, Tyloxapol and Octoxynol-40 as solubilizer, Sodium acetate as buffering agent, Mannitol as tonicity adjusting agent, sodium sulfate as antioxidant and Boric acid as preservative.

TABLE 113

| | | Composition |
|---|---|---|
| Ingredient | Function | (% w/v) |
| Mycophenolate mofetil | Active | 1.0 |
| Hydrochloride equivalent | | |
| to Mycophenolate mofetil | | |
| Mannitol | Tonicity modifier | 5.0 |
| Tyloxapol | Solubilizer | 0.1 |
| Octoxynol-40 | Solubilizer | 0.05 |
| Sodium acetate | Buffering agent | 0.39 |
| Sodium hydroxide | pH Modifier | QS |
| Boric acid | Preservative | 0.06 |
| Sterile water for injection | Aqueous Phase For | q. s to 100% |
| | reconstitution | |

Method of Preparation:
1. Mannitol, Sodium acetate and Boric acid are added to water in a beaker and stirred till clear solution is formed.
2. Mycophenolate mofetil Hydrochloride is added to a mixture of Tyloxapol and Octoxynol-40 under stirring.
3. Both the phases of step 1 and step 2 are mixed to get a clear solution.
4. pH is adjusted using 0.1 M Sodium hydroxide.
5. Step 4 solution is filtered using 0.22 μm PVDF filters and filled in vials.
6. Vials are then loaded into the lyophilizer, with a platform temperature of 25° C.
7. The shelf temperature is decreased from 25° C. to −40° C. in next 45 minutes.
8. The shelf temperature is maintained at −40° C. for 240 minutes.
9. The shelf temperature is then further decreased to −60° C. for 120 minutes.
10. The Lyophilization chamber pressure is reduced to 220 mTorr.
11. After the pressure reaches to 220 mTorr, the temperature is maintained at −60° C. for 60 minutes with further reduction in the chamber pressure to 100 mTorr.
12. The temperature is increased from −40° C. to −20° C., over 180 minutes with chamber pressure at 100 mTorr.
13. The temperature is maintained at −20° C. for 600 minutes.
14. The temperature is increased from −20° C. to 15° C., over 200 minutes along with decrease in the chamber pressure to 80 mTorr.
15. The temperature is increased from 15° C. to 25° C., over 300 minutes along with decrease in the chamber pressure to 40 mTorr.
16. In post heat stage temperature is increased up to 60° C. for 60 minutes with chamber pressure at 50 mTorr before sealing the vials.

### Example 92

An ophthalmic lyophilized powder for reconstitution formulation comprising Mycophenolate sodium as active, Octoxynol-40 as solubilizer, Sodium acetate as buffering agent, Mannitol as tonicity adjusting agent, sodium sulfate as antioxidant and Boric acid as preservative.

TABLE 114

| | | Composition |
|---|---|---|
| Ingredient | Function | (% w/v) |
| Mycophenolate sodium | Active | 1.0 |
| equivalent to | | |
| mycophenolic acid | | |
| Mannitol | Tonicity modifier | 5.0 |
| Octoxynol-40 | Solubilizer | 0.05 |
| Sodium acetate | Buffering agent | 0.39 |
| Sodium hydroxide | pH Modifier | QS |
| Boric acid | Preservative | 0.06 |
| Sterile water for injection | Aqueous Phase | For q.s to 100% |
| | reconstitution | |

Method of Preparation:
1. Mannitol, Sodium acetate and Boric acid are added to water in a beaker and stirred till clear solution is formed.
2. Mycophenolate Sodium is added to Octoxynol-40 under stirring.
3. Both the phases of step 1 and step 2 are mixed to get a clear solution.
4. pH is adjusted using 0.1 M Sodium hydroxide.
5. Step 4 solution is filtered using 0.22 μm PVDF filters and filled in vials.
6. Vials are then loaded into the lyophilizer, with a platform temperature of 25° C.
7. The shelf temperature is decreased from 25° C. to −40° C. in next 45 minutes.
8. The shelf temperature is maintained at −40° C. for 240 minutes.
9. The shelf temperature is then further decreased to −60° C. for 120 minutes.
10. The Lyophilization chamber pressure is reduced to 220 mTorr.
11. After the pressure reaches to 220 mTorr, the temperature is maintained at −60° C. for 60 minutes with further reduction in the chamber pressure to 100 mTorr.
12. The temperature is increased from −40° C. to −20° C., over 180 minutes with chamber pressure at 100 mTorr.
13. The temperature is maintained at −20° C. for 600 minutes.
14. The temperature is increased from −20° C. to 15° C., over 200 minutes along with decrease in the chamber pressure to 80 mTorr.
15. The temperature is increased from 15° C. to 25° C., over 300 minutes along with decrease in the chamber pressure to 40 mTorr.
16. In post heat stage temperature is increased up to 60° C. for 60 minutes with chamber pressure at 50 mTorr before sealing the vials.

### Example 93

An ophthalmic/ocular single use intravitreal injection formulation comprising Mycophenolate sodium as active, Polysorbate 20 as solubilizer, monobasic sodium phosphate monohydrate and dibasic sodium phosphate heptahydrate as buffering agent, Sucrose and Sodium chloride as tonicity adjusting agents along with sodium hydroxide as pH modifier, was prepared.

TABLE 115

| | | Composition |
|---|---|---|
| Ingredient | Function | (% w/v) |
| Mycophenolate sodium | Active | 1.0 |
| Monobasic sodium | buffering agent | 0.01 |
| phosphate monohydrate | | |
| dibasic sodium | buffering agent | 0.1 |
| phosphate heptahydrate | | |
| Sodium chloride | Tonicity adjusting agent | 0.25 |
| Sucrose | Tonicity adjusting agent | 5.0 |
| Polysorbate 20 | Solubilizer | 0.3 |
| Sodium hydroxide | pH modifier | QS |
| Purified water | Aqueous Phase | q. s to 100% |

Method of Preparation:
1. Monobasic sodium phosphate monohydrate, sucrose, sodium chloride and dibasic sodium phosphate heptahydrate were added to water in a beaker and stirred till clear solution was formed.
2. Mycophenolate sodium was added to a solution of step 1 followed by Polysorbate 20 under stirring.
3. Step 2 solution was filtered using 0.22 μm PVDF filters and filled in vials.
4. Vials were then loaded into the lyophilizer, with a platform temperature of 25° C.
5. The shelf temperature was decreased from 25° C. to −40° C. in next 45 minutes.
6. The shelf temperature was maintained at −40° C. for 240 minutes.
7. The shelf temperature was then further decreased to −60° C. for 120 minutes.
8. The lyophilization chamber pressure was reduced to 220 mTorr.
9. After the pressure reached 220 mTorr, the temperature was maintained at −60° C. for 60 minutes with further reduction in the chamber pressure to 100 mTorr.
10. The temperature was increased from −40° C. to −20° C., over 180 minutes with chamber pressure at 100 mTorr.
11. The temperature was maintained at −20° C. for 600 minutes.
12. The temperature was increased from −20° C. to 15° C., over 200 minutes along with decrease in the chamber pressure to 80 mTorr.
13. The temperature was increased from 15° C. to 25° C., over 300 minutes along with decrease in the chamber pressure to 40 mTorr.
14. In post heat stage, temperature was increased up to 60° C. for 60 minutes with chamber pressure at 50 mTorr before sealing the vials.

### Example 94

An ophthalmic/ocular intravitreal injection lyophilized powder formulation was prepared for reconstitution. Mycophenolate mofetil hydrochloride as active, Polysorbate 20 as solubilizer, dibasic sodium phosphate heptahydrate as buffering agent, Mannitol as tonicity adjusting agent and sodium hydroxide as pH modifier were employed.

TABLE 116

| | | Composition |
|---|---|---|
| Ingredient | Function | (% w/v) |
| Mycophenolate mofetil HC1 | Active | 1.0 |
| Mannitol | Tonicity modifier | 5.0 |
| Polysorbate 20 | Solubilizer | 0.1 |
| dibasic sodium phosphate | Buffering agent | 0.39 |
| heptahydrate | | |
| Sodium hydroxide | pH Modifier | QS |
| Sterile water for injection | Aqueous Phase For | q. s to 100% |
| | reconstitution | |

Method of Preparation:
1. Mannitol and dibasic sodium phosphate heptahydrate were added to water in a beaker and stirred till clear solution is formed.
2. Mycophenolate mofetil hydrochloride was added to a mixture of Polysorbate 20 under stirring.
3. Both the phases of step 1 and step 2 were mixed to get a clear solution.
4. pH was adjusted using 0.1 M Sodium hydroxide.
5. Step 4 solution was filtered using 0.22 μm PVDF filters and filled in vials.
6. Vials were then loaded into the lyophilizer with a platform temperature of 25° C.
7. The shelf temperature was decreased from 25° C. to −40° C. in next 45 minutes.
8. The shelf temperature was maintained at −40° C. for 240 minutes.
9. The shelf temperature was then further decreased to −60° C. for 120 minutes.
10. The Lyophilization chamber pressure was reduced to 220 mTorr.
11. After the pressure reaches to 220 mTorr, the temperature was maintained at −60° C. for 60 minutes with further reduction in the chamber pressure to 100 mTorr.
12. The temperature was increased from −40° C. to −20° C., over 180 minutes with chamber pressure at 100 mTorr.
13. The temperature was maintained at −20° C. for 600 minutes.
14. The temperature was increased from −20° C. to 15° C. over 200 minutes along with decrease in the chamber pressure to 80 mTorr.
15. The temperature was increased from 15° C. to 25° C., over 300 minutes along with decrease in the chamber pressure to 40 mTorr.
16. In post heat stage, the temperature was increased up to 60° C. for 60 minutes with chamber pressure at 50 mTorr before sealing the vials.

### Example 95

A vesicular ophthalmic suspension formulation comprising Mycophenolate mofetil as active, Tyloxapol and Polysorbate 80 as surfactant, stearyl amine as charge inducer, Sodium chloride as tonicity adjusting agent, Lanolin as vesicular membrane forming agent and Boric acid as preservative.

TABLE 117

| | | Composition |
|---|---|---|
| Ingredient | Function | (% w/v) |
| Mycophenolate Mofetil | Active | 1.0 |
| Polysorbate 80 | Non-ionic surfactants | 2.65 |
| Stearylamine | Targeting agent | 0.5 |
| Lanolin | Membrane forming agent | 0.76 |
| Sodium hydroxide | pH Modifier | QS |
| Boric acid | Preservative | 0.3 |
| Sodium Chloride | Tonicity adjusting agent | 0.09 |
| Purified water | Aqueous Phase | q. s to 100% |

Method of Preparation:
1. Polysorbate 80, Lanolin and Stearyl amine are added to alcohol and stirred to get a clear solution.
2. Mycophenolate mofetil hydrochloride is added to Step 1 under stirring to get a clear solution.
3. A thin film is prepared by evaporating alcohol from the phase of step 2 in a round bottom flask (RBF) at reduced pressure using a rotavapor.
4. Boric acid and sodium chloride are dissolved in purified water to form the aqueous phase.
5. The aqueous phase is used to hydrate the dried layer of step 3.
6. Vesicles obtained are sonicated for 15 min at 40° C.
7. pH of solution is adjusted using 0.1 M NaOH solution.
8. The pH of the formulation is maintained at 5.8-6.2.
9. The viscosity of the formulation is maintained at 2.8 mPas with a vesicle size of 291 nm with a PDI of 0.311.

### Example 96

An ophthalmic ointment formulation filled in soft gelatin capsules comprising Mycophenolate Mofetil as active. Petrolatum, lanolin oil and light liquid paraffin as ointment base.

TABLE 118

| | Ingredient | Function | Composition (% w/w) |
|---|---|---|---|
| Phase A | Mycophenolate Mofetil | Active | 1.0 |
| | Lanolin alcohol | Solubilizer | 3.0 |
| | Chlorobutanol | Preservative | 0.5 |
| Phase B | Petrolatum | Base | 30.5 |
| | Light Liquid paraffin | Base | 65.0 |

Method of Preparation:
1. Lanolin alcohol is taken in a beaker using water bath between 70±2° C.
2. Mycophenolate Mofetil is added to the heated Lanolin alcohol phase of step 1 and continue mixing to dissolve Mycophenolate Mofetil.
3. Chlorobutanol is added to the API phase and maintain temperature of the phase between 70±2° C.
4. Petrolatum is melted in a beaker using water bath maintained at 70±2° C.
5. Light Liquid paraffin is added to the molten Petrolatum phase and maintain the temperature of the phase between 70±2° C.
6. API phase of step 3 is added to the petrolatum phase of step 5 with constant stirring. Mixing continued for 30±5 minutes.
7. Heating is removed and the bulk is allowed to cool slowly.
8. The final ointment formulation is filled into soft gelatin capsules.

### Example 97

An ophthalmic ointment formulation filled in soft gelatin capsules comprising Mycophenolate Mofetil as active. Petrolatum, and light liquid paraffin as ointment base and Polyethylene glycol 300 and Lanolin alcohol as solubilizer.

TABLE 119

| | Ingredient | Function | Composition (% w/w) |
|---|---|---|---|
| Phase A | Mycophenolate Mofetil | Active | 1.0 |
| | Polyethylene glycol 300 | Solubilizer | 5.0 |
| | Lanolin alcohol | Solubilizer | 10.0 |
| | Chlorobutanol | Preservative | 0.5 |
| Phase B | Petrolatum | Base | 30.5 |
| | Light Liquid paraffin | Base | 53.0 |

Method of Preparation:
1. Lanolin alcohol and Polyethylene glycol 300 are taken in a beaker using water bath between 70±2° C.
2. Mycophenolate Mofetil is added to the heated phase of step 1 and continue mixing to dissolve Mycophenolate Mofetil.
3. Chlorobutanol is added to the API phase and maintain temperature of the phase between 70±2° C.
4. Petrolatum is melted in a beaker using water bath maintained at 70±2° C.
5. Light Liquid paraffin is added to the molten Petrolatum phase and maintain the temperature
6. of the phase between 70±2° C.
7. API phase of step 3 is added to the petrolatum phase of step 5 with constant stirring. Mixing continued for 30±5 minutes.
8. Heating is removed and the bulk is allowed to cool slowly.
9. The final ointment formulation is filled into soft gelatin capsules.

### Example 98

Ophthalmic ointment formulations comprising Mycophenolate Mofetil as active (Tables 120 and 121).

TABLE 120

| | | | | Composition (% w/w) | | |
|---|---|---|---|---|---|---|
| | | | | VLN-F- | VLN-F- | VLN-F- |
| S. No. | | Ingredient | Function | 70/ASR/024 | 70/ASR/026 | 70/ASR/027 |
| 1 | | Mycophenolate | Active | 1.00 | 0.50 | 1.00 |
| | | Mofetil | | | | |
| 2 | Phase A | Lanolin | Solubilizer | 3.00 | 3.00 | 3.00 |
| 3 | | Lanolin Alcohol | Solubilizer | 10.00 | 10.00 | 10.00 |
| 4 | | Glyceryl | Solubilizer | 0.50 | 0.50 | 0.50 |
| | | monostearate | | | | |
| 5 | | Cetyl Alcohol | Solubilizer | — | — | 0.50 |
| 6 | | Ethanol | Solubilizer | — | — | 0.50 |
| 7 | | Castor oil | Solubilizer | — | — | 0.15 |
| 8 | Phase B | Petrolatum | Base | 40.00 | 40.00 | 40.00 |
| 9 | | Light Liquid | Base | 45.50 | 46.00 | 44.35 |
| | | paraffin | | | | |

| | |
|---|---|
| Chemical analysis | Assay of |
| | mycophenolate |
| | mofetil: |
| | 103.90% w/w |

TABLE 121

| | | | | Composition (% w/w) | | |
|---|---|---|---|---|---|---|
| | | | | VLN-F- | VLN-F- | VLN-F- |
| S. No. | | Ingredient | Function | 70/ASR/032 | 70/ASR/050C | 70/ASR/050D |
| 1 | | Mycophenolate | Active | 0.50 | 1.00 | 1.00 |
| | | Mofetil | | | | |
| 2 | Phase A | Lanolin | Solubilizer | 3.00 | 3.00 | 3.00 |
| 3 | | Lanolin Alcohol | Solubilizer | 10.00 | 5.00 | 5.00 |
| 4 | | Glyceryl | Solubilizer | 0.50 | 0.50 | — |
| | | monostearate | | | | |
| 5 | | Cetyl Alcohol | Solubilizer | 0.50 | — | — |
| 6 | | Ethanol | Solubilizer | 0.50 | — | — |
| 7 | | Castor oil | Solubilizer | 0.15 | — | — |
| 8 | Phase B | Petrolatum | Base | 40.00 | 31.50 | 32.00 |
| 9 | | Light Liquid | Base | 44.85 | 59.00 | 59.00 |
| | | paraffin | | | | |

| | |
|---|---|
| Chemical analysis | Assay of |
| | mycophenolate |
| | mofetil: |
| | 104.23% w/w |

Method of Preparation:
All the ingredients of Phase A were taken in a beaker and melted using water bath at 70±2° C.
1. Mycophenolate Mofetil was added to the molten phase A under stirring.
2. All the ingredients of Phase B were taken in a beaker and melted using water bath at 70±2° C.
3. Phase B was added to phase A under stirring and maintaining the temperature at 70±2° C.
4. Heating is removed and the bulk was allowed to cool slowly.

### Example 99

Ophthalmic emulsion formulations comprising Mycophenolate Mofetil as active were prepared (Tables 122 and 123).

TABLE 122

| | | | | Composition (% w/v) | | | | |
|---|---|---|---|---|---|---|---|---|
| | | | | VLN-F- | VLN-F- | VLN-F- | VLN-F- | VLN-F- |
| S. No. | | Ingredient | Function | 70/ASR/030A | 70/ASR/030B | 70/ASR/033 | 70/ASR/034 | 70/ASR/047A |
| 1 | | Mycophenolate | Active | 1.00 | 1.00 | 0.30 | 0.50 | 0.75 |
| | | Mofetil | | | | | | |
| 2 | Phase A | Polysorbate 80 | Surfactant | 4.00 | 4.00 | 4.00 | 4.00 | 4.00 |
| 3 | | Polyoxyl 40 | Surfactant | — | — | — | — | — |
| | | stearate | | | | | | |
| 4 | | Lanolin | Oil phase | — | — | — | — | — |
| 5 | | Lanolin Alcohol | Oil phase | 5.00 | 10.00 | 5.00 | 5.00 | 5.00 |
| 6 | | Cetyl alcohol | Oil phase | — | — | — | — | — |
| 7 | | Glyceryl | Oil phase | — | — | — | — | — |
| | | monostearate | | | | | | |
| 8 | | Castor oil | Oil phase | 0.15 | 0.15 | 0.15 | 0.15 | 0.15 |
| 9 | | Light liquid | Oil phase | — | — | — | — | — |
| | | paraffin | | | | | | |
| 10 | | Ethanol | Solubilizer | — | — | — | — | — |
| 11 | Phase B | Pemulene TR1 | Viscosity | 0.05 | 0.05 | 0.05 | 0.05 | 0.05 |
| | | | modifier | | | | | |
| 12 | | Purified water | Aq phase | Up to | Up to | Up to | Up to | Up to |
| | | | | 100 ml | 100 ml | 100 ml | 100 ml | 100 ml |

| | | |
|---|---|---|
| pH | 6.53 | 6.47 |

TABLE 123

| | | | | Composition (% w/v) | | | | |
|---|---|---|---|---|---|---|---|---|
| | | | | VLN-F- | VLN-F- | VLN-F- | VLN-F- | VLN-F- |
| S. No. | | Ingredient | Function | 70/ASR/047B | 70/ASR/047D | 70/ASR/047E | 70/ASR/047F | 70/ASR/047G |
| 1 | | Mycophenolate | Active | 0.75 | 1.00 | 1.00 | 1.00 | 1.00 |
| | | Mofetil | | | | | | |
| 2 | Phase A | Polysorbate 80 | Surfactant | — | 4.00 | 4.00 | — | — |
| 3 | | Polyoxyl 40 | Surfactant | 7.00 | — | — | 7.00 | 7.00 |
| | | stearate | | | | | | |
| 4 | | Lanolin | Oil phase | — | 1.00 | — | — | — |
| 5 | | Lanolin Alcohol | Oil phase | 5.00 | 5.00 | 5.00 | 5.00 | 5.00 |
| 6 | | Cetyl alcohol | Oil phase | — | — | — | 0.50 | 0.50 |
| 7 | | Glyceryl | Oil phase | — | — | — | 0.50 | 0.50 |
| | | monostearate | | | | | | |
| 8 | | Castor oil | Oil phase | 0.15 | 0.15 | 0.15 | 0.15 | 0.15 |
| 9 | | Light liquid | Oil phase | — | — | 1.00 | — | — |
| | | paraffin | | | | | | |
| 10 | | Ethanol | Solubilizer | — | — | — | — | 0.50 |
| 11 | Phase B | Pemulene TR1 | Viscosity | 0.05 | 0.05 | 0.05 | 0.05 | 0.05 |
| | | | modifier | | | | | |
| 12 | | Purified water | Aq phase | Up to | Up to | Up to | Up to | Up to |
| | | | | 100 ml | 100 ml | 100 ml | 100 ml | 100 ml |

Method of Preparation:
1. All the ingredients of Phase A were taken in a beaker and melted using water bath at 70±2° C.
2. Mycophenolate Mofetil was added to the molten phase A under stirring.
3. Purified water was taken in a beaker and Pemulene TR1 was added with stirring to ensure complete hydration of the polymer maintaining the temperature between 70±2° C.
4. The emulsion was prepared by slowly adding Phase A to phase B under homogenization 5000 rpm for 15-30 minutes while maintaining the temperature between 70±2° C.
5. The emulsion was then cooled to room temperature with continuous stirring.

### Example 100

Following ophthalmic solution formulations comprising Mycophenolate sodium as active were prepared.

TABLE 124

| | | | Composition (% w/v) | | | | | | | |
|---|---|---|---|---|---|---|---|---|---|---|
| | | | VLN- | VLN- | VLN- | VLN- | VLN- | VLN- | VLN- | VLN- |
| | | | F-70/ | F-70/ | F-70/ | F-70/ | F-70/ | F-70/ | F-70/ | F-70/ |
| S. No. | Ingredient | Function | ASR/020G | ASR/020F | ASR/020D | ASR/020E | ASR/020C | ASR/044A | ASR/021A1 | ASR/021A3 |
| 1 | Mycophenolate | Active | 1.00 | 1.00 | 1.00 | 1.00 | 1.00 | 1.00 | 1.00 | 1.00 |
| | sodium | | | | | | | | | |
| 2 | Polysorbate 80 | Surfactant | 3.00 | — | — | — | — | 4.00 | 4.00 | 4.00 |
| 3 | Kolliphor RH 40 | Surfactant | — | — | — | 1.00 | — | — | — | — |
| 4 | Tween 20 | Surfactant | — | — | 0.05 | — | — | — | — | — |
| 5 | Kolliphore EL | Surfactant | — | 3.00 | — | — | — | — | — | — |
| 6 | Poloxomer 407 | Surfactant | — | — | — | — | 2.00 | — | — | — |
| 7 | Boric acid | Preser- | — | — | — | — | — | — | — | — |
| | | vative | | | | | | | | |
| 8 | PVPK30 | Viscosity | — | — | — | — | — | — | — | 2.00 |
| | | modifier | | | | | | | | |
| 9 | PVA | Viscosity | — | — | — | — | — | — | 1.40 | — |
| | | modifier | | | | | | | | |
| 10 | HPMC K4M | Viscosity | — | — | — | — | — | — | — | — |
| | | modifier | | | | | | | | |
| 11 | Hydroxy Ethyl | Viscosity | — | — | — | — | — | — | — | — |
| | cellulose | modifier | | | | | | | | |
| 12 | Carbopol 974P | Viscosity | — | — | — | — | — | — | — | — |
| | | modifier | | | | | | | | |
| 13 | Purified water | Aqueous | Up to | Up to | Up to | Up to | Up to | Up to | Up to | Up to |
| | | phase | 100 ml | 100 ml | 100 ml | 100 ml | 100 ml | 100 ml | 100 ml | 100 ml |

| | | | | | | | | |
|---|---|---|---|---|---|---|---|---|
| pH | 7.34 | 7.34 | 7.13 | 7.17 | 7.11 | 7.42 | 7.38 | 7.27 |
| Assay (% w/v) | | | | | | 97.62 | | |

TABLE 125

| | | | Composition (% w/v) | | | |
|---|---|---|---|---|---|---|
| | | | VLN-F- | VLN-F- | VLN-F- | |
| | | | 70/ASR/044B with | 70/ASR/044A | 70/ASR/044A | VLN-F- |
| S. No. | Ingredient | Function | carbopol 974P | with HEC | with HMPC | 70/ASR/021B |
| 1 | Mycophenolate | Active | 1.00 | 1.00 | 1.00 | 1.00 |
| | sodium | | | | | |
| 2 | Polysorbate 80 | Surfactant | 4.00 | 4.00 | 4.00 | — |
| 3 | Kolhphor RH 40 | Surfactant | — | — | — | — |
| 4 | Tween 20 | Surfactant | — | — | — | — |
| 5 | Kolliphore EL | Surfactant | — | — | — | 5.00 |
| 6 | Poloxomer 407 | Surfactant | — | — | — | — |
| 7 | Boric acid | Preservative | — | — | — | — |
| 8 | PVPK30 | Viscosity | — | — | — | — |
| | | modifier | | | | |
| 9 | PVA | Viscosity | — | — | — | — |
| | | modifier | | | | |
| 10 | HPMC K4M | Viscosity | — | — | 0.50 | — |
| | | modifier | | | | |
| 11 | Hydroxy Ethyl | Viscosity | — | 0.50 | — | — |
| | cellulose | modifier | | | | |
| 12 | Carbopol 974P | Viscosity | 0.40 | — | — | — |
| | | modifier | | | | |
| 13 | Purified water | Aqueous | Up to | Up to | Up to | Up to |
| | | phase | 100 ml | 100 ml | 100 ml | 100 ml |

| | |
|---|---|
| pH | 7.44 |
| Assay (% w/v) | 98.62 |

TABLE 126

| | | | Composition (% w/v) | | | |
|---|---|---|---|---|---|---|
| | | | VLN-F- | VLN-F- | VLN-F- | |
| | | | 70/ASR/044B with | 70/ASR/044B | 70/ASR/044B | VLN-F- |
| S. No. | Ingredient | Function | carbopol 974P | with HEC | with HMPC | 70/ASR/021B1 |
| 1 | Mycophenolate | Active | 1.00 | 1.00 | 1.00 | 1.00 |
| | sodium | | | | | |
| 2 | Polysorbate 80 | Surfactant | — | — | — | — |
| 3 | Kolliphor RH 40 | Surfactant | — | — | — | — |
| 4 | Tween 20 | Surfactant | — | — | — | — |
| 5 | Kolliphore EL | Surfactant | 5.00 | 5.00 | 5.00 | 5.00 |
| 6 | Poloxomer 407 | Surfactant | — | — | — | — |
| 7 | Boric acid | Preservative | — | — | — | — |
| 8 | PVPK30 | Viscosity | — | — | — | — |
| | | modifier | | | | |
| 9 | PVA | Viscosity | — | — | — | 1.40 |
| | | modifier | | | | |
| 10 | HPMC K4M | Viscosity | — | — | 0.50 | — |
| | | modifier | | | | |
| 11 | Hydroxy Ethyl | Viscosity | — | 0.50 | — | — |
| | cellulose | modifier | | | | |
| 12 | Carbopol 974P | Viscosity | 0.40 | — | — | — |
| | | modifier | | | | |
| 13 | Purified water | Aqueous phase | Up to | Up to | Up to | Up to |
| | | | 100 ml | 100 ml | 100 ml | 100 ml |

| | |
|---|---|
| pH | 7.43 |

TABLE 127

| | | | Composition (% w/v) | | | | | | | |
|---|---|---|---|---|---|---|---|---|---|---|
| | | | VLN-F- | VLN-F- | VLN-F- | VLN-F- | VLN-F- | VLN-F- | VLN-F- | VLN-F- |
| | | | 70/ASR/ | 70/ASR/ | 70/ASR/ | 70/ASR/ | 70/ASR/ | 70/ASR/ | 70/ASR/ | 70/ASR/ |
| S. No. | Ingredient | Function | 021B1 | 021C | 021C1 | 021C3 | 021D1 | 021D3 | 021D2 | 021E1 |
| 1 | Mycophenolate | Active | 1.00 | 1.00 | 1.00 | 1.00 | 1.00 | 1.00 | 1.00 | 1.00 |
| | sodium | | | | | | | | | |
| 2 | Polysorbate 80 | Surfactant | — | 2.50 | 2.50 | 2.50 | — | — | — | 3.00 |
| 3 | Kolliphor RH 40 | Surfactant | — | — | — | — | 1.00 | 1.00 | 1.00 | 1.00 |
| 4 | Tween 20 | Surfactant | — | — | — | — | — | — | — | — |
| 5 | Kolliphore EL | Surfactant | 5.00 | 2.50 | 2.50 | 2.50 | 3.00 | 3.00 | 3.00 | — |
| 6 | Poloxomer 407 | Surfactant | — | — | — | — | — | — | — | — |
| 7 | Boric acid | Preservative | — | — | — | — | — | — | — | — |
| 8 | PVPK30 | Viscosity | 2.00 | — | — | 2.00 | — | 2.00 | — | — |
| | | modifier | | | | | | | | |
| 9 | PVA | Viscosity | — | — | 1.40 | — | 1.40 | — | — | 1.40 |
| | | modifier | | | | | | | | |
| 10 | HPMC K4M | Viscosity | — | — | — | — | — | — | 0.50 | — |
| | | modifier | | | | | | | | |
| 11 | Hydroxy Ethyl | Viscosity | — | — | — | — | — | — | — | — |
| | cellulose | modifier | | | | | | | | |
| 12 | Carbopol 974P | Viscosity | — | — | — | — | — | — | — | — |
| | | modifier | | | | | | | | |
| 13 | Purified water | Aqueous | Up to | Up to | Up to | Up to | Up to | Up to | Up to | Up to |
| | | phase | 100 ml | 100 ml | 100 ml | 100 ml | 100 ml | 100 ml | 100 ml | 100 ml |

| | | | | | | | | |
|---|---|---|---|---|---|---|---|---|
| pH | 7.32 | 7.43 | 7.42 | 7.32 | 7.38 | 7.28 | 7.38 | 7.37 |

TABLE 128

| | | | Composition (% w/v) | | | | | | | |
|---|---|---|---|---|---|---|---|---|---|---|
| | | | | | | VLN-F- | VLN-F- | VLN-F- | VLN-F- | VLN-F- |
| | | | VLN-F- | VLN-F- | VLN-F- | 70/ASR/ | 70/ASR/ | 70/ASR/ | 70/ASR/ | 70/ASR/ |
| | | | 70/ASR/ | 70/ASR/ | 70/ASR/ | 044C with | 044C | 044C | 050A | 050B |
| S. No. | Ingredient | Function | 021E3 | 021E2 | 044C | carbopol 974P | with HEC | with HMPC | with HEC | with HMPC |
| 1 | Mycophenolate | Active | 1.00 | 1.00 | 1.00 | 1.00 | 1.00 | 1.00 | 1.00 | 1.00 |
| | sodium | | | | | | | | | |
| 2 | Polysorbate 80 | Surfactant | 3.00 | 3.00 | 4.00 | 4.00 | 4.00 | 4.00 | 4.00 | 4.00 |
| 3 | Kolliphor RH 40 | Surfactant | 1.00 | 1.00 | — | — | — | — | — | — |
| 4 | Tween 20 | Surfactant | — | — | — | — | — | — | — | — |
| 5 | Kolliphore EL | Surfactant | — | — | 5.00 | 5.00 | 5.00 | 5.00 | — | — |
| 6 | Poloxomer 407 | Surfactant | — | — | — | — | — | — | — | — |
| 7 | Boric acid | Preser- | — | — | — | — | — | — | 1.00 | 1.00 |
| | | vative | | | | | | | | |
| 8 | PVPK30 | Viscosity | 2.00 | — | — | — | — | — | — | — |
| | | modifier | | | | | | | | |
| 9 | PVA | Viscosity | — | — | — | — | — | — | — | — |
| | | modifier | | | | | | | | |
| 10 | HPMC K4M | Viscosity | — | 0.50 | — | — | — | 0.50 | — | 0.50 |
| | | modifier | | | | | | | | |
| 11 | Hydroxy Ethyl | Viscosity | — | — | — | — | 0.50 | — | 0.50 | — |
| | cellulose | modifier | | | | | | | | |
| 12 | Carbopol 974P | Viscosity | — | — | — | 0.40 | — | — | — | — |
| | | modifier | | | | | | | | |
| 13 | Purified water | Aqueous | Up to | Up to | Up to | Up to | Up to | Up to | Up to | Up to |
| | | phase | 100 ml | 100 ml | 100 ml | 100 ml | 100 ml | 100 ml | 100 ml | 100 ml |

| | | | | |
|---|---|---|---|---|
| pH | 7.26 | 7.37 | 7.39 | 7.44 |
| Assay (% w/v) | | 98.84 | | |

Method of Preparation:
1. Purified water was taken in a beaker. Preservative was added to it under continuous stirring to get a clear solution.
2. Surfactant was added with stirring to the phase of step 1 to get a clear solution.
3. Active (mycophenolate sodium) was added to the phase of step 2 under stirring to get a clear solution.
4. Viscosity modifier was added to the phase of step 3 under stirring to get a clear solution.
5. Finally, pH was adjusted between 7.0 to 7.5.

### Example 101

Eye Implants/Therapeutic Contact Lenses:

Selection of Contact Lens for the Proposed Study: Different types of lenses can be selected depending on:
Transmittance characteristics. Transmittance will be evaluated by using the UV Spectrophotometer [in all UV-A (320-400 nm), UV-B (280-320 nm) & UV-C (200-280 nm) ranges].
Loading of Vitamin E (Vit E): Lens with maximum amount of Vitamin E loading will be selected for the study.

Example of few lenses that were used for this study are:
1. Acuvue Clear (brand contact lens for daily wear), Johnson & Johnson.
2. Freshlook Colourblends, Ciba Vision.
3. Silicone hydrogel contact lenses (visibly tinted contact lenses), Pure Vision2, Bausch & Lomb.
4. Surevue (Contact lens for daily wear), Johnson & Johnson.
5. Focus Dailies, Ciba Vision.

Method of loading Vitamin E in Contact Lenses

Lenses were rinsed first with deionized water. In order to load Vit. E into the contact lenses, each rinsed lens was soaked in a 3 mL of 0.05 g/mL of Vit E—ethanol solution for 24 h. After the loading step, lenses were submerged in 30 mL of millipore water for at least 3 h to extract ethanol. Lenses were then soaked in fresh Millipore water for further extraction. After this, the lenses were quickly dipped in ethanol for a few seconds and then taken out to remove any excess amount of Vit. E remaining on the surface. These extraction processes lead to diffusion of ethanol from the lenses into the aqueous phase while Vit E is retained due to negligible solubility of Vit E in water. The retained Vit E separates into nanosized barriers in the lenses. The lenses are then soaked in fresh PBS solution (pH 7.4) till further use.

Few lenses are dried and weighed to know the amount of Vit E loading into the lenses. The dried lenses are discarded after the weight measurements.

Drug Loading into the Contact Lenses

Lenses were first taken out from their packaging solution with the help of clean tweezers and then soaked in 30 mL of millipore water for 10 min. They are put in ethanol for 10 min which leads to the swelling and increase in size of lenses which further lead to enhancement in drug uptake by the lenses.

Each lens was then soaked in 5 mL of 4 mg/mL of Mycophenolate sodium-PBS solution for a period of 48 h. After the loading stage, the lenses were blotted to remove any excess drug solution remaining on the surface with the help of clean filter paper, and are subsequently stored in PBS 7.4 solution.

Drug Loading into Vit E Loaded Contact Lenses

Vit E loaded lenses were first removed from PBS solution and the same procedure described above was followed, with the only difference being that the lenses were directly dipped in Mycophenolate sodium drug solution instead of first soaking in ethanol. The reason is that Vit E gets removed from the lenses due to high solubility of Vit E in ethanol.

Preparation of Mycophenolate Mofetil Loaded Niosomes

Niosomes were prepared by thin film hydration method. In this method, Span 60 and cholesterol in different molar ratios of 4:6, 5:5, 6:4, 7:3 and 8:2 along with 50 mg of Mycophenolate mofetil were dissolved in 8:2 mL of chloroform:methanol in a round bottom flask. The flask was then attached to a rotary vacuum evaporator and the temperature of water bath was maintained at 60° C. The flask was rotated at 150 rpm for 10-15 min. The combination of heat and suction evaporated chloroform resulted in the formation of a thin film. The film was kept overnight in hot air oven at a temperature of 50-55° C. 10 ml of phosphate buffered saline solution (PBS, pH 7.4) was added to the flask containing the film and rotated at 70 rpm for another 1 h at 37° C. to peel off the surfactant/cholesterol film. The hydration of film lead to the formation of vesicles. The vesicles were then sonicated for 10 min in a probe sonicator and kept at 25° C.

Preparation of Mycophenolate Sodium Loaded Niosomes

Niosomes can be prepared by thin film hydration method. In this method, Span 60 and cholesterol in different molar ratios of 4:6, 5:5, 6:4, 7:3 and 8:2 will be dissolved in 8:2 mL of chloroform:methanol in a round bottom flask. The flask will be then attached to a rotary vacuum evaporator and the temperature of water bath will be maintained at 60° C. The flask will be rotated at 150 rpm for 10-15 min. The combination of heat and suction evaporated chloroform will result in the formation of a thin film. The film will be kept overnight in hot air oven at a temperature of 50-55° C. Accurately weighed, 50 mg of Mycophenolate sodium will be dissolved in 10 ml of phosphate buffered saline solution (PBS, pH 7.4). The drug solution will be then added to the flask containing the film and rotated at 70 rpm for another 1 h at 37° C. to peel off the surfactant/cholesterol film. The hydration of film will lead to the formation of vesicles. The vesicles will be then sonicated for 10 min in a Probe sonicator and kept at 25° C.

Following ratios of span 60 and cholesterol can be used for the preparation of niosomal formulation. Final selection of optimised vesicles for loading in contact lenses can be done on the basis of Particle size distribution and loading efficiency of all the formulations.

TABLE 129

| | | | | Mycophe- | |
|---|---|---|---|---|---|
| | | | | nolate | |
| | | | | mofetil or | Phosphate |
| | | Span | Choles- | Mycophe- | buffered |
| Formulation | Ratio of Span | 60 | terol | nolate | saline pH |
| trials | 60:Cholesterol | (mg) | (mg) | sodium | 7.4 |
| Formulation-1 | 4:6 | 17.22 | 23.19 | 50 mg | 10 ml |
| Formulation-2 | 5:5 | 21.53 | 19.33 | 50 mg | 10 ml |
| Formulation-3 | 6:4 | 25.83 | 15.46 | 50 mg | 10 ml |
| Formulation-4 | 7:3 | 30.14 | 11.59 | 50 mg | 10 ml |
| Formulation-5 | 8:2 | 34.44 | 7.73 | 50 mg | 10 ml |

Mycophenolate Mofetil/Mycophenolate Sodium Loaded Niosomes Infusion into Contact Lenses

Lenses will be first removed from their packaging solution with the help of clean tweezers and then soaked in 30 mL of millipore water for 10 min. They will be then put in ethanol for 10 min. Each lens will be then soaked in 5 mL of prepared niosomes formulation for a period of 48 h. After the loading stage, the lenses will be blotted to remove any excess drug solution remaining on the surface with the help of clean filter paper, and will be subsequently used stored in PBS 7.4 solution.

Mycophenolate Mofetil/Mycophenolate Sodium Loaded Niosomes Infusion into Vit E Loaded Contact Lenses

Vit E loaded lenses will be first removed from PBS solution and the same above procedure will be followed, only difference will be that lenses will be directly dipped in niosomes formulation instead of first soaking in ethanol.

### Example 102

Preparation of Ocular Insert

Ocular inserts of mycophenolate mofetil or sodium can be prepared by using the solvent casting method. A single or combination of polymers (Chitosan, Sodium alginate, polyvinyl alcohol) can be used in combination with glycerin (plasticizer) in order to provide the uniform strength to the insert.

The required amount of polymer (Chitosan or polyvinyl alcohol) and mycophenolate sodium will be added to the aqueous solution of sodium alginate along with constant stirring. The plasticizer will be added thereafter and the drug polymer solutions will be stirred for 12 h and allowed to stand overnight to remove any entrapped air bubbles. The pH range of the solutions will be around 5 to 8. The solutions will be then poured into glass rings placed over mercury in the glass Petri dishes. Solvent will be allowed to evaporate by placing the Petri dishes in oven (40±2° C.). Dried films will be carefully removed from the Petri dish and then cut into oval shaped inserts with the help of a sharp-edged die (13.2 mm in length and 5.4 mm in width).

TABLE 130

| | Polyvinyl | Sodium | | | Mycophe- |
|---|---|---|---|---|---|
| Formulation | alcohol | alginate | Glycerine | Chitosan | nolate |
| trials | (%) | (%) | (%) | (%) | sodium |
| Formulation-1 | — | 4 | 10 | 4 | 100 mg |
| Formulation-2 | — | 2 | 10 | 4 | 100 mg |
| Formulation-3 | — | 4 | 10 | 2 | 100 mg |
| Formulation-4 | 4 | 2 | 10 | — | 100 mg |
| Formulation-5 | 2 | 4 | 10 | — | 100 mg |
| Formulation-6 | 4 | 4 | 10 | — | 100 mg |

### Example 103

Evaluation of Eye Irritation/Corrosion Potential In Vivo Using Exemplary Mycophenolate Formulations

This study was conducted to evaluate the eye irritation/corrosion potential of mycophenolate mofetil topical ointment 1.0% w/w and 2.0% w/w, mycophenolate sodium topical suspension 1.0% w/v and 2.0% w/v and mycophenolate sodium topical solution 1.0% w/v in New Zealand White rabbits. The objective of this study was to assess the possible irritation/corrosion potential when a single dose of test item/s are instilled in the conjunctival sac of the rabbit's eye. This study can provide a rational basis for risk assessment in human. It provides information on health hazards likely to arise from exposure to eyes and associated mucous membranes. The study was conducted in accordance with the OECD Guideline for Testing of Chemicals, Section 4, Health Effects, Number 405. “Acute Eye Irritation/Corrosion” adopted 9 Oct. 2017.

Mycophenolate Sodium Suspension:

| | | Composition in | |
|---|---|---|---|
| | | Percentage (%) | |
| | | VLN-F- | VLN-F- |
| | | 70/ASR/060 | 70/ASR/061 |
| | | 1% w/v | 2% w/v |
| INGREDIENT | Function | Suspension | Suspension |
| Mycophenolate sodium | API | 1.0 | 2.0 |
| equivalent to | | | |
| mycophenolic acid | | | |
| Polysorbate 80 | Surfactant | 0.250 | 0.250 |
| Carbopol 974P | Thickening | 0.300 | 0.300 |
| | agent | | |
| Glycerol | Tonicity | 0.586 | 0.400 |
| | modifier | | |
| Di-sodium EDTA | Chelating | 0.100 | 0.100 |
| | agent | | |
| Boric acid | Preservative | 1.000 | 1.000 |
| Purified water | | Q.S. upto | Q.S. upto |
| | | 100 ml | 100 ml |

Mycophenolate Mofetil Ointment:

| | | Composition in | |
|---|---|---|---|
| | | Percentage (%) | |
| | | VLN-F- | VLN-F- |
| | | 70/ASR/063 | 70/ASR/064 |
| INGREDIENT | Function | 1% w/w Ointment | 2% w/w Ointment |
| Mycophenolate | API | 1.0 | 2.0 |
| Mofetil | | | |
| Light liquid | Ointment base | 27.3 | 26.3 |
| paraffin | | | |
| White soft | Ointment base | 63.7 | 63.7 |
| paraffin | | | |
| Lanolin | Ointment base | 3.0 | 3.0 |
| Lanolin | Ointment base | 5.0 | 5.0 |
| Alcohol | | | |

Mycophenolate Sodium Solution:

| | | Composition in |
|---|---|---|
| | | Percentage (%) |
| | | VLN-F-70/ASR/065 |
| INGREDIENT | Function | 1% w/v Solution |
| Mycophenolate Sodium | API | 1.0 |
| equivalent to | | |
| Mycophenolic acid | | |
| Tween 80 | Surfactant | 4.0 |
| Boric acid | Preservative | 1.0 |
| Di-sodium EDTA | Chelating agent | 0.1 |
| Sodium chloride | Tonicity modifier | 0.23 |
| HPMC K4M | Thickening agent | 0.5 |
| Purified water | | Q.S. upto 100 ml |

Method:
1. Both eyes of each of the New Zealand White rabbits were examined one day prior to instillation of formulations with an ophthalmoscope (Welch Allyn).
2. On test day, approximately one hour prior to instillation of the test item, a systemic analgesic agent (buprenorphine hydrochloride injection I.P.-0.3 mg/mL) was administered by subcutaneous injection, and approximately 5 minutes prior to instillation of the test item, 2 drops of topical anesthetic agent (proparacaine HCl 0.5%) was used in both the test and control eye.
3. Treatment was done by instilling 0.1 g/0.1 mL of the ready to use test item into the conjunctival sac of the left eye of the respective treatment groups. The lids were held together gently for about one second to prevent loss of test item. The right eye of each animal was treated similarly with respective vehicle formulations to serve as the vehicle control. All the rabbits were treated in a similar manner.
4. After 24 h contact period, the treated eye irrigated with normal saline for one minute to remove the residual test item. The score at 24 h indicated that the test item was not severe irritant to the eye.
5. Clinical signs of toxicity and pre-terminal deaths were observed four times (at hourly intervals) on the day of test item instillation and once daily on days 2 to 5 post-instillation. The eyes of each rabbit were examined at 1, 24, 48 and 72 h, post-instillation and the reactions were recorded.
6. The eyes of each rabbit were examined using an opthalmoscope and scored. In addition, all the treated eyes were examined using ophthalmic fluorescein sodium at 24 h post-instillation for all the rabbits. The grades of ocular lesions were assessed according to scoring pattern provided in Table 131.
7. At the termination of the observation period, all the rabbits were euthanised with an overdose of thiopentone sodium injected intravenously and necropsied by an experienced prosector for gross pathological changes.

TABLE 131
Grading of ocular lesions

| Area of the eye | Parameters | Grade |
|---|---|---|
| Cornea (The | No ulceration or opacity | 0 |
| area of corneal | Scattered or diffuse areas of opacity | 1 |
| opacity) | (other than slight dulling of normal | |
| | lusture); details of iris clearly visible | |
| | Easily discernible translucent areas; | 2 |
| | details of iris slightly obscured | |
| | Nacrous area; no details of iris visible; | 3 |
| | size of pupil barely discernible | |
| | Opaque cornea; iris not discernible | 4 |
| | through the opacity | |
| Iris | Normal | 0 |
| | Markedly deepened rugae, congestion, | 1 |
| | swelling, moderate circumcorneal | |
| | hyperaemia or injection; iris reactive | |
| | to light (a sluggish) reaction is | |
| | considered to be an effect | |
| | Hemorrhage, gross destruction, or no | 2 |
| | reaction to light | |
| Conjunctivae | Normal | 0 |
| Redness (refers | Some blood vessels hyperaemic (injected) | 1 |
| to palpebral and | Diffuse, crimson colour; individual | 2 |
| bulbar | vessels not easily discernible | |
| conjunctivae, | Diffuse beefy red | 3 |
| excluding | | |
| cornea and iris) | | |
| Chemosis | Normal | 0 |
| (Swelling of | Some swelling above normal | 1 |
| lids/nictating | Obvious swelling, with partial | 2 |
| membranes) | eversion of lids | |
| | Swelling, with lids about half closed | 3 |
| | Swelling, with lids more than half closed | 4 |

Results:

There were no clinical signs of toxicity and pre-terminal deaths and no abnormality detected at necropsy in any of the animals. There were no eye reactions observed in any of the tested formulations and vehicle formulations. All the formulations of mycophenolate tested were non-irritant to eyes of New Zealand white rabbits. The individual body weights, clinical signs of toxicity and necropsy findings were noted. The corneal, iris and conjunctival reactions (individual eye reaction scores) and total scores were collected, and mean eye irritation scores are presented in Table 132.

TABLE 132
Mean eye irritation scores (n = 9)

Hours

| Eye reaction | 1 | 24 | 48 | 72 |
|---|---|---|---|---|
| Corneal opacity | 0 | 0 | 0 | 0 |
| Area of Opacity | NA | NA | NA | NA |
| Iris | 0 | 0 | 0 | 0 |
| Redness | 0 | 0 | 0 | 0 |
| Chemosis | 0 | 0 | 0 | 0 |

NA: Not applicable

### Example 104

### Efficacy of Exemplary Mycophenolate Formulations In Vivo

The efficacy of mycophenolate ophthalmic test formulations was evaluated in bovine serum albumin (BSA) induced uveitis in rabbit eye model.

Mycophenolate Mofetil Ointment:

| | | Composition in | |
|---|---|---|---|
| | | Percentage (%) | |
| | | VLN-F- | VLN-F- |
| | | 70/ASR/118A | 70/ASR/118B |
| INGREDIENT | Function | 1% w/w Ointment | 2% w/w Ointment |
| Mycophenolate | API | 1.0 | 2.0 |
| Mofetil | | | |
| Light liquid | Ointment base | 27.3 | 26.3 |
| paraffin | | | |
| White soft | Ointment base | 63.7 | 63.7 |
| paraffin | | | |
| Lanolin | Ointment base | 3.0 | 3.0 |
| Lanolin | Ointment base | 5.0 | 5.0 |
| Alcohol | | | |

Mycophenolate Sodium Suspension:

| | | 2% w/v | 1% w/v |
|---|---|---|---|
| | | Mycophenolate | Mycophenolate |
| | | sodium | sodium |
| | | suspension | suspension |
| | | (VLN-F70/ | (VLN-F70/ |
| | | ASR/120) | ASR/121) |
| | | % w/v | % w/v |
| Mycophenolate | API | 2.00 | 1.00 |
| Sodium | | | |
| equivalent to | | | |
| Mycophenolic | | | |
| acid | | | |
| Tween 80 | Surfactant | 0.25 | 0.25 |
| Boric acid | Preservative | 1.00 | 1.00 |
| Disodium | Chelating | 0.10 | 0.10 |
| EDTA | agent | | |
| Glycerol | Tonicity | 0.40 | 0.59 |
| | modifier | | |
| Carbopol | Thickening | 0.45 | 0.30 |
| 974P | agent | | |
| Purified | | Q.S. upto 100 ml | Q.S. upto 100 ml |
| water | | | |
| pH | | 6.87 | 6.51 |
| Soluble | | 15.55 | 12.21 |
| fraction | | | |

Methods:
1. Male New Zealand white rabbits (>2 kg) were used for the study. Rabbit eye was anesthetized with topical anesthetic agent (2% lidocaine gel) before sensitization.
2. All animals except normal control animals were sensitized with BSA in complete Freund's adjuvant emulsion at 1 mg/animal, subcutaneously at 3-4 sites on the back of rabbit on days 1, 3, and 5. Further on day 9, all animals except normal control animals were challenged with intravitreal eye injection of 50 μg BSA in 100 μL of sterile water.
3. The rabbits were observed for any abnormal inflammation for a period up to 3 h post intravitreal injection. On day 9, ˜3 h post intravitreal injection, animals were randomized after confirming uveitis in one eye (in contralateral eye, no convincing signs of uveitis were clinically demonstrated) with approximately equal grading score into 4 different groups as per study plan.
4. 100 μL of vehicle formulation, test formulation and reference formulation were instilled twice daily with an interval of 6 h between two doses by topical route into one eye (which developed uveitis) of the animals of respective treatment groups from day 9 to day 13.
5. The rabbits were monitored and scored for the induction of flare and accumulation of inflammatory cells, causing opacity of cornea (Table 133, FIG. 3).
6. The scoring of eye under slit lamp was recorded at 0 h (Before intravitreal injection), 3 h, 24 h, 48 h, 72 h and 96 h post intravitreal injection of BSA that is on Days 9, 10, 11, 12 and 13. On Day 13, animals were treated with respective treatment group and were sacrificed post ˜6 h of last dose.
7. The following parameters were monitored during the study period: uveitis scoring using slit lamp; representative eye photographs from each treatment group; white blood cell and differential leukocyte count in aqueous humor at terminal (n=3/group at the end of study period) and histopathology of eye (n=3/group at the end of study period).
8. Animals were euthanized with excessive anesthesia at the end of study. Uveitis developed eye was removed using a surgical blade from each treatment group, aqueous humor was collected from eyes by making a small incision on the eyeball and draining the aqueous humor into a prelabelled microfuge tubes.

TABLE 133
Uveitis scoring and grading system

| Area of uveitis | Score | Grading |
|---|---|---|
| Zero | 0 | Absent |
| One quarter (or less) but not zero | 1 | Mild |
| Greater than one quarter, but less than half | 2 | Moderate |
| Greater than half, but less than three quarters | 3 | Severe |
| Greater than three quarters, up to whole area | 4 | Very Severe |

Results:
1. Significant increase in uveitis clinical score was observed in BSA induced groups compared to normal control within first 3 h after intravitreal injection, which indicated development of phenotype in these groups. Representative pictures of rabbit eye showing effect of formulations on BSA induced uveitis is illustrated in FIG. 3.
2. The inflammatory reaction significantly increased so that severe clinical signs of uveitis were observed in 24 h post-intravitreal injection and were sustained till 96 h post-intravitreal injection. Vehicle group exhibited significant increase in uveitis compared to normal control. Rabbits treated with mycophenolate mofetil ointment 2% w/w group showed significant reduction in the uveitis score compared to vehicle control group on day 10 and day 11 of treatment period. Mycophenolate sodium suspension 2% w/v group showed significant reduction in the uveitis score compared to vehicle control group on day 10, 11 and 12 of treatment period.
3. Vehicle group exhibited increase in WBC count compare to normal control. Treatment with mycophenolate formulations 2% w/w, showed reduction in the WBC count compared to vehicle control group on day 13 of treatment period.
4. Histopathology analysis of eyes showed BSA induced uveitis animals exhibited increased leukocyte infiltration grading compared to normal saline treated animals. The infiltration comprised mononuclear cells and macrophages. There were no additional changes observed in cornea and sclera junction, ciliary body and/or iris. Further, all other sub-anatomical areas of eyes were unaffected (normal).

TABLE 134
Uveitis clinical score in BSA induced rabbit uveitis model
after treatment with mycophenolate topical formulations

| | Area of Uveitis (flare)_Mean ± SEM | | | | |
|---|---|---|---|---|---|
| Day | NC | IC | Pred (1%) | MMF (2%) | MSS (2%) |
| 9 | 0 ± 0 | 2.0 ± 0.3 | 1.8 ± 0.3 | 2.0 ± 0.3 | 2.0 ± 0.4 |
| 10 | 0 ± 0 | 3.2 ± 0.2 | 1.8 ± 0.3 | 2.2 ± 0.2 | 2.0 ± 0.3 |
| 11 | 0 ± 0 | 3.0 ± 0.3 | 1.7 ± 0.2 | 2.0 ± 0.3 | 1.8 ± 0.2 |
| 12 | 0 ± 0 | 3.0 ± 0.3 | 1.3 ± 0.3 | 2.0 ± 0.3 | 1.8 ± 0.3 |
| 13 | 0 ± 0 | 2.8 ± 0.3 | 1.5 ± 0.2 | 2.0 ± 0.3 | 1.8 ± 0.3 |

(NC: normal control; IC: induction control; Pred: prednisolone (reference standard), MMF mycophenolate mofetil ointment; MSS: mycophenolate sodium suspension)

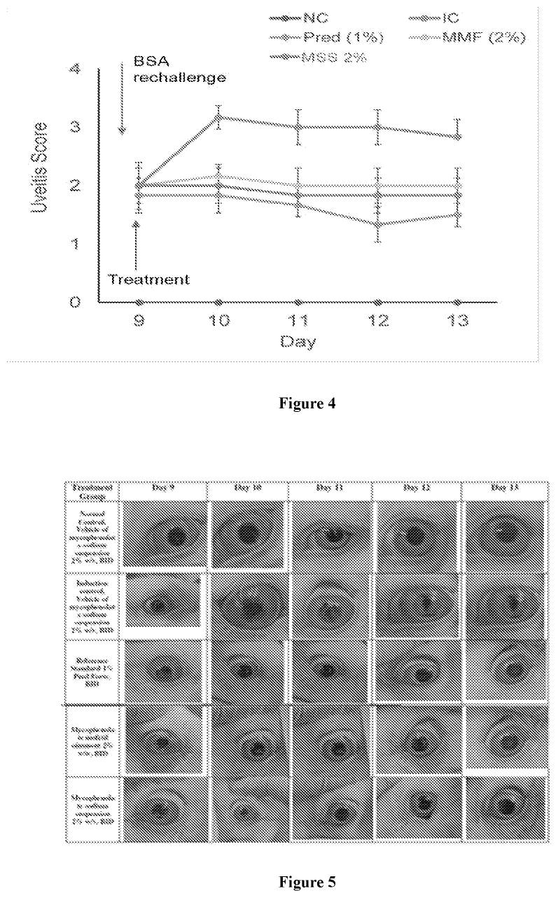

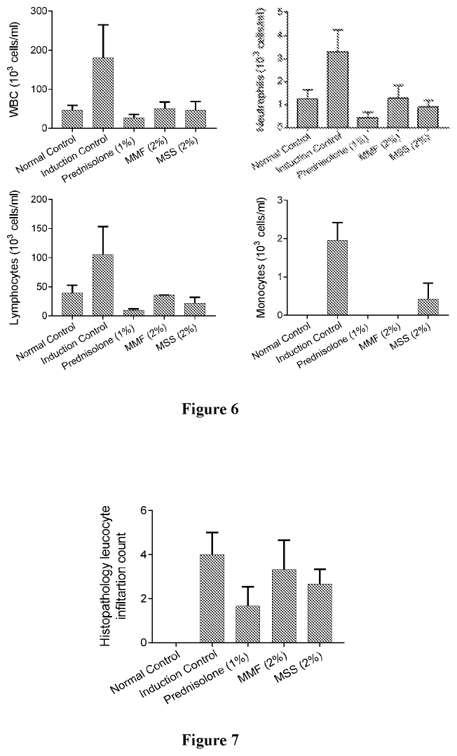

Based on the results (Table 134; FIGS. 4-7), it can be concluded that administration of both mycophenolate formulations (mycophenolate mofetil ointment and mycophenolate sodium suspension) showed improvement in experimental BSA induced uveitis in rabbit model.

### INCORPORATION BY REFERENCE

All references, articles, publications, patents, patent publications, and patent applications (if any) cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

Further, all patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A formulation comprising:
mycophenolic acid (MPA) or a pharmaceutically acceptable salt or derivative thereof; and
at least one component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, suspending agent, and therapeutic agent,
wherein the formulation is an ophthalmic formulation.

2. The ophthalmic formulation as claimed in claim 1, wherein:
the pharmaceutically acceptable derivative is an ester or an analog of mycophenolic acid; or
the pharmaceutically acceptable derivative is mycophenolate mofetil (MMF) or an analog thereof.

3. (canceled)

4. The ophthalmic formulation as claimed in claim 1, wherein:
the pharmaceutically acceptable salt is selected from the group consisting of mycophenolate sodium (MPS), mycophenolate mofetil hydrochloride (MMF.HCl), mycophenolic acid di-tris(hydroxymethyl) aminomethane (Tris) salt, mycophenolic acid meglumine salt, trolamine mycophenolate or mycophenolic acid trolamine salt, triethylamine mycophenolate or mycophenolic acid triethylamine salt, hyaluronic acid salt of mycophenolate mofetil, and combinations thereof; or
the pharmaceutically acceptable salt is mycophenolate sodium (MPS) or mycophenolate mofetil hydrochloride (MMF.HCl).

5. (canceled)

6. The ophthalmic formulation as claimed in claim 1, wherein the mycophenolic acid (MPA) or a pharmaceutically acceptable salt or derivative thereof has a particle size of about 10 nm to 100 μm, preferably about or less than 25 μm.

7. The ophthalmic formulation as claimed in claim 1, wherein:
said formulation is:
an ointment formulation, or an ointment formulation comprising mycophenolate mofetil hydrochloride (MMF.HCl); or
a suspension formulation, or a suspension formulation having a pH of 4-7; or
a suspension formulation comprising mycophenolate sodium (MPS); or
an injectable formulation; or
a topical formulation having a pH ranging between 4 to 8, and
said formulation is stable for more than 3 months.

8.-14. (canceled)

15. The ophthalmic formulation as claimed in claim 1, wherein the pharmaceutically acceptable derivative is selected from the group consisting of:
compound 1 having the structure: 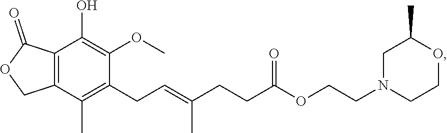 [SMILES: COc1c(O)c2c(c(C)c1C/C=C(\C)CCC(=O)OCCN1CCO[C@H](C)C1)COC2=O]
compound 2 having the structure: 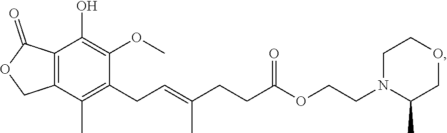 [SMILES: COc1c(O)c2c(c(C)c1C/C=C(\C)CCC(=O)OCCN1CCOC[C@H]1C)COC2=O]
compound 3 having the structure: 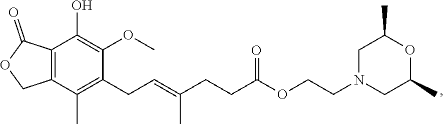 [SMILES: COc1c(O)c2c(c(C)c1C/C=C(\C)CCC(=O)OCCN1C[C@@H](C)O[C@@H](C)C1)COC2=O]
compound 4 having the structure: 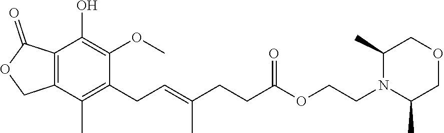 [SMILES: COc1c(O)c2c(c(C)c1C/C=C(\C)CCC(=O)OCCN1[C@H](C)COC[C@@H]1C)COC2=O]
compound 5 having the structure: 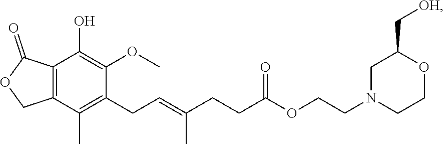 [SMILES: COc1c(O)c2c(c(C)c1C/C=C(\C)CCC(=O)OCCN1CCO[C@H](CO)C1)COC2=O]
compound 6 having the structure: 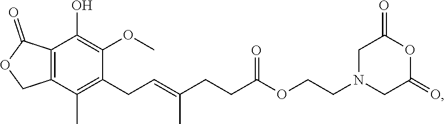 [SMILES: COc1c(O)c2c(c(C)c1C/C=C(\C)CCC(=O)OCCN1CC(=O)OC(=O)C1)COC2=O]
compound 7 having the structure: 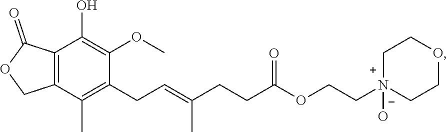 [SMILES: COc1c(O)c2c(c(C)c1C/C=C(\C)CCC(=O)OCC[N+]1([O-])CCOCC1)COC2=O]
compound 8 having the structure: 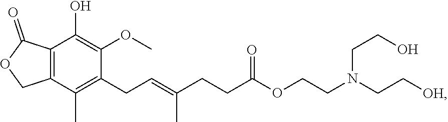 [SMILES: COc1c(O)c2c(c(C)c1C/C=C(\C)CCC(=O)OCCN(CCO)CCO)COC2=O]
and
compound 9 having the structure:

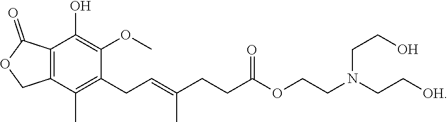 [SMILES: COc1c(O)c2c(c(C)c1C/C=C(\C)CCC(=O)OCCN(CCO)CCO)COC2=O]

16. The ophthalmic formulation as claimed in claim 1, wherein:
the preservative is selected from the group consisting of boric acid, benzalkonium chloride, benzethonium chloride, benzododecinium bromide, cetylpyridinium chloride, chlorobutanol, thimerosol, phenylmercuric nitrate, phenylmercuric acetate, methylparaben, propylparaben, butylparaben, phenylethyl alcohol, sodium benzoate, sodium propionate, sorbic acid, sodium sorbate, sodium borate, sodium perborate and combinations thereof; or
the formulation does not comprise any preservative; or
the chelating agent is selected from the group consisting of ethylenediamine tetracetic acid (EDTA), edetate disodium (disodium EDTA salt), edetate sodium (EDTA tetrasodium salt), sodium EDTA and combinations thereof; or
the buffering agent or the pH modifier is selected from the group consisting of acetic acid, boric acid, anhydrous citric acid, citric acid monohydrate, hydrochloric acid, phosphoric acid, potassium phosphate monobasic, sodium acetate, sodium acetate anhydrous, sodium carbonate, sodium carbonate monohydrate, sodium hydroxide, sodium phosphate, sodium phosphate (heptahydrate), sodium phosphate dibasic, sodium phosphate dibasic (anhydrous), sodium phosphate dibasic (dihydrate), sodium phosphate dibasic (dodecahydrate), sodium phosphate monobasic, sodium phosphate monobasic (anhydrous), sodium phosphate monobasic (dihydrate), sodium phosphate monobasic (monohydrate), sulfuric acid, trisodium citrate dihydrate, tromethamine and combinations thereof; or
the thickening agent, the viscosity modifier or the viscosity enhancer is selected from the group consisting of acrylic acid polymer (carbopol), dextran 40 (molecular weight of 40,000 Daltons), dextran 70 (molecular weight of 70,000 Daltons), gelatin, glycerin, carboxymethylcellulose (CMC), hydroxyethyl cellulose (HEC), hydroxypropyl methylcellulose (HPMC), methyl cellulose, ethyl cellulose, polyethylene glycol (PEG), poloxamer 407, polysorbate 80, propylene glycol, polyvinyl alcohol (PVA), polyvinylpyrrolidone (povidone), povidone K30, povidone K90, carbomer 940, carbomer copolymer Type A (allyl pentaerythritol crosslinked), carbomer copolymer Type B (allyl pentaerythritol crosslinked), carbomer homopolymer Type B (allyl pentaerythritol crosslinked), carbomer homopolymer Type B (allyl sucrose crosslinked), a crosslinked copolymer of acrylic acid and a hydrophobic C10-30 alkyl acrylate co-monomer, carboxymethyl cellulose sodium, crospovidone, dextran, guar gum, hydroxypropyl cellulose (HPC), hydroxymethyl cellulose (HMC), hydroxymethyl cellulose (2000 mPa·S at 1%), hydroxymethyl cellulose (4000 mPa·S at 1%), hypromellose, hypromellose 2906 (4000 mPa·S), hypromellose 2910 (15000 mPa·S), hypromellose 2910 (3 mPa·S), hypromellose 2910 (5 mPa·S), polycarbophil, xanthan gum, sodium hyaluronate, and combinations thereof; or
the antioxidant is selected from the group consisting of alpha-tocopherol, EDTA, sulfate, sodium bisulfite, sodium metabisulfite, sodium sulfate (anhydrous), sodium thiosulfate, sodium thiosulfite, thimerosal, thiourea, and combinations thereof; or
the tonicity modifier is selected from the group consisting of dextrose, glycerin, potassium chloride, propylene glycol, sodium chloride, calcium chloride, magnesium chloride, mannitol, and combinations thereof; or
the surfactant is selected from the group consisting of non-ionic surfactant, amphoteric surfactant, anionic surfactant, cationic surfactant and combinations thereof; or
the surfactant is selected from the group consisting of polyoxyethylene (POE)-polyoxypropylene (POP) block copolymer, poloxamer 407, poloxamer 235, poloxamer 188, ethylenediamine POE-POP block copolymer adduct, poloxamine, POE sorbitan fatty acid ester, polysorbate 20, polysorbate 60, polysorbate 65, polysorbate 80, POE hydrogenated castor oil, POE (5) hydrogenated castor oil, POE (10) hardened castor oil, POE (20) hardened castor oil, POE (40) hardened castor oil, POE (50) hardened castor oil, POE (60) hardened castor oil, POE (100) hardened castor oil, POE castor oil, POE (3) castor oil, POE (10) castor oil, POE (35) castor oil, POE (40) castor oil, polyoxyl 40 hydrogenated castor oil, POE alkyl ether, polyoxyethylene (9) lauryl ether, polyoxyethylene (20), polyoxypropylene (4), cetyl ether, POE alkylphenyl ether, POE (10) nonylphenyl ether, polyoxyl stearate, polyoxyl 40 stearate, polyoxydiethyl castor oil, polyoxyl-35 castor oil, cremophor, glycine type amphoteric surfactant, alkyldiaminoethylglycine, alkylpolyaminoethylglycine, betaine type amphoteric surfactant, lauryldimethylaminoacetic acid betaine, imidazolinium betaine, quaternary ammonium salt, alkyl tertiary ammonium salt, benzalkonium chloride, benzethonium chloride, polydronium chloride, biguanide compound, polyhexamethylene biguanide hydrochloride, sodium alkylbenzene sulfonate, tyloxapol, poloxomer 407, tween 20, and combinations thereof; or
the humectant is selected from the group consisting of glycerin, hyaluronic acid, sorbitol, urea, alpha hydroxy acid, sugar, lactic acid, polyethylene glycol (PEG), PEG-4, PEG-8, propylene glycol, glyceryl triacetate, lithium chloride, polyol, sorbitol, xylitol, maltitol, polydextrose, quillaia, hexadecyl, myristyl, isodecyl and isopropyl esters of adipic, lactic, oleic, stearic, isostearic, myristic and linoleic acids, sodium isostearoyl-2-lactylate, sodium capryl lactylate, hydrolyzed protein, collagen-derived protein, aloe vera gel, acetamide monoethanolamide (MEA), sodium pyrrolidone carboxylic acid, L-proline, guanidine, pyrrolidone, acetamido propyl trimmonium chloride, calcium stearoyl lactylate, chitosan pyrrolidone carboxylic acid (PCA), diglycerol lactate, ethyl ester of hydrolyzed silk, fatty quaternary amine chloride complex, glycereth-7, glycereth-12, glycereth-26, glycereth-4.5 lactate, diglycerin, polyglycerin, hydrolyzed fibronectin, lactamide MEA, lactamide N-(2-hydroxyetheryl), mannitol, methyl gluceth-10, methyl gluceth-20, panthenol, pyrrolidone carboxylic acid (PCA), methylsilanol PCA, polyamino sugar condensate, quaternium-22, sea salt, sodium caproyl lactylate, sodium hyaluronate, sodium isostearoyl lactylate, sodium lactate, sodiumlauroyl lactylate, sodium PCA, sodium polyglutamate, sodium stearoyl lactylate, soluble collagen, sorbitan laurate, sorbitan oleate, sorbitansesquiisostearate, sorbitan stearate, sphingolipids, TEA-PCA (5-oxo-DL-proline, compound with 2,2′,2″-nitrilotriethanol), ethylene glycol, diethylene glycol, triethylene glycol, dipropylene glycol, 1,3-butylene glycol, 1,4-butylene glycol, glycerol, diglycerol, polyglycerol, glycerol ethylene oxide (EO) and propylene oxide (PO) adduct, sugar alcohol EO and PO adduct, adduct of EO or PO and monosaccharide such as galactose and fructose, adduct of EO or PO and polysaccharide such as maltose and lactose, sodium pyrrolidone carboxylate, polyoxyethylene methyl glycoside, hexylene glycol, maltose, D-mannitol, gluten, glucose, fructose, lactose, sodium chondroitin sulfate, sodium adenosine phosphate, gallates, pyrrolidone carbonates, glucosamine, cyclodextrin, alpha hydroxy acids, 2-methyl-1,3-propane diol, and combinations thereof; or
the solvent or co-solvent is selected from the group consisting of water, alcohol, glycerin, propylene glycol, propylene glycol diacetate, polypropylene glycol, sorbitol and combinations thereof; or
the emulsifier or the co-emulsifier is selected from the group consisting of silicone-based emulsifier, a polyethylene glycol emulsifier, a polysiloxane emulsifier, a glycoside emulsifier, an acrylic-based emulsifier, and combinations thereof; or
the emulsifier or the co-emulsifier is selected from the group consisting of polysorbate, carbomer, castor oil, ethyl alcohol, isopropyl alcohol, ethyl carbonate, ethyl acetate, benzyl alcohol, benzyl benzoate, propylene glycol, polyethylene glycols, 1,3-butyleneglycol, dimethylformamide, sodium lauryl sulfate, sodium docusate, cholesterol, cholesterol esters, taurocholic acid, phosphatidylcholine, oils, cottonseed oil, groundnut oil, corn germ oil, olive oil, castor oil, sesame oil, glycerol, tetrahydrofurfuryl alcohol, fatty acid esters of sorbitan, cetyl alcohol, glyceryl monostearate, nonoxynol-9, octoxynol-40, poloxamer 188, poloxamer 407, polyethylene glycol 400, polyethylene glycol 8000, polyoxyl 35 castor oil, polyoxyl 40 hydrogenated castor oil, polyoxyl 15 hydroxystearate, polyoxyl 40 stearate, polysorbate 20, polysorbate 80, tyloxapol, TEA/K stearate (triethanolamine/potassium stearate), sodium lauryl stearate, sodium cetearyl sulfate, beeswax/borax, glycerol di-stearate, PEG (polyethyleneglycol)-100 stearate, polysorbate 20, steareth 2, steareth 20, distearyldimethylammonium chloride, benzalkonium chloride, steapyrium chloride, acrylates/C 10-30 alkyl acrylate crosspolymer, polyacrylamide, polyquaternium-37, dicaprylate/dicaparate, PPG-1 Trideceth-6, alkyl modified dimethiconecopolyols, polyglyceryl esters, ethoxylated di-fatty esters, ionic polysorbate surfactant, nonylphenol polyethylene glycol ethers, alkylphenol-hydroxypolyoxyethylene, Poly(oxy-1,2-ethanediyl), alpha-(4-nonylphenol)-omega-hydroxy-, branched, nonylphenol polyethylene glycol ether mixtures, phenoxypolyethoxyethanols or polymers thereof, sodium dodecyl sulfate, and combinations thereof.

17.-28. (canceled)

29. The ophthalmic formulation as claimed in claim 1, wherein:
the ointment base is selected from the group consisting of light liquid paraffin, white soft paraffin, petrolatum, lanolin, lanolin alcohol, chlorobutanol, methylparaben, propylparaben, and combinations thereof; or
said petrolatum has a concentration of 5% to 80% of microcrystalline wax based on the total composition of petrolatum; or
said petrolatum has a concentration of 20% to 60% of microcrystalline wax based on the total composition of petrolatum.

30.-31. (canceled)

32. The ophthalmic formulation as claimed in claim 1, wherein:
the targeting agent is selected from the group consisting of didodecyldimethylammonium bromide, stearylamine, N-[1-(2,3-dioleoyloxy)propyl]-N,N,N-trimethylammonium chloride, and combinations thereof; or
the polymer is selected from the group consisting of poly (lactide), poly (lactideglycolide), poly (glycolide), poly (caprolactone), poly (amides), poly (anhydrides), poly (amino acids), poly (esters), poly (cyanoacrylates), poly (phosphazines), poly (phosphoesters), poly (esteramides), poly (dioxanones), poly (acetals), poly (cetals), poly (carbonates), poly (orthocarbonates), degradable poly (urethanes), chitins, chitosans, poly (hydroxybutyrates), poly (hydroxyvalerates), poly (maleic acid), poly (alkylene oxalates), poly (alkylene succinates), poly (hydroxybutyrates-co-hydroxyvalerates), copolymers, terpolymers or oxidised cellulose thereof, poly (e-caprolactone) (PCL), methacrylate acid copolymer, methacrylate esters, acrylic esters, poly (alkyl methacrylate), poly (methyl methacrylate), sodium alginate, chitosan and combinations thereof; or
the wetting agent is selected from the group consisting of hydrophilic polymers, polysorbate 20, polysorbate 80, poloxamer 282, tyloxapol, cellulose based polymers, HPMC, CMC, polyvinylpyrrolidone (PVP), polyvinyl alcohol (PVA), and combinations thereof; or
the lubricating agent is selected from the group consisting of non-phospholipid based agent, phospholipid based agent, petrolatum, mineral oil, propylene glycol, ethylene glycol, polyethylene glycol, hydroxypropylmethylcellulose (HPMC), carboxymethylcellulose (CMC), hydroxypropylcellulose, dextrans, dextran 70, water soluble proteins, gelatin, vinyl polymers, polyvinyl alcohol (PVA), polyvinylpyrrolidone (PVP), povidone, carbomers, carbomer 934P, carbomer 941, carbomer 940, carbomer 974P, vitamin E, and combinations thereof; or
the suspending agent is selected from the group consisting of pH independent polymers, pH dependent polymers and combinations thereof; or
the therapeutic agent is selected from the group consisting of an antibacterial agent, an anti-fungal agent, an anti-viral agent, anti-acanthamoebal agent, an anti-inflammatory agent, an immunosuppressive agent, an anti-glaucoma agent, an anti-VEGF agent, a growth factor, and combinations thereof, and wherein said antibacterial agent is selected from the group consisting of penicillins, cephalosporins, penems, carbapenems, monobactams, aminoglycosides, sulfonamides, macrolides, tetracyclines, lincosamides, quinolones, chloramphenicol, vancomycin, metronidazole, rifampin, isoniazid, spectinomycin, trimethoprim sulfamethoxazole, chitosan, ansamycins, daptomycin, nitrofurans, oxazolidinones, bacitracin, colistin, polymixin B, clindamycin, and combinations thereof; the anti-fungal agent is selected from the group consisting of amphotericin B, natamycin, candicin, filipin, hamycin, nystatin, rimocidin, voriconazole, imidazoles, triazoles, thiazoles, allylamines, echinocandins, benzoic acid, ciclopirox, flucytosine, griseofulvin, haloprogin, tolnaftate, undecylenic acid, povidone-iodine and combinations thereof; the anti-viral agent is selected from the group consisting of acyclovir, valacyclovir, famciclovir, penciclovir, trifluridine, vidarabine, and combinations thereof; the anti-acanthamoebal agent is selected from the group consisting of chlorohexidine, polyhexamethylen biguanide, propamidine, hexamidine, and combinations thereof; the anti-inflammatory agent is selected from the group consisting of corticosteroids, salicylates, propionic acid derivatives, acetic acid derivatives, enolic acid derivatives, anthranilic acid derivatives, selective cox-2 inhibitors, sulfonanilides, antibodies, tumor necrosis factor-alpha inhibitors, dominant negative ligands, interleukin-1 receptor antagonists, and combinations thereof; the immunosuppressive agent is selected from the group consisting of alkylating agents, antimetabolites, mycophenolate, cyclosporine, tacrolimus, rapamycin, and combinations thereof; the anti-glaucoma agent is selected from the group consisting of prostaglandin analogs, beta blockers, adrenergic agonists, carbonic anhydrase inhibitors, parasympathomimetic (miotic) agents, and combinations thereof; the anti-vascular endothelial growth factor (anti-VEGF) agent is selected from the group consisting of bevacizumab, ranibizumab, aflibercept, and combinations thereof; the growth factor is selected from the group consisting of epidermal growth factor (EGF), platelet-derived growth factor (PDGF), vitamin A, vitamin E, fibronectin, annexin a5, albumin, alpha-2 macroglobulin, fibroblast growth factor b, insulin-like growth factor-I, nerve growth factor, hepatocyte growth factor, and combinations thereof.

33.-38. (canceled)

39. The ophthalmic formulation as claimed in claim 1, wherein:
the mycophenolic acid (MPA) or a pharmaceutically acceptable salt or derivative thereof is present in an amount up to 5% weight/volume (w/v) or weight/weight (w/w) of the formulation; or
the mycophenolic acid (MPA) or a pharmaceutically acceptable salt or derivative thereof is present in an amount of about 0.005% to 5% w/v or w/w, or about 0.005% to 4% w/v or w/w, or about 0.5% to 4% w/v or w/w of the formulation.

40.-42. (canceled)

43. The ophthalmic formulation as claimed in claim 1, wherein:
each of the at least one component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, suspending agent and therapeutic agent is present in an amount up to 99.99% w/v or w/w of the formulation.

44. The ophthalmic formulation as claimed in claim 1, wherein:
the preservative is present in an amount of about 0.005% to 10% w/v or w/w of the formulation,
the chelating agent is present in an amount of about 0.005% to 5% w/v or w/w of the formulation,
the buffering agent is present in an amount of about 0.005% to 5% w/v or w/w of the formulation,
the pH modifier is present in an amount of about 0.005% to 5% w/v or w/w of the formulation,
the thickening agent is present in an amount of about 0.005% to 5% w/v or w/w of the formulation,
the viscosity enhancer or viscosity modifier is present in an amount of about 0.005% to 5% w/v or w/w of the formulation,
the antioxidant is present in an amount of about 0.005% to 5% w/v or w/w of the formulation,
the tonicity modifier is present in an amount of about 0.005% to 5% w/v or w/w of the formulation,
the surfactant is present in an amount of about 0.005% to 5% w/v or w/w of the formulation,
the humectant is present in an amount of about 0.005% to 5% w/v or w/w of the formulation,
the solvent or co-solvent is present in an amount of about 0.005% to 20% w/v or w/w of the formulation,
the emulsifier is present in an amount of about 0.005% to 10% w/v or w/w of the formulation,
the co-emulsifier is present in an amount of about 0.005% to 10% w/v or w/w of the formulation,
the ointment base is present in an amount of about 0.005% to 99.99% w/v or w/w of the formulation,
the targeting agent is present in an amount of about 0.005% to 10% w/v or w/w of the formulation,
the polymer is present in an amount of about 0.005% to 5% w/v or w/w of the formulation,
the wetting agent is present in an amount of about 0.005% to 10% w/v or w/w of the formulation,
the lubricating agent is present in an amount of about 0.005% to 30% w/v or w/w of the formulation,
the suspending agent is present in an amount of about 0.005% to 5% w/v or w/w of the formulation, and
the therapeutic agent is present in an amount of about 0.005% to 5% w/v or w/w of the formulation.

45. The ophthalmic formulation as claimed in claim 1, wherein:
the formulation is a non-aqueous formulation or an aqueous formulation; and wherein the non-aqueous formulation comprises water in an amount of less than 50% by weight of the formulation, and the aqueous formulation comprises water in an amount of more than 50% by weight of the formulation; or
the formulation is in a form of liquid, fluid, emulsion, gel, semi-solid or solid; or
the formulation is in a form of solution, suspension, ointment, emulsion, ocular injection, nanoparticulate system, nano suspension, eye or intraocular implant, ocular insert, pellet, gel, colloidal system, or hydrogel; or
the formulation is in a form of self-emulsifying drug delivery system or an in situ gel-forming system; or
the formulation is an ointment having a viscosity of about 7000 to 20000 mPa and particle size ranging from about 1 μm to 10 μm; or
the formulation is a suspension having a viscosity less than 2000 mPa and particle size ranging less than 10 μm.

46.-50. (canceled)

51. The ophthalmic formulation as claimed in claim 1, wherein the formulation is:
an ointment formulation selected from:
a) mycophenolate mofetil, light liquid paraffin, white soft paraffin, lanolin, and lanolin alcohol;
b) mycophenolate sodium, light liquid paraffin, white soft paraffin, lanolin, and lanolin alcohol; or
c) mycophenolate mofetil and white soft paraffin,
an emulsion formulation selected from:
a) mycophenolate mofetil, light liquid paraffin, polysorbate 80, polyoxyl 35 castor oil, and water;
b) mycophenolate mofetil, light liquid paraffin, polysorbate 80, polyoxyl 35 castor oil, hydroxyethyl cellulose, and water;
c) mycophenolate mofetil, polysorbate 80, lanolin alcohol, castor oil, crosslinked copolymer of acrylic acid and a hydrophobic C10-30 alkyl acrylate co-monomer, and water;
d) mycophenolate mofetil, polyoxyl 40 stearate, lanolin alcohol, castor oil, crosslinked copolymer of acrylic acid and a hydrophobic C10-30 alkyl acrylate co-monomer, and water;
e) mycophenolate mofetil, polysorbate 80, lanolin, lanolin alcohol, castor oil, crosslinked copolymer of acrylic acid and a hydrophobic C10-30 alkyl acrylate co-monomer, and water;
f) mycophenolate mofetil, polysorbate 80, lanolin alcohol, castor oil, light liquid paraffin, crosslinked copolymer of acrylic acid and a hydrophobic C10-30 alkyl acrylate co-monomer, and water;
g) mycophenolate mofetil, polyoxyl 40 stearate, lanolin alcohol, cetyl alcohol, glyceryl monostearate, castor oil, crosslinked copolymer of acrylic acid and a hydrophobic C10-30 alkyl acrylate co-monomer, and water; or
h) mycophenolate mofetil, polyoxyl 40 stearate, lanolin alcohol, cetyl alcohol, glyceryl monostearate, castor oil, ethanol, crosslinked copolymer of acrylic acid and a hydrophobic C10-30 alkyl acrylate co-monomer, and water,
a solution formulation selected from:
a) mycophenolate sodium, tween 80, boric acid, di-sodium EDTA, sodium chloride, hydroxypropyl methylcellulose, and water;
b) mycophenolate sodium, polysorbate 80, and water;
c) mycophenolate sodium, polyoxyl-35 castor oil, and water;
d) mycophenolate sodium, tween 20, and water;
e) mycophenolate sodium, polyoxyl 40 hydrogenated castor oil, and water;
f) mycophenolate sodium, poloxomer 407, and water;
g) mycophenolate sodium, polysorbate 80, polyvinyl alcohol, and water;
h) mycophenolate sodium, polysorbate 80, polyvinylpyrrolidone, and water;
i) mycophenolate sodium, polysorbate 80, acrylic acid polymer, and water;
j) mycophenolate sodium, polysorbate 80, hydroxyethyl cellulose, and water;
k) mycophenolate sodium, polysorbate 80, hydroxypropyl methylcellulose, and water;
l) mycophenolate sodium, polyoxyl-35 castor oil, and water;
m) mycophenolate sodium, polyoxyl-35 castor oil, acrylic acid polymer, and water;
n) mycophenolate sodium, polyoxyl-35 castor oil, hydroxyethyl cellulose, and water;
o) mycophenolate sodium, polyoxyl-35 castor oil, hydroxypropyl methylcellulose, and water;
p) mycophenolate sodium, polyoxyl-35 castor oil, polyvinyl alcohol, and water;
q) mycophenolate sodium, polyoxyl-35 castor oil, polyvinylpyrrolidone, and water;
r) mycophenolate sodium, polysorbate 80, polyoxyl-35 castor oil, and water;
s) mycophenolate sodium, polysorbate 80, polyoxyl-35 castor oil, polyvinyl alcohol, and water;
t) mycophenolate sodium, polysorbate 80, polyoxyl-35 castor oil, polyvinylpyrrolidone, and water;
u) mycophenolate sodium, polyoxyl 40 hydrogenated castor oil, polyoxyl-35 castor oil, polyvinyl alcohol, and water;
v) mycophenolate sodium, polyoxyl 40 hydrogenated castor oil, polyoxyl-35 castor oil, polyvinylpyrrolidone, and water;
w) mycophenolate sodium, polyoxyl 40 hydrogenated castor oil, polyoxyl-35 castor oil, hydroxypropyl methylcellulose, and water;
x) mycophenolate sodium, polysorbate 80, polyoxyl 40 hydrogenated castor oil, polyvinyl alcohol, and water;
y) mycophenolate sodium, polysorbate 80, polyoxyl 40 hydrogenated castor oil polyvinylpyrrolidone, and water;
z) mycophenolate sodium, polysorbate 80, polyoxyl 40 hydrogenated castor oil hydroxypropyl methylcellulose, and water;
aa) mycophenolate sodium, polysorbate 80, polyoxyl-35 castor oil, and water;
bb) mycophenolate sodium, polysorbate 80, polyoxyl-35 castor oil, acrylic acid polymer, and water;
cc) mycophenolate sodium, polysorbate 80, polyoxyl-35 castor oil, hydroxyethyl cellulose, and water;
dd) mycophenolate sodium, polysorbate 80, polyoxyl-35 castor oil, hydroxypropyl methylcellulose, and water;
ee) mycophenolate sodium, polysorbate 80, boric acid, hydroxyethyl cellulose, and water; or
ff) mycophenolate sodium, polysorbate 80, boric acid, hydroxypropyl methylcellulose, and water,
a suspension formulation selected from:
a) mycophenolic acid, polysorbate 80, acrylic acid polymer, glycerol, di-sodium EDTA, boric acid, and water;
b) mycophenolic acid, polysorbate 80, acrylic acid polymer, glycerol, boric acid, and water;
c) mycophenolic acid, polysorbate 80, acrylic acid polymer, glycerol, boric acid, citric acid, and water;
d) mycophenolic acid, polycarbophil, glycerol, boric acid, ortho phosphoric acid, and water; or
e) mycophenolic acid, polysorbate 80, di-sodium EDTA, polycarbophil, glycerol, boric acid, citric acid, and water,
a nanosuspension formulation comprising:
mycophenolate sodium, glycerol, di-sodium EDTA, polysorbate 80, polycarbophil, boric acid, acrylate co-polymer, and water,
an ocular injection formulation selected from:
a) mycophenolate sodium, monobasic sodium phosphate monohydrate, dibasic sodium phosphate heptahydrate, sodium chloride, sucrose, polysorbate 20, sodium hydroxide, and water; or
b) mycophenolate mofetil hydrochloride, mannitol, polysorbate 20, dibasic sodium phosphate heptahydrate, sodium hydroxide, and water, or
an eye implant or ocular insert formulation selected from:
a) mycophenolate mofetil or mycophenolate sodium or a combination thereof, sorbitan stearate, cholesterol, phosphate buffered saline, and vitamin E;
b) mycophenolate mofetil or mycophenolate sodium or a combination thereof, sodium alginate, glycerine, polyvinyl alcohol and chitosan;
c) mycophenolate mofetil or mycophenolate sodium or a combination thereof, sodium alginate, glycerine, and chitosan; or
d) mycophenolate mofetil or mycophenolate sodium or a combination thereof, sodium alginate, glycerine, and polyvinyl alcohol.

52. A method for preparing the ophthalmic formulation defined in claim 1, said method comprising combining:
a) the mycophenolic acid (MPA) or a pharmaceutically acceptable salt or derivative thereof with
b) the one or more component selected from the group consisting of preservative, chelating agent, buffering agent, pH modifier, thickening agent, viscosity enhancer or viscosity modifier, antioxidant, tonicity modifier, surfactant, humectant, solvent or co-solvent, emulsifier, co-emulsifier, ointment base, targeting agent, polymer, wetting agent, lubricating agent, suspending agent, and therapeutic agent.

53. A pharmaceutical kit or package comprising the ophthalmic formulation defined in claim 1, and an instruction manual for application of the ophthalmic formulation thereof.

54. A method for treating an ocular disease, the method comprising administering the ophthalmic formulation defined in claim 1, to a subject in need thereof.

55. The method as claimed in claim 17, wherein:
the ocular disease is selected from the group consisting of uveitis, macular edema, angiographic cystoid macular edema, retinal ischemia, choroidal neovascularization, macular degeneration, retinal disease, diabetic retinopathy, diabetic retinal edema, retinal detachment, inflammatory disease, choroiditis, multifocal choroiditis, episcleritis, scleritis, birdshot retinochoroidopathy, vascular disease, retinal ischemia, retinal vasculitis, choroidal vascular insufficiency, choroidal thrombosis, neovascularization of the optic nerve, optic neuritis, blepharitis, keratitis, rubeosis iritis, Fuchs' heterochromic iridocyclitis, chronic uveitis, anterior uveitis, conjunctivitis, allergic conjunctivitis, keratoconjunctivitis sicca (dry eye syndrome), iridocyclitis, iritis, scleritis, episcleritis, corneal edema, scleral disease, ocular cicatricial pemphigoid, pars planitis, Posner Schlossman syndrome, Behcet's disease, Vogt-Koyanagi-Harada syndrome, hypersensitivity reactions, conjunctival edema, conjunctival venous congestion, periorbital cellulitis, acute dacryocystitis, non-specific vasculitis, sarcoidosis, and combinations thereof; or
the ocular disease is uveitis; or the uveitis is anterior, intermediate, posterior or panuveitis; or
the subject is a mammal including a human.

56.-61. (canceled)

62. The ophthalmic formulation as claimed in claim 1,
wherein said formulation is used as an ophthalmic insert, or ophthalmic implant, or use in keratoplasty or other ophthalmic surgery.

63.-64. (canceled)

65. A Use of mycophenolic acid (MPA) or a pharmaceutically acceptable salt or derivative thereof for the treatment of Lichen sclerosus.
